SEQUENCE LISTING (3 sequences)

SEQ ID NO: 1 - DNA, 20 bp, Artificial
  gRNA sequence that targets exon 3 of the SERPINB9 (human) gene
acctgctgagaacggccaac

SEQ ID NO: 2 - DNA, 20 bp, Artificial
  gRNA sequence that targets exon 3 of the SERPINB9 (human) gene
tcacttcagtgagaagcgac

SEQ ID NO: 3 - DNA, 20 bp, Artificial
  gRNA sequence that targets exon 6 of the serpinb9 (mouse) gene
gcacctccttcacataggcg

(19) United States
(12) Patent Application Publication
(10) Pub. No.: US 2023/0255906 A1
(43) Pub. Date: Aug. 17, 2023

(54) Targeting Serpin B9 in Cancer

(71) Applicant: The Brigham and Women`s Hospital, Inc., Boston, MA (US)

(72) Inventor: Reza Abdi, Newton, MA (US)

(21) Appl. No.: 18/013,751

(22) PCT Filed: Jul. 2, 2021
(86) PCT No.: PCT/US2021/040330

§ 371 Date: Dec. 29, 2022

Related U.S. Application Data
(60) Provisional application No. 63/047,599, filed on Jul. 2, 2020.

(51) Int. Cl. A61K 31/166; A61K 31/423; A61K 31/341; A61P 35/00

(52) U.S. Cl. CPC A61K 31/166; A61K 31/423; A61K 31/341; A61P 35/00

(57) ABSTRACT

Provided are compounds that are Sb9 inhibitors and analogs thereof and pharmaceutical compositions thereof that can be used in the treatment of diseases or disorders associated with Sb9 expression and/or activity. Thus, also provided are methods of treating diseases or disorders associated with Sb9 expression and/or activity.

### RELATED APPLICATIONS

This application claims the benefit of U.S. Provisional Application No. 63/047,599, filed Jul. 2, 2020, which is incorporated herein by reference in its entirety.

### TECHNICAL FIELD

The present disclosure relates to compounds and pharmaceutical compositions thereof that are Sb9 inhibitors and analogs thereof. The compounds and pharmaceutical compositions thereof are useful in the treatment of diseases or disorders associated with Sb9 expression and/or activity.

### SEQUENCE LISTING

The instant application contains a Sequence Listing which has been filed electronically in ASCII format and is hereby incorporated by reference in its entirety. Said ASCII copy, created on Jul. 1, 2021, is named 29618_0227WO1SEQ.txt and is 919 bytes in size.

### BACKGROUND

Tumor recognition of immune cells and their destruction as a concept has transformed the world of tumor therapies. The overall tumor cancer immunotherapy market is anticipated to reach ˜$119.39B by 2021. This is a 14% increase from 2016 with a market cap around $61.9B in 2016. There are a number of factors that have contributed to this astonishing increase, including the clinical efficacy of checkpoint inhibitors in slowing down growth of tumors and a steady increase in the incidence of cancer and lack of effective therapy in general for refractory cancers.

### SUMMARY

As shown herein, genetic ablation of Serpin B9 (Sb9) sensitized tumors to killing by not only CL-derived GrB but also unexpectedly from endogenously produced GrB together resulting in the control of cancer in mice. The role of Sb9 in the anti-tumor host response was examined in Sb9 KO mice, which exhibited increased immunity to tumors. This was a consequence of impaired survival of immunosuppressive TAMs, MDSCs, Tregs, and cancer-associated fibroblasts (CAFs) in the TME that resulted in increased activity of anti-tumor CL. Described herein are small molecule inhibitors of Sb9 and analogs thereof, treatment of mice with these compounds could control tumor growth by direct sensitization to GrB and the activation of protective immunity.

The present discovery of the dual mechanism of action of Sb9 inhibition on direct tumor killing and the de-repression of host anti-tumor immunity supports a therapeutic approach that can include using one modality to treat immunologically inert tumors (although the present methods can be used in combination with other therapies as well).

In some embodiments, the small molecule is a compound of Formula (I)

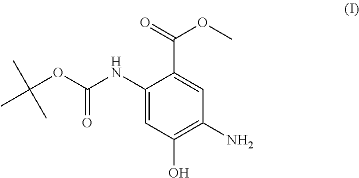 [SMILES: COC(=O)c1cc(N)c(O)cc1NC(=O)OC(C)(C)C]

or a pharmaceutically acceptable salt thereof. In some embodiments, the compound of Formula (I) is methyl 5-amino-2-((tert-butoxycarbonyl)amino)-4-hydroxybenzoate. In some embodiments, the compound of Formula (I) is a pharmaceutically acceptable salt of methyl 5-amino-2-((tert-butoxycarbonyl)amino)-4-hydroxybenzoate. Also provided in the present disclosure is a pharmaceutical composition containing a compound of Formula (I) and a pharmaceutically acceptable carrier.

In some embodiments, the small molecule is a compound of Formula (II)

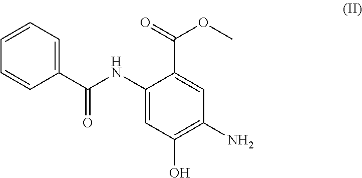 [SMILES: COC(=O)c1cc(N)c(O)cc1NC(=O)c1ccccc1]

or a pharmaceutically acceptable salt thereof. In some embodiments, the compound of Formula (II) is methyl 5-amino-2-benzamido-4-hydroxybenzoate. In some embodiments, the compound of Formula (II) is a pharmaceutically acceptable salt of methyl 5-amino-2-benzamido-4-hydroxybenzoate. Also provided in the present disclosure is a pharmaceutical composition containing a compound of Formula (II) and a pharmaceutically acceptable carrier.

Provided in the present disclosure is a method of treating a cancer in an individual in need thereof. In some embodiments, the method includes administering a therapeutically effective amount of a compound of Formula (I) or Formula (II) or a pharmaceutically acceptable salt thereof, or a pharmaceutical composition containing a compound of Formula (I) or Formula (II) and a pharmaceutically acceptable carrier. In some embodiments, the cancer is a solid tumor. In some embodiments, the cancer is selected from melanoma, colorectal, pancreatic, lung, non-small cell lung cancer, breast, kidney, thyroid, lymphoid, gastrointestinal, genitourinary tract cancer, Hodgkin lymphoma, colon, renal cell carcinoma, ovarian, prostate cancer and/or testicular tumors, small intestine, and esophagus cancer.

Provided in the present disclosure is a method of treating a cancer in a subject in need thereof, the method including administering a therapeutically effective amount of a compound of Formula (III)

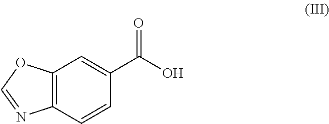 [SMILES: O=C(O)c1ccc2ncoc2c1]

or a pharmaceutically acceptable salt thereof.

Also provided in the present disclosure is a method of treating a cancer in a subject in need thereof, the method including administering a therapeutically effective amount of a compound of Formula (IV)

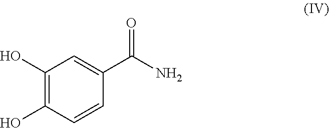 [SMILES: NC(=O)c1ccc(O)c(O)c1]

or a pharmaceutically acceptable salt thereof.

Provided in the present disclosure is a method of treating a cancer in a subject in need thereof, the method including administering a therapeutically effective amount of a compound of Formula (V)

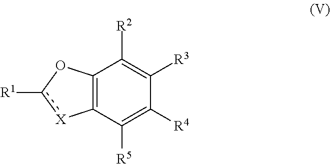

or a pharmaceutically acceptable salt thereof, wherein:

X is N or O; wherein when X is N, [symbol] is a double bond and when X is O, [symbol] is a single bond;

R¹ is H, —C₁-C₃ alkyl, —C₁-C₃ haloalkyl, —C₃-C₆ cycloalkyl, 4-6 membered heterocyclic ring, phenyl optionally substituted with 1-5 halogen, —SH, oxo (═O), or —N(C₁-C₃ alkyl)₂;

one of R², R³, R⁴, and R⁵ is —(CH₂)ₙC(═O)NR⁶R⁷ and the others are each independently H or OH;

R⁶ is H or —C₁-C₃ alkyl;

R⁷ is —C₂-C₄ alkyl, —(C₁-C₃ alkylene)-O—(C₁-C₃ alkyl), —(C₁-C₃ alkylene)-N(C₁-C₃ alkyl)₂, —C₁-C₃ alkylene-phenyl, —C₃-C₆ cycloalkyl, or —OH;

or R⁶ and R⁷, together with the nitrogen to which they are attached, form a 4-6 membered heterocyclic ring; and

n is 0 or 1.

In some embodiments, the compound is selected from:

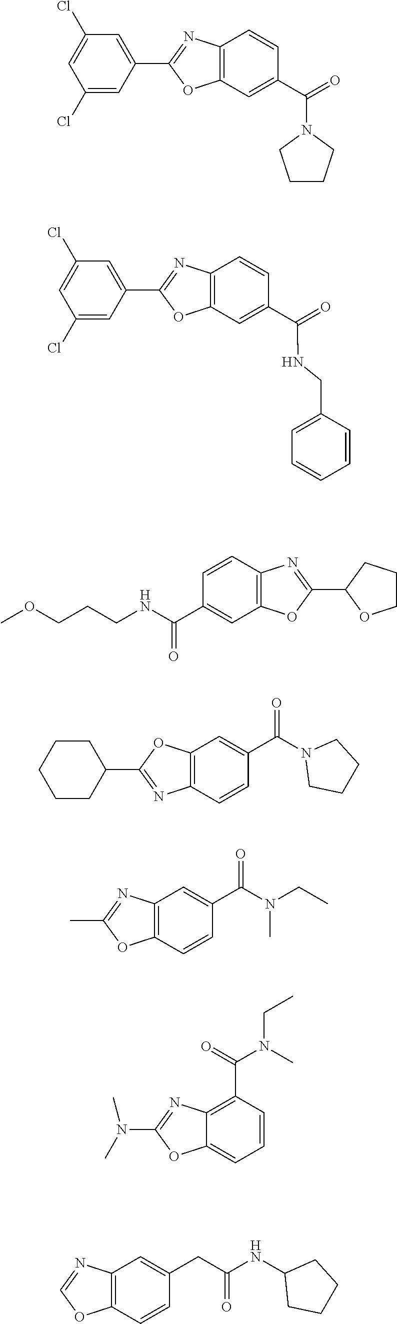

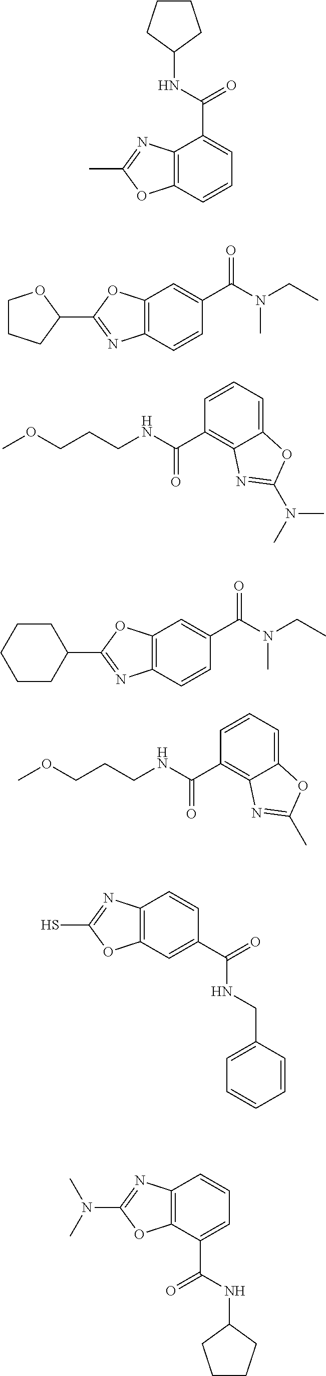

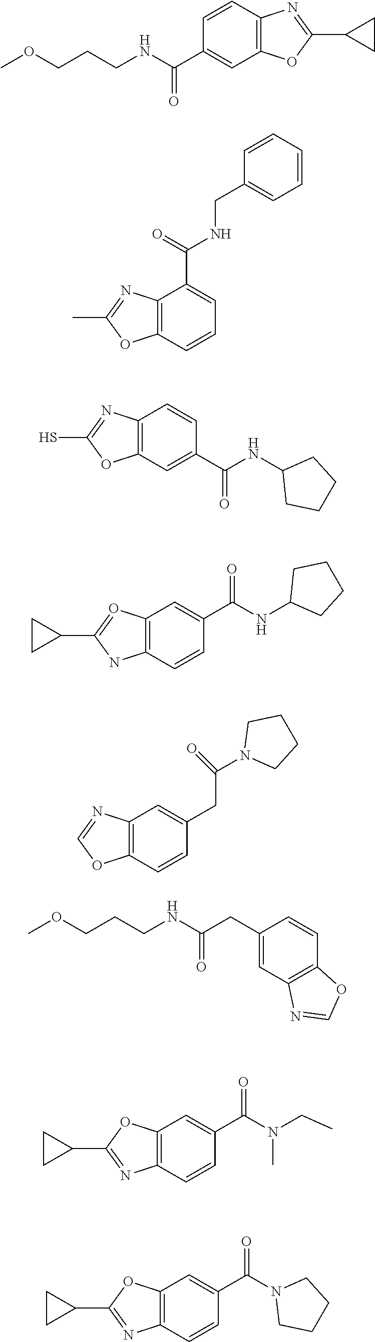

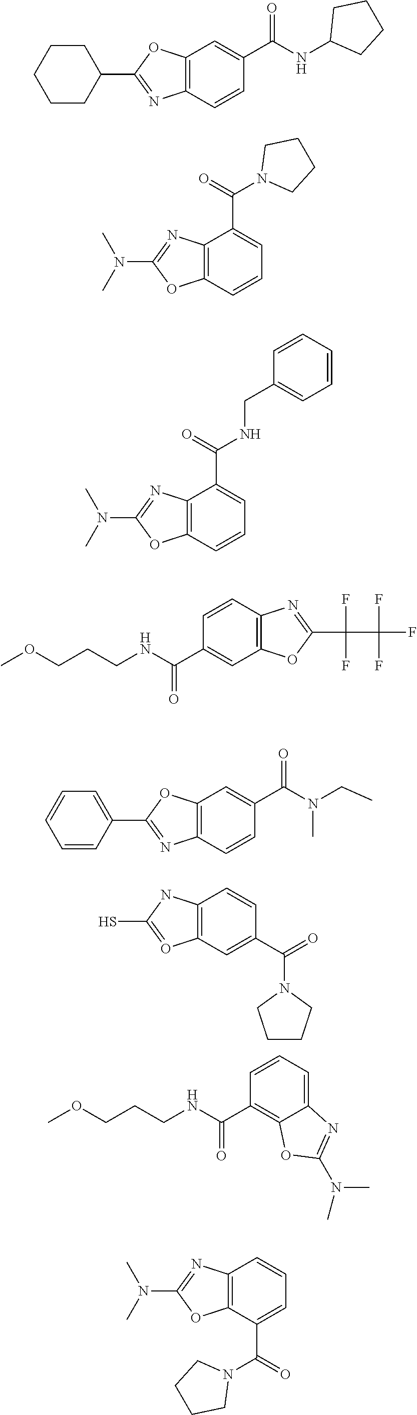

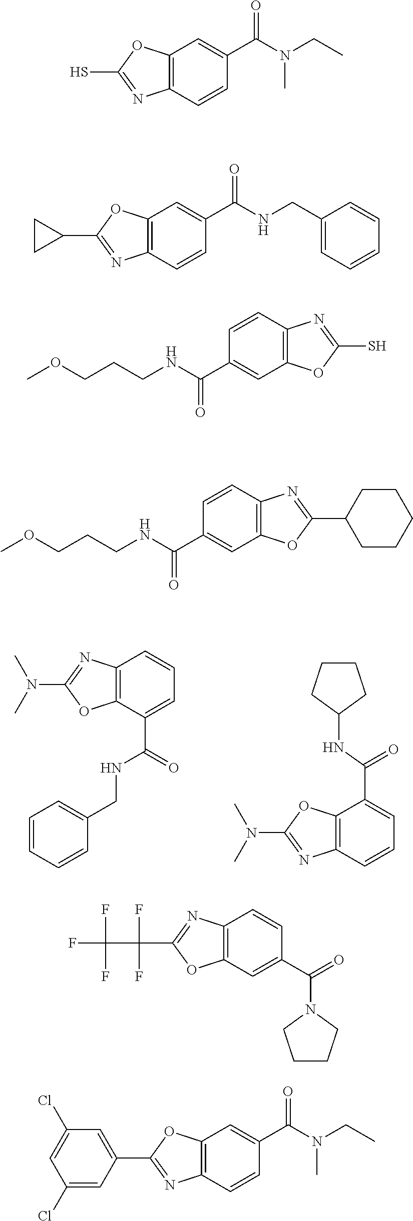

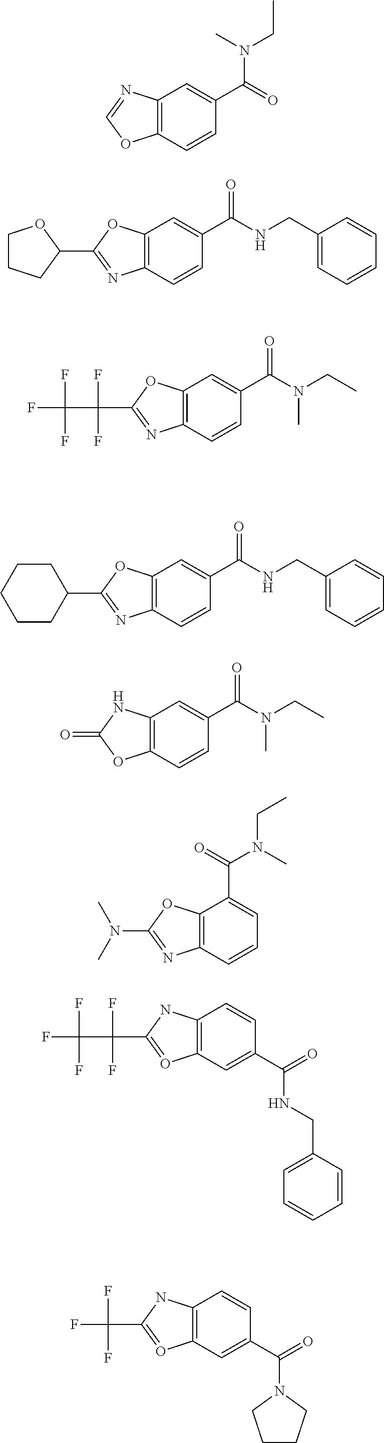

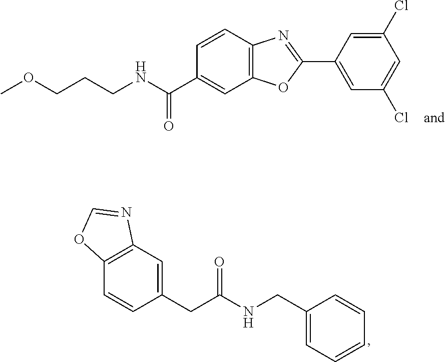 [SMILES: COCCCNC(=O)c1ccc2nc(-c3cc(Cl)cc(Cl)c3)oc2c1 ; O=C(Cc1ccc2ocnc2c1)NCc1ccccc1]

or a pharmaceutically acceptable salt thereof.

Also provided is a method of treating a cancer in a subject in need thereof, the method including administering a therapeutically effective amount of a compound of Formula (VI)

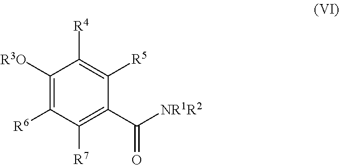

or a pharmaceutically acceptable salt thereof, wherein:

R¹ is H or —C₁-C₃ alkyl;

R² is —C₂-C₄ alkyl, —(C₁-C₃ alkylene)-O—(C₁-C₃ alkyl), —(C₁-C₃ alkylene)-N(C₁-C₃ alkyl)₂, —(C₁-C₃ alkylene)-(C₃-C₆ cycloalkyl), —(C₁-C₃ alkylene)-(4-6 membered heterocyclic ring), —C₁-C₃ alkylene-phenyl, or —C₃-C₆ cycloalkyl; or

or R¹ and R², taken together with the nitrogen to which they are attached, form a 4-6 membered heterocyclic ring;

R³ is H or —C₁-C₆ alkyl;

R⁴ is H, —OH, or —O—C₁-C₃ alkyl;

R⁵ is H, C₁-C₃ alkyl, or halogen;

R⁶ is H, —OH, or —O—C₁-C₃ alkyl; and

R⁷ is H, C₁-C₃ alkyl, or halogen.

In some embodiments, the compound is selected from:

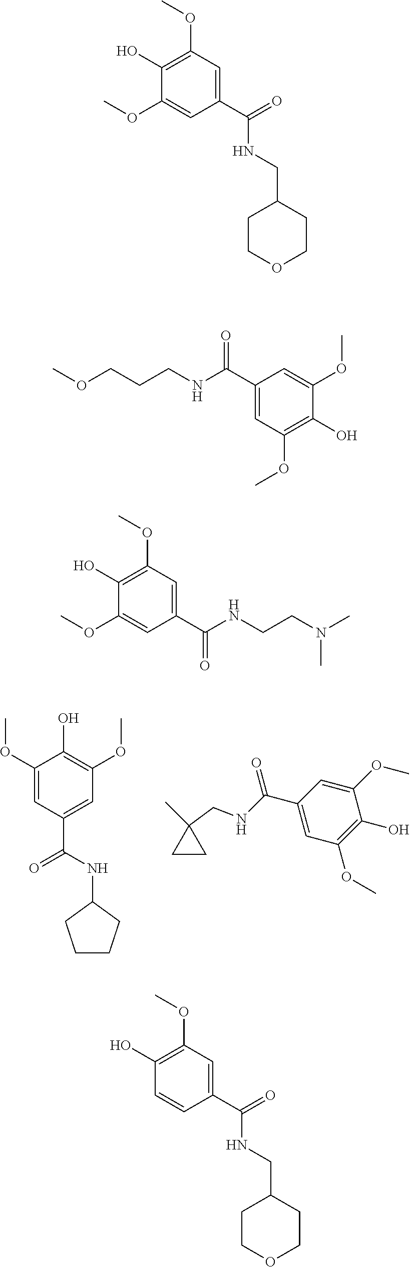

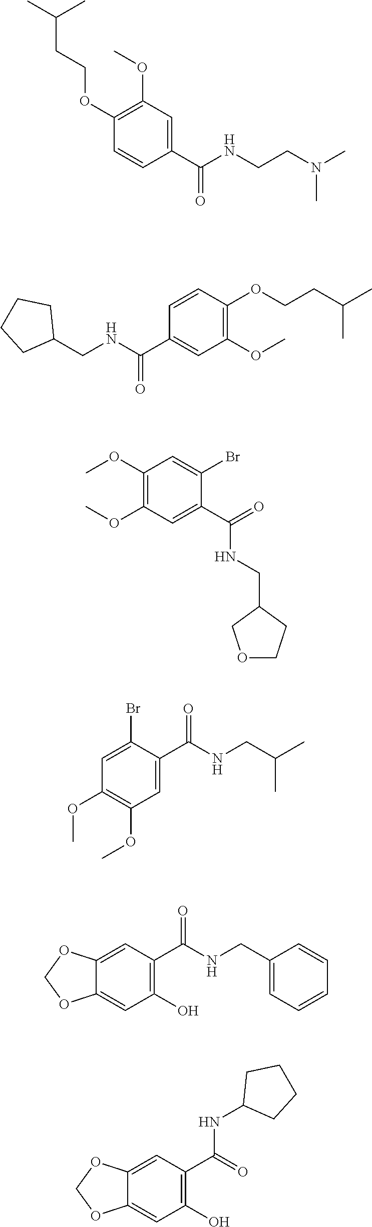

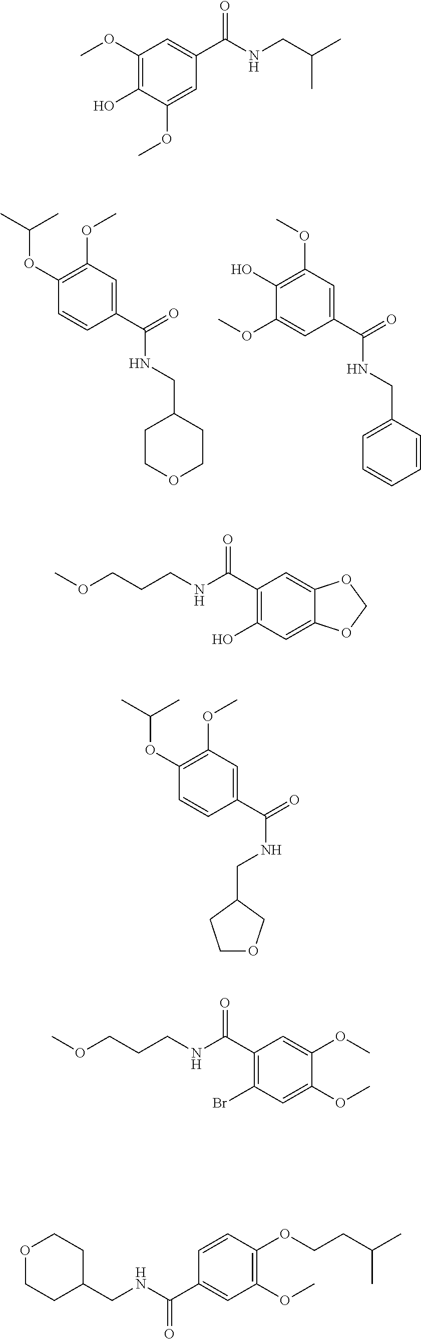

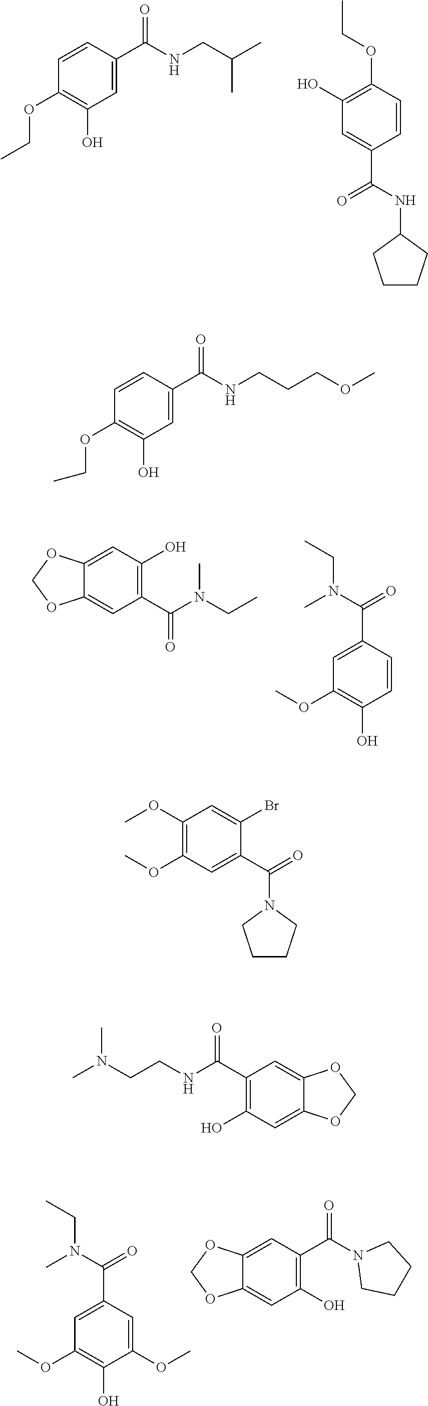

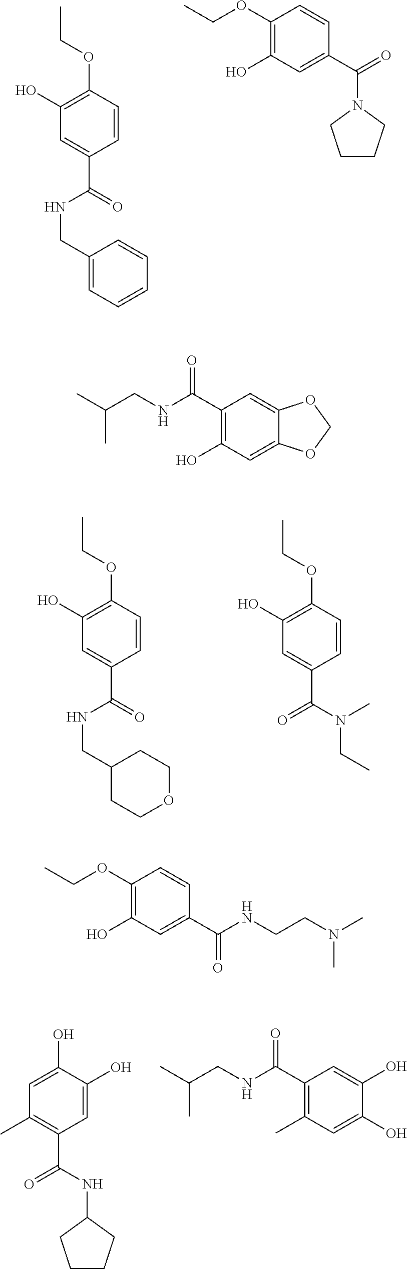

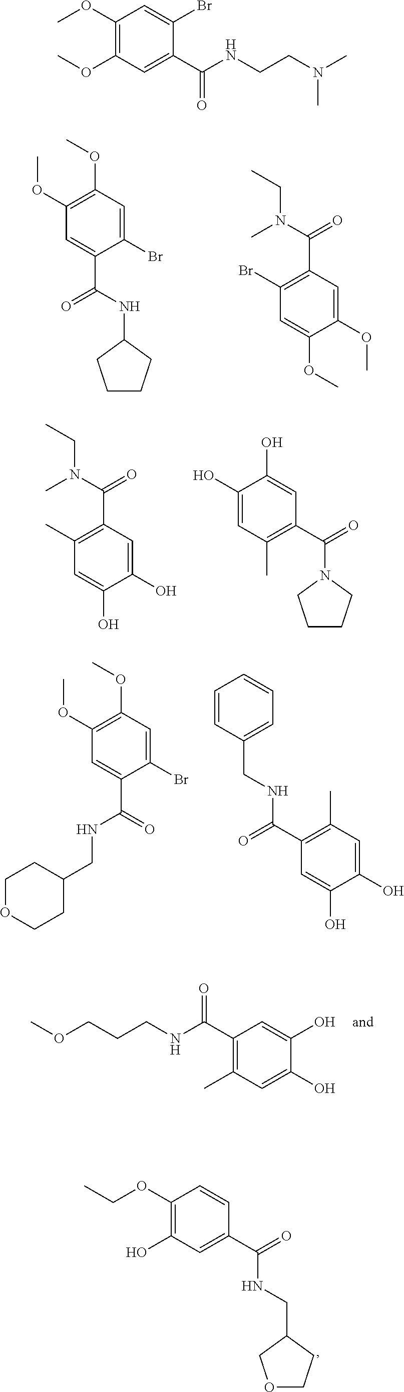

or a pharmaceutically acceptable salt thereof.

In some embodiments of the methods of the present disclosure, the cancer is selected from melanoma, colorectal, pancreatic, lung, non-small cell lung cancer, breast, kidney, thyroid, lymphoid, gastrointestinal, genitourinary tract cancer, Hodgkin lymphoma, colon, renal cell carcinoma, ovarian, prostate cancer and/or testicular tumors, small intestine, and esophagus cancer.

In some embodiments, the method further includes administering one or more additional treatment modalities. In some embodiments, the additional treatment modality is selected from chemotherapy and immunotherapy. In some embodiments, the method includes administering a checkpoint inhibitor. In some embodiments, the checkpoint inhibitor is selected from an anti-PD-1 antibody, an anti-CD40 antibody, a CTLA-4 antibody, an anti-Tim3 antibody, and an anti-Lag3 antibody. In some embodiments, the additional treatment modality is administered prior to, after, or concurrently with administration of the compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1K. Sb9 is required to protect melanoma tumors from GrB-induced apoptosis.

FIG. 1A. Fluorescence micrographs demonstrate the presence of Sb9 and GrB in primary human malignant melanoma, breast adenocarcinoma, lung adenocarcinoma, and colorectal carcinoma. DAPI was used to counterstain the cell nuclei. Scale bar: 20 μm.

FIG. 1B. Western blotting shows three forms of Sb9 protein expression in B16 melanoma cells, indicated by arrows: Sb9-GrB complex (com), unbound Sb9 (mono), and complex degradation products or cleaved Sb9 (deg).

FIG. 1C. Western blotting shows three forms of GrB protein expression in B16 melanoma cells, indicated by arrows: Sb9-GrB complex (com), unbound GrB (mono), and complex degradation products (deg).

FIG. 1D. GrB RNAscope results demonstrate the expression of GrB by both B16 and 4T1 cells. DAPI was used to counterstain the cell nuclei. Scale bar: 10 μm.

FIG. 1E. Fluorescence micrographs show co-expression of Sb9 with melanoma stem cell markers CD271 (left image) and Nestin (right image) in B16 cells.

FIG. 1F. CRIPSR/Cas9 gene editing system was used to disrupt the serpinb9 gene in the B16 melanoma cells. The 20-nucleotide sequences upstream of the protospacer adjacent motif (PAM) sequences in exon 6 of the serpinb9 gene were targeted.

FIG. 1G. Western blotting confirmed the successful knockout of Sb9 protein in the B16 melanoma cells. Sb9-GrB complex (com), unbound Sb9 (mono), and complex degradation products or cleaved Sb9 (deg) are indicated by arrows. GAPDH was used as a loading control for protein normalization.

FIG. 1H. Flow cytometric analysis demonstrates that IL2 (70 ng/mL) versus PBS treatment for 48 h results in significantly higher GrB expression by B16-WT and B16-Sb9 KO cells. Data are expressed as means±SD from three independent experiments. ***P<0.001 (Student's t-test).

FIG. 1I. Representative fluorescence micrographs show that treatment of B16-Sb9 KO cells with IL2 (70 ng/mL) for 48 h results in higher expression of cleaved caspase-3 in comparison to B16-WT cells. Cells treated with PBS alone were used as a negative control. DAPI was used to counterstain the cell nuclei. Scale bar: 20 μm.

FIGS. 1J-1K. Representative flow cytometry plots (FIG. 1J) and quantitative analysis (FIG. 1K) reveal that treatment of B16-Sb9 KO cells with IL2 (70 ng/mL) for 48 h results in a significantly higher percentage of apoptotic cells, as indicated by expressions of Annexin-V and viability dye, in comparison to B16-WT cells, and addition of the GrB inhibitor (368050) (10 μM) suppresses apoptosis in both. Cells treated with PBS alone were used as a negative control. Data are expressed as means±SD from three independent experiments. *P<0.05, ***P<0.001 (Student's t-test).

FIGS. 2A-2P. Sb9 acts cell intrinsically to control tumor growth in vivo.

FIG. 2A. Tumor growth curve through 27 days following implantation in C57BL/6 mice indicates significantly slower growth rate of B16-Sb9 KO (n=10) than B16-WT (n=10) mouse melanoma tumors. Data are expressed as means±SEM. *P<0.05, **P<0.01, ***P<0.001 (Student's t-test).

FIGS. 2B-2C. Representative fluorescence micrographs of MelanA⁺ melanoma cells demonstrates significantly smaller MelanA⁺ area in the B16-Sb9 KO tumors than the B16-WT tumors at 17 days post-implantation, as demonstrated by semi-quantitative analysis (FIG. 2C). Data are expressed as means±SEM. ***P<0.001 (Student's t-test). Scale bar: 1000 μm.

FIG. 2D. Semi-quantitative analysis of fluorescence micrographs indicates a significantly higher density of cleaved caspase-3 (C-CAS-3)⁺ apoptotic cells in the B16-Sb9 KO tumors than the B16-WT tumors at 17 days post-implantation. Data are expressed as means±SEM. ***P<0.001 (Student's t-test).

FIGS. 2E-2F. Representative flow cytometry plots (FIG. 2E) and quantitative analysis (FIG. 2F) reveal a significantly higher percentage of apoptotic cells in the B16-Sb9 KO tumors, as indicated by expressions of Annexin-V and viability dye, in comparison to the B16-WT tumors at 17 days post-implantation. Data are expressed as means±SEM. ***P<0.001 (Student's t-test).

FIGS. 2G-2I. Flow cytometric analysis demonstrates that the percentages of tumor-infiltrating CD4⁺ CD25⁺Foxp3⁺ Treg populations (FIG. 2G), CD45⁺ CD11b⁺F4/80⁺Ly6C⁻Ly6G⁻ TAM populations (FIG. 2H), and CD45⁺ CD11b⁺Gr1⁺CD3⁻ MDSC populations (FIG. 2I) are similar between the B16-WT and B16-Sb9 KO melanoma tumors at 17 days post-implantation. Data are expressed as means±SEM. NS (no significant difference) (Student's t-test).

FIGS. 2J-2K. Comparison between tumor-draining lymph nodes (TDLNs) demonstrates significantly lower percentage of TDLN with metastatic lesions in mice implanted with B16-Sb9 KO (n=10) tumors than B16-WT (n=10) tumors. Representative photographs of TDLN from the B16-WT group and the B16-Sb9 KO group are provided (K). Data are expressed as means±SD from two independent experiments. ***P<0.001 (Student's t-test).

FIG. 2L. Representative fluorescence micrographs demonstrate presence of MelanA in TDLN from the B16-WT group and absence in TDLN from the B16-Sb9 KO group.

FIG. 2M. Comparison between TDLNs demonstrates significantly smaller size of TDLNs in the B16-Sb9 KO group than that in the B16-WT group. Data are expressed as means±SD from two independent experiments. ***P<0.001 (Student's t-test).

FIG. 2N. Fluorescence micrographs demonstrate the presence of Sb9 and MelanA in metastatic human melanoma lesions in a lymph node. DAPI was used to counterstain the cell nuclei. Scale bar: 100 μm.

FIG. 2O. Western blotting indicates that the expression of Sb9 protein is significantly higher in B16-Sb9⁺⁺ cells than B16-WT cells. Sb9-GrB complex (com), unbound Sb9 (mono), and complex degradation products or cleaved Sb9 (deg) are indicated by arrows. GAPDH was used as a loading control for protein normalization.

FIG. 2P. Tumor growth curve indicates significantly more rapid growth of B16-Sb9⁺⁺ (n=14) than B16-WT (n=14) mouse melanoma tumors in C57BL/6 mice. Data were expressed as means±SEM. *P<0.05 (Student's t-test).

FIGS. 3A-3S. Sb9 deletions restore host immunity to tumors and disrupt stroma in the TME.

FIG. 3A. Tumor growth curve demonstrates significantly slower growth of B16-WT mouse melanoma tumors in Sb9 KO mice (n=10) than C57BL/6-WT mice (n=10). Data are expressed as means±SEM. **P<0.01, ***P<0.001 (Student's t-test).

FIG. 3B. Survival curve shows significantly longer survival of Sb9 KO mice (n=10) than C57BL/6-WT mice (n=10) bearing B16-WT mouse melanoma tumors. ***P<0.001 (Student's t-test).

FIG. 3C. Tumor growth curve displays significantly slower growth of B16-Sb9 KO melanoma tumors in the Sb9 KO mice (n=10, Sb9 KO/Sb9 KO) than B16-WT mouse melanoma tumors in the C57BL/6-WT mice (n=10, WT/WT). Data are expressed as means±SEM. ***P<0.001 (Student's t-test).

FIG. 3D. Survival curve shows significantly longer survival of Sb9 KO mice bearing the B16-Sb9 KO melanoma tumors (n=10, Sb9 KO/Sb9 KO) than C57BL/6-WT mice bearing the B16-WT melanoma tumors (n=10, WT/WT). ***P<0.001 (Student's t-test).

FIGS. 3E-3H. Flow cytometric analyses demonstrate significantly higher ratios of CD44^{high}CD62L^{low} CD8⁺ effector memory T cells/Treg (FIG. 3E), TNFα⁺ CD8⁺ T cells/Treg (FIG. 3F), GrB⁺ CD8⁺ T cells/Treg (FIG. 3G), and IFNγCD8⁺ T cells/Treg (FIG. 3H) in melanoma tumors from the Sb9 KO/Sb9 KO group than the WT/WT group at 17 days post-implantation. Treg cells were defined at the CD4⁺CD25⁺Foxp3⁺ population, and all listed populations were gated under CD45⁺ CD3⁺ cells. Data are expressed as means±SEM. **P<0.01, ***P<0.001 (Student's t-test).

FIGS. 3I-3J. Flow cytometric analyses show significantly lower percentages of tumor-infiltrating CD45⁺ CD11b⁺F4/80⁺Ly6C⁻Ly6G⁻ TAM populations (FIG. 3I) and CD45⁺ CD11b⁺Gr1⁺CD3⁻ MDSC populations (FIG. 3J) in melanoma tumors from the Sb9 KO/Sb9 KO group than the WT/WT group at 17 days post-implantation. Data are expressed as means±SEM. *P<0.05, **P<0.01 (Student's t-test).

FIG. 3K. Flow cytometric analysis shows significantly lower percentages of tumor-infiltrating IL10⁺CD4⁺ population in melanoma tumors from the Sb9 KO/Sb9 KO group than the WT/WT group at 17 days post-implantation. Data are expressed as means±SEM. ***P<0.001 (Student's t-test).

FIG. 3L. Flow cytometric analysis shows significantly higher percentages of tumor-infiltrating IL2⁺ CD4⁺ population in melanoma tumors from the Sb9 KO/Sb9 KO group than the WT/WT group at 17 days post-implantation. Data are expressed as means±SEM. ***P<0.001 (Student's t-test).

FIGS. 3M-3P. Semi-quantitative analysis of fluorescence micrographs demonstrates significantly smaller surface area occupied by fibronectin (FIG. 3M), collagen I (FIG. 3N), PDGFR-β⁺ fibroblasts (FIG. 3O), and α-SMA⁺ fibroblasts (FIG. 3P) in melanoma tumor sections from the Sb9 KO/Sb9 KO group than in the WT/WT group at 17 days post-implantation. Data are expressed as means±SEM. ***P<0.001 (Student's t-test).

FIGS. 3Q-3S. Crystal violet staining (FIG. 3Q, top) of MSC-WT cells co-cultured with B16-GFP cells for 48 hr reveals multiple dense clusters comprised of both MSC-WT and B16 tumor cells, while co-cultured MSC-Sb9 KO and B16 cells are scattered and not situated in proximity to each other. Scale bar: 200 μm. Semi-quantitative analysis of representative fluorescence micrographs shows significantly larger amounts of fibronectin (FIG. 3Q, middle; FIG. 3R) and collagen I (FIG. 3Q, bottom; FIG. 3S) produced by MSC-WT cells in co-culture with B16-GFP melanoma cells than the MSC-Sb9 KO and B16-GFP co-culture group after 48 hrs. DAPI was used to counterstain the cell nuclei. Scale bar: 100 μm.

FIGS. 4A-4H. Small molecules evoke protective immunity to tumors.

FIG. 4A. The upper panels show the TSA results of the MBP-Sb9 protein incubated with 5 mM 3,4-dihydroxybenzoic acid (3,4-DHBA; left) and 3-oxo-1-indancarboxylic acid (right), or with 2.5% DMSO. The thermal shifts of the compounds 3,4-dihydroxybenzoic acid and 3-oxo-1-indancarboxylic acid are shown in a thick dark line against a baseline control shown as a thin gray line. The bottom panels show the TSA results of the MBP-Sb9 protein incubated with 3,4-dihydroxybenzoic acid (left) or 3-oxo-1-indancarboxylic acid (right) at different concentrations, including 5000, 1667, 556, 185, 62, and 21 μM. The concentration-response curves are plotted in a black/gray color scale (higher intensity is higher concentration) and the straight line plots represent the compound background signal.

FIG. 4B. Sensorgrams of compounds 3,4-dihydroxybenzoic acid (top), 3-oxo-1-indancarboxylic acid (middle), and the compound of Formula (III) (bottom) show the binding of 3,4-dihydroxybenzoic acid and the compound of Formula (III) to Sb9 (top), but not to MBP (bottom) by Surface Plasmon Resonance (SPR).

FIG. 4C. Comparison of caspase-3 activity among B16 melanoma cells treated with 3,4-dihydroxybenzoic acid (white) and 200 μM of the compound of Formula (III) (gray) for 24 h demonstrated that the compound of Formula (III) (gray) induced highest activity, as determined by the EnzChek® Caspase-3 Assay Kit.

FIGS. 4D-4E. Saturation-Transfer Difference (STD) NMR assays of the compound of Formula (III) (450 μM) and Sb9 protein (45 μM) at various pH values are provided. (FIG. 4D) Aromatic region of STD NMR spectrum showing the compound of Formula (III) in the presence of Sb9 protein at pH 4.0 and 10° C., exhibiting large saturation difference comparing on-resonance and off-resonance 1D 1H spectra. (FIG. 4E) Plots of averaged STD scores (%) of aromatic peaks showing binding of the compound of Formula (III) to MBP-Sb9 protein is enhanced significantly at lower pH in a pH-dependent manner.

FIG. 4F. Fluorescence micrographs demonstrate the presence of Sb9 (left) and GrB (right) in the lysosomes of B16 melanoma cells. DAPI was used to counterstain the cell nuclei. Scale bar: 10 μm.

FIG. 4G. Western blot shows that the compound of Formula (III) reduces the expression of Sb9-GrB complex in a concentration-dependent manner (50 μM, 200 μM, 600 μM) in B16 cells as compared to control. Sb9-GrB complex (com), unbound Sb9 (mono), and complex degradation products or cleaved Sb9 (deg) are indicated by arrows. GAPDH was used as a loading control for protein normalization.

FIG. 4H. Fluorescence micrographs indicate that the compound of Formula (III) (right) significantly increases the expression of LAMP1 and expands the lysosomes. DAPI was used to counterstain the cell nuclei. Scale bar: 10 μm.

FIGS. 5A-5P. Treatment with a small molecule suppresses melanoma progression in vivo.

FIGS. 5A-5B. Tumor growth curve demonstrates significantly slower growth of B16-WT mouse melanoma tumors in the C57BL/6-WT mice treated with the compound of Formula (III) (300 μg i.p. bid for 14 days following implantation) (n=17) than the control group treated with 10% DMSO (n=17) (FIG. 5A). Data are expressed as means±SEM. *P<0.05, **P<0.01 (Student's t-test). Mean survival time (MST) is increased in mice treated with the compound of Formula (III) as compared to Control group (FIG. 5B).

FIGS. 5C-5E. Flow cytometric analyses demonstrate significantly higher percentages of tumor-infiltrating CD44^{high} CD62L^{low} CD8⁺ effector memory T cells (FIG. 5C), GrB⁺ CD8⁺ T cells (FIG. 5D), and IFNγ⁺ CD8⁺ T cells (FIG. 5E) in melanoma tumors from the compound of Formula (III)-treatment group than the control group at 17 days post-implantation. All listed populations were gated under CD45⁺ CD3⁺ cells. Data are expressed as means±SEM. **P<0.01, ***P<0.001 (Student's t-test).

FIGS. 5F-5H. Flow cytometric analyses show significantly lower percentages of tumor-infiltrating CD4⁺ CD25⁺Foxp3⁺ Treg populations (FIG. 5F), CD45⁺ CD11b⁺F4/80⁺Ly6C⁻Ly6G⁻ TAM populations (FIG. 5G), and CD45⁺ CD11b⁺Gr1⁺CD3⁻ MDSC populations (FIG. 5H) in melanoma tumors from the compound of Formula (III)-treatment group than the control group at 17 days post-implantation. Data are expressed as means±SEM. *P<0.05, **P<0.01 (Student's t-test).

FIG. 5I. Flow cytometric analysis shows significantly lower percentages of tumor-infiltrating IL10⁺CD4⁺ population in melanoma tumors from the compound of Formula (III)-treatment group than the control group at 17 days post-implantation. Data are expressed as means±SEM. ***P<0.001 (Student's t-test).

FIG. 5J. Caspase-3 activity assay demonstrates that the compound of Formula (III) (200 μM) induces GrB-mediated apoptosis in B16 melanoma cells, as determined by the EnzChek® Caspase-3 Assay Kit. The GrB inhibitor (368050) was used at 10 μM.

FIGS. 5K-5L. Grantoxilux assay demonstrates increased activity of GrB in B16 cells treated with the compound of Formula (III) (200 μM) for 24 h.

FIG. 5M. Quantitative flow cytometric analysis shows that the compound of Formula (III) does not induce additional apoptosis at various concentrations (50 μM, 100 μM, and 200 μM) in the B16-Sb9 KO melanoma cells, as indicated by expressions of Annexin-V and viability dye. Cells treated with PBS alone were used as a negative control. Data are expressed as means±SD from three independent experiments. NS (no significant difference) (Student's t-test).

FIG. 5N. Tumor growth curve indicates no difference in antitumor effect of the compound of Formula (III) on B16-Sb9 KO tumors in Sb9 KO mice, as compared to the control group. Data are expressed as means±SEM. NS (no significant difference) (Student's t-test).

FIGS. 5O-5P. RT-PCR results show that the compound of Formula (III) (black) has no significant effect on the gene expression of caspase-3 and Bcl-2 in the B16-Sb9 KO cells. Data are expressed as means±SD from three independent experiments. NS (no significant difference) (Student's t-test).

FIGS. 6A-6Q. Treatment with a small molecule restrains breast tumor growth in vivo.

FIG. 6A. Semi-quantitative analysis of fluorescence micrographs show that the compound of Formula (III) (black triangles) reduces the production of ECM fiber fibronectin by MSC-WT cells significantly. Data are expressed as means±SD. ***P<0.001 (Student's t-test).

FIGS. 6B-6C. Representative fluorescence micrographs (FIG. 6C) and semi-quantitative analysis (FIG. 6B) show that the compound of Formula (III) (black triangles, right) decreases significantly the expression of ECM fiber fibronectin by MSC-WT cells following co-culture with B16-GFP cells. Data are expressed as means±SD. ***P<0.001 (Student's t-test). Scale bar: 100 μm.

FIGS. 6D-6E. Fluorescence micrographs of consecutive primary human breast tumor sections demonstrate high expression of Sb9 (FIG. 6D) by α-SMA⁺ fibroblasts (FIG. 6E). Tumor marker pan-cytokeratin was used to stain. DAPI was used to counterstain the cell nuclei. Scale bar: 50 μm.

FIGS. 6F-6G. Tumor growth curve shows significantly slower growth of 4T1 mouse breast tumors in BALB/c mice treated with the compound of Formula (III) (300 μg i.p. bid for 14 days following implantation) (n=7) than the control group treated with 10% DMSO (n=7), as demonstrated by representative photographs of the tumors from both groups at 17 days post-implantation provided in panel H (FIG. 6F). Data are expressed as means±SEM. *P<0.05, ***P<0.001 (Student's t-test). Mean survival time (MST) is increased in mice treated with the compound of Formula (III) as compared to Control group (FIG. 6G).

FIGS. 6H-6L. Semi-quantitative analysis of fluorescence micrographs shows significantly lower expression (MFI) of cancer-associated fibroblast (CAF) markers α-SMA (FIG. 6H), PDGFR-α (FIG. 6I), PDGFR-β (FIG. 6J), FAP (FIG. 6K), and FSP-1 (FIG. 6L) in breast tumor sections from the compound of Formula (III)-treatment group in comparison to the control group (10% DMSO) at 17 days post-implantation (black triangles). Data are expressed as means±SEM. ***P<0.001 (Student's t-test).

FIG. 6M. Representative fluorescence micrographs show significantly lower expression of cancer-associated fibroblast (CAF) marker FAP in breast tumor sections from the compound of Formula (III) treatment group (right) in comparison to the control group (10% DMSO) at 17 days post-implantation. DAPI was used to counterstain the cell nuclei. Scale bar: 100 μm.

FIGS. 6N-6O. Tumor growth curve demonstrates significantly slower growth of RENCA mouse kidney tumors in the male BALB/c mice treated with the compound of Formula (III) (300 μg i.p. bid for 21 days following implantation) (n=8) (gray) than the control group treated with 10% DMSO (n=8) (FIG. 6N). Data are expressed as means±SEM. *P<0.05, **P<0.01 (Student's t-test). Mean survival time (MST) is increased in mice treated with the compound of Formula (III) as compared to Control group (FIG. 6O).

FIGS. 6P-6Q. Tumor growth curve demonstrates significantly slower growth of LLC1 mouse lung tumors in the C57BL/6-WT mice treated with the compound of Formula (III) (300 μg i.p. bid for 21 days following implantation) (n=8) (gray) than the control group treated with 10% DMSO (n=8) (FIG. 6P). Data are expressed as means±SEM. *P<0.05 (Student's t-test). Mean survival time (MST) is increased in mice treated with the compound of Formula (III) as compared to Control group (FIG. 6Q).

FIGS. 7A-7F. Sb9 is required to protect melanoma tumors from GrB-induced apoptosis.

FIG. 7A. Immunohistochemistry (TIC) micrographs demonstrate the presence of Sb9 in primary human malignant melanoma, breast adenocarcinoma, pancreatic adenocarcinoma, Hodgkin lymphoma, prostate adenocarcinoma, and ovarian carcinoma. Scale bar: 50 μm.

FIGS. 7B-7C. Representative micrographs of Crystal Violet cell staining (FIG. 7B) and quantitative analysis (FIG. 7C) indicate that the cell proliferation rate of B16-Sb9 KO cells is similar to that of B16-WT cells. NS (no significant difference) (Student's t-test).

FIG. 7D. RT-qPCR analysis demonstrates that Ki67 mRNA expression in B16-Sb9 KO cells is similar to that in B16-WT cells. NS (no significant difference) (Student's t-test).

FIG. 7E. Grantoxilux assay reveals significantly higher GrB activity in B16-Sb9 KO cells, as compared to B16-WT cells. Data are expressed as means±SD from three independent experiments. *P<0.05 (Student's t-test).

FIG. 7F. Representative fluorescence micrographs demonstrate that treatment of B16-WT cells with IL2 (70 ng/mL) versus PBS for 48 h results in higher GrB expression in B16 mouse melanoma cells. DAPI was used to counterstain the cell nuclei. Scale bar: 20 μm.

FIGS. 8A-8F. Sb9 acts cell intrinsically to control tumor growth in vivo.

FIG. 8A. Representative photographs of melanoma tumors in C57BL/6 mice show significantly smaller size of B16-Sb9 KO (n=10) than B16-WT (n=10) mouse melanoma tumors.

FIG. 8B. Survival curve of C57BL/6 mice bearing melanoma tumors indicates significantly longer survival in those with B16-Sb9 KO (n=10) tumors in comparison to B16-WT (n=10) tumors. ***P<0.001 (Student's t-test).

FIG. 8C. Quantitative analysis of fluorescence micrographs reveal that the expressions of Nestin are lower in B16-Sb9 KO tumors in comparison to B16-WT tumors at 17 days post-implantation. Data are expressed as means±SEM. ***P<0.001 (Student's t-test).

FIG. 8D. Western blotting indicates that the expression of cleaved caspase-3 (C-CAS3) is significantly higher in B16-Sb9 KO tumors (n=5) in comparison to B16-WT (n=5) tumors at 17 days post-implantation. GAPDH was used as a loading control for protein normalization.

FIG. 8E. Western blotting confirms the successful knockout of Sb9 protein in the A375 human melanoma cells. Sb9-GrB complex (com), unbound Sb9 (mono), and complex degradation products or cleaved Sb9 (deg) are indicated by arrows. GAPDH was used as a loading control for protein normalization.

FIG. 8F. Tumor growth curve through 25 days following implantation in NSG mice indicates significantly slower rate of A375-Sb9 KO (n=7) than A375-WT (n=7) human melanoma tumors. Data are expressed as means±SEM. **P<0.01 (Student's t-test).

FIGS. 9A-9I. Sb9 deletions restore host immunity to tumors and disrupt stroma in the TME.

FIG. 9A. Quantitative analysis of fluorescence micrographs display significantly higher expression of GrB (MFI) in melanoma tumor sections from the Sb9 KO/Sb9 KO group than the WT/WT group at 17 days post-implantation. Data are expressed as means±SEM. ***P<0.001 (Student's t-test).

FIG. 9B. Quantitative analysis of fluorescence micrographs display significantly higher density (MFI) of cleaved caspase-3 (C-CAS-3)⁺ apoptotic cells in melanoma tumor sections from the Sb9 KO/Sb9 KO group than the WT/WT group at 17 days post-implantation. Data are expressed as means±SEM. ***P<0.001 (Student's t-test).

FIG. 9C. Western blotting indicates that the expression of cleaved caspase-3 (C-CAS3) is significantly higher in melanoma tumor sections from the Sb9 KO/Sb9 KO group (n=5) in comparison to the WT/WT group (n=5) at 17 days post-implantation. GAPDH was used as a loading control for protein normalization.

FIGS. 9D-9E. Fluorescence micrographs of consecutive melanoma tumor sections demonstrate that CD11b⁺, Ly-6C⁻ TAMs (FIG. 9D) and CD11b⁺, Gr-1⁺ MDSCs (FIG. 9E) express Sb9 within mouse melanoma tumors. DAPI was used to counterstain the cell nuclei. Scale bar: 10 μm.

FIGS. 9F-9G. Fluorescence micrographs of consecutive breast tumor sections demonstrate that CD11b⁺, Ly-6C⁻ TAMs (FIG. 9F) and CD11b⁺, Gr-1⁺ MDSCs (FIG. 9G) express Sb9 within mouse breast tumors. DAPI was used to counterstain the cell nuclei. Scale bar: 10 μm.

FIG. 9H. Flow cytometric analyses demonstrate significantly higher ratios of CD8⁺ T cells/Treg in melanoma tumors from the Sb9 KO/Sb9 KO group than the WT/WT group at 17 days post-implantation. Treg cells were defined at the CD4⁺ CD25⁺Foxp3⁺ population, and all listed populations were gated under CD45⁺ CD3⁺ cells. Data are expressed as means±SEM. *P 0.05 (Student's t-test).

FIG. 9I. Flow cytometric analyses show significantly lower percentages of tumor-infiltrating CD8⁺IL10⁺ population in melanoma tumors from the Sb9 KO/Sb9 KO group than the WT/WT group at 17 days post-implantation. Data are expressed as means±SEM. **P<0.01 (Student's t-test).

FIGS. 10A-10J. Treatment with a small molecule suppresses melanoma progression in vivo.

FIG. 10A. Representative photographs show significantly smaller size of B16-WT mouse melanoma tumors in the C57BL/6-WT mice treated with the compound the compound of Formula (III) (300 μg i.p. bid for 14 days following implantation) than the control group treated with 10% DMSO, at 27 days post-implantation.

FIG. 10B. Flow cytometric analyses demonstrate significantly higher percentages of IFNγ⁺ CD4⁺ T cells in melanoma tumors from the compound of Formula (III)-treatment group (triangles) than the control group at 17 days post-implantation. All listed populations were gated under CD45⁺ CD3⁺ cells. Data are expressed as means±SEM. **P<0.01 (Student's t-test).

FIG. 10C. Flow cytometric analyses show significantly lower percentages of tumor-infiltrating IL10⁺CD8⁺ population in melanoma tumors from the compound of Formula (III)-treatment group (blue triangles) than the control group at 17 days post-implantation. Data are expressed as means±SEM. *P<0.05 (Student's t-test).

FIGS. 10D-10E. Tumor growth curve displays significantly slower growth of A375-WT human melanoma tumors in NSG mice treated with the compound of Formula (III) (300 μg i.p. bid for 14 days following implantation) (n=7) (gray) than the control group treated with 10% DMSO (n=7) (FIG. 10D). Data are expressed as means±SEM. *P<0.05 (Student's t-test). Mean survival time (MST) is increased in mice treated with the compound of Formula (III) as compared to Control group (FIG. 10E).

FIGS. 10F-10G. RT-PCR results show that the compound of Formula (III) (black) has no significant effect on the gene expression of caspase-8 and P53 in the B16-Sb9 KO cells. Data are expressed as means±SD from three independent experiments. NS (no significant difference) (Student's t-test).

FIG. 10H. The CBC results show no significant difference in WBC, HGB, and PLT between C57BL/6-WT mice treated with the compound of Formula (III) (300 μg i.p. bid for 14 days following implantation) (n=5) (lighter gray) and the control group treated with 10% DMSO (n=5). Data are expressed as means±SEM. NS (no significant difference) (Student's t-test).

FIG. 10I. The serum results show no significant difference in AST, ALT, CRE, and ALB levels between C57BL/6-WT mice treated with the compound of Formula (III) (300 μg i.p. bid for 14 days following implantation) (n=5) (lighter gray) and the control group treated with 10% DMSO (n=5). Data are expressed as means±SEM. NS (no significant difference) (Student's t-test).

FIG. 10J. Representative H&E micrographs show no significant morphology difference in liver, kidney, lung, and heart tissues between C57BL/6-WT mice treated with the compound of Formula (III) (300 μg i.p. bid for 14 days following implantation) (n=5) (bottom) and the control group treated with 10% DMSO (n=5) (top).

FIGS. 11A-11C. Treatment with a small molecule restrains breast tumor growth in vivo.

FIG. 11A. Quantitative analysis of fluorescence micrographs show that the compound of Formula (III) (black triangles) significantly reduces the production of ECM fiber collagen I by MSC-WT cells. Data are expressed as means±SD. ***P<0.001 (Student's t-test).

FIGS. 11B-11C. Representative fluorescence micrographs (FIG. 11C) and quantitative analysis (FIG. 11B) show that the compound of Formula (III) (black triangles, right) significantly decreases the expression of ECM fiber collagen I by MSC-WT cells co-cultured with B16-GFP cells. Data are expressed as means±SD. ***P<0.001 (Student's t-test). Scale bar: 100 μm.

FIG. 12. Representative flow cytometry figures exemplify the gating strategies used in the study.

FIGS. 13A-13B. Saturation-Transfer Difference (STD) NMR assays of the compound of Formula (I) (FIG. 13A) and the compound of Formula (II) (FIG. 13B) (420 μM) and Sb9 protein (45 μM).

FIG. 14. Fluorescence micrographs of cancer cells. The results showed that the compounds of Formulas (I) (top) and (II) (bottom) significantly increased caspase-3 expression, comparing to the equal amount of DMSO. Scale bar: 100 m.

FIGS. 15A-15B. The compounds of Formula (I) and (II) significantly decreased tumor size in an in vivo model. (FIG. 15A) Tumor size was significantly decreased at Day 11, Day 14 post-tumor implantation in the compound of Formula (I) group compared to Control group. (FIG. 15B) Tumor size was significantly decreased at Day 14 post-tumor implantation in the compound of Formula (II) group compared to Control group.

FIG. 16. Saturation-Transfer Difference (STD) NMR assay of the compound of Formula (IV) and Sb9 protein.

FIGS. 17A-17B. Treatment of mice having B16 melanoma with the compound of Formula (IV) increases MST and suppresses melanoma progression in vivo. (FIG. 17A) Tumor size was decreased at Day 22 post-tumor implantation in the compound of Formula (IV) group (gray) compared to Control group. (FIG. 17B) Mean survival time (MST) is increased in mice treated with the compound of Formula (IV) as compared to Control group.

FIG. 18. Treatment of mice having B16 melanoma with the compound of Formula (III) as compared to Control group and mice treated with anti-PD1. Median survival time (MST) for the mice that received the compound of Formula (III) (36 days) was longer than those that received either no treatment (28.5 days) or anti-PD1 (28 days) (*p<0.05).

### DETAILED DESCRIPTION

The majority of malignant tumors are immunologically inert. In order to grow in an immune-competent host, tumor cells acquire genetic mutations and undergo epigenetic changes that result in immune-resistant phenotypes (Kather et al., 2018; Trujillo et al., 2018). In an effort to make tumors immunologically active, genotoxic therapies (such as radio- and chemo-therapy) have been used as adjuvants for immunotherapy by evoking innate immune responses such as type I interferon (IFN) signaling. However, these adjuvant strategies are hampered by the induction of suppressors of type I-IFN pathway by genotoxic agents (Trujillo et al., 2018). To circumvent this problem combinations of up to three treatments may have to be used, complicating clinical trials.

Serine proteases participate in a wide range of physiological processes, which are regulated by a large family of peptidase inhibitors referred to as serine protease inhibitors (serpins) (Silverman et al., 2001). Serpins inhibit by acting as a suicide substrate for a serine protease that results in a characteristic covalent inhibitory complex (Huntington et al., 2000; Mangan et al., 2008). In contrast to most serpins, which are extracellular, Serpinf19 (Sb9) (PI9 in human, Spi6 in mice) is a member of the ovalbumin family of serpins, which reside within the nuclei and cytoplasm of cells (Bird et al., 1998; Bots and Medema, 2008; Sun et al., 1996; Sun et al., 1997). Sb9 proteins are physiological inhibitors of granzyme B (GrB), which following delivery into target cells by cytotoxic lymphocytes (CLs), triggers apoptosis by activating caspases-3 and -8 (Pinkoski et al., 2001). Sb9 has been shown to protect pro-inflammatory CLs from self-inflicted damage by their own GrB (Hirst et al., 2003; Sun et al., 1996). Sb9 also protects other leukocytes, which are both pro-inflammatory (dendritic cells and neutrophils (Medema et al., 2001b; Rizzitelli et al., 2012)) or anti-inflammatory (Treg and MDSC (Azzi et al., 2013; Kumar et al., 2016; Lindau et al., 2013)) from GrB that originates either from CL or is produced endogenously.

Sb9 is also thought to protect tumor cells from GrB delivered by CLs, but this has not been tested directly in vivo (Bots and Medema, 2008; Mangan et al., 2008; Medema et al., 2001a). Immunosuppressive tumor-associated macrophages (TAMs), myeloid-derived suppressor cells (MDSCs), and regulatory T cells (Tregs) in the tumor microenvironment (TME) allow tumor progression and metastasis (Kumar et al., 2016; Lindau et al., 2013). The potential effect of GrB inhibition by Sb9 on both the anti-tumor cellular effectors (such as CL) and immunosuppressive components of the TME is not known (Quail and Joyce, 2013). Despite the potential for immunomodulation, whether or not inhibition of Sb9 results in beneficial elimination of tumors, either by direct killing or by increased host immunity, remains to be determined. In addition to its implications on immune cells and the intrinsic survival of tumor cells, the Sb9-GrB axis can also have a major impact on the tumor stroma as well. Stromal cells, including cancer-associated fibroblasts (CAFs), constitute a major cellular component of the diverse TME and play a critical role in tumor development (Kalluri, 2016; Quail and Joyce, 2013). Stromal cells can create an optimal milieu by producing numerous growth factors, cytokines, and chemokines that promote tumor growth (Nilendu et al., 2018; Wei et al., 2018).

In this report, genetic ablation was used to demonstrate a direct requirement of Sb9 for tumor cell growth through the inhibition of cell death caused by GrB. The expression of Sb9 has been shown in both effector and immune-suppressive cells (Azzi et al., 2013; Mangan et al., 2008; Phillips et al., 2004). The ablation of Sb9 results in the impaired survival of CTL specific to intracellular pathogens (Zhang et al., 2006). However, as shown herein, the aggregate effect of Sb9 ablation is increased CTL immunity to tumors in vivo. Without wishing to be bound by any theory, it is believed that the increased immunity to tumors in Sb9 KO mice is more due to the impaired survival of immuno-suppressive cells in the TME than any decrease in cell intrinsic viability of GrB⁺ CTL. Sb9 is also known to inhibit caspase-1 (interleukin-1β-converting enzyme), which is involved in the inflammatory response by cleaving the precursors of inflammatory cytokines, including IL-1β, IL-18, and IL-33 (Annand et al., 1999; Young et al., 2000). The inflammatory microenvironment can modulate directly the number, function, migration, and maintenance of effector immune cells (Kim and Harty, 2014). Therefore, alteration of this milieu may constitute an alternative pathway by which Sb9 suppresses the inflammatory response and escapes from immune surveillance against tumors.

Notably, metastasis of melanoma to the TDLNs was significantly less frequent in the B16-Sb9 KD group than in the B16-WT group. Given that the metastatic melanoma within the LN also expressed Sb9, one could speculate that Sb9 could protect metastatic niches against the CTL of LN, hence introducing a novel mechanism behind the spreading of neoplasms through lymphatics. Furthermore, various cancers have been associated with a heterogeneous and adaptive TME, and their growth can be driven by the local TME in which they thrive. Besides lymphocytes and other immune cells, stromal cells are a major cellular component of the diverse TME that plays a critical role in tumor development (Nilendu et al., 2018). Our finding that Sb9 regulates the function of the stroma is supported by our previous report for Sb9 in protecting mesenchymal stem cells (MSCs) from exogenous GrB (El Haddad et al., 2011a; El Haddad et al., 2011b). CAFs have received increasing attention as the key cellular player within the tumor stroma that supports the initiation, progression and metastasis of cancers (Kalluri, 2016; Wei et al., 2018). Importantly in a melanoma mouse model, suppression of CAF activity was seen with less accumulation and generation of matrix proteins as well as lower neovascularization in the Sb9 KO/Sb9 KO group. In addition, GrB expression was higher in melanoma sections from the Sb9 KO/Sb9 KO group, as compared with the WT/WT group. These findings suggest that lack of Sb9 in the stromal cells, including CAFs and MSCs, of the Sb9-deficient host could increase their susceptibility to the lethal effect of GrB released within the stroma. Notably, GrB has also been reported to be an extracellular substrate with ability to also cleave ECM materials, and hence promoting tumor cell migration and metastasis. This further highlights the importance of Sb9 inhibition in suppressing tumor metastasis. Altogether, pharmacological inhibition of Sb9 target tumors in an unprecedented fashion on multiple levels, inducing death of the tumor cells by their own GrB, downregulating the immunosuppressive cells in the TME, eliminating tumor angiogenesis and stroma, and thereby transforming the TME less conducive for tumor growth and metastasis.

The present disclosure is a starting point for the introduction of first-in-class small molecule inhibitors of Sb9. Like any other field in its infancy, the field of Sb9 inhibition therapeutics requires more work to characterize and optimize their efficacy. For instance, one key challenge that remains is to generate drugs with much improved affinity through high-throughput screening of fragments. Although the affinity of our compounds remains in the expected range of small molecules, they may have much better affinity in vivo for many reasons. For example, the low pH of the lysosome confers a much higher affinity of the disclosed compounds to Sb9. Sb9 produced by the tumor likely undergoes significant post-translational modifications (not recapitulated in our screening assays) that may increase its affinity to the disclosed compounds. Though the generation of selective ligands with high binding affinity defines the current paradigm, many low-affinity drugs could also be effective (Csermely et al., 2005; Lipton, 2006; Mestres and Gregori-Puigjane, 2009). In the case of Sb9, the development of similar drugs with a larger size and better affinity than the disclosed compounds may not result in an improvement of clinical efficacy, as the larger size may limit entry inside cells. Nonetheless, the fact that genetic or pharmacological inhibition of Sb9 in mice did not result in significant anomalies or side effects highlights the selectivity of the disclosed compounds and the potential applicability for clinical translation. These findings also demonstrate the potential importance of disseminating our data to create the momentum needed to advance discoveries of novel Sb9 inhibitors. Immune checkpoint inhibitors are considered the most recent therapeutic breakthrough in the cancer world (Peoples, 2019). However, Sb9 inhibition could be superior due to its multifaceted actions on key players responsible for the growth and metastasis of tumors. Furthermore, Sb9 inhibitors may prove to be safer than immune checkpoint inhibitors (Peoples, 2019; Postow et al., 2018).

Methods of Treatment

The methods described herein include methods for the treatment of disorders associated with hyperproliferation. In some embodiments, the disorder is cancer, e.g., metastatic cancer. Generally, the methods include administering a therapeutically effective amount of an Sb9 inhibitor or analog thereof as described herein, to a subject who is in need of, or who has been determined to be in need of, such treatment.

As used in this context, to “treat” means to ameliorate at least one symptom of the disorder associated with hyperproliferation. Administration of a therapeutically effective amount of a compound described herein for the treatment of a condition associated with hyperproliferation can result in one or more of decreased tumor size, decreased tumor number, decreased tumor growth rate, decreased rate or risk of metastasis, and/or increased survival time.

Examples of cellular proliferative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer,” “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as solid tumors including carcinomas and sarcomas affecting lung, breast, thyroid, lymphoid, gastrointestinal, and genitourinary tract, as well as adenocarcinomas that include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

As used in this context, to “treat” means to ameliorate at least one symptom of the cancer. Administration of a therapeutically effective amount of a compound described herein for the treatment of a cancer can result in one or more of decreased tumor size or growth rate or decreased tumor burden, and/or an increased life span or increased time to progression or reoccurrence.

In some embodiments, the methods can include a step of identifying a subject as having a cancer associated with increased expression of Sb9, e.g., by obtaining a sample from the subject and detecting the presence or level of Sb9, e.g., Sb9 protein or transcript. As used herein the term “sample”, when referring to the material to be tested for the presence of a mutation, can include inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The sample can comprise cell-free RNA or DNA. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR, i.e., BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current edition; Sambrook et al., Molecular Cloning: A Laboratory Manual (Sup. 3rd Edition, 2001); Bernard (2002) Clin Chem 48(8):1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence or level of Sb9. In some embodiments a technique suitable for the detection of alterations in the structure or sequence of nucleic acids, such as the presence of deletions, amplifications, or substitutions, can be used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

The methods can include using next generation sequencing or other methods to identify cancers with levels of Sb9 above a reference level, e.g., by sequencing DNA or RNA from a tumor or cell free nucleic acids.

In some embodiments the methods include identifying and selecting a subject on the basis that they have a cancer with a level of Sb9 above a reference level.

Sb9 Inhibitors and Analogs

The methods and compositions described herein can include the use of compounds that are inhibitors of Sb9 and analogs thereof. In some embodiments, the compound is a compound of Formula (I)

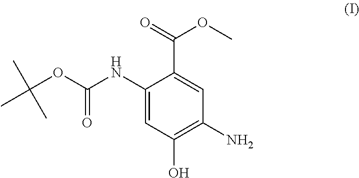 [SMILES: COC(=O)c1cc(N)c(O)cc1NC(=O)OC(C)(C)C]

and pharmaceutically acceptable salts thereof. The compound of Formula (I) (methyl 5-amino-2-((tert-butoxycarbonyl)amino)-4-hydroxybenzoate) and pharmaceutically acceptable salts thereof are useful as inhibitors of Sb9.

In some embodiments, the compound is a compound of Formula (II)

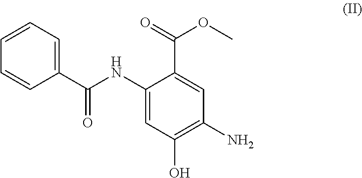 [SMILES: COC(=O)c1cc(N)c(O)cc1NC(=O)c1ccccc1]

and pharmaceutically acceptable salts thereof. The compound of Formula (II) (methyl 5-amino-2-benzamido-4-hydroxybenzoate) and pharmaceutically acceptable salts thereof are useful as inhibitors of Sb9.

Example processes and intermediates of the present disclosure are provided below in Schemes 1 and 2. As will be appreciated by those skilled in the art, the compounds provided herein, including salts thereof, can be prepared using known organic synthesis techniques and can be synthesized according to any of numerous possible synthetic routes, such as that provided in Schemes 1 and 2.

Scheme 1 shows an exemplary process for synthesizing the compound of Formula (I).

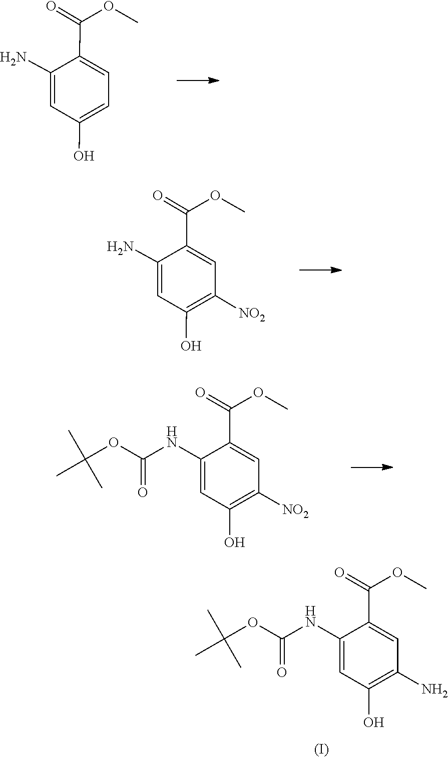 [SMILES: COC(=O)c1ccc(O)cc1N ; COC(=O)c1cc([N+](=O)[O-])c(O)cc1N ; COC(=O)c1cc([N+](=O)[O-])c(O)cc1NC(=O)OC(C)(C)C ; COC(=O)c1cc(N)c(O)cc1NC(=O)OC(C)(C)C ; "(I)"]

Scheme 2 shows an exemplary process for synthesizing the compound of Formula (II).

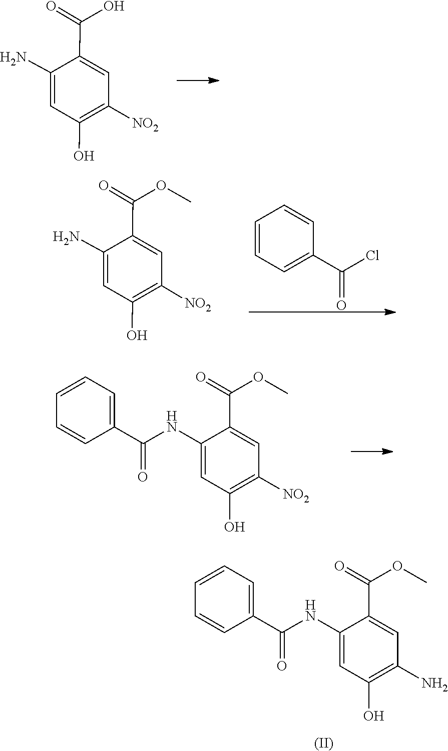

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Schemes 1 and 2 provide general guidance in connection with preparing the compounds of the present disclosure. For instance, the compounds of Formulas (I) and (II) can be prepared as shown in Schemes 1 and 2.

In some embodiments, the compound is a compound of Formula (III)

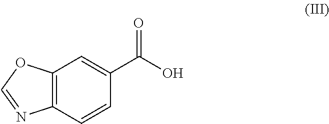 [SMILES: O=C(O)c1ccc2ncoc2c1]

and pharmaceutically acceptable salts and derivatives thereof. The compound of Formula (III) (1,3-benzoxazole-6-carboxylic acid) and pharmaceutically acceptable salts and derivatives thereof can be useful as inhibitors of Sb9. In some embodiments, the Sb9 inhibitor is an analog or derivative of the compound of Formula (III) or a pharmaceutically acceptable salt thereof.

In some embodiments, the compound is a compound of Formula (IV)

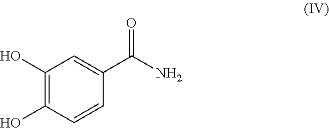 [SMILES: NC(=O)c1ccc(O)c(O)c1]

and pharmaceutically acceptable salts and derivatives thereof. The compound of Formula (IV) (3,4-dihydroxybenzamide) and pharmaceutically acceptable salts and derivatives thereof can be useful as inhibitors of Sb9. In some embodiments, the Sb9 inhibitor is an analog or derivative of the compound of Formula (IV) or a pharmaceutically acceptable salt thereof.

In some embodiments, the compound is a compound of Formula (V)

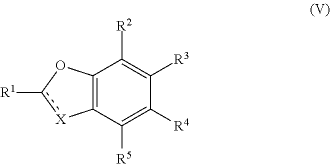

and pharmaceutically acceptable salts thereof, wherein:

X is N, NH, or O; wherein when X is N, [symbol] is a double bond and when X is NH or O, [symbol] is a single bond;

R¹ is H, C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂,

wherein the phenyl is optionally substituted with 1-5 substituents independently selected from halogen, C₁-C₃ alkyl, —O—(C₁-C₃ alkyl), and —C₁-C₃ haloalkyl;

one of R², R³, R⁴, and R⁵ is —(CH₂)ₙC(═O)NR⁶R⁷ and the others are each independently H or OH;

R⁶ is H, —C₁-C₃ alkyl, or —(C₁-C₃ alkylene)-(C₃-C₆ cycloalkyl);

R⁷ is —C₁-C₆ alkyl, —C₁-C₃ haloalkyl, —(C₁-C₃ alkylene)-O—(C₁-C₃ alkyl), —(C₁-C₃ alkylene)-N(C₁-C₃ alkyl)₂, —(C₁-C₃ alkylene)C(═O)NR⁸R⁹, —(C₁-C₃ alkylene)-phenyl, —C₃-C₆ cycloalkyl, 4-7 membered heterocyclic ring, —(C₁-C₃ alkylene)-(4-7 membered heterocyclic ring), —(C₁-C₃ alkylene)-NR⁸R⁹, —(C₁-C₃ alkylene)-C(═O)—O—(C₁-C₃ alkyl), or —OH,

wherein the —C₁-C₆ alkyl, —C₁-C₃ alkylene, and 4-7 membered heterocyclic ring are each optionally substituted with 1-2 substituents independently selected from —C₁-C₃ alkyl, OH, oxo (═O), and —O—(C₁-C₃ alkyl);

or R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁—C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹;

R⁸ and R⁹ are each independently H or —C₁-C₃ alkyl; and

n is 0 or 1.

In some embodiments of the compound of Formula (V), and pharmaceutically acceptable salts thereof:

X is N or O; wherein when X is N, [symbol] is a double bond and when X is O, [symbol] is a single bond;

R¹ is H, —C₁-C₃ alkyl, —C₁-C₃ haloalkyl, —C₃-C₆ cycloalkyl, 4-6 membered heterocyclic ring, phenyl optionally substituted with 1-5 halogen, —SH, oxo (═O), or —N(C₁-C₃ alkyl)₂;

one of R², R³, R⁴, and R⁵ is —(CH₂)ₙC(═O)NR⁶R⁷ and the others are each independently H or OH;

R⁶ is H or —C₁-C₃ alkyl;

R⁷ is —C₂-C₄ alkyl, —(C₁-C₃ alkylene)-O—(C₁-C₃ alkyl), —(C₁-C₃ alkylene)-N(C₁-C₃ alkyl)₂, —C₁-C₃ alkylene-phenyl, —C₃-C₆ cycloalkyl, or —OH;

or R⁶ and R⁷, together with the nitrogen to which they are attached, form a 4-6 membered heterocyclic ring; and

n is 0 or 1.

In some embodiments of the compound of Formula (V), and pharmaceutically acceptable salts thereof:

X is N and [symbol] is a double bond;

R¹ is phenyl optionally substituted with 1-5 halogen;

one of R², R³, R⁴, and R⁵ is —(CH₂)ₙC(═O)NR⁶R⁷ and the others are each H;

R⁶ is C₁-C₃ alkyl;

R⁷ is —C₂-C₄ alkyl; and

n is 0 or 1.

In some embodiments, X is N.

In some embodiments, X is NH.

In some embodiments, X is O.

In some embodiments, R¹ is H.

In some embodiments, R¹ is —C₁-C₃ alkyl. In some embodiments, R¹ is methyl. In some embodiments, R¹ is ethyl. In some embodiments, R¹ is propyl.

In some embodiments, R¹ is —C₁-C₃ haloalkyl. In some embodiments, R¹ is trifluoromethyl. In some embodiments, R¹ is perfluoroethyl.

In some embodiments, R¹ is —C₃-C₆ cycloalkyl. In some embodiments, R¹ is cyclopropyl.

In some embodiments, R¹ is cyclobutyl. In some embodiments, R¹ is cyclopentyl. In some embodiments, R¹ is cyclohexyl.

In some embodiments, R¹ is a 4-6 membered heterocyclic ring. In some embodiments, R¹ is tetrahydrofuranyl. In some embodiments, R¹ is pyrrolidinyl.

In some embodiments, R¹ is phenyl optionally substituted with 1-5 substituents independently selected from halogen, C₁-C₃ alkyl, —O—(C₁-C₃ alkyl), and —C₁-C₃ haloalkyl.

In some embodiments, R¹ is phenyl optionally substituted with 1-5 C₁-C₃ alkyl substituents. In some embodiments, R¹ is phenyl optionally substituted with 1-2 C₁-C₃ alkyl substituents. In some embodiments, R¹ is phenyl optionally substituted with 1-2 methyl substituents.

In some embodiments, R¹ is phenyl optionally substituted with 1-5 —O—(C₁-C₃ alkyl) substituents. In some embodiments, R¹ is phenyl optionally substituted with 1-2 —O—(C₁-C₃ alkyl) substituents. In some embodiments, R¹ is phenyl optionally substituted with 1-2 methoxy substituents.

In some embodiments, R¹ is phenyl optionally substituted with 1-5 —C₁-C₃ haloalkyl substituents. In some embodiments, R¹ is phenyl optionally substituted with 1-2 —C₁-C₃ haloalkyl substituents. In some embodiments, R¹ is phenyl optionally substituted with 1-2 trifluoromethyl substituents.

In some embodiments, R¹ is phenyl optionally substituted with 1-5 halogen.

In some embodiments, R¹ is phenyl optionally substituted with 1-5 halogen. In some embodiments, R¹ is phenyl optionally substituted with 1-2 halogen. In some embodiments, R¹ is phenyl optionally substituted with 1-2 chloro. In some embodiments, R¹ is phenyl optionally substituted with 1-2 bromo. In some embodiments, R¹ is phenyl optionally substituted with 1-2 fluoro.

In some embodiments, R¹ is H, —C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂, wherein the phenyl is optionally substituted with 1-2 substituents independently selected from halogen, methyl, methoxy, and trifluoromethyl. In some embodiments, R¹ is phenyl optionally substituted with 1-2 substituents independently selected from halogen, methyl, methoxy, and trifluoromethyl.

In some embodiments, R¹ is phenyl.

In some embodiments, R¹ is —SH.

In some embodiments, R¹ is oxo (═O).

In some embodiments, R¹ is —N(C₁-C₃ alkyl)₂. In some embodiments, R¹ is —N(CH₃)₂.

In some embodiments, R² is —(CH₂)C(═O)NR⁶R⁷ and R³, R⁴, and R⁵ are each independently H or OH. In some embodiments, R² is —C(═O)NR⁶R⁷ and R³, R⁴, and R⁵ are each independently H or OH.

In some embodiments, R³ is —(CH₂)C(═O)NR⁶R⁷ and R², R⁴, and R⁵ are each independently H or OH. In some embodiments, R³ is —C(═O)NR⁶R⁷ and R², R⁴, and R⁵ are each independently H or OH.

In some embodiments, R³ is —C(═O)NR⁶R⁷ and R², R⁴, and R⁵ are each independently H.

In some embodiments, R⁴ is —(CH₂)C(═O)NR⁶R⁷ and R², R³, and R⁵ are each independently H or OH. In some embodiments, R⁴ is —C(═O)NR⁶R⁷ and R², R³, and R⁵ are each independently H or OH.

In some embodiments, R⁵ is —(CH₂)C(═O)NR⁶R⁷ and R², R³, and R⁴ are each independently H or OH. In some embodiments, R⁵ is —C(═O)NR⁶R⁷ and R², R³, and R⁴ are each independently H or OH.

In some embodiments, R⁶ is H.

In some embodiments, R⁶ is —C₁-C₃ alkyl. In some embodiments, R⁶ is methyl. In some embodiments, R⁶ is ethyl. In some embodiments, R⁶ is propyl.

In some embodiments, R⁶ is —(C₁-C₃ alkylene)-(C₃-C₆ cycloalkyl). In some embodiments, R⁶ is —(CH₂)-(cyclopropyl).

In some embodiments, R⁶ is H, methyl, ethyl, propyl, or —(CH₂)-(cyclopropyl).

In some embodiments, R⁷ is —C₁-C₆ alkyl optionally substituted with 1-2 substituents independently selected from —C₁-C₃ alkyl, OH, oxo (═O), and —O—(C₁-C₃ alkyl). In some embodiments, R⁷ is —C₁-C₆ alkyl optionally substituted with 1-2 OH substituents. In some embodiments, R⁷ is ethyl. In some embodiments, R⁷ is —(CH₂)₂—OH. In some embodiments, R⁷ is propyl. In some embodiments, R⁷ is —CH₂CH(OH)CH₃. In some embodiments, R⁷ is —CH₂CH(OH)CH₂OH. In some embodiments, R⁷ is isopropyl. In some embodiments, R⁷ is butyl.

In some embodiments, R⁷ is isobutyl. In some embodiments, R⁷ is —CH₂C(CH₃)₂CH₂OH.

In some embodiments, R⁷ is —C₂-C₄ alkyl. In some embodiments, R⁷ is ethyl. In some embodiments, R⁷ is propyl. In some embodiments, R⁷ is isopropyl. In some embodiments, R⁷ is butyl. In some embodiments, R⁷ is isobutyl.

In some embodiments, R⁷ is —C₁-C₃ haloalkyl. In some embodiments, R⁷ is —CH₂CF₃.

In some embodiments, R⁷ is —(C₁-C₃ alkylene)-O—(C₁-C₃ alkyl). In some embodiments, R⁷ is —(CH₂)₃O—CH₃.

In some embodiments, R⁷ is —(C₁-C₃ alkylene)-N(C₁-C₃ alkyl)₂. In some embodiments, R⁷ is —(CH₂)₂—N(CH₃)₂.

In some embodiments, R⁷ is, —(C₁-C₃ alkylene)C(═O)NR⁸R⁹. In some embodiments, R⁷ is —(CH₂)₂—C(═O)NR⁸R⁹.

In some embodiments, R⁷ is —C₁-C₃ alkylene-phenyl, wherein the —C₁-C₃ alkylene is optionally substituted with 1-2 substituents independently selected from —C₁-C₃ alkyl, OH, oxo (═O), and —O—(C₁-C₃ alkyl).

In some embodiments, R⁷ is —C₁-C₃ alkylene-phenyl, wherein the —C₁-C₃ alkylene is optionally substituted with 1-2 OH substituents. In some embodiments, R⁷ is —CH₂-phenyl. In some embodiments, R⁷ is —(CH₂)₂-phenyl. In some embodiments, R⁷ is —(CH₂)₃-phenyl. In some embodiments, R⁷ is —(CH₂CH(OH))-phenyl.

In some embodiments, R⁷ is —C₁-C₃ alkylene-phenyl. In some embodiments, R⁷ is —CH₂— phenyl. In some embodiments, R⁷ is —(CH₂)₂-phenyl. In some embodiments, R⁷ is —(CH₂)₃-phenyl.

In some embodiments, R⁷ is —C₃-C₆ cycloalkyl. In some embodiments, R⁷ is cyclopropyl.

In some embodiments, R⁷ is cyclobutyl. In some embodiments, R⁷ is cyclopentyl. In some embodiments, R⁷ is cyclohexyl.

In some embodiments, R⁷ is a 4-7 membered heterocyclic ring optionally substituted with 1-2 substituents independently selected from —C₁-C₃ alkyl, OH, oxo (═O), and —O—(C₁-C₃ alkyl).

In some embodiments, R⁷ is tetrahydrofuranyl optionally substituted with 1-2 —C₁-C₃ alkyl substituents.

In some embodiments, R⁷ is pyrrolidinyl optionally substituted with 1-2 substituents independently selected from —C₁-C₃ alkyl and —O—(C₁-C₃ alkyl).

In some embodiments, R⁷ is dihydrothiophenyl optionally substituted with 1-2 oxo (C═O) substituents.

In some embodiments, R⁷ is piperidinyl optionally substituted with 1-2 oxo (C═O) substituents.

In some embodiments, R⁷ is caprolactamyl.

In some embodiments, R⁷ is —(C₁-C₃ alkylene)-(4-7 membered heterocyclic ring), wherein the 4-7 membered heterocyclic ring is optionally substituted with 1-2 substituents independently selected from —C₁-C₃ alkyl, OH, oxo (═O), and —O—(C₁-C₃ alkyl). In some embodiments, R⁷ is —(C₁-C₃ alkylene)-(4-7 membered heterocyclic ring), wherein the 4-7 membered ring is optionally substituted with 1-2 —C₁-C₃ alkyl.

In some embodiments, R⁷ is —(C₁-C₃ alkylene)-(4-7 membered heterocyclic ring), wherein the 4-7 membered heterocyclic ring is pyrazolyl, wherein the pyrazolyl is optionally substituted with 1-2 substituents independently selected from methyl and ethyl. In some embodiments, R⁷ is —(CH₂)(pyrazolyl), wherein the pyrazolyl is optionally substituted with 1-2 —C₁-C₃ alkyl substituents. In some embodiments, R⁷ is —(CH₂)(methylpyrazolyl). In some embodiments, R⁷ is —(CH₂)(ethylpyrazolyl). In some embodiments, R⁷ is —(CH₂)₂(pyrazolyl), wherein the pyrazolyl is optionally substituted with 1-2 —C₁-C₃ alkyl substituents. In some embodiments, R⁷ is —(CH₂)₂(pyrazolyl). In some embodiments, R⁷ is —(CH₂)₂(methylpyrazolyl). In some embodiments, R⁷ is —(CH₂)₂(pyrazolyl). In some embodiments, R⁷ is —(CH₂)₃(pyrazolyl).

In some embodiments, R⁷ is —(CH₂)₂(triazolyl), wherein the triazolyl is optionally substituted with 1-2 —C₁-C₃ alkyl substituents. In some embodiments, R⁷ is —(CH₂)₂(methyltriazolyl). In some embodiments, R⁷ is —CH(CH₃)(triazolyl). In some embodiments, R⁷ is —(CH(CH₃)CH₂)(triazolyl).

In some embodiments, R⁷ is —(CH₂)₂(tetrazolyl).

In some embodiments, R⁷ is —(CH₂)(thiazolyl), wherein the thiazolyl is optionally substituted with 1-2 —C₁-C₃ alkyl substituents.

In some embodiments, R⁷ is —(CH₂)₂(furanyl).

In some embodiments, R⁷ is —(CH₂)(tetrahydrofuranyl). In some embodiments, R⁷ is —(CH₂)₂(tetrahydrofuranyl).

In some embodiments, R⁷ is —(CH₂)(isoxazolyl), wherein the isoxazolyl is optionally substituted with 1-2 —C₁-C₃ alkyl substituents. In some embodiments, R⁷ is —(CH₂)(isoxazolyl).

In some embodiments, R⁷ is —(CH₂)(methylisoxazolyl).

In some embodiments, R⁷ is —(CH₂)(oxadiazolyl), wherein the oxadiazolyl is optionally substituted with 1-2 —C₁-C₃ alkyl substituents.

In some embodiments, R⁷ is —(CH₂)(pyridinyl), wherein the pyridinyl is optionally substituted with 1-2 —C₁-C₃ alkyl substituents. In some embodiments, R⁷ is —(CH₂)(ethylpyridinyl). In some embodiments, R⁷ is —(CH₂)(pyridinyl).

In some embodiments, R⁷ is —(CH₂)(pyrazinyl), wherein the pyrazinyl is optionally substituted with 1-2 —C₁-C₃ alkyl substituents. In some embodiments, R⁷ is —(CH₂)(methylpyrazinyl).

In some embodiments, R⁷ is —(CH₂)₂(piperidinyl).

In some embodiments, R⁷ is —(CH₂)(tetrahydropyranyl). In some embodiments, R⁷ is —(CH₂)₂(tetrahydropyranyl).

In some embodiments, R⁷ is —(CH₂)₂(morpholinyl).

In some embodiments, R⁷ is —(C₁-C₃ alkylene)-NR⁸R⁹. In some embodiments, R⁷ is —(CH₂)₂—NR⁸R⁹.

In some embodiments, R⁷ is —(C₁-C₃ alkylene)-C(═O)—O—(C₁-C₃ alkyl). In some embodiments, R⁷ is —(CH₂)₃—C(═O)—O—CH₃. In some embodiments, R⁷ is —(CH₂)₂—C(═O)—O—(CH₂)₂.

In some embodiments, R⁷ is —OH.

In some embodiments, R⁶ is C₁-C₃ alkyl and R⁷ is C₁-C₆ alkyl. In some embodiments, R⁶ is methyl and R⁷ is ethyl.

In some embodiments, R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, C₃-C₆ cycloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹.

In some embodiments, R⁶ and R⁷, together with the nitrogen to which they are attached, form an azetidinyl ring optionally substituted with OH.

In some embodiments, R⁶ and R⁷, together with the nitrogen to which they are attached, form a pyrrolidinyl ring optionally substituted with 1-2 substituents independently selected from OH, —(C₁-C₃ alkylene)-OH, halogen, —C(═O)NR⁸R⁹, —NR⁸R⁹, and —(C₁-C₃ alkylene)-O—(C₁-C₃ alkyl).

In some embodiments, R⁶ and R⁷, together with the nitrogen to which they are attached, form a piperidinyl ring optionally substituted with 1-2 substituents independently selected from OH, phenyl, —(C₁-C₃ alkylene)-OH, —C(═O)NR⁸R⁹, —C(═O)—O—(C₁-C₃ alkyl), —O—(C₁-C₃ alkylene)-O—(C₁-C₃ alkyl), —C(═O)—(C₁-C₃ alkyl), —(C₁-C₃ alkylene)-C(═O)—O—(C₁-C₃ alkyl), and —O—(C₁-C₃ alkyl).

In some embodiments, R⁶ and R⁷, together with the nitrogen to which they are attached, form a piperazinyl ring optionally substituted with 1-3 substituents independently selected from —C₁-C₃ alkyl, —C₁-C₃ haloalkyl, C₃-C₆ cycloalkyl, oxo (C═O), —(C₁-C₃ alkylene)-(4-6 membered heterocyclic ring) and —(C₁-C₃ alkylene)-C(═O)NR⁸R⁹.

In some embodiments, R⁶ and R⁷, together with the nitrogen to which they are attached, form a morpholinyl ring optionally substituted with —C(═O)NR⁸R⁹.

In some embodiments, R⁶ and R⁷, together with the nitrogen to which they are attached, form a thiomorpholinyl ring optionally substituted with 1-2 oxo (═O).

In some embodiments, R⁶ and R⁷, together with the nitrogen to which they are attached, form a diazepanyl ring optionally substituted with 1-2 substituents selected from —C₁-C₃ alkyl and oxo (═O).

In some embodiments, R⁶ and R⁷, together with the nitrogen to which they are attached, form an azabicyclo[3.2.1]octanyl group.

In some embodiments, R⁶ and R⁷, together with the nitrogen to which they are attached, form a diazaspiro[2.5]octanyl group optionally substituted with —C₁-C₃ alkyl.

In some embodiments, R⁶ and R⁷, together with the nitrogen to which they are attached, form an oxaazaspiro[3.4]octanyl group.

In some embodiments, R⁶ and R⁷, together with the nitrogen to which they are attached, form a dihydrobenzo[1,4]oxazepinyl group.

In some embodiments, R⁶ and R⁷, together with the nitrogen to which they are attached, form a 4-6 membered heterocyclic ring optionally substituted with 1-3 substituents independently selected from —C₁-C₃ alkyl, —OH, —C₁-C₃ haloalkyl, C₃-C₆ cycloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹.

In some embodiments, R⁶ and R⁷, together with the nitrogen to which they are attached, form a heterocyclic ring selected from pyrrolidinyl, piperidinyl, and piperazinyl;

wherein the pyrrolidinyl is optionally substituted with 1-2 substituents independently selected from OH, —(C₁-C₃ alkylene)-OH, halogen, —C(═O)NR⁸R⁹, —NR⁸R⁹, and —(C₁-C₃ alkylene)-O—(C₁-C₃ alkyl);

wherein the piperidinyl is optionally substituted with 1-2 substituents independently selected from OH, phenyl, —(C₁-C₃ alkylene)-OH, —C(═O)NR⁸R⁹, —C(═O)—O—(C₁-C₃ alkyl), —O—(C₁-C₃ alkylene)-O—(C₁-C₃ alkyl), —C(═O)—(C₁-C₃ alkyl), —(C₁-C₃ alkylene)-C(═O)—O—(C₁-C₃ alkyl), and —O—(C₁-C₃ alkyl); and

wherein the piperazinyl ring is optionally substituted with 1-3 substituents independently selected from —C₁-C₃ alkyl, —C₁-C₃ haloalkyl, C₃-C₆ cycloalkyl, oxo (C═O), —(C₁-C₃ alkylene)-(4-6 membered heterocyclic ring) and —(C₁-C₃ alkylene)-C(═O)NR⁸R⁹.

In some embodiments, R⁸ is H and R⁹ is H.

In some embodiments, R⁸ is H and R⁹ is —C₁-C₃ alkyl. In some embodiments, R⁸ is H and R⁹ is methyl. In some embodiments, R⁸ is H and R⁹ is isopropyl.

In some embodiments, R⁸ and R⁹ are each independently —C₁-C₃ alkyl. In some embodiments, R⁸ is methyl and R⁹ is methyl. In some embodiments, R⁸ is ethyl and R⁹ is ethyl.

In some embodiments, X is N; R¹ is phenyl; R², R⁴, and R⁵ are each H; R³ is —C(═O)NR⁶R⁷; and R⁶ and R⁷ are each —C₁-C₃ alkyl.

In some embodiments, X is N; R¹ is phenyl; R², R⁴, and R⁵ are each H; R³ is —C(═O)NR⁶R⁷; R⁶ is methyl; and R⁷ is ethyl.

In some embodiments, the compound is a compound of Formula (Va)

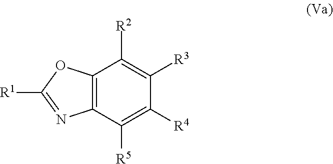

and pharmaceutically acceptable salts thereof, wherein:

R¹ is H, —C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂,

wherein the phenyl is optionally substituted with 1-5 substituents independently selected from halogen, C₁-C₃ alkyl, —O—(C₁-C₃ alkyl), and —C₁-C₃ haloalkyl;

one of R², R³, R⁴, and R⁵ is —(CH₂)ₙC(═O)NR⁶R⁷ and the others are each independently H or —OH;

R⁶ is H, —C₁-C₃ alkyl, or —(C₁-C₃ alkylene)-(C₃-C₆ cycloalkyl);

R⁷ is —C₁-C₆ alkyl, —C₁-C₃ haloalkyl, —(C₁-C₃ alkylene)-O—(C₁-C₃ alkyl), —(C₁-C₃ alkylene)-N(C₁-C₃ alkyl)₂, —(C₁-C₃ alkylene)C(═O)NR⁸R⁹, —(C₁-C₃ alkylene)-phenyl, —C₃-C₆ cycloalkyl, 4-7 membered heterocyclic ring, —(C₁-C₃ alkylene)-(4-7 membered heterocyclic ring), —(C₁-C₃ alkylene)-NR⁸R⁹, —(C₁-C₃ alkylene)-C(═O)—O—(C₁-C₃ alkyl), or —OH,

wherein the —C₁-C₆ alkyl, —C₁-C₃ alkylene, and 4-7 membered heterocyclic ring are each optionally substituted with 1-2 substituents independently selected from —C₁-C₃ alkyl, OH, oxo (═O), and —O—(C₁-C₃ alkyl);

or R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹;

R⁸ and R⁹ are each independently H or —C₁-C₃ alkyl; and

n is 0 or 1.

In some embodiments, the compound is a compound of Formula (Va)

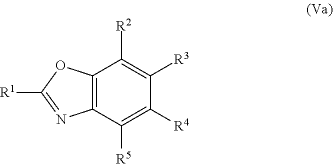

and pharmaceutically acceptable salts thereof, wherein:

R¹ is H, —C₁-C₃ alkyl, —C₁-C₃ haloalkyl, —C₃-C₆ cycloalkyl, 4-6 membered heterocyclic ring, phenyl optionally substituted with 1-5 halogen, —SH, oxo (═O), or —N(C₁-C₃ alkyl)₂; one of R², R³, R⁴, and R⁵ is —(CH₂)ₙC(═O)NR⁶R⁷ and the others are each independently H or —OH;

R⁶ is H or —C₁-C₃ alkyl;

R⁷ is selected fro —C₂-C₄ alkyl, —(C₁-C₃ alkylene)-O—(C₁-C₃ alkyl), —(C₁-C₃ alkylene)-N(C₁-C₃ alkyl)₂, —C₁-C₃ alkylene-phenyl, —C₃-C₆ cycloalkyl, or —OH;

or R⁶ and R⁷, together with the nitrogen to which they are attached, form a 4-6 membered heterocyclic ring; and

n is 0 or 1.

In some embodiments, the compound is a compound of Formula (Vb)

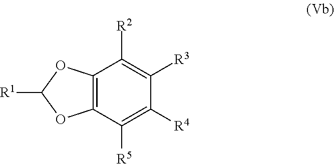

and pharmaceutically acceptable salts thereof, wherein:

R¹ is H, —C₁-C₃ alkyl, —C₁-C₃ haloalkyl, —C₃-C₆ cycloalkyl, 4-6 membered heterocyclic ring, phenyl optionally substituted with 1-5 halogen, —SH, oxo (═O), or —N(C₁-C₃ alkyl)₂;

one of R², R³, R⁴, and R⁵ is —(CH₂)ₙC(═O)NR⁶R⁷ and the others are each independently H or —OH;

R⁶ is H or —C₁-C₃ alkyl;

R⁷ is —C₂-C₄ alkyl, —(C₁-C₃ alkylene)-O—(C₁-C₃ alkyl), —(C₁-C₃ alkylene)-N(C₁-C₃ alkyl)₂, —C₁-C₃ alkylene-phenyl, —C₃-C₆ cycloalkyl, or —OH;

or R⁶ and R⁷, together with the nitrogen to which they are attached, form a 4-6 membered heterocyclic ring; and

n is 0 or 1.

In some embodiments, the compound is a compound of Formula (V) or a pharmaceutically acceptable salt thereof. In some embodiments, the compound is selected from:

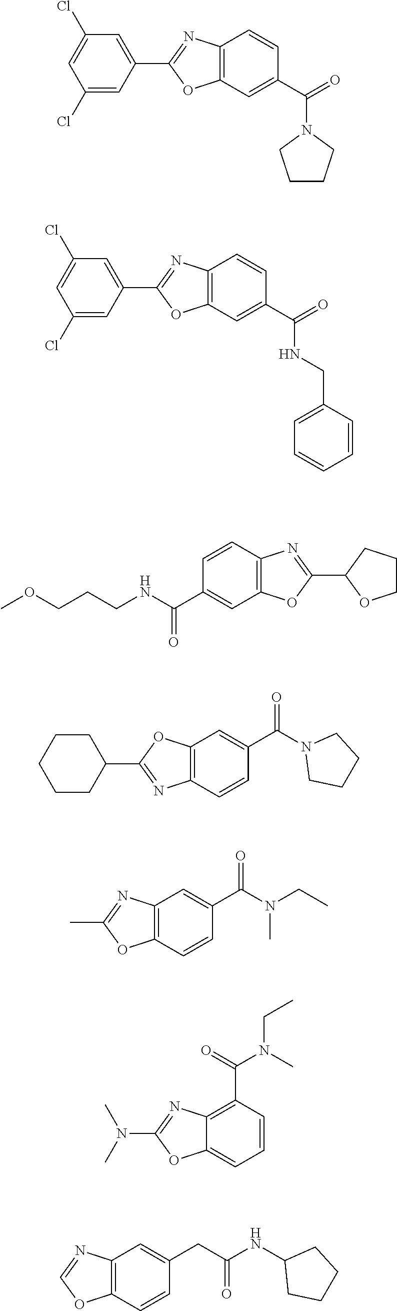

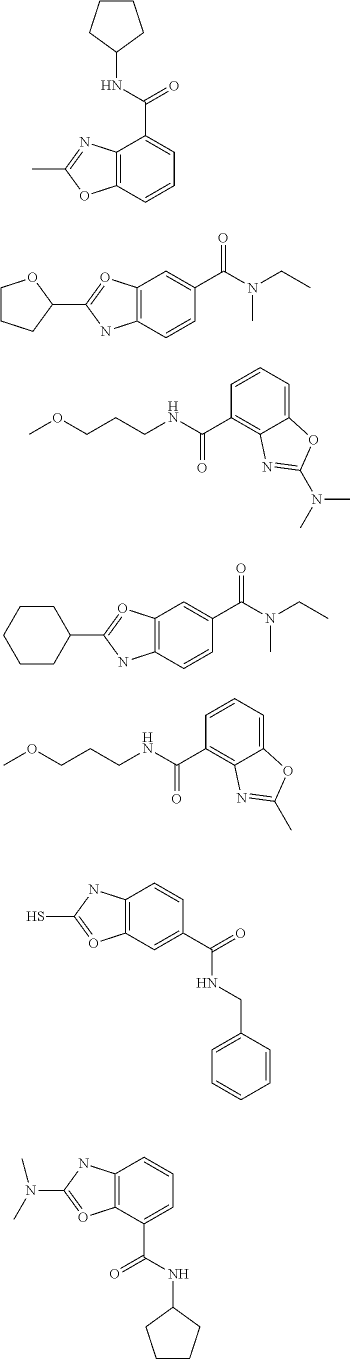

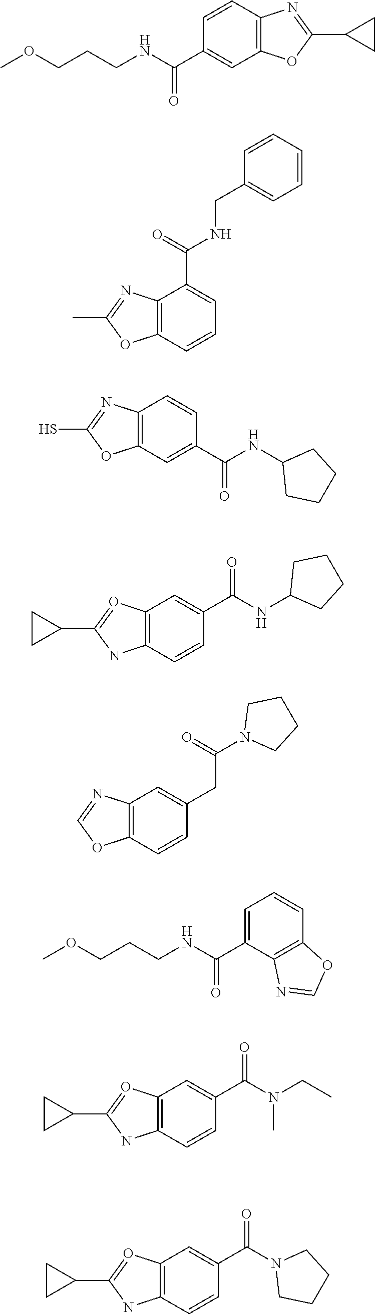

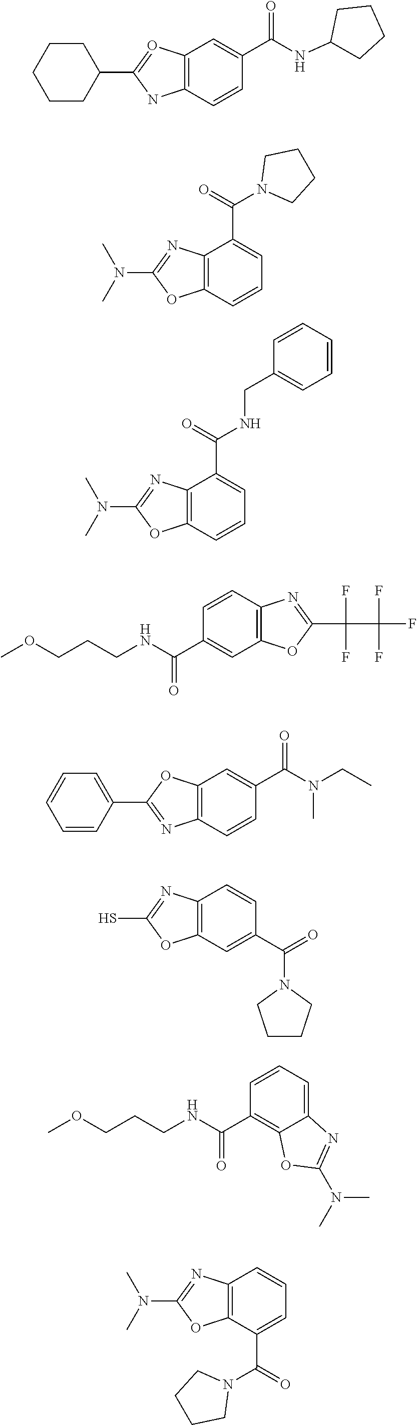

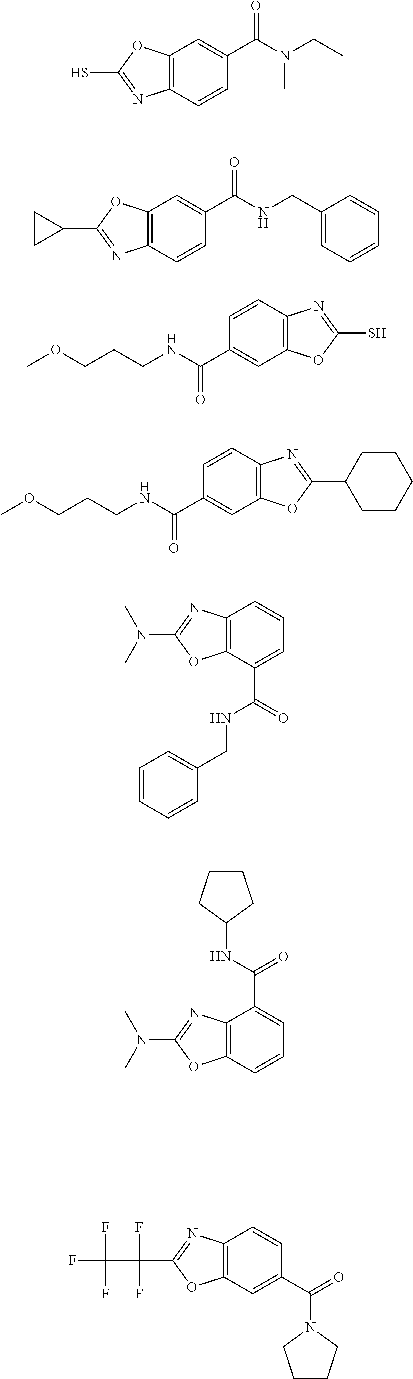

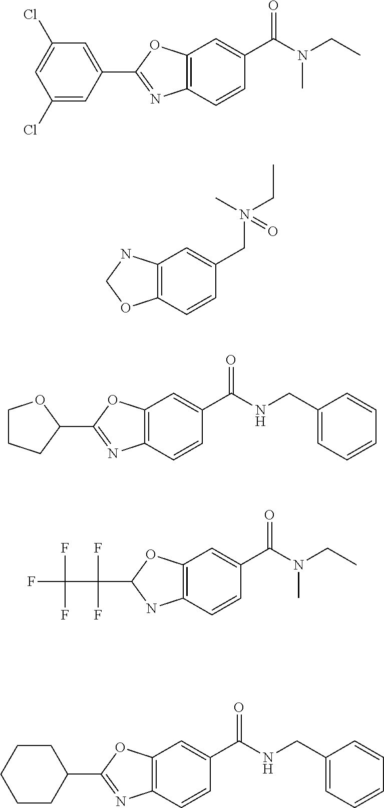 [SMILES: CCN(C)C(=O)c1ccc2nc(-c3cc(Cl)cc(Cl)c3)oc2c1 ; CC[N+](C)([O-])Cc1ccc2c(c1)NCO2 ; O=C(NCc1ccccc1)c1ccc2nc(C3CCCO3)oc2c1 ; CCN(C)C(=O)c1ccc2c(c1)OC(C(F)(F)C(F)(F)F)N2 ; O=C(NCc1ccccc1)c1ccc2nc(C3CCCCC3)oc2c1]

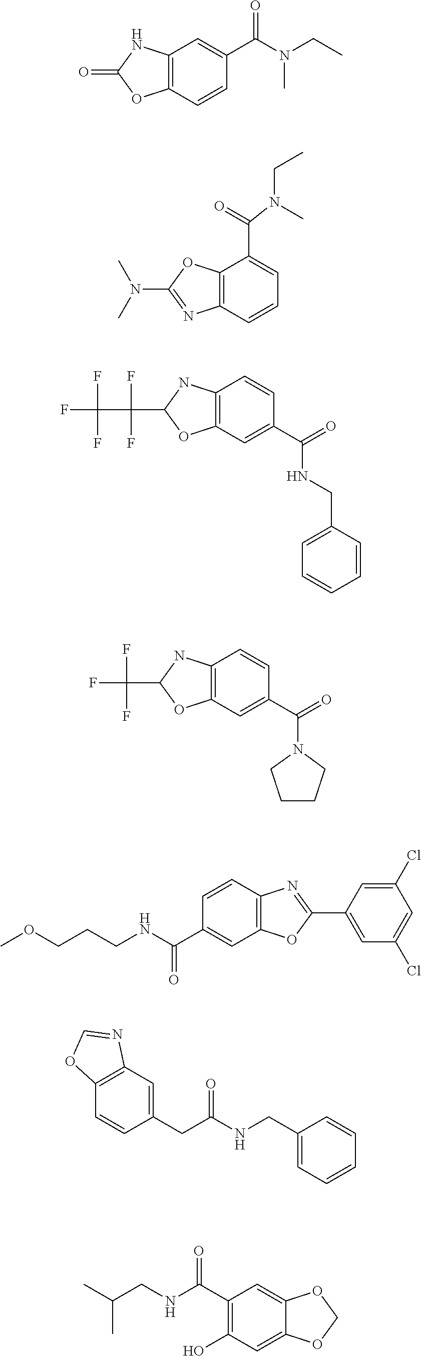

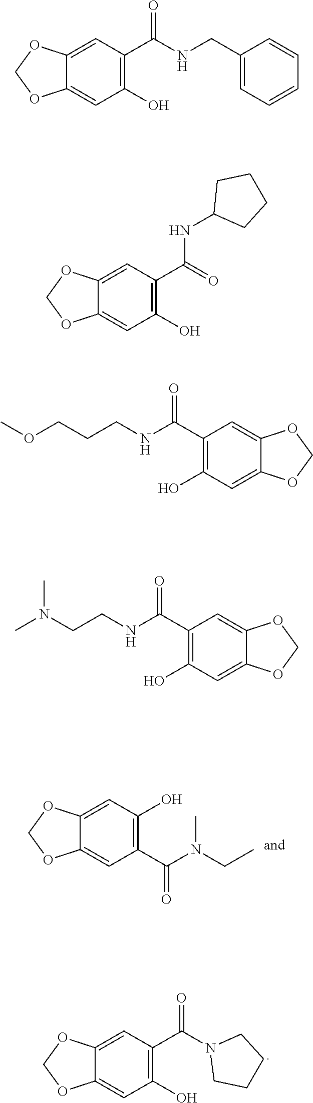 [SMILES: O=C(NCc1ccccc1)c1cc2c(cc1O)OCO2 ; O=C(NC1CCCC1)c1cc2c(cc1O)OCO2 ; COCCCNC(=O)c1cc2c(cc1O)OCO2 ; CN(C)CCNC(=O)c1cc2c(cc1O)OCO2 ; CCN(C)C(=O)c1cc2c(cc1O)OCO2 ; O=C(c1cc2c(cc1O)OCO2)N1CCCC1]

Illustrative compounds of Formula (V) are shown in Table 1.

TABLE 1

| | |
|---|---|
| 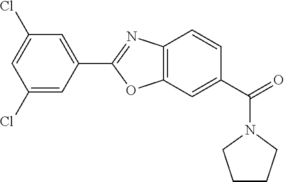 [SMILES: O=C(c1ccc2nc(-c3cc(Cl)cc(Cl)c3)oc2c1)N1CCCC1] | 1 |
| 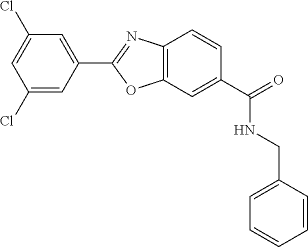 [SMILES: O=C(NCc1ccccc1)c1ccc2nc(-c3cc(Cl)cc(Cl)c3)oc2c1] | 2 |
| 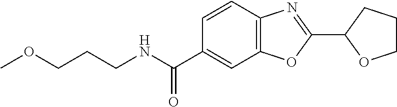 [SMILES: COCCCNC(=O)c1ccc2nc(C3CCCO3)oc2c1] | 3 |
| 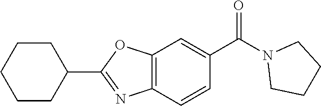 [SMILES: O=C(c1ccc2nc(C3CCCCC3)oc2c1)N1CCCC1] | 4 |
| 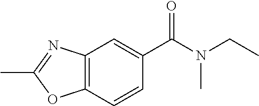 [SMILES: CCN(C)C(=O)c1ccc2oc(C)nc2c1] | 5 |
| 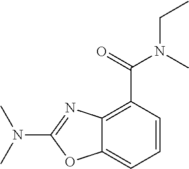 [SMILES: CCN(C)C(=O)c1cccc2oc(N(C)C)nc12] | 6 |
| 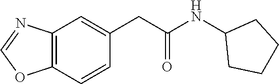 [SMILES: O=C(Cc1ccc2ocnc2c1)NC1CCCC1] | 7 |
| 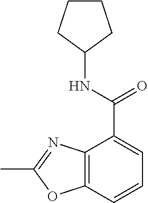 [SMILES: Cc1nc2c(C(=O)NC3CCCC3)cccc2o1] | 8 |
| 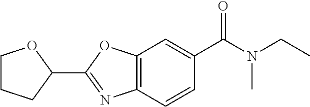 [SMILES: CCN(C)C(=O)c1ccc2nc(C3CCCO3)oc2c1] | 9 |
| 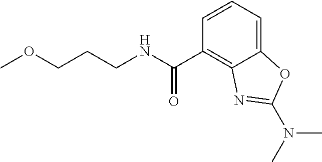 | 10 |
| 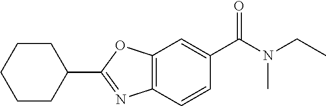 [SMILES: CCN(C)C(=O)c1ccc2nc(C3CCCCC3)oc2c1] | 11 |
| 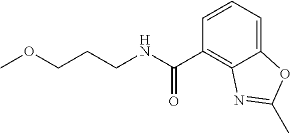 | 12 |
| 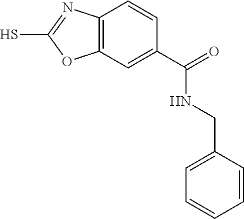 [SMILES: O=C(NCc1ccccc1)c1ccc2nc(S)oc2c1] | 13 |
| 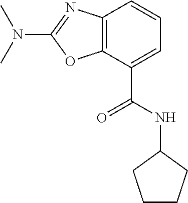 [SMILES: CN(C)c1nc2cccc(C(=O)NC3CCCC3)c2o1] | 14 |
| 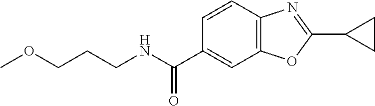 [SMILES: COCCCNC(=O)c1ccc2nc(C3CC3)oc2c1] | 15 |
| 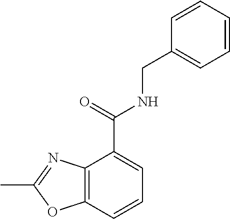 [SMILES: Cc1nc2c(C(=O)NCc3ccccc3)cccc2o1] | 16 |
| 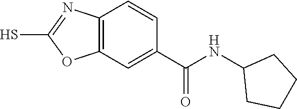 [SMILES: O=C(NC1CCCC1)c1ccc2nc(S)oc2c1] | 17 |
| 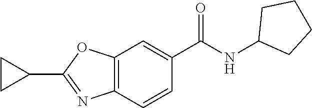 [SMILES: O=C(NC1CCCC1)c1ccc2nc(C3CC3)oc2c1] | 18 |
| 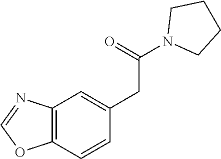 [SMILES: O=C(Cc1ccc2ocnc2c1)N1CCCC1] | 19 |
| 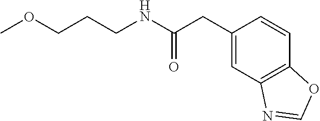 [SMILES: COCCCNC(=O)Cc1ccc2ocnc2c1] | 20 |
| 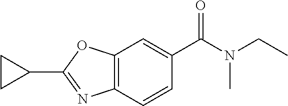 [SMILES: CCN(C)C(=O)c1ccc2nc(C3CC3)oc2c1] | 21 |
| 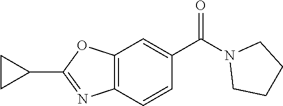 [SMILES: O=C(c1ccc2nc(C3CC3)oc2c1)N1CCCC1] | 22 |
| 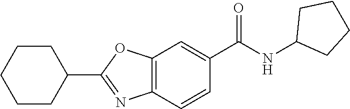 [SMILES: O=C(NC1CCCC1)c1ccc2nc(C3CCCCC3)oc2c1] | 23 |
| 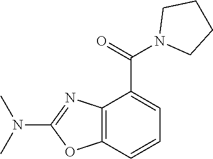 [SMILES: CN(C)c1nc2c(C(=O)N3CCCC3)cccc2o1] | 24 |
| 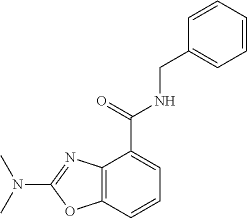 [SMILES: CN(C)c1nc2c(C(=O)NCc3ccccc3)cccc2o1] | 25 |
| 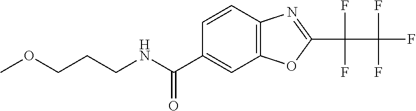 [SMILES: COCCCNC(=O)c1ccc2nc(C(F)(F)C(F)(F)F)oc2c1] | 26 |
| 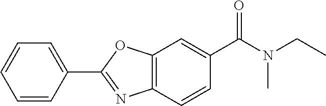 [SMILES: CCN(C)C(=O)c1ccc2nc(-c3ccccc3)oc2c1] | 27 |
| 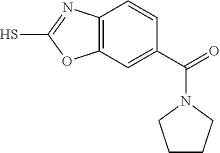 [SMILES: O=C(c1ccc2nc(S)oc2c1)N1CCCC1] | 28 |
| 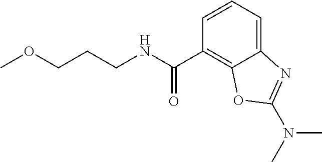 | 29 |
| 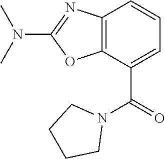 [SMILES: CN(C)c1nc2cccc(C(=O)N3CCCC3)c2o1] | 30 |
| 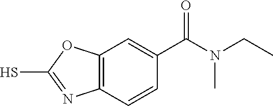 [SMILES: CCN(C)C(=O)c1ccc2nc(S)oc2c1] | 31 |
| 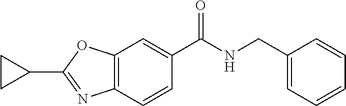 [SMILES: O=C(NCc1ccccc1)c1ccc2nc(C3CC3)oc2c1] | 32 |
| 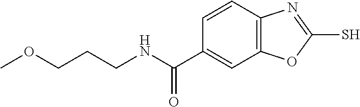 [SMILES: COCCCNC(=O)c1ccc2nc(S)oc2c1] | 33 |
| 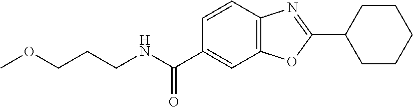 [SMILES: COCCCNC(=O)c1ccc2nc(C3CCCCC3)oc2c1] | 34 |
| 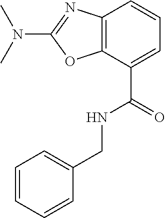 [SMILES: CN(C)c1nc2cccc(C(=O)NCc3ccccc3)c2o1] | 35 |
| 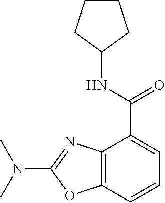 [SMILES: CN(C)c1nc2c(C(=O)NC3CCCC3)cccc2o1] | 36 |
| 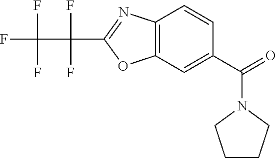 [SMILES: O=C(c1ccc2nc(C(F)(F)C(F)(F)F)oc2c1)N1CCCC1] | 37 |
| 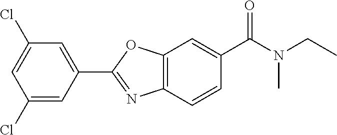 [SMILES: CCN(C)C(=O)c1ccc2nc(-c3cc(Cl)cc(Cl)c3)oc2c1] | 38 |
| 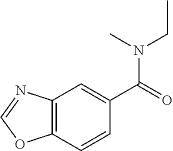 | 39 |
| 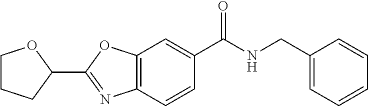 [SMILES: O=C(NCc1ccccc1)c1ccc2nc(C3CCCO3)oc2c1] | 40 |
| 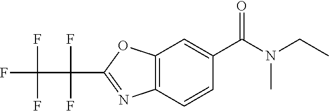 [SMILES: CCN(C)C(=O)c1ccc2nc(C(F)(F)C(F)(F)F)oc2c1] | 41 |
| 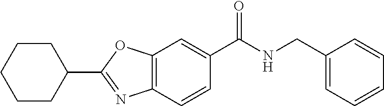 [SMILES: O=C(NCc1ccccc1)c1ccc2nc(C3CCCCC3)oc2c1] | 42 |
| 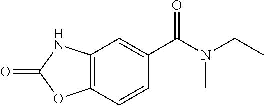 [SMILES: CCN(C)C(=O)c1ccc2oc(=O)[nH]c2c1] | 43 |
| 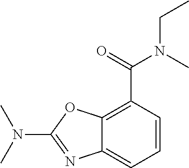 [SMILES: CCN(C)C(=O)c1cccc2nc(N(C)C)oc12] | 44 |
| 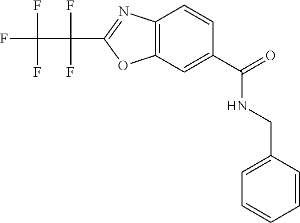 [SMILES: O=C(NCc1ccccc1)c1ccc2nc(C(F)(F)C(F)(F)F)oc2c1] | 45 |
| 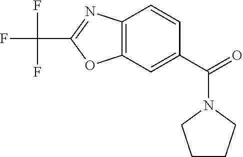 [SMILES: O=C(c1ccc2nc(C(F)(F)F)oc2c1)N1CCCC1] | 46 |
| 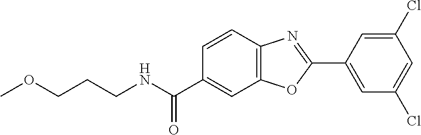 [SMILES: COCCCNC(=O)c1ccc2nc(-c3cc(Cl)cc(Cl)c3)oc2c1] | 47 |
| 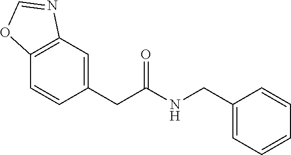 [SMILES: O=C(Cc1ccc2ocnc2c1)NCc1ccccc1] | 48 |
| 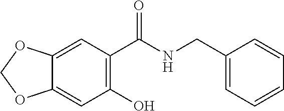 [SMILES: O=C(NCc1ccccc1)c1cc2c(cc1O)OCO2] | 49 |
| 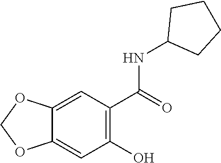 [SMILES: O=C(NC1CCCC1)c1cc2c(cc1O)OCO2] | 50 |
| 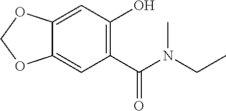 [SMILES: CCN(C)C(=O)c1cc2c(cc1O)OCO2] | 51 |
| 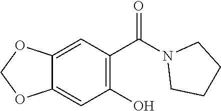 [SMILES: O=C(c1cc2c(cc1O)OCO2)N1CCCC1] | 52 |
| 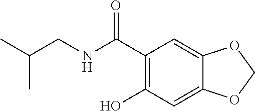 [SMILES: CC(C)CNC(=O)c1cc2c(cc1O)OCO2] | 53 |
| 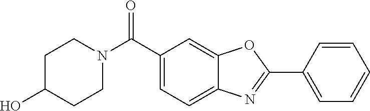 [SMILES: O=C(c1ccc2nc(-c3ccccc3)oc2c1)N1CCC(O)CC1] | 54 |
| 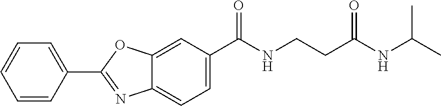 [SMILES: CC(C)NC(=O)CCNC(=O)c1ccc2nc(-c3ccccc3)oc2c1] | 55 |
| 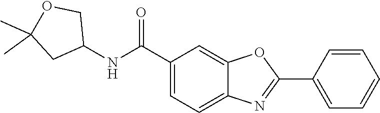 [SMILES: CC1(C)CC(NC(=O)c2ccc3nc(-c4ccccc4)oc3c2)CO1] | 56 |
| 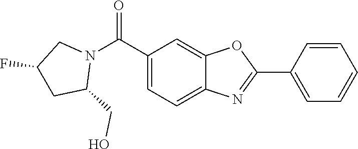 [SMILES: O=C(c1ccc2nc(-c3ccccc3)oc2c1)N1C[C@@H](F)C[C@H]1CO] | 57 |
| 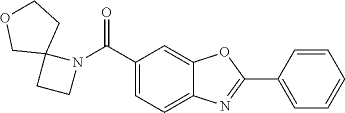 [SMILES: O=C(c1ccc2nc(-c3ccccc3)oc2c1)N1CCC12CCOC2] | 58 |
| 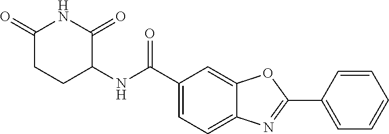 [SMILES: O=C1CCC(NC(=O)c2ccc3nc(-c4ccccc4)oc3c2)C(=O)N1] | 59 |
| 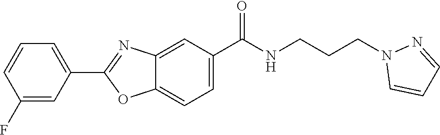 [SMILES: O=C(NCCCn1cccn1)c1ccc2oc(-c3cccc(F)c3)nc2c1] | 60 |
| 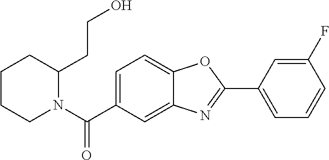 [SMILES: O=C(c1ccc2oc(-c3cccc(F)c3)nc2c1)N1CCCCC1CCO] | 61 |
| 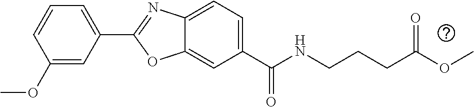 | 62 |
| 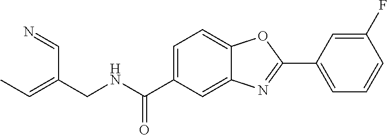 [SMILES: C/C=C(\C=N)CNC(=O)c1ccc2oc(-c3cccc(F)c3)nc2c1] | 63 |
| 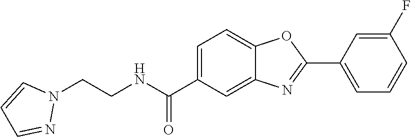 [SMILES: O=C(NCCn1cccn1)c1ccc2oc(-c3cccc(F)c3)nc2c1] | 64 |
| 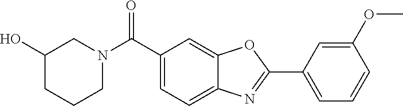 [SMILES: COc1cccc(-c2nc3ccc(C(=O)N4CCCC(O)C4)cc3o2)c1] | 65 |
| 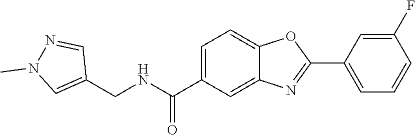 [SMILES: Cn1cc(CNC(=O)c2ccc3oc(-c4cccc(F)c4)nc3c2)cn1] | 66 |
| 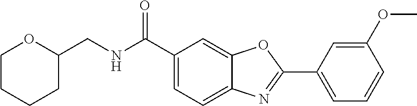 [SMILES: COc1cccc(-c2nc3ccc(C(=O)NCC4CCCCO4)cc3o2)c1] | 67 |
| 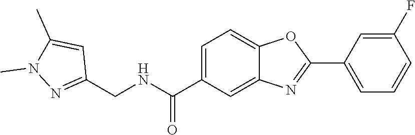 [SMILES: Cc1cc(CNC(=O)c2ccc3oc(-c4cccc(F)c4)nc3c2)nn1C] | 68 |
| 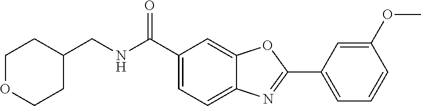 [SMILES: COc1cccc(-c2nc3ccc(C(=O)NCC4CCOCC4)cc3o2)c1] | 69 |
| 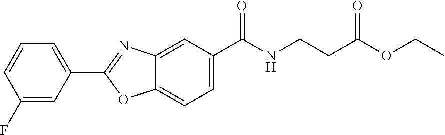 [SMILES: CCOC(=O)CCNC(=O)c1ccc2oc(-c3cccc(F)c3)nc2c1] | 70 |
| 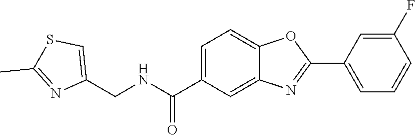 [SMILES: Cc1nc(CNC(=O)c2ccc3oc(-c4cccc(F)c4)nc3c2)cs1] | 71 |
| 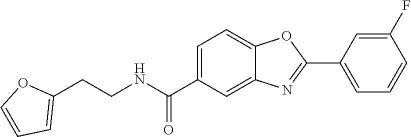 [SMILES: O=C(NCCc1ccco1)c1ccc2oc(-c3cccc(F)c3)nc2c1] | 72 |
| 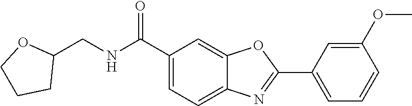 [SMILES: COc1cccc(-c2nc3ccc(C(=O)NCC4CCCO4)cc3o2)c1] | 73 |
| 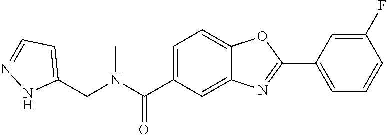 [SMILES: CN(Cc1ccn[nH]1)C(=O)c1ccc2oc(-c3cccc(F)c3)nc2c1] | 74 |
| 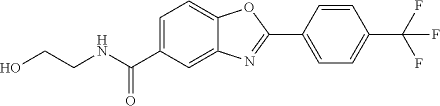 [SMILES: O=C(NCCO)c1ccc2oc(-c3ccc(C(F)(F)F)cc3)nc2c1] | 75 |
| 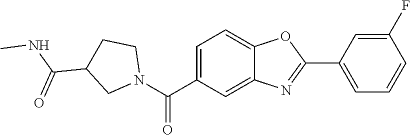 [SMILES: CNC(=O)C1CCN(C(=O)c2ccc3oc(-c4cccc(F)c4)nc3c2)C1] | 76 |
| 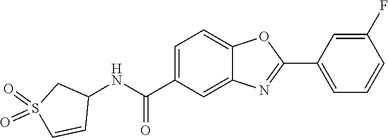 [SMILES: O=C(NC1C=CS(=O)(=O)C1)c1ccc2oc(-c3cccc(F)c3)nc2c1] | 77 |
| 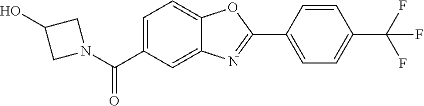 [SMILES: O=C(c1ccc2oc(-c3ccc(C(F)(F)F)cc3)nc2c1)N1CC(O)C1] | 78 |
| 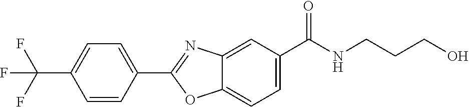 [SMILES: O=C(NCCCO)c1ccc2oc(-c3ccc(C(F)(F)F)cc3)nc2c1] | 79 |
| 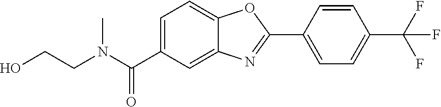 [SMILES: CN(CCO)C(=O)c1ccc2oc(-c3ccc(C(F)(F)F)cc3)nc2c1] | 80 |
| 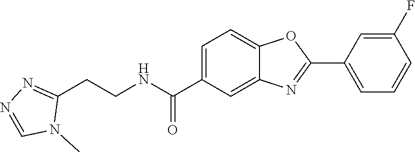 [SMILES: Cn1cnnc1CCNC(=O)c1ccc2oc(-c3cccc(F)c3)nc2c1] | 81 |
| 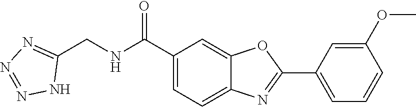 [SMILES: COc1cccc(-c2nc3ccc(C(=O)NCc4nnn[nH]4)cc3o2)c1] | 82 |
| 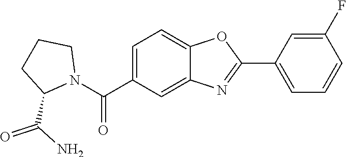 [SMILES: NC(=O)[C@@H]1CCCN1C(=O)c1ccc2oc(-c3cccc(F)c3)nc2c1] | 83 |
| 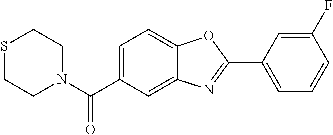 [SMILES: O=C(c1ccc2oc(-c3cccc(F)c3)nc2c1)N1CCSCC1] | 84 |
| 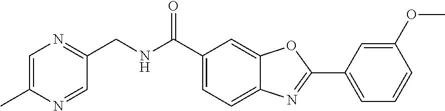 | 85 |
| 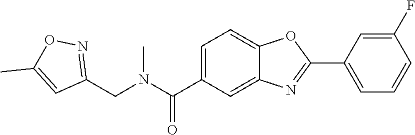 [SMILES: Cc1cc(CN(C)C(=O)c2ccc3oc(-c4cccc(F)c4)nc3c2)no1] | 86 |
| 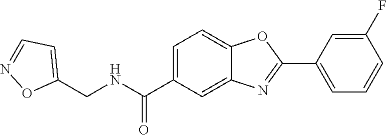 [SMILES: O=C(NCc1ccno1)c1ccc2oc(-c3cccc(F)c3)nc2c1] | 87 |
| 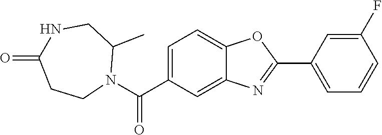 [SMILES: CC1CNC(=O)CCN1C(=O)c1ccc2oc(-c3cccc(F)c3)nc2c1] | 88 |
| 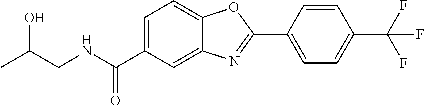 [SMILES: CC(O)CNC(=O)c1ccc2oc(-c3ccc(C(F)(F)F)cc3)nc2c1] | 89 |
| 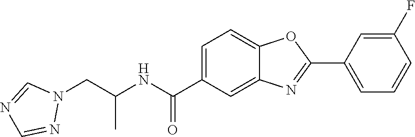 [SMILES: CC(Cn1cncn1)NC(=O)c1ccc2oc(-c3cccc(F)c3)nc2c1] | 90 |
| 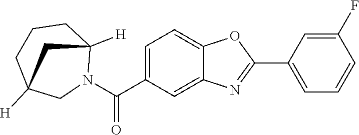 [SMILES: O=C(c1ccc2oc(-c3cccc(F)c3)nc2c1)N1C[C@@H]2CCC[C@H]1C2] | 91 |
| 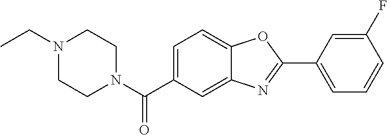 [SMILES: CCN1CCN(C(=O)c2ccc3oc(-c4cccc(F)c4)nc3c2)CC1] | 92 |
| 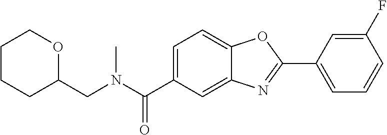 [SMILES: CN(CC1CCCCO1)C(=O)c1ccc2oc(-c3cccc(F)c3)nc2c1] | 93 |
| 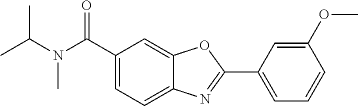 [SMILES: COc1cccc(-c2nc3ccc(C(=O)N(C)C(C)C)cc3o2)c1] | 94 |
| 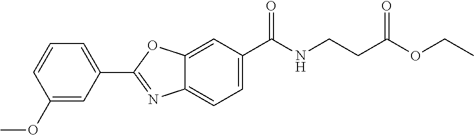 [SMILES: CCOC(=O)CCNC(=O)c1ccc2nc(-c3cccc(OC)c3)oc2c1] | 95 |
| 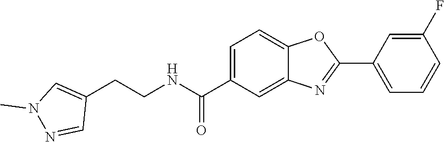 [SMILES: Cn1cc(CCNC(=O)c2ccc3oc(-c4cccc(F)c4)nc3c2)cn1] | 96 |
| 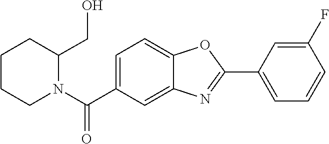 [SMILES: O=C(c1ccc2oc(-c3cccc(F)c3)nc2c1)N1CCCCC1CO] | 97 |
| 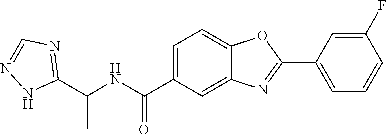 [SMILES: CC(NC(=O)c1ccc2oc(-c3cccc(F)c3)nc2c1)c1ncn[nH]1] | 98 |
| 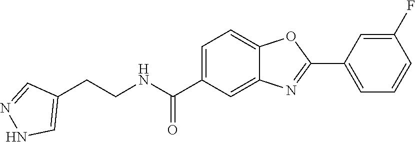 [SMILES: O=C(NCCc1cn[nH]c1)c1ccc2oc(-c3cccc(F)c3)nc2c1] | 99 |
| 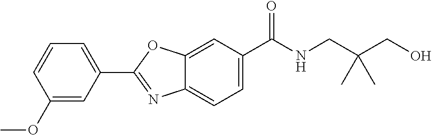 [SMILES: COc1cccc(-c2nc3ccc(C(=O)NCC(C)(C)CO)cc3o2)c1] | 100 |
| 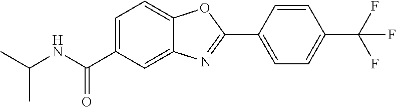 [SMILES: CC(C)NC(=O)c1ccc2oc(-c3ccc(C(F)(F)F)cc3)nc2c1] | 101 |
| 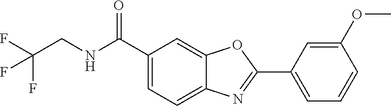 [SMILES: COc1cccc(-c2nc3ccc(C(=O)NCC(F)(F)F)cc3o2)c1] | 102 |
| 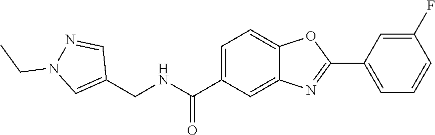 [SMILES: CCn1cc(CNC(=O)c2ccc3oc(-c4cccc(F)c4)nc3c2)cn1] | 103 |
| 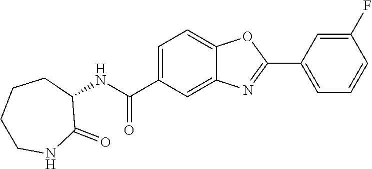 [SMILES: O=C(N[C@H]1CCCCNC1=O)c1ccc2oc(-c3cccc(F)c3)nc2c1] | 104 |
| 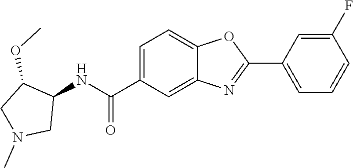 [SMILES: CO[C@H]1CN(C)C[C@@H]1NC(=O)c1ccc2oc(-c3cccc(F)c3)nc2c1] | 105 |
| 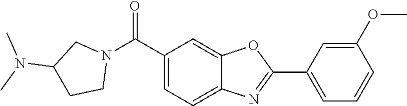 | 106 |
| 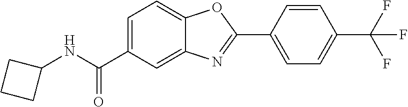 [SMILES: O=C(NC1CCC1)c1ccc2oc(-c3ccc(C(F)(F)F)cc3)nc2c1] | 107 |
| 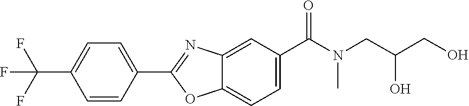 [SMILES: CN(CC(O)CO)C(=O)c1ccc2oc(-c3ccc(C(F)(F)F)cc3)nc2c1] | 108 |
| 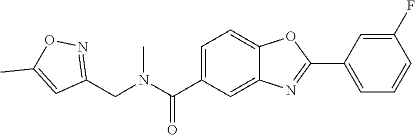 [SMILES: Cc1cc(CN(C)C(=O)c2ccc3oc(-c4cccc(F)c4)nc3c2)no1] | 109 |
| 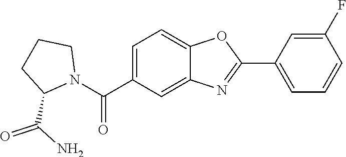 [SMILES: NC(=O)[C@@H]1CCCN1C(=O)c1ccc2oc(-c3cccc(F)c3)nc2c1] | 110 |
| 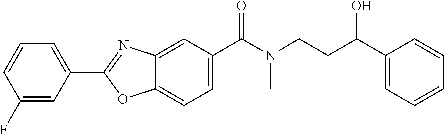 [SMILES: CN(CCC(O)c1ccccc1)C(=O)c1ccc2oc(-c3cccc(F)c3)nc2c1] | 111 |
| 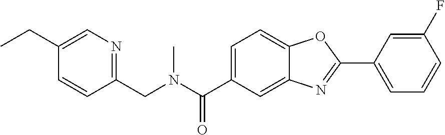 [SMILES: CCc1ccc(CN(C)C(=O)c2ccc3oc(-c4cccc(F)c4)nc3c2)nc1] | 112 |
| 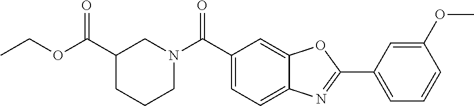 [SMILES: CCOC(=O)C1CCCN(C(=O)c2ccc3nc(-c4cccc(OC)c4)oc3c2)C1] | 113 |
| 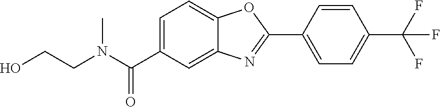 [SMILES: CN(CCO)C(=O)c1ccc2oc(-c3ccc(C(F)(F)F)cc3)nc2c1] | 114 |
| 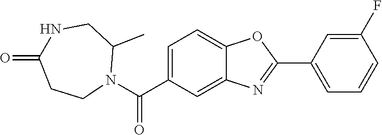 [SMILES: CC1CNC(=O)CCN1C(=O)c1ccc2oc(-c3cccc(F)c3)nc2c1] | 115 |
| 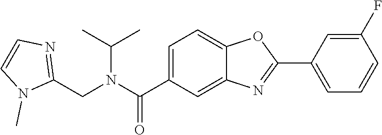 [SMILES: CC(C)N(Cc1nccn1C)C(=O)c1ccc2oc(-c3cccc(F)c3)nc2c1] | 116 |
| 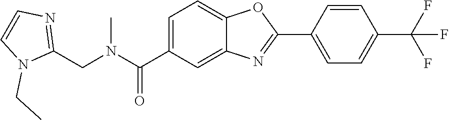 [SMILES: CCn1ccnc1CN(C)C(=O)c1ccc2oc(-c3ccc(C(F)(F)F)cc3)nc2c1] | 117 |
| 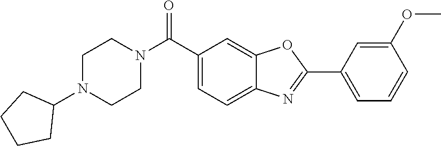 [SMILES: COc1cccc(-c2nc3ccc(C(=O)N4CCN(C5CCCC5)CC4)cc3o2)c1] | 118 |
| 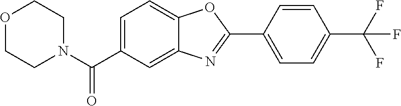 [SMILES: O=C(c1ccc2oc(-c3ccc(C(F)(F)F)cc3)nc2c1)N1CCOCC1] | 119 |
| 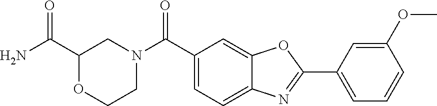 [SMILES: COc1cccc(-c2nc3ccc(C(=O)N4CCOC(C(N)=O)C4)cc3o2)c1] | 120 |
| 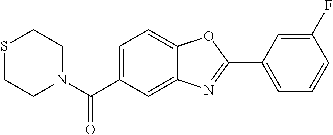 [SMILES: O=C(c1ccc2oc(-c3cccc(F)c3)nc2c1)N1CCSCC1] | 121 |
| 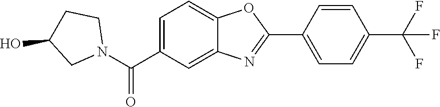 [SMILES: O=C(c1ccc2oc(-c3ccc(C(F)(F)F)cc3)nc2c1)N1CC[C@H](O)C1] | 122 |
| 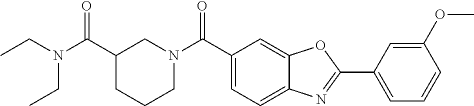 [SMILES: CCN(CC)C(=O)C1CCCN(C(=O)c2ccc3nc(-c4cccc(OC)c4)oc3c2)C1] | 123 |
| 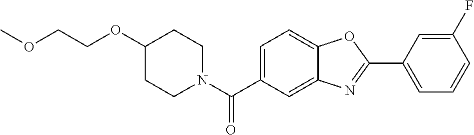 [SMILES: COCCOC1CCN(C(=O)c2ccc3oc(-c4cccc(F)c4)nc3c2)CC1] | 124 |
| 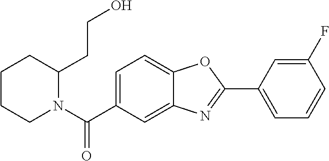 [SMILES: O=C(c1ccc2oc(-c3cccc(F)c3)nc2c1)N1CCCCC1CCO] | 125 |
| 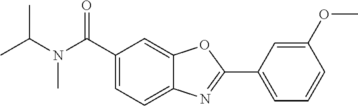 [SMILES: COc1cccc(-c2nc3ccc(C(=O)N(C)C(C)C)cc3o2)c1] | 126 |
| 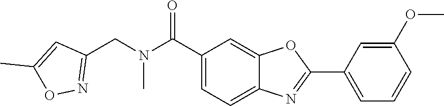 | 127 |
| 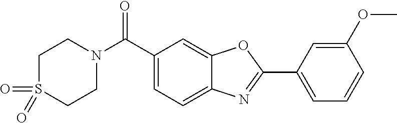 [SMILES: COc1cccc(-c2nc3ccc(C(=O)N4CCS(=O)(=O)CC4)cc3o2)c1] | 128 |
| 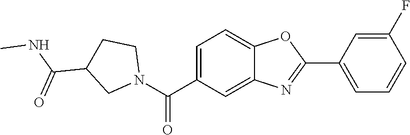 [SMILES: CNC(=O)C1CCN(C(=O)c2ccc3oc(-c4cccc(F)c4)nc3c2)C1] | 129 |
| 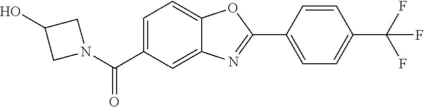 [SMILES: O=C(c1ccc2oc(-c3ccc(C(F)(F)F)cc3)nc2c1)N1CC(O)C1] | 130 |
| 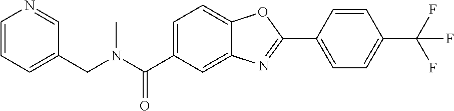 [SMILES: CN(Cc1cccnc1)C(=O)c1ccc2oc(-c3ccc(C(F)(F)F)cc3)nc2c1] | 131 |
| 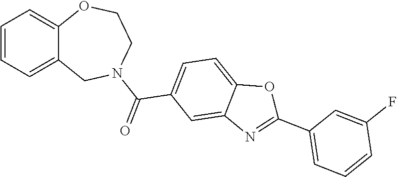 [SMILES: O=C(c1ccc2oc(-c3cccc(F)c3)nc2c1)N1CCOc2ccccc2C1] | 132 |
| 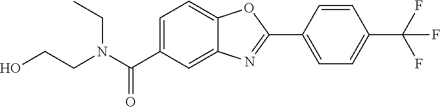 [SMILES: CCN(CCO)C(=O)c1ccc2oc(-c3ccc(C(F)(F)F)cc3)nc2c1] | 133 |
| 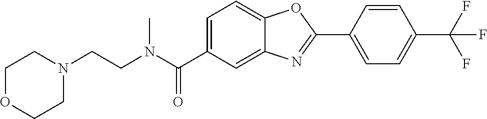 [SMILES: CN(CCN1CCOCC1)C(=O)c1ccc2oc(-c3ccc(C(F)(F)F)cc3)nc2c1] | 134 |
| 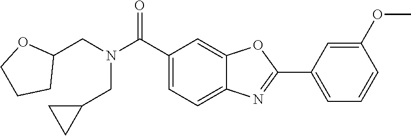 [SMILES: COc1cccc(-c2nc3ccc(C(=O)N(CC4CC4)CC4CCCO4)cc3o2)c1] | 135 |
| 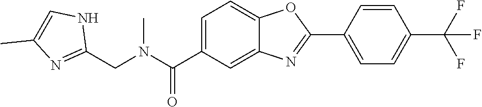 [SMILES: Cc1c[nH]c(CN(C)C(=O)c2ccc3oc(-c4ccc(C(F)(F)F)cc4)nc3c2)n1] | 136 |
| 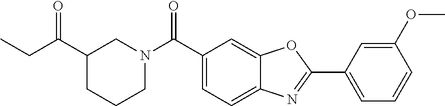 [SMILES: CCC(=O)C1CCCN(C(=O)c2ccc3nc(-c4cccc(OC)c4)oc3c2)C1] | 137 |
| 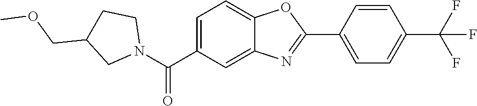 [SMILES: COCC1CCN(C(=O)c2ccc3oc(-c4ccc(C(F)(F)F)cc4)nc3c2)C1] | 138 |
| 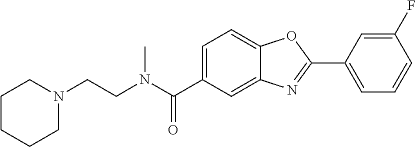 [SMILES: CN(CCN1CCCCC1)C(=O)c1ccc2oc(-c3cccc(F)c3)nc2c1] | 139 |
| 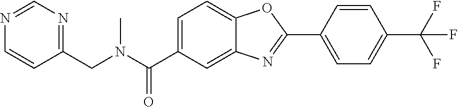 [SMILES: CN(Cc1ccncn1)C(=O)c1ccc2oc(-c3ccc(C(F)(F)F)cc3)nc2c1] | 140 |
| 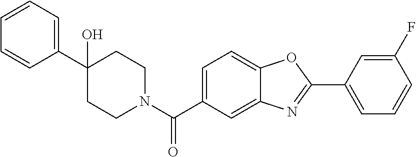 [SMILES: O=C(c1ccc2oc(-c3cccc(F)c3)nc2c1)N1CCC(O)(c2ccccc2)CC1] | 141 |
| 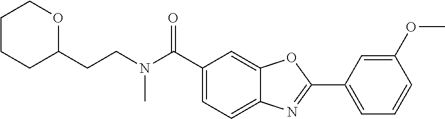 [SMILES: COc1cccc(-c2nc3ccc(C(=O)N(C)CCC4CCCCO4)cc3o2)c1] | 142 |
| 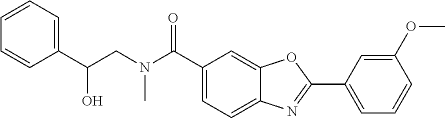 [SMILES: COc1cccc(-c2nc3ccc(C(=O)N(C)CC(O)c4ccccc4)cc3o2)c1] | 143 |
| 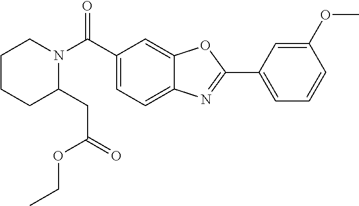 [SMILES: CCOC(=O)CC1CCCCN1C(=O)c1ccc2nc(-c3cccc(OC)c3)oc2c1] | 144 |
| 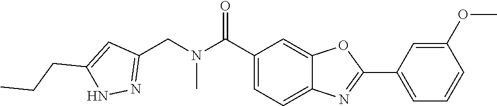 [SMILES: CCCc1cc(CN(C)C(=O)c2ccc3nc(-c4cccc(OC)c4)oc3c2)n[nH]1] | 145 |
| 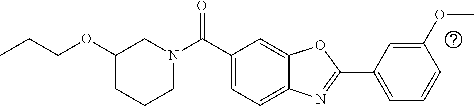 [SMILES: CCCOC1CCCN(C(=O)c2ccc3nc(-c4cccc(OC)c4)oc3c2)C1] | 146 |
| 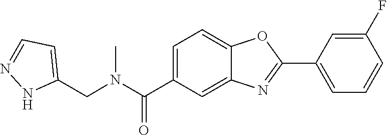 [SMILES: CN(Cc1ccn[nH]1)C(=O)c1ccc2oc(-c3cccc(F)c3)nc2c1] | 147 |
| 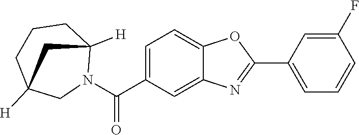 [SMILES: O=C(c1ccc2oc(-c3cccc(F)c3)nc2c1)N1C[C@@H]2CCC[C@H]1C2] | 148 |
| 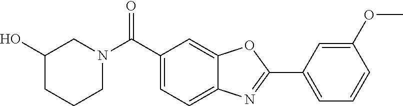 [SMILES: COc1cccc(-c2nc3ccc(C(=O)N4CCCC(O)C4)cc3o2)c1] | 149 |
| 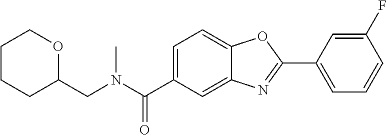 [SMILES: CN(CC1CCCCO1)C(=O)c1ccc2oc(-c3cccc(F)c3)nc2c1] | 150 |
| 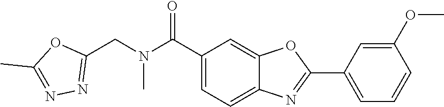 | 151 |
| 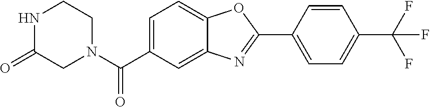 [SMILES: O=C1CN(C(=O)c2ccc3oc(-c4ccc(C(F)(F)F)cc4)nc3c2)CCN1] | 152 |
| 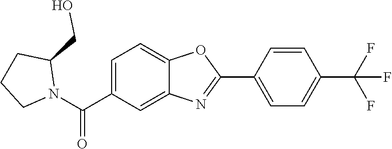 [SMILES: O=C(c1ccc2oc(-c3ccc(C(F)(F)F)cc3)nc2c1)N1CCC[C@H]1CO] | 153 |
| 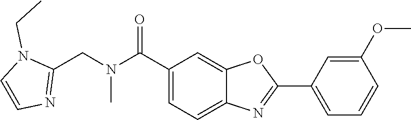 [SMILES: CCn1ccnc1CN(C)C(=O)c1ccc2nc(-c3cccc(OC)c3)oc2c1] | 154 |
| 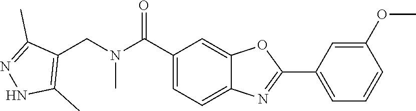 [SMILES: COc1cccc(-c2nc3ccc(C(=O)N(C)Cc4c(C)n[nH]c4C)cc3o2)c1] | 155 |
| 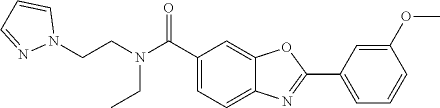 [SMILES: CCN(CCn1cccn1)C(=O)c1ccc2nc(-c3cccc(OC)c3)oc2c1] | 156 |
| 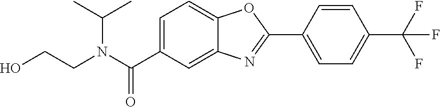 [SMILES: CC(C)N(CCO)C(=O)c1ccc2oc(-c3ccc(C(F)(F)F)cc3)nc2c1] | 157 |
| 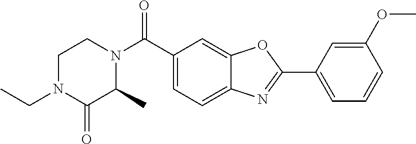 [SMILES: CCN1CCN(C(=O)c2ccc3nc(-c4cccc(OC)c4)oc3c2)[C@@H](C)C1=O] | 158 |
| 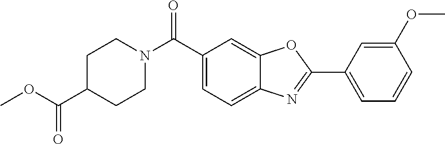 | 159 |
| 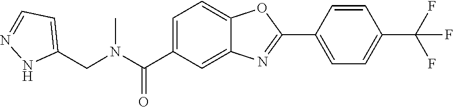 [SMILES: CN(Cc1ccn[nH]1)C(=O)c1ccc2oc(-c3ccc(C(F)(F)F)cc3)nc2c1] | 160 |
| 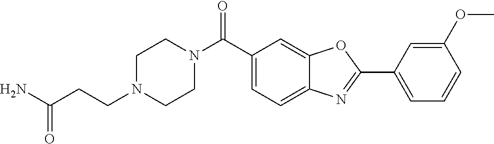 [SMILES: COc1cccc(-c2nc3ccc(C(=O)N4CCN(CCC(N)=O)CC4)cc3o2)c1] | 161 |
| 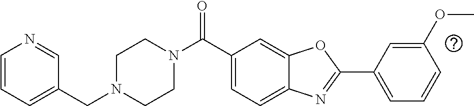 [SMILES: COc1cccc(-c2nc3ccc(C(=O)N4CCN(Cc5cccnc5)CC4)cc3o2)c1] | 162 |
| 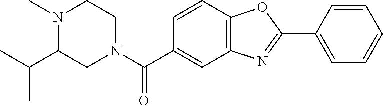 [SMILES: CC(C)C1CN(C(=O)c2ccc3oc(-c4ccccc4)nc3c2)CCN1C] | 163 |
| 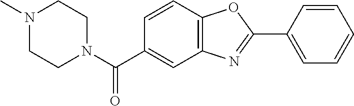 [SMILES: CN1CCN(C(=O)c2ccc3oc(-c4ccccc4)nc3c2)CC1] | 164 |
| 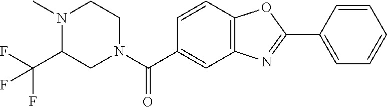 [SMILES: CN1CCN(C(=O)c2ccc3oc(-c4ccccc4)nc3c2)CC1C(F)(F)F] | 165 |
| 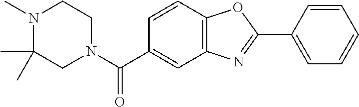 [SMILES: CN1CCN(C(=O)c2ccc3oc(-c4ccccc4)nc3c2)CC1(C)C] | 166 |
| 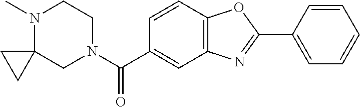 [SMILES: CN1CCN(C(=O)c2ccc3oc(-c4ccccc4)nc3c2)CC12CC2] | 167 |
| 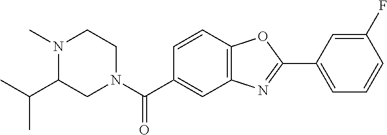 [SMILES: CC(C)C1CN(C(=O)c2ccc3oc(-c4cccc(F)c4)nc3c2)CCN1C] | 168 |
| 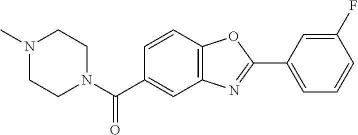 [SMILES: CN1CCN(C(=O)c2ccc3oc(-c4cccc(F)c4)nc3c2)CC1] | 169 |
| 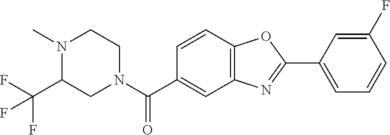 [SMILES: CN1CCN(C(=O)c2ccc3oc(-c4cccc(F)c4)nc3c2)CC1C(F)(F)F] | 170 |
| 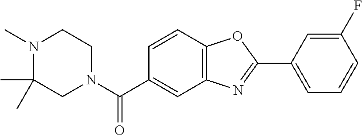 [SMILES: CN1CCN(C(=O)c2ccc3oc(-c4cccc(F)c4)nc3c2)CC1(C)C] | 171 |
| 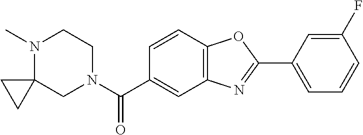 [SMILES: CN1CCN(C(=O)c2ccc3oc(-c4cccc(F)c4)nc3c2)CC12CC2] | 172 |
| 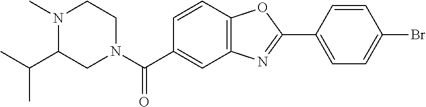 [SMILES: CC(C)C1CN(C(=O)c2ccc3oc(-c4ccc(Br)cc4)nc3c2)CCN1C] | 173 |
| 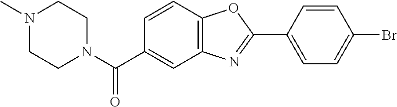 [SMILES: CN1CCN(C(=O)c2ccc3oc(-c4ccc(Br)cc4)nc3c2)CC1] | 174 |
| 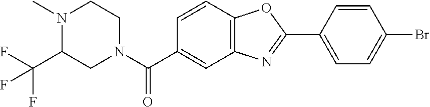 [SMILES: CN1CCN(C(=O)c2ccc3oc(-c4ccc(Br)cc4)nc3c2)CC1C(F)(F)F] | 175 |
| 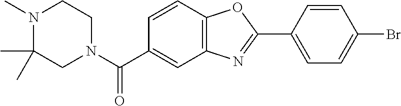 [SMILES: CN1CCN(C(=O)c2ccc3oc(-c4ccc(Br)cc4)nc3c2)CC1(C)C] | 176 |
| 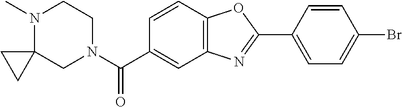 [SMILES: CN1CCN(C(=O)c2ccc3oc(-c4ccc(Br)cc4)nc3c2)CC12CC2] | 177 |
| 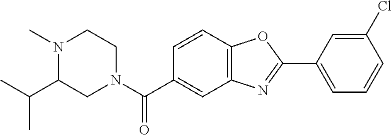 [SMILES: CC(C)C1CN(C(=O)c2ccc3oc(-c4cccc(Cl)c4)nc3c2)CCN1C] | 178 |
| 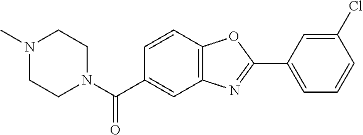 [SMILES: CN1CCN(C(=O)c2ccc3oc(-c4cccc(Cl)c4)nc3c2)CC1] | 179 |
| 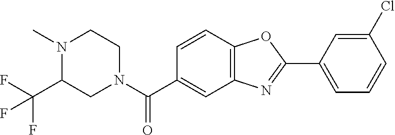 [SMILES: CN1CCN(C(=O)c2ccc3oc(-c4cccc(Cl)c4)nc3c2)CC1C(F)(F)F] | 180 |
| 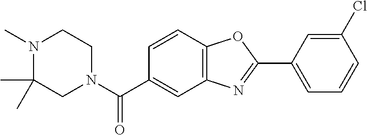 [SMILES: CN1CCN(C(=O)c2ccc3oc(-c4cccc(Cl)c4)nc3c2)CC1(C)C] | 181 |
| 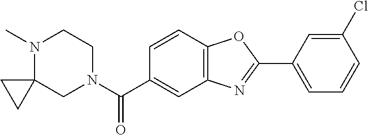 [SMILES: CN1CCN(C(=O)c2ccc3oc(-c4cccc(Cl)c4)nc3c2)CC12CC2] | 182 |
| 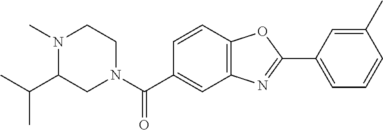 [SMILES: Cc1cccc(-c2nc3cc(C(=O)N4CCN(C)C(C(C)C)C4)ccc3o2)c1] | 183 |
| 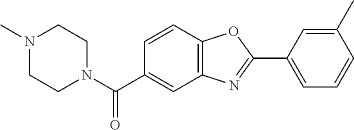 | 184 |
| 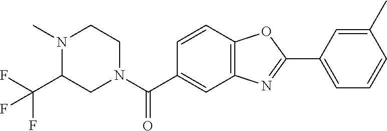 [SMILES: Cc1cccc(-c2nc3cc(C(=O)N4CCN(C)C(C(F)(F)F)C4)ccc3o2)c1] | 185 |
| 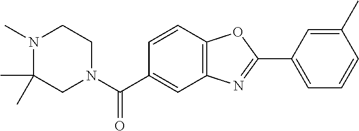 | 186 |
| 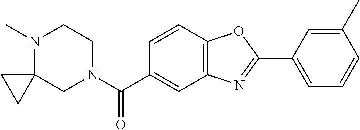 [SMILES: Cc1cccc(-c2nc3cc(C(=O)N4CCN(C)C5(CC5)C4)ccc3o2)c1] | 187 |
| 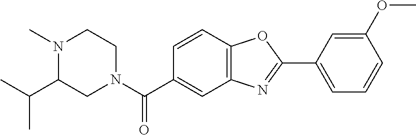 [SMILES: COc1cccc(-c2nc3cc(C(=O)N4CCN(C)C(C(C)C)C4)ccc3o2)c1] | 188 |
| 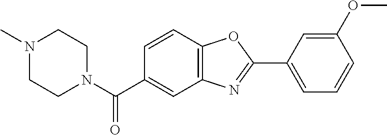 | 189 |
| 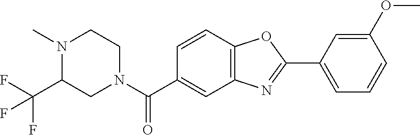 [SMILES: COc1cccc(-c2nc3cc(C(=O)N4CCN(C)C(C(F)(F)F)C4)ccc3o2)c1] | 190 |
| 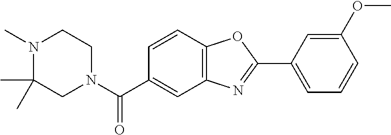 | 191 |
| 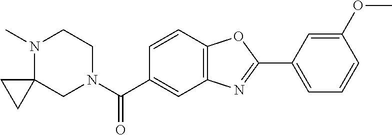 [SMILES: COc1cccc(-c2nc3cc(C(=O)N4CCN(C)C5(CC5)C4)ccc3o2)c1] | 192 |
| 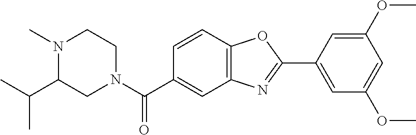 [SMILES: COc1cc(OC)cc(-c2nc3cc(C(=O)N4CCN(C)C(C(C)C)C4)ccc3o2)c1] | 193 |
| 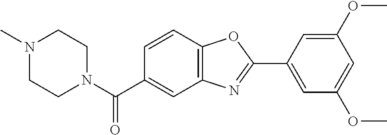 | 194 |
| 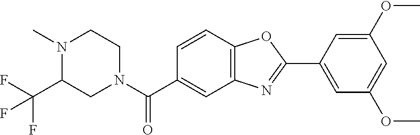 [SMILES: COc1cc(OC)cc(-c2nc3cc(C(=O)N4CCN(C)C(C(F)(F)F)C4)ccc3o2)c1] | 195 |
| 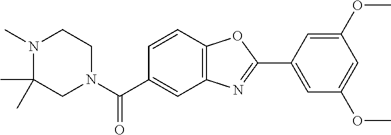 | 196 |
| 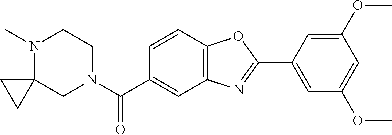 [SMILES: COc1cc(OC)cc(-c2nc3cc(C(=O)N4CCN(C)C5(CC5)C4)ccc3o2)c1] | 197 |
| 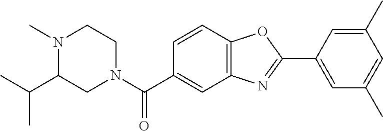 [SMILES: Cc1cc(C)cc(-c2nc3cc(C(=O)N4CCN(C)C(C(C)C)C4)ccc3o2)c1] | 198 |
| 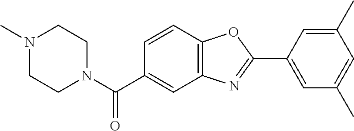 | 199 |
| 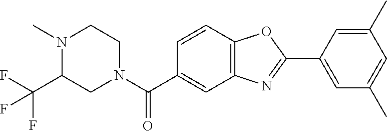 [SMILES: Cc1cc(C)cc(-c2nc3cc(C(=O)N4CCN(C)C(C(F)(F)F)C4)ccc3o2)c1] | 200 |
| 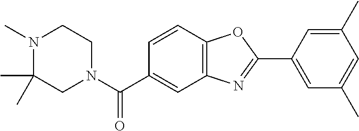 | 201 |
| 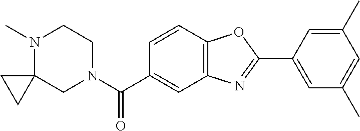 [SMILES: Cc1cc(C)cc(-c2nc3cc(C(=O)N4CCN(C)C5(CC5)C4)ccc3o2)c1] | 202 |
| 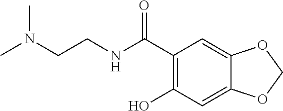 [SMILES: CN(C)CCNC(=O)c1cc2c(cc1O)OCO2] | 203 |
| 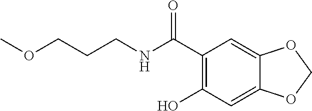 [SMILES: COCCCNC(=O)c1cc2c(cc1O)OCO2] | 204 |

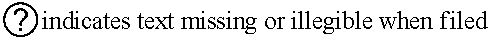

In some embodiments, the compound is a compound of Formula (VI)

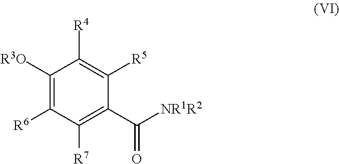

and pharmaceutically acceptable salts thereof, wherein:

R¹ is H or —C₁-C₃ alkyl;

R² is —C₂-C₄ alkyl, —(C₁-C₃ alkylene)-O—(C₁-C₃ alkyl), —(C₁-C₃ alkylene)-N(C₁-C₃ alkyl)₂, —(C₁-C₃ alkylene)-(C₃-C₆ cycloalkyl), —(C₁-C₃ alkylene)-(4-6 membered heterocyclic ring), —C₁-C₃ alkylene-phenyl, or —C₃-C₆ cycloalkyl; or

or R¹ and R², taken together with the nitrogen to which they are attached, form a 4-6 membered heterocyclic ring;

R³ is H or —C₁-C₆ alkyl;

R⁴ is H, —OH, or —O—C₁-C₃ alkyl;

R⁵ is H, C₁-C₃ alkyl, or halogen;

R⁶ is H, —OH, or —O—C₁-C₃ alkyl; and

R⁷ is H, C₁-C₃ alkyl, or halogen.

In some embodiments of the compound of Formula (VI):

R¹ is H;

R² is —C₁-C₃ alkylene-phenyl;

R³ is —C₁-C₆ alkyl;

R⁴ is H;

R⁵ is H;

R⁶ is —OH; and

R⁷ is H.

In some embodiments, R¹ is H.

In some embodiments, R¹ is —C₁-C₃ alkyl. In some embodiments, R¹ is methyl. In some embodiments, R¹ is ethyl. In some embodiments, R¹ is propyl.

In some embodiments, R² is —C₂-C₄ alkyl. In some embodiments, R² is ethyl. In some embodiments, R² is propyl. In some embodiments, R² is isopropyl. In some embodiments, R² is butyl. In some embodiments, R² is isobutyl.

In some embodiments, R² is —(C₁-C₃ alkylene)-O—(C₁-C₃ alkyl). In some embodiments, R² is —(CH₂)₃O—CH₃.

In some embodiments, R² is —(C₁-C₃ alkylene)-N(C₁-C₃ alkyl)₂. In some embodiments, R² is —(CH₂)₂—N(CH₃)₂.

In some embodiments, R² is —(C₁-C₃ alkylene)-(C₃-C₆ cycloalkyl). In some embodiments, the C₃-C₆ cycloalkyl is cyclopropyl. In some embodiments, the C₃-C₆ cycloalkyl is cyclobutyl. In some embodiments, the C₃-C₆ cycloalkyl is cyclopentyl. In some embodiments, the C₃-C₆ cycloalkyl is cyclohexyl.

In some embodiments, R² is —(C₁-C₃ alkylene)-(4-6 membered heterocyclic ring). In some embodiments, the 4-6 membered heterocyclic ring is tetrahydrofuranyl. In some embodiments, the 4-6 membered heterocyclic ring is tetrahydropyranyl.

In some embodiments, R² is —C₁-C₃ alkylene-phenyl. In some embodiments, R² is —CH₂— phenyl. In some embodiments, R² is —(CH₂)₂-phenyl. In some embodiments, R² is —(CH₂)₃-phenyl.

In some embodiments, R² is —C₃-C₆ cycloalkyl. In some embodiments, R² is cyclopropyl.

In some embodiments, R² is cyclobutyl. In some embodiments, R² is cyclopentyl. In some embodiments, R² is cyclohexyl.

In some embodiments, R¹ and R², taken together with the nitrogen to which they are attached, form a 4-6 membered heterocyclic ring. In some embodiments, R¹ and R², taken together with the nitrogen to which they are attached, form an azetidinyl ring. In some embodiments, R¹ and R², taken together with the nitrogen to which they are attached, form a pyrrolidinyl ring. In some embodiments, R¹ and R², taken together with the nitrogen to which they are attached, form a piperidinyl ring.

In some embodiments, R³ is H.

In some embodiments, R³ is —C₁-C₆ alkyl. In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is pentyl. In some embodiments, R³ is hexyl.

In some embodiments, R⁴ is H.

In some embodiments, R⁴ is —OH.

In some embodiments, R⁴ is —O—C₁-C₃ alkyl. In some embodiments, R⁴ is —O—CH₃. In some embodiments, R⁴ is —O—CH₂CH₃. In some embodiments, R⁴ is —O—CH₂CH₂CH₃.

In some embodiments, R⁵ is H.

In some embodiments, R⁵ is C₁-C₃ alkyl. In some embodiments, R⁵ is methyl. In some embodiments, R⁵ is ethyl. In some embodiments, R⁵ is propyl.

In some embodiments, R⁵ is halogen. In some embodiments, R⁵ is chlorine. In some embodiments, R⁵ is fluorine. In some embodiments, R⁵ is bromine.

In some embodiments, R⁶ is H.

In some embodiments, R⁶ is —OH.

In some embodiments, R⁶ is —O—C₁-C₃ alkyl. In some embodiments, R⁶ is —O—CH₃. In some embodiments, R⁶ is —O—CH₂CH₃. In some embodiments, R⁶ is —O—CH₂CH₂CH₃.

In some embodiments, R⁷ is H.

In some embodiments, R⁷ is C₁-C₃ alkyl. In some embodiments, R⁷ is methyl. In some embodiments, R⁷ is ethyl. In some embodiments, R⁷ is propyl.

In some embodiments, R⁷ is halogen. In some embodiments, R⁷ is chlorine. In some embodiments, R⁷ is fluorine. In some embodiments, R⁷ is bromine.

In some embodiments, R¹ is H; R² is —(CH₂)-phenyl; R³ is C₁-C₃ alkyl; R⁴, R⁵, and R⁷ are each H; and R⁶ is —OH.

In some embodiments, R¹ is H; R² is —(CH₂)-phenyl; R³ is ethyl; R⁴, R⁵, and R⁷ are each H; and R⁶ is —OH.

In some embodiments, the compound is a compound of Formula (VI) or a pharmaceutically acceptable salt thereof. In some embodiments, the compound is selected from:

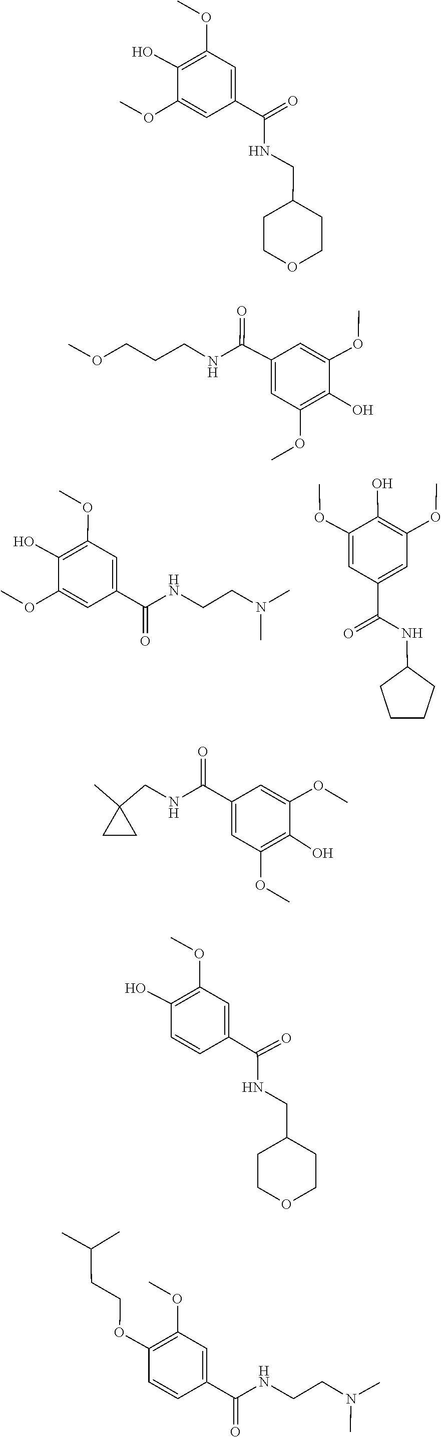

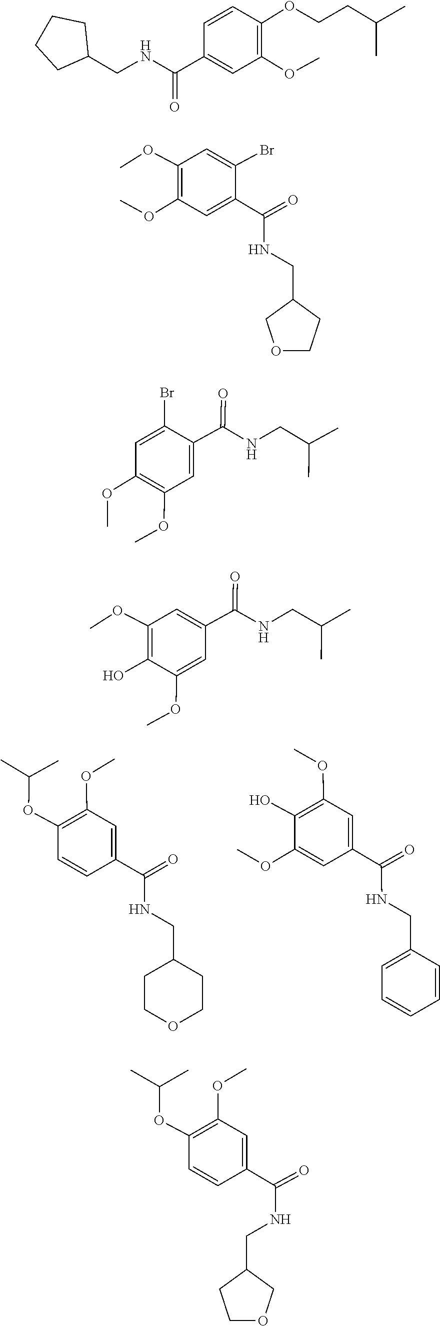

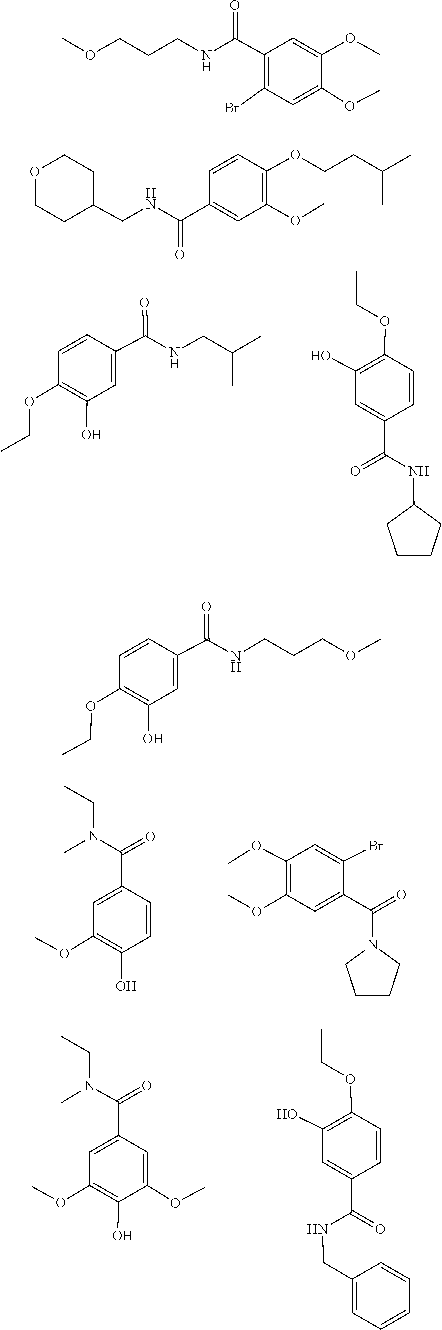

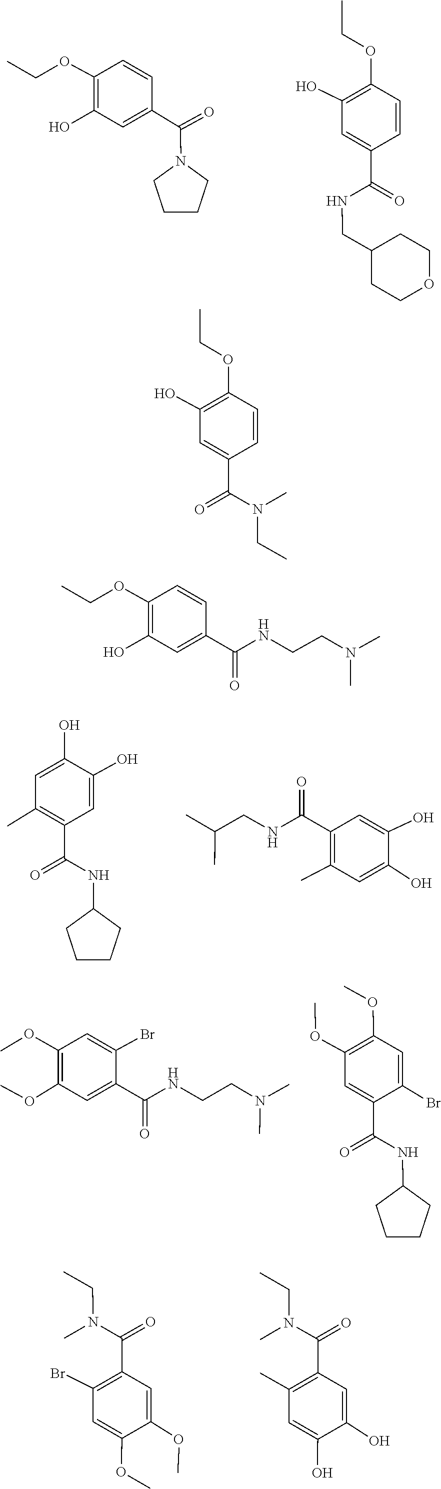

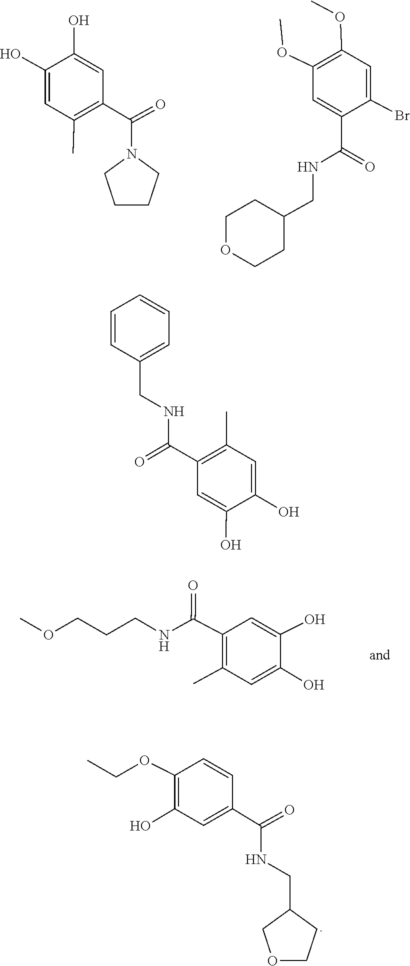 [SMILES: Cc1cc(O)c(O)cc1C(=O)N1CCCC1 ; COc1cc(Br)c(C(=O)NCC2CCOCC2)cc1OC ; Cc1cc(O)c(O)cc1C(=O)NCc1ccccc1 ; COCCCNC(=O)c1cc(O)c(O)cc1C ; CCOc1ccc(C(=O)NCC2CCOC2)cc1O]

Illustrative compounds of Formula (VI) are shown in Table 2.

TABLE 2

| | |
|---|---|
| 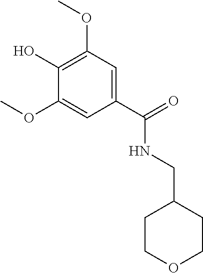 [SMILES: COc1cc(C(=O)NCC2CCOCC2)cc(OC)c1O] | 205 |
| 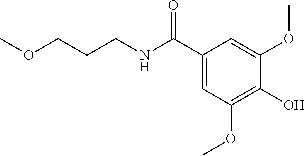 [SMILES: COCCCNC(=O)c1cc(OC)c(O)c(OC)c1] | 206 |
| 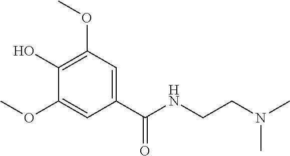 | 207 |
| 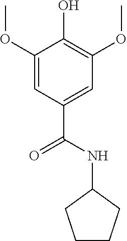 [SMILES: COc1cc(C(=O)NC2CCCC2)cc(OC)c1O] | 208 |
| 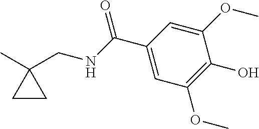 | 209 |
| 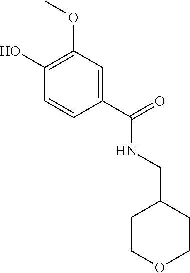 [SMILES: COc1cc(C(=O)NCC2CCOCC2)ccc1O] | 210 |
| 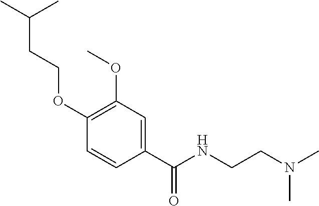 [SMILES: COc1cc(C(=O)NCCN(C)C)ccc1OCCC(C)C] | 211 |
| 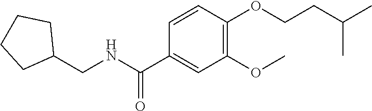 [SMILES: COc1cc(C(=O)NCC2CCCC2)ccc1OCCC(C)C] | 212 |
| 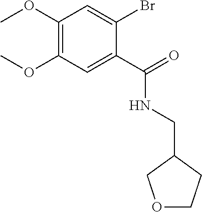 [SMILES: COc1cc(Br)c(C(=O)NCC2CCOC2)cc1OC] | 213 |
| 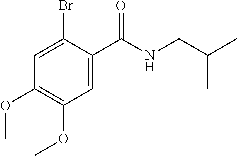 [SMILES: COc1cc(Br)c(C(=O)NCC(C)C)cc1OC] | 214 |
| 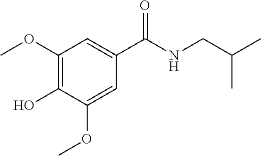 [SMILES: COc1cc(C(=O)NCC(C)C)cc(OC)c1O] | 215 |
| 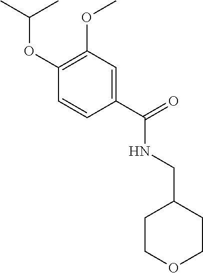 [SMILES: COc1cc(C(=O)NCC2CCOCC2)ccc1OC(C)C] | 216 |
| 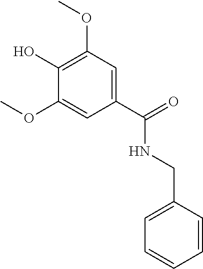 [SMILES: COc1cc(C(=O)NCc2ccccc2)cc(OC)c1O] | 217 |
| 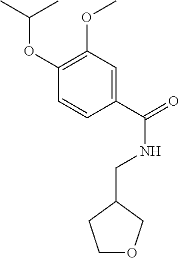 [SMILES: COc1cc(C(=O)NCC2CCOC2)ccc1OC(C)C] | 218 |
| 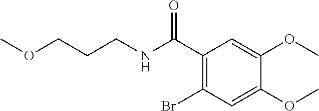 | 219 |
| 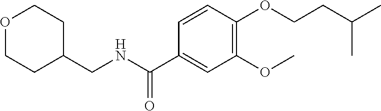 [SMILES: COc1cc(C(=O)NCC2CCOCC2)ccc1OCCC(C)C] | 220 |
| 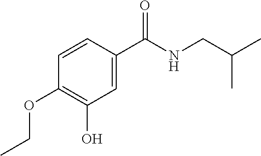 [SMILES: CCOc1ccc(C(=O)NCC(C)C)cc1O] | 221 |
| 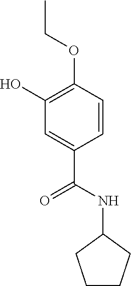 [SMILES: CCOc1ccc(C(=O)NC2CCCC2)cc1O] | 222 |
| 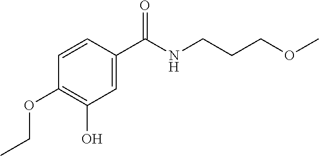 [SMILES: CCOc1ccc(C(=O)NCCCOC)cc1O] | 223 |
| 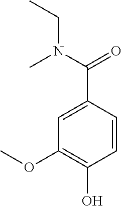 | 224 |
| 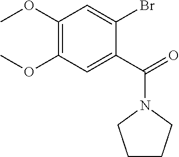 [SMILES: COc1cc(Br)c(C(=O)N2CCCC2)cc1OC] | 225 |
| 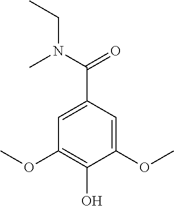 | 226 |
| 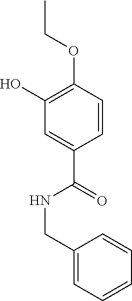 [SMILES: CCOc1ccc(C(=O)NCc2ccccc2)cc1O] | 227 |
| 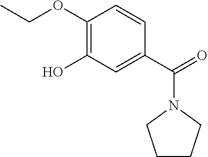 [SMILES: CCOc1ccc(C(=O)N2CCCC2)cc1O] | 228 |
| 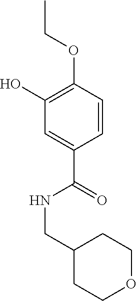 [SMILES: CCOc1ccc(C(=O)NCC2CCOCC2)cc1O] | 229 |
| 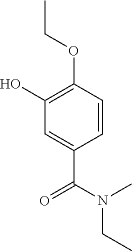 [SMILES: CCOc1ccc(C(=O)N(C)CC)cc1O] | 230 |
| 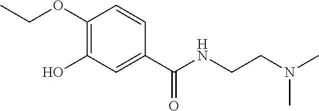 [SMILES: CCOc1ccc(C(=O)NCCN(C)C)cc1O] | 231 |
| 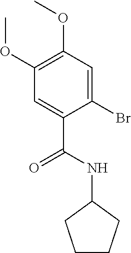 [SMILES: COc1cc(Br)c(C(=O)NC2CCCC2)cc1OC] | 232 |
| 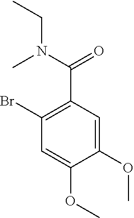 | 233 |
| 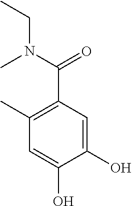 | 234 |
| 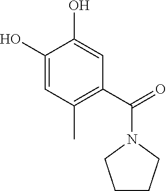 [SMILES: Cc1cc(O)c(O)cc1C(=O)N1CCCC1] | 235 |
| 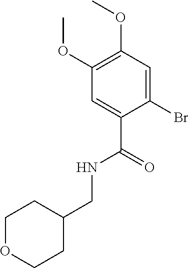 [SMILES: COc1cc(Br)c(C(=O)NCC2CCOCC2)cc1OC] | 236 |
| 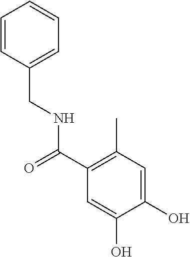 [SMILES: Cc1cc(O)c(O)cc1C(=O)NCc1ccccc1] | 237 |
| 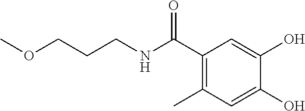 [SMILES: COCCCNC(=O)c1cc(O)c(O)cc1C] | 238 |
| 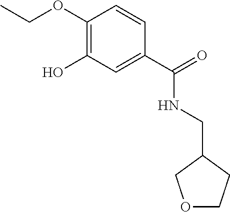 [SMILES: CCOc1ccc(C(=O)NCC2CCOC2)cc1O] | 239 |
| 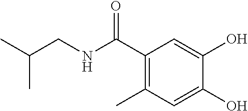 [SMILES: Cc1cc(O)c(O)cc1C(=O)NCC(C)C] | 240 |
| 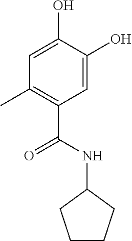 [SMILES: Cc1cc(O)c(O)cc1C(=O)NC1CCCC1] | 241 |
| 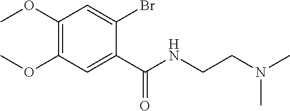 | 242 |

In some embodiments, the compound is selected from:
methyl 5-amino-2-((tert-butoxycarbonyl)amino)-4-hydroxybenzoate;
methyl 5-amino-2-benzamido-4-hydroxybenzoate;
1,3-benzoxazole-6-carboxylic acid;
3,4-dihydroxybenzamide;
N-benzyl-2-(dimethyl amino)-1,3-benzoxazole-4-carboxamide;
2-cyclopropyl-N-ethyl-N-methyl-1,3-benzoxazole-6-carboxamide;
2-cyclohexyl-N-cyclopentyl-1,3-benzoxazole-6-carboxamide;
N-benzyl-4-ethoxy-3-hydroxybenzamide;
1-(2-bromo-4,5-dimethoxybenzoyl)pyrrolidine;
N-(3-methoxypropyl)-2-sulfanyl-1,3-benzoxazole-6-carboxamide;
N-ethyl-N-methyl-2-(oxolan-2-yl)-1,3-benzoxazole-6-carboxamide;
N-[2-(dimethylamino)ethyl]-3-methoxy-4-(3-methylbutoxy)benzamide;
N-cyclopentyl-4-ethoxy-3-hydroxybenzamide;
2-(1,3-benzoxazol-5-yl)-1-(pyrrolidin-1-yl)ethan-1-one;
N-[2-(dimethylamino)ethyl]-6-hydroxy-2H-1,3-benzodioxole-5-carboxamide;
2-cyclohexyl-N-ethyl-N-methyl-1,3-benzoxazole-6-carboxamide;
4-hydroxy-3,5-dimethoxy-N-[(1-methylcyclopropyl)methyl]benzamide;
2-bromo-4,5-dimethoxy-N-[(oxolan-3-yl)methyl]benzamide;
3-methoxy-N-[(oxolan-3-yl)methyl]-4-(propan-2-yloxy)benzamide;
N-ethyl-6-hydroxy-N-methyl-2H-1,3-benzodioxole-5-carboxamide;
4-ethoxy-3-hydroxy-N-(2-methylpropyl)benzamide;
2-(1,3-benzoxazol-5-yl)-N-cyclopentylacetamide;
2-cyclopropyl-N-(3-methoxypropyl)-1,3-benzoxazole-6-carboxamide;
N-[2-(dimethylamino)ethyl]-4-hydroxy-3,5-dimethoxybenzamide;
N-ethyl-N-methyl-2-sulfanyl-1,3-benzoxazole-6-carboxamide;
2-(3,5-dichlorophenyl)-6-(pyrrolidine-1-carbonyl)-1,3-benzoxazole;
N-benzyl-6-hydroxy-2H-1,3-benzodioxole-5-carboxamide;
N-benzyl-4-hydroxy-3,5-dimethoxybenzamide;
N-benzyl-2-sulfanyl-1,3-benzoxazole-6-carboxamide;
N-cyclopentyl-2-(dimethylamino)-1,3-benzoxazole-4-carboxamide;
N,N-dimethyl-7-(pyrrolidine-1-carbonyl)-1,3-benzoxazol-2-amine;
2-ethoxy-5-(pyrrolidine-1-carbonyl)phenol;
N-cyclopentyl-2-cyclopropyl-1,3-benzoxazole-6-carboxamide;
N-ethyl-N-methyl-2-oxo-2,3-dihydro-1,3-benzoxazole-5-carboxamide;
N,N-dimethyl-4-(pyrrolidine-1-carbonyl)-1,3-benzoxazol-2-amine;
N-(3-methoxypropyl)-2-(1,1,2,2,2-pentafluoroethyl)-1,3-benzoxazole-6-carboxamide;
4-hydroxy-3,5-dimethoxy-N-(3-methoxypropyl)benzamide;
N-cyclopentyl-4-hydroxy-3,5-dimethoxybenzamide;
4-ethoxy-3-hydroxy-N-[(oxolan-3-yl)methyl]benzamide;
4-hydroxy-3,5-dimethoxy-N-(2-methylpropyl)benzamide;
2-(dimethylamino)-N-ethyl-N-methyl-1,3-benzoxazole-4-carboxamide;
N-benzyl-2-(dimethylamino)-1,3-benzoxazole-7-carboxamide;
2-cyclopropyl-6-(pyrrolidine-1-carbonyl)-1,3-benzoxazole;
4,5-dihydroxy-2-methyl-N-(2-methylpropyl)benzamide;
N-ethyl-N-methyl-2-(1,1,2,2,2-pentafluoroethyl)-1,3-benzoxazole-6-carboxamide;
2-cyclohexyl-6-(pyrrolidine-1-carbonyl)-1,3-benzoxazole;
N-cyclopentyl-2-methyl-1,3-benzoxazole-4-carboxamide;
6-(pyrrolidine-1-carbonyl)-1,3-benzoxazole-2-thiol;
4-ethoxy-3-hydroxy-N-[(oxan-4-yl)methyl]benzamide;
2-(dimethylamino)-N-ethyl-N-methyl-1,3-benzoxazole-7-carboxamide;
2-(dimethylamino)-N-(3-methoxypropyl)-1,3-benzoxazole-4-carboxamide;
2-(1,3-benzoxazol-5-yl)-N-(3-methoxypropyl)acetamide;
6-hydroxy-N-(2-methylpropyl)-2H-1,3-benzodioxole-5-carboxamide;
2-(3,5-dichlorophenyl)-N-(3-methoxypropyl)-1,3-benzoxazole-6-carboxamide;
3-methoxy-4-(3-methylbutoxy)-N-[(oxan-4-yl)methyl]benzamide;
N-benzyl-2-cyclopropyl-1,3-benzoxazole-6-carboxamide;
N-ethyl-4,5-dihydroxy-N,2-dimethylbenzamide;
N-ethyl-4-hydroxy-3-methoxy-N-methylbenzamide;
2-(1,3-benzoxazol-5-yl)-N-benzylacetamide;
N-benzyl-2-(oxolan-2-yl)-1,3-benzoxazole-6-carboxamide;
N-ethyl-4-hydroxy-3,5-dimethoxy-N-methylbenzamide;
4-ethoxy-3-hydroxy-N-(3-methoxypropyl)benzamide;
6-(pyrrolidine-1-carbonyl)-2H-1,3-benzodioxol-5-ol;
N-benzyl-2-(3,5-dichlorophenyl)-1,3-benzoxazole-6-carboxamide;
2-(1,1,2,2,2-pentafluoroethyl)-6-(pyrrolidine-1-carbonyl)-1,3-benzoxazole;
N-[2-(dimethylamino)ethyl]-4-ethoxy-3-hydroxybenzamide;
2-(dimethylamino)-N-(3-methoxypropyl)-1,3-benzoxazole-7-carboxamide;
4-methyl-5-(pyrrolidine-1-carbonyl)benzene-1,2-diol;
6-hydroxy-N-(3-methoxypropyl)-2H-1,3-benzodioxole-5-carboxamide;
2-cyclohexyl-N-(3-methoxypropyl)-1,3-benzoxazole-6-carboxamide;
N-benzyl-4,5-dihydroxy-2-methylbenzamide;
2-bromo-4,5-dimethoxy-N-[(oxan-4-yl)methyl]benzamide;
N-benzyl-2-methyl-1,3-benzoxazole-4-carboxamide;
N-cyclopentyl-2-(dimethylamino)-1,3-benzoxazole-7-carboxamide;
N-benzyl-2-(1,1,2,2,2-pentafluoroethyl)-1,3-benzoxazole-6-carboxamide;
2-bromo-N-cyclopentyl-4,5-dimethoxybenzamide;
N-(cyclopentylmethyl)-3-methoxy-4-(3-methylbutoxy)benzamide;
4,5-dihydroxy-N-(3-methoxypropyl)-2-methylbenzamide;
2-bromo-4,5-dimethoxy-N-(2-methylpropyl)benzamide;
N-benzyl-2-cyclohexyl-1,3-benzoxazole-6-carboxamide;
N-cyclopentyl-6-hydroxy-2H-1,3-benzodioxole-5-carboxamide;
N-ethyl-N,2-dimethyl-1,3-benzoxazole-5-carboxamide;
3-methoxy-N-[(oxan-4-yl)methyl]-4-(propan-2-yloxy)benzamide;
N-cyclopentyl-4,5-dihydroxy-2-methylbenzamide;
N-(3-methoxypropyl)-2-(oxolan-2-yl)-1,3-benzoxazole-6-carboxamide;
4-ethoxy-N-ethyl-3-hydroxy-N-methylbenzamide;
2-bromo-N-[2-(dimethylamino)ethyl]-4,5-dimethoxybenzamide;
N-(3-methoxypropyl)-2-methyl-1,3-benzoxazole-4-carboxamide;
6-(pyrrolidine-1-carbonyl)-2-(trifluoromethyl)-1,3-benzoxazole;
2-bromo-N-ethyl-4,5-dimethoxy-N-methylbenzamide;
N-ethyl-N-methyl-1,3-benzoxazole-5-carboxamide;
4-hydroxy-3-methoxy-N-[(oxan-4-yl)methyl]benzamide;
N-ethyl-N-methyl-2-phenyl-1,3-benzoxazole-6-carboxamide;
4-hydroxy-3,5-dimethoxy-N-[(oxan-4-yl)methyl]benzamide;
N-cyclopentyl-2-sulfanyl-1,3-benzoxazole-6-carboxamide;
2-(3,5-dichlorophenyl)-N-ethyl-N-methyl-1,3-benzoxazole-6-carboxamide; and
2-bromo-4,5-dimethoxy-N-(3-methoxypropyl)benzamide; and pharmaceutically acceptable salts thereof.

In some embodiments, the compound is selected from:
methyl 5-amino-2-((tert-butoxycarbonyl)amino)-4-hydroxybenzoate;
methyl 5-amino-2-benzamido-4-hydroxybenzoate;
1,3-benzoxazole-6-carboxylic acid;
3,4-dihydroxybenzamide;
N-benzyl-2-(dimethylamino)-1,3-benzoxazole-4-carboxamide;
2-cyclopropyl-N-ethyl-N-methyl-1,3-benzoxazole-6-carboxamide;
2-cyclohexyl-N-cyclopentyl-1,3-benzoxazole-6-carboxamide;
N-benzyl-4-ethoxy-3-hydroxybenzamide;
1-(2-bromo-4,5-dimethoxybenzoyl)pyrrolidine;
N-(3-methoxypropyl)-2-sulfanyl-1,3-benzoxazole-6-carboxamide;
N-ethyl-N-methyl-2-(oxolan-2-yl)-1,3-benzoxazole-6-carboxamide;
N-[2-(dimethylamino)ethyl]-3-methoxy-4-(3-methylbutoxy)benzamide;
N-cyclopentyl-4-ethoxy-3-hydroxybenzamide;
2-(1,3-benzoxazol-5-yl)-1-(pyrrolidin-1-yl)ethan-1-one;
N-[2-(dimethylamino)ethyl]-6-hydroxy-2H-1,3-benzodioxole-5-carboxamide;
2-cyclohexyl-N-ethyl-N-methyl-1,3-benzoxazole-6-carboxamide;
4-hydroxy-3,5-dimethoxy-N-[(1-methylcyclopropyl)methyl]benzamide;
2-bromo-4,5-dimethoxy-N-[(oxolan-3-yl)methyl]benzamide;
3-methoxy-N-[(oxolan-3-yl)methyl]-4-(propan-2-yloxy)benzamide;
N-ethyl-6-hydroxy-N-methyl-2H-1,3-benzodioxole-5-carboxamide;
4-ethoxy-3-hydroxy-N-(2-methylpropyl)benzamide;
2-(1,3-benzoxazol-5-yl)-N-cyclopentylacetamide; and
2-cyclopropyl-N-(3-methoxypropyl)-1,3-benzoxazole-6-carboxamide; and pharmaceutically acceptable salts thereof.

In some embodiments, the compound is selected from:
methyl 5-amino-2-((tert-butoxycarbonyl)amino)-4-hydroxybenzoate;
methyl 5-amino-2-benzamido-4-hydroxybenzoate;
1,3-benzoxazole-6-carboxylic acid;
3,4-dihydroxybenzamide;
N-benzyl-2-(dimethylamino)-1,3-benzoxazole-4-carboxamide;
2-cyclopropyl-N-ethyl-N-methyl-1,3-benzoxazole-6-carboxamide;
2-cyclohexyl-N-cyclopentyl-1,3-benzoxazole-6-carboxamide;
N-benzyl-4-ethoxy-3-hydroxybenzamide;
1-(2-bromo-4,5-dimethoxybenzoyl)pyrrolidine;
N-(3-methoxypropyl)-2-sulfanyl-1,3-benzoxazole-6-carboxamide;
N-ethyl-N-methyl-2-(oxolan-2-yl)-1,3-benzoxazole-6-carboxamide; and
N-[2-(dimethylamino)ethyl]-3-methoxy-4-(3-methylbutoxy)benzamide; and pharmaceutically acceptable salts thereof.

In some embodiments, the compound is selected from:
1-(2-phenyl-1,3-benzoxazole-6-carbonyl)piperidin-4-ol;
3-[(2-phenyl-1,3-benzoxazol-6-yl)formamido]-N-(propan-2-yl)propanamide;
N-(5,5-dimethyloxolan-3-yl)-2-phenyl-1,3-benzoxazole-6-carboxamide;
[(2S,4S)-4-fluoro-1-(2-phenyl-1,3-benzoxazole-6-carbonyl)pyrrolidin-2-yl]methanol;
6-{6-oxa-1-azaspiro[3.4]octane-1-carbonyl}-2-phenyl-1,3-benzoxazole;
N-(2,6-dioxopiperidin-3-yl)-2-phenyl-1,3-benzoxazole-6-carboxamide;
2-(3-fluorophenyl)-N-[3-(1H-pyrazol-1-yl)propyl]-1,3-benzoxazole-5-carboxamide;
2-(1-{[2-(3-fluorophenyl)-1,3-benzoxazol-5-yl]carbonyl}-2-piperidinyl)ethanol;
methyl 4-({[2-(3-methoxyphenyl)-1,3-benzoxazol-6-yl]carbonyl}amino)butanoate;
2-(3-fluorophenyl)-N-(3-pyridinylmethyl)-1,3-benzoxazole-5-carboxamide;
2-(3-fluorophenyl)-N-[2-(1H-pyrazol-1-yl)ethyl]-1,3-benzoxazole-5-carboxamide;
1-{[2-(3-methoxyphenyl)-1,3-benzoxazol-6-yl]carbonyl}-3-piperidinol;
2-(3-fluorophenyl)-N-[(1-methyl-1H-pyrazol-4-yl)methyl]-1,3-benzoxazole-5-carboxamide;
2-(3-methoxyphenyl)-N-(tetrahydro-2H-pyran-2-ylmethyl)-1,3-benzoxazole-6-carboxamide;
N-[(1,5-dimethyl-1H-pyrazol-3-yl)methyl]-2-(3-fluorophenyl)-1,3-benzoxazole-5-carboxamide;
2-(3-methoxyphenyl)-N-(tetrahydro-2H-pyran-4-ylmethyl)-1,3-benzoxazole-6-carboxamide;
ethyl N-{[2-(3-fluorophenyl)-1,3-benzoxazol-5-yl]carbonyl}-beta-alaninate;
2-(3-fluorophenyl)-N-[(2-methyl-1,3-thiazol-4-yl)methyl]-1,3-benzoxazole-5-carboxamide;
2-(3-fluorophenyl)-N-[2-(2-furyl)ethyl]-1,3-benzoxazole-5-carboxamide;
2-(3-methoxyphenyl)-N-(tetrahydro-2-furanylmethyl)-1,3-benzoxazole-6-carboxamide;
2-(3-fluorophenyl)-N-methyl-N-(1H-pyrazol-5-ylmethyl)-1,3-benzoxazole-5-carboxamide;
N-(2-hydroxyethyl)-2-[4-(trifluoromethyl)phenyl]-1,3-benzoxazole-5-carboxamide;
1-{[2-(3-fluorophenyl)-1,3-benzoxazol-5-yl]carbonyl}-N-methylpyrrolidine-3-carboxamide;
N-(1,1-dioxido-2,3-dihydro-3-thienyl)-2-(3-fluorophenyl)-1,3-benzoxazole-5-carboxamide;
1-({2-[4-(trifluoromethyl)phenyl]-1,3-benzoxazol-5-yl}carbonyl)azetidin-3-ol;
N-(3-hydroxypropyl)-2-[4-(trifluoromethyl)phenyl]-1,3-benzoxazole-5-carboxamide;
N-(2-hydroxyethyl)-N-methyl-2-[4-(trifluoromethyl)phenyl]-1,3-benzoxazole-5-carboxamide;
2-(3-fluorophenyl)-N-[2-(4-methyl-4H-1,2,4-triazol-3-yl)ethyl]-1,3-benzoxazole-5-carboxamide;
2-(3-methoxyphenyl)-N-(1H-tetrazol-5-ylmethyl)-1,3-benzoxazole-6-carboxamide;
1-{[2-(3-fluorophenyl)-1,3-benzoxazol-5-yl]carbonyl}-L-prolinamide;
2-(3-fluorophenyl)-5-(thiomorpholin-4-ylcarbonyl)-1,3-benzoxazole;
2-(3-methoxyphenyl)-N-[(5-methylpyrazin-2-yl)methyl]-1,3-benzoxazole-6-carboxamide;
2-(3-fluorophenyl)-N-methyl-N-[(5-methylisoxazol-3-yl)methyl]-1,3-benzoxazole-5-carboxamide;
2-(3-fluorophenyl)-N-(isoxazol-5-ylmethyl)-1,3-benzoxazole-5-carboxamide;
1-{[2-(3-fluorophenyl)-1,3-benzoxazol-5-yl]carbonyl}-2-methyl-1,4-diazepan-5-one;
N-(2-hydroxypropyl)-2-[4-(trifluoromethyl)phenyl]-1,3-benzoxazole-5-carboxamide;
2-(3-fluorophenyl)-N-[1-methyl-2-(1H-1,2,4-triazol-1-yl)ethyl]-1,3-benzoxazole-5-carboxamide;
5-[(1R*,5S*)-6-azabicyclo[3.2.1]oct-6-ylcarbonyl]-2-(3-fluorophenyl)-1,3-benzoxazole;
5-[(4-ethyl-1-piperazinyl)carbonyl]-2-(3-fluorophenyl)-1,3-benzoxazole;
2-(3-fluorophenyl)-N-methyl-N-(tetrahydro-2H-pyran-2-ylmethyl)-1,3-benzoxazole-5-carboxamide;
N-isopropyl-2-(3-methoxyphenyl)-N-methyl-1,3-benzoxazole-6-carboxamide;
ethyl N-{[2-(3-methoxyphenyl)-1,3-benzoxazol-6-yl]carbonyl}-beta-alaninate;
2-(3-fluorophenyl)-N-[2-(1-methyl-1H-pyrazol-4-yl)ethyl]-1,3-benzoxazole-5-carboxamide;
(1-{[2-(3-fluorophenyl)-1,3-benzoxazol-5-yl]carbonyl}-2-piperidinyl)methanol;
2-(3-fluorophenyl)-N-[1-(1H-1,2,4-triazol-5-yl)ethyl]-1,3-benzoxazole-5-carboxamide;
2-(3-fluorophenyl)-N-[2-(1H-pyrazol-4-yl)ethyl]-1,3-benzoxazole-5-carboxamide;
N-(3-hydroxy-2,2-dimethylpropyl)-2-(3-methoxyphenyl)-1,3-benzoxazole-6-carboxamide;
N-isopropyl-2-[4-(trifluoromethyl)phenyl]-1,3-benzoxazole-5-carboxamide;
2-(3-methoxyphenyl)-N-(2,2,2-trifluoroethyl)-1,3-benzoxazole-6-carboxamide;
N-[(1-ethyl-1H-pyrazol-4-yl)methyl]-2-(3-fluorophenyl)-1,3-benzoxazole-5-carboxamide;
2-(3-fluorophenyl)-N-[(3S)-2-oxo-3-azepanyl]-1,3-benzoxazole-5-carboxamide;
2-(3-fluorophenyl)-N-[(3S*,4S*)-4-methoxy-1-methylpyrrolidin-3-yl]-1,3-benzoxazole-5-carboxamide;
1-{[2-(3-methoxyphenyl)-1,3-benzoxazol-6-yl]carbonyl}-N,N-dimethyl-3-pyrrolidinamine;
N-cyclobutyl-2-[4-(trifluoromethyl)phenyl]-1,3-benzoxazole-5-carboxamide;
N-(2,3-dihydroxypropyl)-N-methyl-2-[4-(trifluoromethyl)phenyl]-1,3-benzoxazole-5-carboxamide;
2-(3-fluorophenyl)-N-methyl-N-[(5-methylisoxazol-3-yl)methyl]-1,3-benzoxazole-5-carboxamide;
1-{[2-(3-fluorophenyl)-1,3-benzoxazol-5-yl]carbonyl}-L-prolinamide;
2-(3-fluorophenyl)-N-(3-hydroxy-3-phenylpropyl)-N-methyl-1,3-benzoxazole-5-carboxamide;
N-[(5-ethyl-2-pyridinyl)methyl]-2-(3-fluorophenyl)-N-methyl-1,3-benzoxazole-5-carboxamide;
ethyl 1-{[2-(3-methoxyphenyl)-1,3-benzoxazol-6-yl]carbonyl}-3-piperidinecarboxylate;
N-(2-hydroxyethyl)-N-methyl-2-[4-(trifluoromethyl)phenyl]-1,3-benzoxazole-5-carboxamide;
1-{[2-(3-fluorophenyl)-1,3-benzoxazol-5-yl]carbonyl}-2-methyl-1,4-diazepan-5-one;
2-(3-fluorophenyl)-N-isopropyl-N-[(1-methyl-1H-imidazol-2-yl)methyl]-1,3-benzoxazole-5-carboxamide;
N-[(1-ethyl-1H-imidazol-2-yl)methyl]-N-methyl-2-[4-(trifluoromethyl)phenyl]-1,3-benzoxazole-5-carboxamide;
6-[(4-cyclopentyl-1-piperazinyl)carbonyl]-2-(3-methoxyphenyl)-1,3-benzoxazole;
5-(4-morpholinylcarbonyl)-2-[4-(trifluoromethyl)phenyl]-1,3-benzoxazole;
4-{[2-(3-methoxyphenyl)-1,3-benzoxazol-6-yl]carbonyl}morpholine-2-carboxamide;
2-(3-fluorophenyl)-5-(thiomorpholin-4-ylcarbonyl)-1,3-benzoxazole;
(3S)-1-({2-[4-(trifluoromethyl)phenyl]-1,3-benzoxazol-5-yl}carbonyl)-3-pyrrolidinol;
N,N-diethyl-1-{[2-(3-methoxyphenyl)-1,3-benzoxazol-6-yl]carbonyl}-3-piperidinecarboxamide;
2-(3-fluorophenyl)-5-{[4-(2-methoxyethoxy)-1-piperidinyl]carbonyl}-1,3-benzoxazole;
2-(1-{[2-(3-fluorophenyl)-1,3-benzoxazol-5-yl]carbonyl}-2-piperidinyl)ethanol;
N-isopropyl-2-(3-methoxyphenyl)-N-methyl-1,3-benzoxazole-6-carboxamide;
2-(3-methoxyphenyl)-N-methyl-N-[(5-methylisoxazol-3-yl)methyl]-1,3-benzoxazole-6-carboxamide;
6-[(1,1-dioxidothiomorpholin-4-yl)carbonyl]-2-(3-methoxyphenyl)-1,3-benzoxazole;
1-{[2-(3-fluorophenyl)-1,3-benzoxazol-5-yl]carbonyl}-N-methylpyrrolidine-3-carboxamide;
1-({2-[4-(trifluoromethyl)phenyl]-1,3-benzoxazol-5-yl}carbonyl)azetidin-3-ol;
N-methyl-N-(3-pyridinylmethyl)-2-[4-(trifluoromethyl)phenyl]-1,3-benzoxazole-5-carboxamide;
4-{[2-(3-fluorophenyl)-1,3-benzoxazol-5-yl]carbonyl}-2,3,4,5-tetrahydro-1,4-benzoxazepine;
N-ethyl-N-(2-hydroxyethyl)-2-[4-(trifluoromethyl)phenyl]-1,3-benzoxazole-5-carboxamide;
N-methyl-N-[2-(4-morpholinyl)ethyl]-2-[4-(trifluoromethyl)phenyl]-1,3-benzoxazole-5-carboxamide;
N-(cyclopropylmethyl)-2-(3-methoxyphenyl)-N-(tetrahydro-2-furanylmethyl)-1,3-benzoxazole-6-carboxamide;
N-methyl-N-[(4-methyl-1H-imidazol-2-yl)methyl]-2-[4-(trifluoromethyl)phenyl]-1,3-benzoxazole-5-carboxamide;
1-(1-{[2-(3-methoxyphenyl)-1,3-benzoxazol-6-yl]carbonyl}-3-piperidinyl)-1-propanone;
5-{[3-(methoxymethyl)-1-pyrrolidinyl]carbonyl}-2-[4-(trifluoromethyl)phenyl]-1,3-benzoxazole;
2-(3-fluorophenyl)-N-methyl-N-[2-(1-piperidinyl)ethyl]-1,3-benzoxazole-5-carboxamide;
N-methyl-N-(4-pyrimidinylmethyl)-2-[4-(trifluoromethyl)phenyl]-1,3-benzoxazole-5-carboxamide;
1-{[2-(3-fluorophenyl)-1,3-benzoxazol-5-yl]carbonyl}-4-phenyl-4-piperidinol;
2-(3-methoxyphenyl)-N-methyl-N-[2-(tetrahydro-2H-pyran-2-yl)ethyl]-1,3-benzoxazole-6-carboxamide;
N-(2-hydroxy-2-phenylethyl)-2-(3-methoxyphenyl)-N-methyl-1,3-benzoxazole-6-carboxamide;
ethyl (1-{[2-(3-methoxyphenyl)-1,3-benzoxazol-6-yl]carbonyl}-2-piperidinyl)acetate;
2-(3-methoxyphenyl)-N-methyl-N-[(5-propyl-1H-pyrazol-3-yl)methyl]-1,3-benzoxazole-6-carboxamide;
2-(3-methoxyphenyl)-6-[(3-propoxy-1-piperidinyl)carbonyl]-1,3-benzoxazole;
2-(3-fluorophenyl)-N-methyl-N-(1H-pyrazol-5-ylmethyl)-1,3-benzoxazole-5-carboxamide;
5-[(1R*,5S*)-6-azabicyclo[3.2.1]oct-6-ylcarbonyl]-2-(3-fluorophenyl)-1,3-benzoxazole;
1-{[2-(3-methoxyphenyl)-1,3-benzoxazol-6-yl]carbonyl}-3-piperidinol;
2-(3-fluorophenyl)-N-methyl-N-(tetrahydro-2H-pyran-2-ylmethyl)-1,3-benzoxazole-5-carboxamide;
2-(3-methoxyphenyl)-N-methyl-N-[(5-methyl-1,3,4-oxadiazol-2-yl)methyl]-1,3-benzoxazole-6-carboxamide;
4-({2-[4-(trifluoromethyl)phenyl]-1,3-benzoxazol-5-yl}carbonyl)-2-piperazinone;
[(2S)-1-({2-[4-(trifluoromethyl)phenyl]-1,3-benzoxazol-5-yl}carbonyl)-2-pyrrolidinyl]methanol;
N-[(1-ethyl-1H-imidazol-2-yl)methyl]-2-(3-methoxyphenyl)-N-methyl-1,3-benzoxazole-6-carboxamide;
N-[(3,5-dimethyl-1H-pyrazol-4-yl)methyl]-2-(3-methoxyphenyl)-N-methyl-1,3-benzoxazole-6-carboxamide;
N-ethyl-2-(3-methoxyphenyl)-N-[2-(1H-pyrazol-1-yl)ethyl]-1,3-benzoxazole-6-carboxamide;
N-(2-hydroxyethyl)-N-isopropyl-2-[4-(trifluoromethyl)phenyl]-1,3-benzoxazole-5-carboxamide;
(3S)-1-ethyl-4-{[2-(3-methoxyphenyl)-1,3-benzoxazol-6-yl]carbonyl}-3-methyl-2-piperazinone;
methyl 1-{[2-(3-methoxyphenyl)-1,3-benzoxazol-6-yl]carbonyl}-4-piperidinecarboxylate;
N-methyl-N-(1H-pyrazol-5-ylmethyl)-2-[4-(trifluoromethyl)phenyl]-1,3-benzoxazole-5-carboxamide;
3-(4-{[2-(3-methoxyphenyl)-1,3-benzoxazol-6-yl]carbonyl}-1-piperazinyl)propanamide;
2-(3-methoxyphenyl)-6-{[4-(3-pyridinylmethyl)-1-piperazinyl]carbonyl}-1,3-benzoxazole;
(3-isopropyl-4-methyl-piperazin-1-yl)-(8-phenyl-7-oxa-9-aza-bicyclo[4.3.0]nona-1(6),2,4,8-tetraen-3-yl)-methanone;
(4-methyl-piperazin-1-yl)-(8-phenyl-7-oxa-9-aza-bicyclo[4.3.0]nona-1(6),2,4,8-tetraen-3-yl)-methanone;
(4-methyl-3-(trifluoro-methyl)-piperazin-1-yl)-(8-phenyl-7-oxa-9-aza-bicyclo[4.3.0]nona-1(6),2,4,8-tetraen-3-yl)-methanone;
(8-phenyl-7-oxa-9-aza-bicyclo[4.3.0]nona-1(6),2,4,8-tetraen-3-yl)-(3,3,4-trimethyl-piperazin-1-yl)-methanone;
(4-methyl-4,7-diaza-spiro[2.5]octan-7-yl)-(8-phenyl-7-oxa-9-aza-bicyclo[4.3.0]nona-1(6),2,4,8-tetraen-3-yl)-methanone;
(8-(3-fluoro-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-fluoro-phenyl)-7-oxa-9-aza-bicyclo[4.3.0]nona-1(6),2,4,8-tetraen-3-yl)-(3,3,4-trimethyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(4-bromo-phenyl)-7-oxa-9-aza-bicyclo[4.3.0]nona-1(6),2,4,8-tetraen-3-yl)-(3-isopropyl-4-methyl-piperazin-1-yl)-methanone;
(8-(4-bromo-phenyl)-7-oxa-9-aza-bicyclo[4.3.0]nona-1(6),2,4,8-tetraen-3-yl)-(4-methyl-piperazin-1-yl)-methanone;
(8-(4-bromo-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(4-bromo-phenyl)-7-oxa-9-aza-bicyclo[4.3.0]nona-1(6),2,4,8-tetraen-3-yl)-(3,3,4-trimethyl-piperazin-1-yl)-methanone;
(8-(4-bromo-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3-chloro-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3-chloro-phenyl)-7-oxa-9-aza-bicyclo[4.3.0]nona-1(6),2,4,8-tetraen-3-yl)-(4-methyl-piperazin-1-yl)-methanone;
(8-(3-chloro-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-chloro-phenyl)-7-oxa-9-aza-bicyclo[4.3.0]nona-1(6),2,4,8-tetraen-3-yl)-(3,3,4-trimethyl-piperazin-1-yl)-methanone;
(8-(3-chloro-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(3-isopropyl-4-methyl-piperazin-1-yl)-(8-m-tolyl-7-oxa-9-aza-bicyclo[4.3.0]nona-1(6),2,4,8-tetraen-3-yl)-methanone;
(4-methyl-piperazin-1-yl)-(8-m-tolyl-7-oxa-9-aza-bicyclo[4.3.0]nona-1(6),2,4,8-tetraen-3-yl)-methanone;
(4-methyl-3-(trifluoro-methyl)-piperazin-1-yl)-(8-m-tolyl-7-oxa-9-aza-bicyclo[4.3.0]nona-1(6),2,4,8-tetraen-3-yl)-methanone;
(8-m-tolyl-7-oxa-9-aza-bicyclo[4.3.0]nona-1(6),2,4,8-tetraen-3-yl)-(3,3,4-trimethyl-piperazin-1-yl)-methanone;
(4-methyl-4,7-diaza-spiro[2.5]octan-7-yl)-(8-m-tolyl-7-oxa-9-aza-bicyclo[4.3.0]nona-1(6),2,4,8-tetraen-3-yl)-methanone;
(3-isopropyl-4-methyl-piperazin-1-yl)-(8-(3-methoxy-phenyl)-7-oxa-9-aza-bicyclo[4.3.0]nona-1(6),2,4,8-tetraen-3-yl)-methanone;
(8-(3-methoxy-phenyl)-7-oxa-9-aza-bicyclo[4.3.0]nona-1(6),2,4,8-tetraen-3-yl)-(4-methyl-piperazin-1-yl)-methanone;
(8-(3-methoxy-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-methoxy-phenyl)-7-oxa-9-aza-bicyclo[4.3.0]nona-1(6),2,4,8-tetraen-3-yl)-(3,3,4-trimethyl-piperazin-1-yl)-methanone;
(8-(3-methoxy-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3,5-dimethoxy-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3,5-dimethoxy-phenyl)-7-oxa-9-aza-bicyclo[4.3.0]nona-1(6),2,4,8-tetraen-3-yl)-(3,3,4-trimethyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3,5-dimethyl-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3,5-dimethyl-phenyl)-7-oxa-9-aza-bicyclo[4.3.0]nona-1(6),2,4,8-tetraen-3-yl)-(4-methyl-piperazin-1-yl)-methanone;
(8-(3,5-dimethyl-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3,5-dimethyl-phenyl)-7-oxa-9-aza-bicyclo[4.3.0]nona-1(6),2,4,8-tetraen-3-yl)-(3,3,4-trimethyl-piperazin-1-yl)-methanone; and
(8-(3,5-dimethyl-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;

and pharmaceutically acceptable salts thereof.

In some embodiments, the compound is methyl 5-amino-2-((tert-butoxycarbonyl)amino)-4-hydroxybenzoate or a pharmaceutically acceptable salt thereof.

In some embodiments, the compound is methyl 5-amino-2-benzamido-4-hydroxybenzoate or a pharmaceutically acceptable salt thereof.

In some embodiments, the compound is 1,3-benzoxazole-6-carboxylic acid or a pharmaceutically acceptable salt thereof.

In some embodiments, the compound is 3,4-dihydroxybenzamide or a pharmaceutically acceptable salt thereof.

In some embodiments, the compound is N-benzyl-2-(dimethylamino)-1,3-benzoxazole-4-carboxamide or a pharmaceutically acceptable salt thereof.

In some embodiments, the compound is 2-cyclopropyl-N-ethyl-N-methyl-1,3-benzoxazole-6-carboxamide or a pharmaceutically acceptable salt thereof.

In some embodiments, the compound is 2-cyclohexyl-N-cyclopentyl-1,3-benzoxazole-6-carboxamide or a pharmaceutically acceptable salt thereof.

In some embodiments, the compound is N-benzyl-4-ethoxy-3-hydroxybenzamide or a pharmaceutically acceptable salt thereof.

In some embodiments, the compound is 1-(2-bromo-4,5-dimethoxybenzoyl)pyrrolidine or a pharmaceutically acceptable salt thereof.

In some embodiments, the compound is N-(3-methoxypropyl)-2-sulfanyl-1,3-benzoxazole-6-carboxamide or a pharmaceutically acceptable salt thereof.

In some embodiments, the compound is N-ethyl-N-methyl-2-(oxolan-2-yl)-1,3-benzoxazole-6-carboxamide or a pharmaceutically acceptable salt thereof.

In some embodiments, the compound is N-[2-(dimethylamino)ethyl]-3-methoxy-4-(3-methylbutoxy)benzamide or a pharmaceutically acceptable salt thereof.

In some embodiments, the compounds described herein, e.g., compounds of any one of Formulas (I), (II), (III), (IV), (V), (Va), (Vb) or (VI), and pharmaceutically acceptable salts and derivatives thereof, are useful as Sb9 inhibitors for the treatment of diseases or disorders associated with Sb9 expression and/or activity.

It is appreciated that certain features of the described compounds and methods, which are, for clarity, described in the context of separate embodiments, can also be provided in combination in a single embodiment as if the embodiments were claims written in multiple dependent form. Conversely, various features of the described compounds and methods which are, for brevity, described in the context of a single embodiment, can also be provided separately or in any suitable sub-combination.

At various places in the present specification, divalent linking substituents are described.

Unless otherwise specified, it is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl,” employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms.

In some embodiments, the aryl group has 6 to 10 carbon atoms. In some embodiments, the aryl group is phenyl or naphthyl. In some embodiments, the aryl is phenyl.

As used herein, “halogen” refers to F, Cl, Br, or I. In some embodiments, halogen is F, Cl, or Br. In some embodiments, halogen is F. In some embodiments, halogen is Cl. In some embodiments, halogen is Br.

As used herein, the term “Cₙ₋ₘ haloalkyl,” employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group).

Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heterocyclic ring” refers to a monocyclic or polycyclic ring (e.g., having 2, 3, or 4 fused rings) having at least one heteroatom ring member selected from N, O, or S. In some embodiments, the heterocyclic ring is a heteroaryl ring having at least one aromatic heterocyclic ring. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridinyl, pyrimidinyl, pyrazinyl, pyridazinyl, pyrrolyl, pyrazolyl, azolyl, oxazolyl, isoxazolyl, thiazolyl, isothiazolyl, imidazolyl, oxadiazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

In some embodiments, the heterocyclic ring is a heterocycloalkyl having at least one non-aromatic ring (saturated or partially unsaturated ring) or, wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, dihydrothiophenyl, piperidinyl, pyrrolidinyl, morpholinyl, thiomorpholinyl, isoxazolidinyl, isothiazolidinyl, pyrazolidinyl, oxazolidinyl, thiazolidinyl, imidazolidinyl, caprolactamyl, azepanyl, diazepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, oxa-azaspiro[3.4]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, dihydrobenzo[1,4]oxazepinyl, and the like.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C=O or C(O)), or attached to a nitrogen or sulfur heteroatom forming a nitroso, sulfinyl or sulfonyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration.

In some embodiments, the compounds described herein, e.g., compounds of any one of Formulas (I), (II), (III), (IV), (V), (Va), (Vb) or (VI), and pharmaceutically acceptable salts and derivatives thereof, are useful for treating diseases or disorders associated with hyperproliferation (i.e., cellular proliferative disorders). Examples of hyperproliferative diseases or disorders include, but are not limited to, cancer, for example, carcinoma, sarcoma, adenocarcinoma, and metastatic disorders, and hematopoietic neoplastic disorders, for example, leukemias. In some embodiments, the disorder is cancer. In some embodiments, the cancer is metastatic cancer. In some embodiments, the compounds of Formulas (I), (II), (III), (IV), (V), (Va), (Vb), and (VI), and pharmaceutically acceptable salts and derivatives thereof, are useful for treating a metastatic tumor. A metastatic tumor can arise from a multitude of primary tumor types, including, but not limited to, those of prostate, colon, lung, breast, and liver origin.

In some embodiments, the compounds described herein, e.g., compounds of any one of Formulas (I), (II), (III), (IV), (V), (Va), (Vb) or (VI), and pharmaceutically acceptable salts and derivatives thereof, that are useful as Sb9 inhibitors are used for the treatment of cancer. In some embodiments, the cancer is selected from melanoma, colorectal, pancreatic, lung, including non-small cell lung cancer, breast, kidney, thyroid, lymphoid, gastrointestinal, genitourinary tract cancers, Hodgkin lymphoma, colon, renal cell carcinoma, ovarian, prostate cancer and/or testicular tumors, small intestine, and esophagus cancer. In some embodiments, the cancer is melanoma. In some embodiments, the cancer is breast cancer.

In some embodiments, the compounds described herein, e.g., compounds of any one of Formulas (I), (II), (III), (IV), (V), (Va), (Vb) or (VI), and pharmaceutically acceptable salts and derivatives thereof, are useful for treating a carcinoma. In some embodiments, the carcinoma is a respiratory system carcinoma, gastrointestinal system carcinoma, genitourinary system carcinoma, testicular carcinoma, breast carcinoma, prostatic carcinoma, endocrine system carcinoma, and melanoma. In some embodiments, the disease is renal carcinoma or melanoma. Exemplary carcinomas include those forming from tissue of the cervix, lung, prostate, breast, head and neck, colon, and ovary. In some embodiments, the carcinoma is a carcinosarcoma. In some embodiments, the carcinoma is an adenocarcinoma.

Thus, provided herein are methods of treating a disease or disorder associated with Sb9 expression and/or activity in an individual in need thereof, the method including administering to the individual a compound described herein, e.g., compounds of any one of Formulas (I), (II), (III), (IV), (V), (Va), (Vb) or (VI), or a pharmaceutically acceptable salt or derivative thereof. In some embodiments, the disease or disorder is a hyperproliferative disorder. In some embodiments, the disease or disorder is cancer. In some embodiments, the cancer is selected from melanoma, colorectal, pancreatic, lung, including non-small cell lung cancer, breast, kidney, thyroid, lymphoid, gastrointestinal, genitourinary tract cancers, Hodgkin lymphoma, colon, renal cell carcinoma, ovarian, prostate cancer and/or testicular tumors, small intestine, and esophagus cancer. In some embodiments, the cancer is melanoma. In some embodiments, the cancer is breast cancer. In some embodiments, the compound is a compound of Formula (I) or a pharmaceutically acceptable salt thereof. In some embodiments, the compound is a compound of Formula (II) or a pharmaceutically acceptable salt thereof. In some embodiments, the compound is a compound of Formula (III) or a pharmaceutically acceptable salt thereof. In some embodiments, the compound is a derivative of a compound of Formula (III) or a pharmaceutically acceptable salt thereof, such as the compounds provided in the present disclosure. In some embodiments, the compound is a compound of Formula (IV) or a pharmaceutically acceptable salt thereof. In some embodiments, the compound is a derivative of a compound of Formula (IV) or a pharmaceutically acceptable salt thereof, such as the compounds provided in the present disclosure. In some embodiments, the compound is a compound of Formula (V) or a pharmaceutically acceptable salt thereof. In some embodiments, the compound is a compound of Formula (Va) or a pharmaceutically acceptable salt thereof. In some embodiments, the compound is a compound of Formula (Vb) or a pharmaceutically acceptable salt thereof. In some embodiments, the compound is a compound of Formula (VI) or a pharmaceutically acceptable salt thereof.

In some embodiments, the compounds described herein, e.g., compounds of any one of Formulas (I), (II), (III), (IV), (V), (Va), (Vb) or (VI), and pharmaceutically acceptable salts and derivatives thereof, are useful for treating or controlling tumor growth, for example, by direct sensitization to GrB and the activation of protective immunity. In some embodiments, the tumor is immunologically inert. In some embodiments, the compounds described herein, e.g., compounds of any one of Formulas (I), (II), (III), (IV), (V), (Va), (Vb) or (VI), and pharmaceutically acceptable salts and derivatives thereof, inhibit Sb9, thereby directly killing the tumor. In some embodiments, the compounds described herein, e.g., compounds of any one of Formulas (I), (II), (III), (IV), (V), (Va), (Vb) or (VI), and pharmaceutically acceptable salts and derivatives thereof, inhibit Sb9, thereby de-repressing the host anti-tumor immunity. In some embodiments, inhibition of Sb9 by the compounds described herein, e.g., compounds of any one of Formulas (I), (II), (III), (IV), (V), (Va), (Vb) or (VI), and pharmaceutically acceptable salts and derivatives thereof, directly kills the tumor and de-represses the host-anti-tumor immunity.

In some embodiments, the inhibitor is 1,3-benzoxazole-6-carboxylic acid (CAS Number 154235-77-5). See U.S. Pat. No. 8,710,048 and U.S. Pub. No. 2005/0014767, each of which are incorporated by reference herein in their entireties. In some embodiments, the inhibitor is not 1,3-benzoxazole-6-carboxylic acid.

Combination Therapies

The methods described herein can also include administering the Sb9 inhibitor or an analog thereof in combination with other treatment modalities, e.g., chemotherapy or immunotherapy. In some embodiments, the Sb9 inhibitor or analog thereof is a compound described herein, e.g., compounds of Formula (I), (II), (III), (IV), (V), or (VI), or a pharmaceutically acceptable salt thereof. For example, chemotherapy can include one or more agents used in XELOX or FOLFOX/FOLFIRI/FOLFOXRI or related regimens, e.g., fluorouracil (5-FU) (e.g., oral form capecitabine), preferably in combination with one or more of leucovorin, irinotecan and oxaliplatin; luoropyrimidine such as 5-FU or capecitabine; irinotecan or oxaliplatin in combination with a fluoropyrimidine; or EMICORON (Porru et al., J Exp Clin Cancer Res. 2018; 37: 57). Immunotherapies can include checkpoint inhibitors, e.g., as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., Histol Histopathol. 2007 June; 22(6):687-96; Eggermont et al., Semin Oncol. 2010 October; 37(5):455-9; Klinke D J., Mol Cancer. 2010 Sep. 15; 9:242; Alexandrescu et al., J Immunother. 2010 July-August; 33(6):570-90; Moschella et al., Ann N Y Acad Sci. 2010 April; 1194:169-78; Ganesan and Bakhshi, Natl Med J India. 2010 January-February; 23(1):21-7; Golovina and Vonderheide, Cancer J. 2010 July-August; 16(4):342-7. Preferably, agents that target EGFR are not used.

In some embodiments, e.g., wherein the subject has colorectal cancer, 5-fluorouracil (5-FU) (e.g., oral form capecitabine), preferably in combination with one or more of leucovorin, irinotecan and oxaliplatin, e.g., in a FOLFOX/FOLFIRI/FOLFOXRI regimen; panitumumab, cetuximab, bevacizumab, ramucirumab, and aflibercept can also be combined with 5-FU, plus irinotecan or oxaliplatin, for metastatic colorectal cancer. Regorafenib can also be used in the present methods.

In some embodiments, e.g., wherein the subject has pancreatic cancer, gemcitabine, 5-fluorouracil (5-FU) (e.g., oral form capecitabine), preferably in combination with one or more of leucovorin, irinotecan and oxaliplatin, e.g., in a FOLFOX/FOLFIRI/FOLFOXRI regimen; paclitaxel (e.g., ABRAXANE® (albumin-bound)), and/or irinotecan (ONIVYDE®, liposome injection) can be used.

In some embodiments, e.g., wherein the subject has non-small cell lung cancer, the methods can include administering chemotherapy comprising one, two, or more of Cisplatin; Carboplatin; Paclitaxel (Taxol); Albumin-bound paclitaxel (nab-paclitaxel, Abraxane); Docetaxel (Taxotere); Gemcitabine (Gemzar); Vinorelbine (Navelbine); Irinotecan (Camptosar); Etoposide (VP-16); Vinblastine; and Pemetrexed (Alimta). Some preferred combinations include cisplatin or carboplatin plus one other drug, or gemcitabine with vinorelbine or paclitaxel. Targeted therapy drugs including bevacizumab (Avastin), ramucirumab (Cyramza), or necitumumab (Portrazza) can be added as well.

Preferred embodiments include administration of numerous additional anticancer agents such as, but not limited to, agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g. interferons for example. IFN-α, etc.) and interleukins (for example IL-2, etc.); adoptive immunotherapy agents; hematopoietic growth factors; agents that induce tumor cell differentiation (e.g. all-trans-retinoic acid, etc.); gene therapy reagents; antisense therapy reagents and nucleotides; tumor vaccines; inhibitors of angiogenesis, and the like. Numerous other examples of chemotherapeutic compounds and anticancer therapies suitable for co-administration are known to those skilled in the art.

In preferred embodiments, additional anticancer agents to be used in combination with compounds of the preferred embodiments comprise agents that induce or stimulate apoptosis. Agents that induce apoptosis include, but are not limited to, radiation (e.g., w); kinase inhibitors (e.g., epidermal growth factor receptor kinase inhibitor, vascular endothelial growth factor receptor kinase inhibitor, fibroblast growth factor receptor kinase inhibitor, platelet-derived growth factor receptor I kinase inhibitor, and Bcr-Abl kinase inhibitors such as STI-571, Gleevec, and Glivec); antisense molecules; antibodies (e.g., Herceptin and Rituxan); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., Celecoxib, meloxicam, NS-398, and non-steroidal anti-inflammatory drugs); and cancer chemotherapeutic drugs, e.g., irinotecan (Camptosar), CPT-11, fludarabine (Fludara), dacarbazine (DTIC), dexamethasone, mitoxantrone, Mylotarg, VP-16, cisplatinum, 5-FU, doxrubicin, taxotere or taxol; cellular signaling molecules; ceramides and cytokines; and staurosprine.

The present methods can include a combinations of administering a compound described herein together with an immunotherapy. Immunotherapies can include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, Prostaglandin E2 Inhibitors (e.g., using SC-50) and/or checkpoint inhibitors. In some embodiments, the methods include administering a composition comprising tumor-pulsed dendritic cells, e.g., as described in WO2009/114547 and references cited therein. See also Shiao et al., Genes & Dev. 2011, 25:2559-2572.

In some embodiments, the methods can include administering a checkpoint inhibitor, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 January; 8(1): 10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, tremelimumab, and EPR1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US Pub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1):80-96; Antoni et al., Am Soc Clin Oncol Educ Book. 2016; 35:e450-8; US Pub. Nos. 20180326054; 20180251767; 20180230431; 20170334995; 20170290914; 20170101472; 20170022273; 20160303124, and include BMS-986016.

See, e.g., Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7.

In some embodiments, the additional treatment modality is administered prior to, after, or concurrently with administration of the compound that is an inhibitor of Sb9 or analog thereof, such as a compound described herein, e.g., compounds of Formulas (I), (II), (III), (IV), (V), or (VI), or pharmaceutically acceptable salts or derivatives thereof. In some embodiments, the additional treatment modality is administered prior to administration of the Sb9 inhibitor or analog thereof. In some embodiments, the additional treatment modality is administered after administration of the Sb9 inhibitor or analog thereof. In some embodiments, the additional treatment modality is administered concurrently with administration of the Sb9 inhibitor or analog thereof. In some embodiments, the additional treatment modality is chemotherapy. In some embodiments, the additional treatment modality is immunotherapy. In some embodiments, the additional treatment modality involves administering a checkpoint inhibitor, such as an anti-PD-1 antibody, an anti-CD40 antibody, a CTLA-4 antibody, an anti-Tim3 antibody, and an anti-Lag3 antibody. In some embodiments, the Sb9 inhibitor or analog thereof is a compound of Formula (I) or a pharmaceutically acceptable salt thereof. In some embodiments, the Sb9 inhibitor or analog thereof is a compound of Formula (II) or a pharmaceutically acceptable salt thereof. In some embodiments, the Sb9 inhibitor or analog thereof is a compound of Formula (III) or a pharmaceutically acceptable salt thereof. In some embodiments, the compound is a derivative or analog of a compound of Formula (III) or a pharmaceutically acceptable salt thereof, such as the compounds provided in the present disclosure. In some embodiments, the Sb9 inhibitor or analog thereof is a compound of Formula (IV) or a pharmaceutically acceptable salt thereof. In some embodiments, the compound is a derivative or analog of a compound of Formula (IV) or a pharmaceutically acceptable salt thereof, such as the compounds provided in the present disclosure. In some embodiments, the Sb9 inhibitor or analog thereof is a compound of Formula (V) or a pharmaceutically acceptable salt thereof. In some embodiments, the Sb9 inhibitor or analog thereof is a compound of Formula (VI) or a pharmaceutically acceptable salt thereof.

Pharmaceutical Compositions and Methods of Administration

The methods described herein include the use of pharmaceutical compositions comprising one or more inhibitors of Serpin B9 or an analog thereof, e.g., a compound described herein, as an active ingredient. In some embodiments, the compound is the compound of Formula (I) or a pharmaceutically acceptable salt thereof. In some embodiments, the compound is the compound of Formula (II) or a pharmaceutically acceptable salt thereof. In some embodiments, the compound is the compound of Formula (III) or a pharmaceutically acceptable salt thereof. In some embodiments, the compound is an analog or derivative of a compound of Formula (III) or a pharmaceutically acceptable salt thereof, such as the compounds provided in the present disclosure. In some embodiments, the compound is the compound of Formula (IV) or a pharmaceutically acceptable salt thereof. In some embodiments, the compound is an analog or derivative of a compound of Formula (IV) or a pharmaceutically acceptable salt thereof, such as the compounds provided in the present disclosure. In some embodiments, the compound is the compound of Formula (V) or a pharmaceutically acceptable salt thereof. In some embodiments, the compound is the compound of Formula (VI) or a pharmaceutically acceptable salt thereof.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., checkpoint inhibitors, such as an anti-PD-1 antibody, an anti-CD40 antibody, a CTLA-4 antibody, an anti-Tim3 antibody, and an anti-Lag3 antibody.

Pharmaceutical compositions are typically formulated to be compatible with the intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, compositions comprising a LOXL1 enhancer for transdermal application can further comprise cosmetically-acceptable carriers or vehicles and any optional components. A number of such cosmetically acceptable carriers, vehicles and optional components are known in the art and include carriers and vehicles suitable for application to skin (e.g., sunscreens, creams, milks, lotions, masks, serums, etc.), see, e.g., U.S. Pat. Nos. 6,645,512 and 6,641,824. In particular, optional components that may be desirable include, but are not limited to absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

The LOXL1 enhancer compositions can also be prepared in the form of suppositories (e.g., with conventional suppository bases such as cocoa butter and other glycerides) or retention enemas for rectal or vaginal delivery. Such suppositories can be used particularly for the treatment of conditions associated with the loss of in elastic fibers that affect the pelvic organs, e.g., pelvic organ prolapse and/or urinary incontinence, inter alia.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36(1):49-62 (2008).

### EXAMPLES

The compounds and methods of the present disclosure are further described in the following examples, which do not limit the scope of the claims.

Materials and Methods

The following materials and methods were used in the Examples set forth herein.

Chemicals and Reagents

Dulbecco's modified Eagle's medium (DMEM), RPMI-1640 medium, fetal bovine serum (FBS), penicillin/streptomycin, and trypsin 0.25% were products of Hyclone, Thermo Scientific (Logan, Utah). Dimethylsulfoxide (DMSO), Tris-HCl, sodium chloride, glycerol, and p-mercaptoethanol were purchased from Sigma-Aldrich (MO, USA). Zenobia Library 1 (352 compounds), Zenobia Library 2.2 (288 compounds), Life Chemical Fragments (436 compounds), and the compound of Formula (III) (1,3-benzoxazole-6-carboxylic acid, purity 99% (CAS Number 154235-77-5)) were obtained from Enamine LLC (NJ, USA). Recombinant murine IL-2 was purchased from PeproTech (NJ, USA). Granzyme B inhibitor I—Calbiochem (368050) was purchased from Millipore Sigma (MA, USA). The antibiotics puromycin, blasticidin, and G418 were purchased from InvivoGen (CA, USA). Compounds 60-162, as shown in Table 1, are commercially available and were obtained from ChemBridge (CA, USA). Compounds 1-59 and 163-202, as shown in Table 1, were provided by Enamine (Kyiv, Ukraine). The compounds of Formula (VI) were provided by Enamine (Kyiv, Ukraine).

Cell Lines and Cell Culture

B16-F10 mouse melanoma, RENCA mouse renal adenocarcinoma, 4T1 mouse breast cancer, LLC1 mouse lung cancer, and A375 human melanoma cell lines were purchased from American Type Culture Collection (VA, USA). B16-F10, RENCA, and 4T1 cell lines were cultured in RPMI-1640 medium with 10% FBS and 1% penicillin/streptomycin. All other cell lines were grown in DMEM medium with 10% FBS and 1% penicillin/streptomycin. Cells were maintained in a 37° C. incubator at 5% CO₂.

Immunofluorescence Staining

5 μm sections of tissue were cut by cryosectioning and stained with conjugated or purified antibodies. Purified antibodies were detected using secondary antibodies. The antibodies used include anti-Sb9 (PA5-51038, Invitrogen), anti-mouse GrB (16G6, #14-8822-82, Invitrogen), anti-human GrB (2C5, sc-8022, SCBT), anti-CD271 (EP1039Y, ab52987, Abcam), anti-Nestin (7A3, sc-101541, SCBT), anti-MelanA (EPR20380, ab210546, Abcam), anti-α-SMA (D4K9N, #19245S, CST), anti-CD31 (clone 390, #14-0311-82, Invitrogen), anti-CD3 (SP7, ab16669, Abcam), anti-cleaved caspase-3 (Asp175, #9661, CST), anti-Collagen I (NB600-408, Novus), anti-Fibronectin (NBP1-91258, Novus), anti-PDGFR-α (APA5, ab90967, Abcam), anti-PDGFR-β (APB5, #14-1402-82, Invitrogen), anti-pan-cytokeratin (AE1/AE3, sc-81714, SCBT), anti-FAP (ab53068, Abcam), anti-FSP-1 (S100A4, ABF32, Millipore), anti-ER-TR7 (ab51824, Abcam), FITC anti-CD11b (M1/70, #101206, Biolegend), Alexa Fluor® 488 anti-Gr-1 (RB6-8C5, #108417, Biolegend), and PerCP/Cy5.5 anti-Ly-6C (HK1.4, #128012, Biolegend). DAPI (VECTASHIELD, Vector Laboratories) was used to counterstain the cell nuclei.

The stained tissue sections were visualized using an EVOS™ FL Auto 2 Imaging System (Thermo Fisher Scientific) for whole images and a fluorescence confocal microscope (Nikon) for high-resolution images. Quantification was performed on 2-3 sections from at least 3 separate mice using image analysis software Celleste (Invitrogen) and image J (NCBI, 1.8.0_112).

RNA In Situ Hybridization (RNAscope)

Granzyme B mRNA in situ hybridization (ISH) was measured with RNAscope assay (Advanced Cell Diagnostics, ACD, Hayward, Calif.), according to the manufacturer's protocols. Briefly, 4T1 cells and B16 cells were hybridized with Granzyme B probe (ACD, Cat. No. 490191), positive control probe (ACD, Cat. No. 320881), and negative control probe (ACD, Cat. No. 320871) at 40° C. for 2 h. Hybridization signals were amplified and visualized with RNAscope® Multiplex Fluorescent Reagent Kit v2 (ACD, Cat. No. 323100). Images were captured with a fluorescence microscope (EVOS™ FL AUTO 2).

Western Blotting

Lysates of cells and tissues were measured using the Bradford assay. Equal amounts of protein were separated by SDS-PAGE and transferred to a PVDF membrane. The membranes were immunoblotted with the following specific antibodies: anti-rabbit IgG-HRP (Sigma-Aldrich), anti-rat IgG-HRP (Sigma-Aldrich), and anti-GAPDH (Sigma-Aldrich), using standard protocols. The blots were developed with West Dura chemiluminescent substrates using a Bio-Rad ChemiDoc imaging system.

Transfection and Lentivirus Transduction

Cells were transfected with Lipofectamine 2000 (Invitrogen), according to the manufacturer's instructions. Briefly, cells were plated at 20-30% density in 12-well plates 24 hrs prior to transfection. For plasmid transfection, the equivalent of 0.4 μg of plasmid per well of a 12-well plate was utilized. Plasmids utilized included Control Double Nickase Plasmid (control plasmid, sc-437281, SCBT), PI-9 Double Nickase Plasmid (h) (sc-404486-NIC, SCBT), and serpinB9 expression plasmid in pcDNA3.1+/C-(K)DYK (OMu03805D, GenScript). After 24-72 hrs incubation, selective medium was used to complete screening for successfully transfected cells. PI-9 Double Nickase Plasmid (h) is designed to disrupt gene expression by causing highly specific Cas9-mediated double nicking of the SERPINB9 (human) gene, which mimics a double-strand break. It consists of a pair of plasmids, each encoding a D10A mutated Cas9 nuclease and a unique, target-specific 20 nt guide RNA (gRNA). This pair of gRNAs target exon 3 of the SERPINB9 (human) gene, and their sequences are ACCTGCTGAGAACGGCCAAC (SEQ ID NO:1) and TCACTTCAGTGAGAAGCGAC (SEQ ID NO:2). Control Double Nickase Plasmid was used as the negative control.

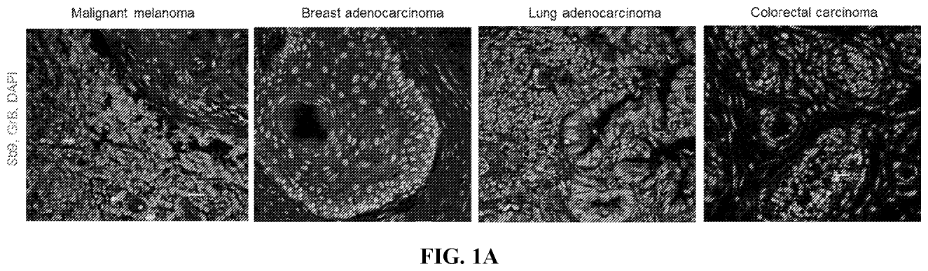

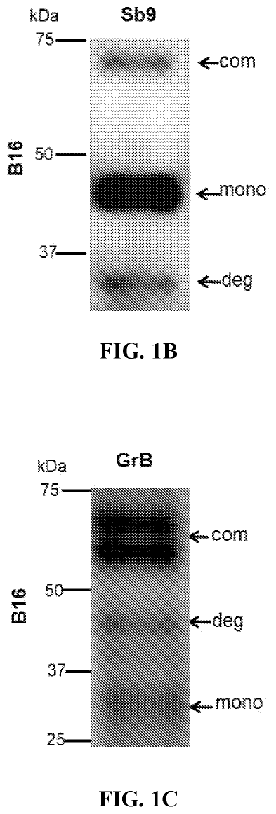

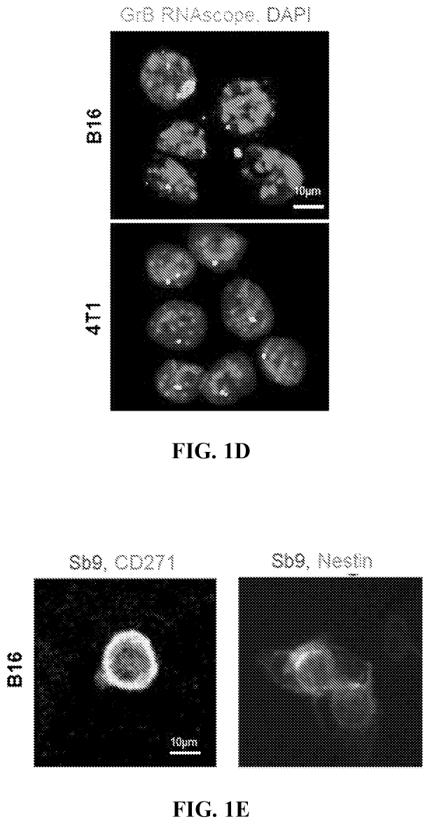

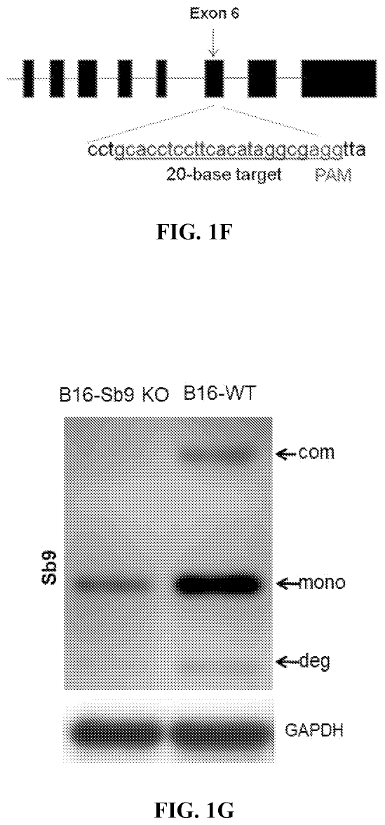

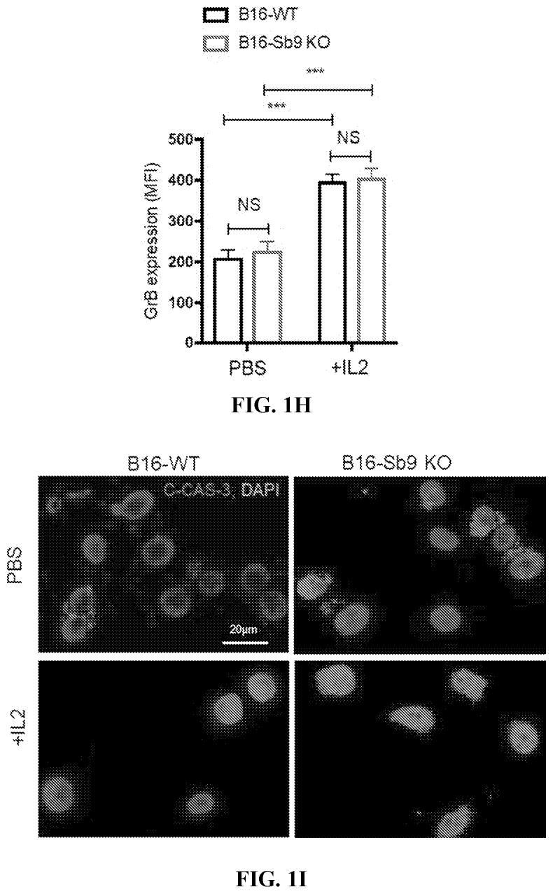

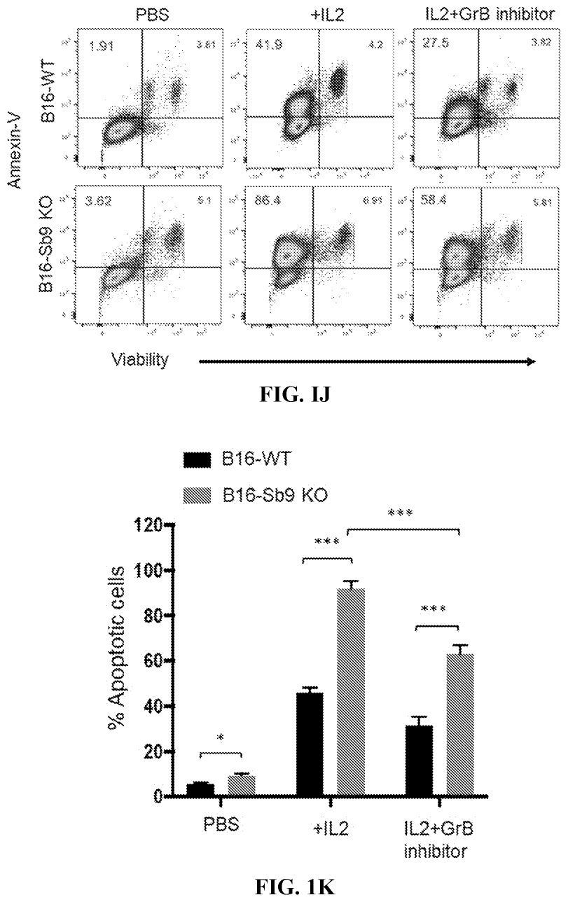

To disrupt gene expression of the serpinb9 (mouse) gene, we targeted the 20-nucleotide sequence upstream of the protospacer-adjacent motif (PAM) sequence in exon 6 of the serpinb9 (mouse) gene (FIG. 1F). The gRNA (sequence GCACCTCCTTCACATAGGCG (SEQ ID NO:3)) was cloned at the BsmBI site into the LentiCRISPRv2-blast (#98293, Addgene) vector, and pLentiCRIPSRv2 gRNA was subjected to sequence analysis to confirm the inserted oligonucleotide. Empty LentiCRISPRv2-blast vector was used as negative control. Lentiviral particles were generated by co-transfecting LentiCRISPRv2-blast plasmid into HEK293T cells with packaging plasmids pVSVg (#8485, Addgene) and psPAX2 (#12260, Addgene). For lentivirus transduction, cells were infected at either 20 or 40 MOI for 24 hrs with a Cas9-expressing lentivirus. Following blasticidin selection, the limiting dilution method in 96-well plates was used to select single-cell clones. The expression of Sb9 in the puromycin/G418/blasticidine-resistant clones was then analyzed by Western blotting.

Parental B16 cells were transduced with LV-GFP-PGK-Puro encoding the enhanced green fluorescent protein (GFP) cDNA under the spleen focus-forming virus (SFFV) promoter and the puromycin resistance gene (Puro) under the phosphoglycerate kinase (PGK) promoter. High GFP-expressing cells were selected using puromycin. The lentiviral vectors are self-inactivating vectors in which the viral enhancer and promoter have been deleted, and this transcription inactivation increases biosafety.

Reverse Transcription (RT) and Quantitative PCR

Total RNA was prepared from cells using the Quick-RNA MiniPrep kit (Zymo Research, Irvine, Calif., USA), according to the manufacturer's instructions. cDNA was prepared from total RNA using SuperScript III Reverse Transcriptase (Invitrogen). Following reverse transcription, quantitative PCR was performed using a cycling profile consisting of 95° C. for 2 min (Stage I), 30 cycles of 95° C. for 20 s, 58° C. for 30 s, and 70° C. for 30 s (Stage II), and 65° C. for 5 s (Stage III). Fold changes were determined by subtracting Cq values of the loading control from the Cq values of the gene of interest. The results were normalized to untreated controls.

Mice

All animal experiments and methods were performed in accordance with the relevant guidelines and regulations approved by the Institutional Animal Care and Use Committee of Brigham and Women's Hospital, Harvard Medical School, Boston, Mass. (protocol number: 2016N000167/04977). C57BL/6J (WT) (#000664), BALB/c (WT) (#000651), and C57BL/6-Serpinb9^{tm1Arp}/J (Spi6^{-/-}) (#008158) mice were purchased from Jackson Laboratory (Bar Harbor, Me., USA) and used at 8-10 weeks of age. NOD. Cg-Prkdc^{scid}/l2^{tm1Wj1}/SzJ (NSG) (#005557) mice were provided by Dr. Leonard D. Shultz from the Jackson Laboratory.

Tumor Implantation

Mice were anesthetized with isoflurane, and tumor cell lines (B16, A375, and 4T1) were gently injected subcutaneously in the flanks or mammary glands of mice. One hundred thousand cells (1×10⁵ cells) were injected per mouse for most of the experiments. However, twenty-five thousand cells (25×10³ cells) were injected per mouse for the experiments using the compound of Formula (III). Upon 4 hrs post-implantation, 300 μg of the compound of Formula (III) was administered twice a day intraperitoneally for 14 days. Tumor growth was monitored three times per week by digital caliper (Fisherbrand™ Traceable™ Digital Calipers).

Preparation of Tumor Tissues for Flow Cytometry

Tumor tissues from mice were minced into small pieces using a razor blade, then transferred to 70 μm cell strainers (BD) and separated mechanically using the plunger of a 5 mL syringe. Cells that passed through the cell strainer were collected in a 50 mL conical tube and resuspended in the RPMI-1640 complete growth medium. The single-cell suspensions from tumors were then ready for staining.

Flow Cytometry

Cells were plated in 96-well round-bottom plates (Corning, N.Y.) for intracellular cytokine staining and 96-well flat-bottom plates (Corning, N.Y.) for cell-surface and intracellular transcription factor staining. The cell samples that underwent intracellular cytokine staining were first incubated with 100 ng/mL PMA and 1 μg/mL ionomycin (Sigma-Aldrich), and GolgiStop™ protein transport inhibitor (BD Bioscience) at 37° C. for 4 hr.

All samples were washed with DPBS prior to incubation with Fixable Viability Dye eFluor™ 780 (Thermo Fisher Scientific), diluted 1:1000 in DPBS for 30 min at 4° C. Then, the cells were washed with FACS buffer (DPBS+2% fetal bovine serum+1 mM EDTA+0.1% sodium azide) and incubated for 30 min at 4° C. with the following cell-surface antibodies: PB anti-CD4 (GK1.5, #100428, Biolegend), BV510 anti-CD8 (53-6.7, #100752, Biolegend), APC anti-CD44 (IM7, #103012, Biolegend), PE/Cy7 anti-CD62L (MEL-14, #104418, Biolegend), PE anti-CD25 (7D4, #558642, BD Pharmingen), BV510 anti-CD45 (30-F11, #103138, Biolegend), PE/Cy7 anti-CD11b (M1/70, #101216, Biolegend), PB anti-F4/80 (BM8, #123124, Biolegend), PE anti-Ly-6G (1A8, #127608, Biolegend), Alexa Fluor® 488 anti-Gr-1 (RB6-8C5, #108417, Biolegend), PerCP/Cy5.5 anti-Ly-6C (HK1.4, #128012, Biolegend), BV510 anti-CD4 (RM4-5, #100559, Biolegend), FITC anti-CD8 (53-6.7, #553031, BD Pharmingen), APC anti-CD3 (17A2, #100236, Biolegend), PerCP/Cy5.5 anti-CD73 (TY/11.8, #127213, Biolegend), PB anti-CD105 (MJ7/18, #120411, Biolegend), BV510 anti-CD90 (30-H12, #105335, Biolegend), APC anti-CD117 (c-Kit) (2B8, #105812, Biolegend), PE/Cy7 anti-Ly6A/E (Sca-1) (D7, #108114, Biolegend), FITC anti-CD29 (HM31-1, #102205, Biolegend), and PE anti-CD44 (BJ18, #338808, Biolegend). All of the cell-surface antibodies were diluted 1:300 in FACS buffer.

The cells were permeabilized using the eBioscience Intracellular Fixation and Permeabilization Buffer Set (Thermo Fisher Scientific) for 30 min at 4° C. Then they were incubated with the following intracellular antibodies: PerCP/Cy5.5 anti-FOXP3 (FJK-16s, #45-5773-82, Invitrogen), PB anti-GrB (GB11, #515408, Biolegend), APC anti-IFNγ (XMG1.2, #505810, Biolegend), FITC anti-TNFα (MP6-XT22, #506304, Biolegend), PE anti-IL2 (JES6-5H4, #554428, BD Pharmingen), PE/Cy7 anti-IL10 (JES5-16E3, #505026, Biolegend), and PerCP/Cy5.5 anti-IL17A (TC11-18H10.1). All of the intracellular antibodies were diluted 1:300 in the eBioscience Permeabilization Buffer (1×) (Thermo Fisher Scientific). Cells were washed once with Permeabilization Buffer and fixed in FACS buffer containing 1% formalin. Flow cytometry was performed using a BD FACSCanto™ II flow cytometer (BD Biosciences). Analysis of flow cytometry results was performed via FlowJo software (FlowJo LLC, Ashland, Oreg.).

Isolation and Culture of Mouse Bone Marrow-Derived Mesenchymal Stem Cells (MSCs)

Isolation and harvest of murine MSCs from either C57BL/6-WT mice or Sb9 KO mice were performed according to the protocol as described previously (Zhu et al., 2010).

Protein Expression and Purification

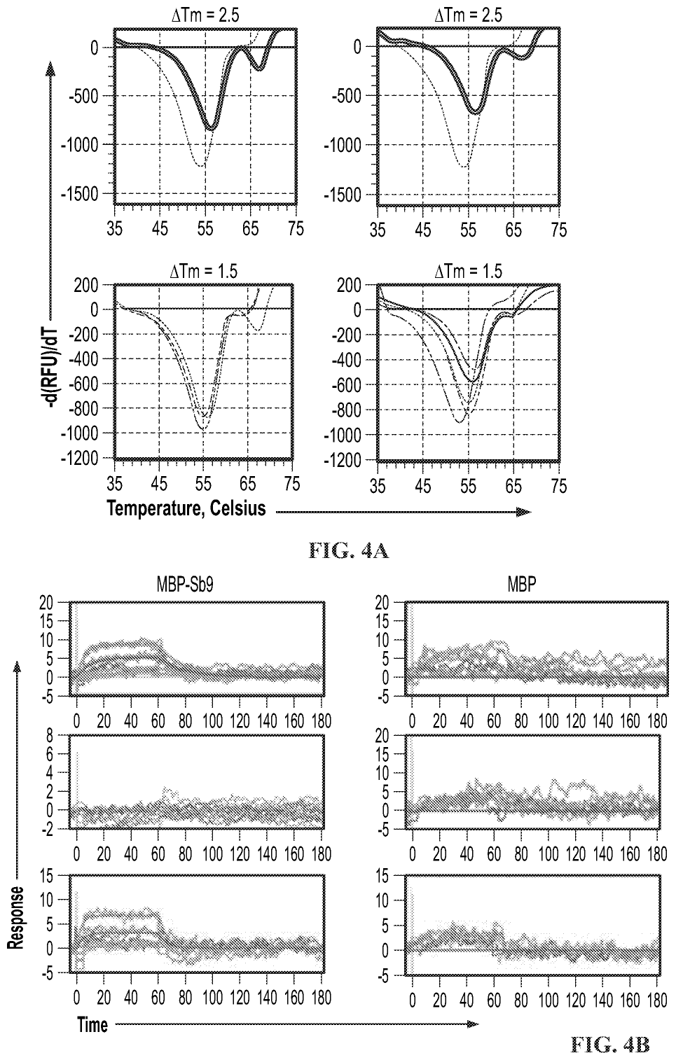

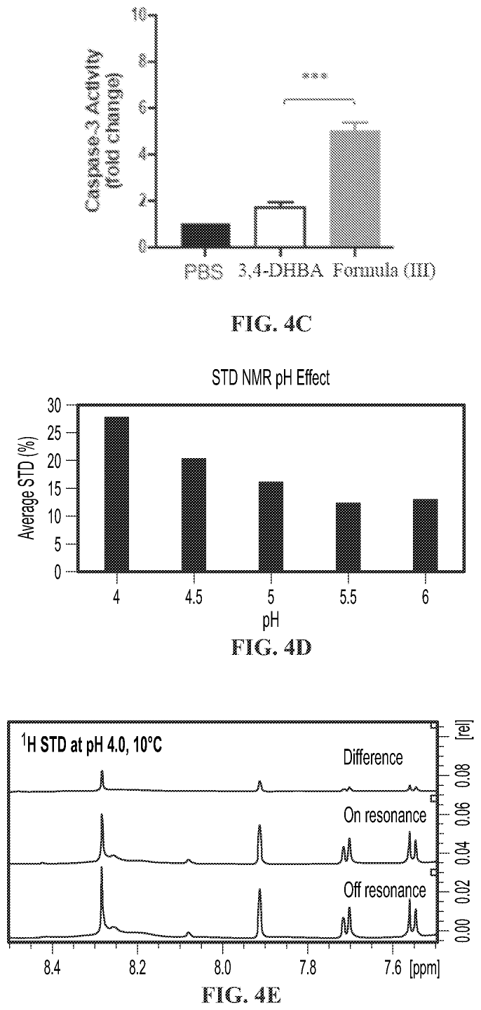

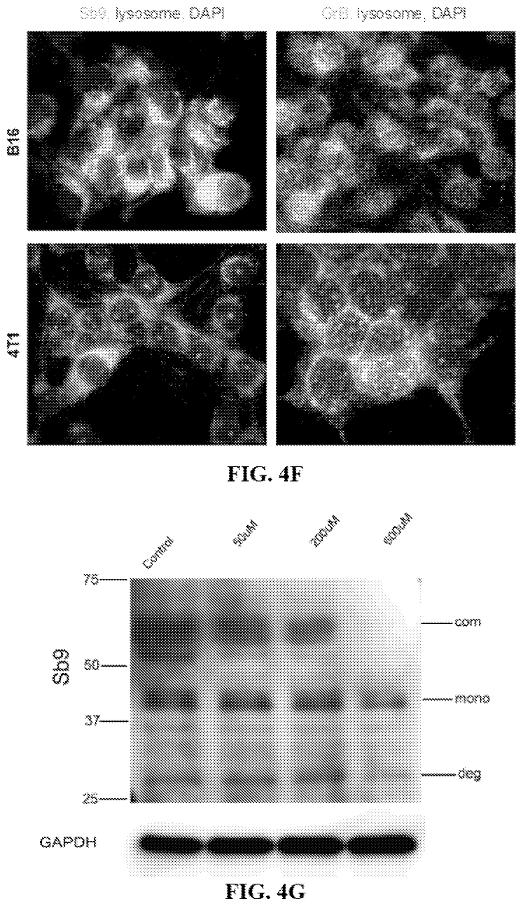

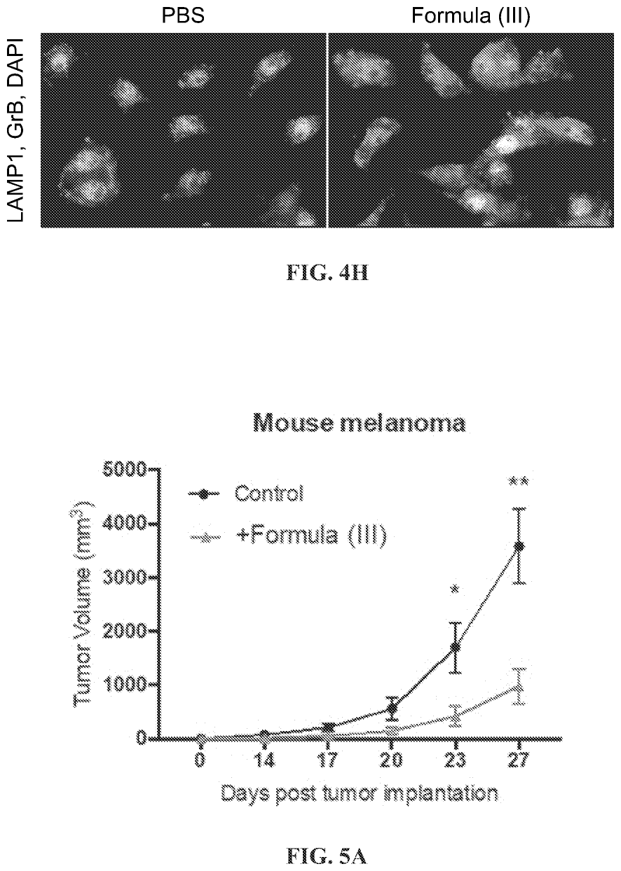

In an attempt to produce a soluble variant of Sb9, a construct was designed in which a maltose-binding protein (MBP) was added at the N-terminus (FIG. 4B). Sb9 was soluble in multiple growth media conditions using this strategy. The condition (1 mM IPTG, 37° C., 3 h) for best expression was selected for biomass production with 5 L culture. The cells were lysed and clarified by centrifugation. The fusion protein was then captured by Ni-NTA affinity purification, such that it was eluted using high concentration imidazole. Then, the resulting protein was further purified by anionic exchange chromatography using a MonoQ column and a NaCl gradient for elution. An amount of 31.4 mg protein was obtained. The purified Sb9 protein was characterized by SDS-PAGE and MALDI-TOF mass spectrometry (MS). A construct of human SERPINB9 covering residues 2-376 in the pFastBacl vector (Invitrogen) was overexpressed in insect Tni (Trichoplusia ni.) cells adapted for suspension culture in ESF921 medium (Expression systems). Cells were grown at 27° C. to a density of ˜2 million cells per mL, infected with baculovirus containing SERPINB9 construct, collected by centrifugation around 60 hrs post-infection, and stored at −80° C. Cell pellets were microfluidized at 15000 psi in buffer A (25 mM HEPES, pH 7.5, 200 mM NaCl, 10% glycerol, 20 mM imidazole, 7 mM BME) and the resulting lysate was centrifuged at 13,000 rpm for 30 min. Ni-NTA beads (Qiagen) were mixed with lysate supernatant for 30 min and washed with buffer A and eluted with buffer B (25 mM HEPES, pH 7.5, 200 mM NaCl, 10% glycerol, 400 mM imidazole, 7 mM BME). The sample was further purified by size exclusion chromatography using an Superdex-200 16/60 column in buffer C (20 mM HEPES, pH 7.5, 200 mM NaCl, 10% glycerol, 0.5 mM TCEP, 3 mM DTT). Protein containing fractions were pooled, concentrated, and stored at −80° C.

MALDI-TOF Mass Spectrometry

Analyses were performed on a Finnigan Laser MAT 2000 time-of-flight mass spectrometer (Finnigan MAT, Hemel Hempstead, Herts, UK). The system used a nitrogen laser (337 nm, 2 ns pulse) to desorb ions from the sample specimen. The desorbed ions were accelerated to 20 kV into a free long tube. The time recorded for a molecule to travel the length of the tube to a detector was proportional to the mass of the ion, which was its molecular weight. All spectra were obtained using the positive-ion mode. Standard stainless-steel targets (with a sample application area of about 3.14 mm²) obtained from the manufacturer were employed for all analyses. The lasermat software allowed the user to irradiate one of the four possible target regions or quadrants of about 0.02 mm². The spectra in this study were calibrated using instrumental calibration, based on the parameters determined from analysis of a number of standard proteins and peptides.

Thermal Stability Assay (TSA) and Initial Fragment (Small Organic Molecules) Screen

TSA is an analytic tool to estimate the overall stability of a protein by monitoring the shift in its melting temperature upon changing the buffer conditions or titrating various ligands. As the protein unfolds with increasing temperature in a thermal cycler (CFX384 Touch Real-Time PCR Detection System), SYPRO orange dye binds to the exposed hydrophobic core and fluoresces, leading to the signal that is measured in comparison to temperature to produce a melt curve. The rate of dye uptake peaks at the melting transition and signals the melting temperature (Tₘ). As an initial assay, MBP-Sb9 was tested at 1 mg/mL and 0.25 mg/mL. Four tests were performed at each concentration in Buffer A1 (20 mM Tris-HCl, pH 8.0, 20 mM sodium chloride, 5 mM 2-mercaptoethanol, and 10% glycerol): no additive, plus 10 mM maltose, plus 5% glycerol, and plus 10% glycerol. Dye was added at 2×strength, and the readings were performed from 25° C. to 95° C. at 0.5° C. intervals.

To set up the fragment screen assay, a plate was loaded with protein by manually pipetting 19 μL per well, well-by-well. Then, 0.5 μL of fragment solutions were transferred from the 384-well library stock plate in a replicate manner, using an Integra ViaFlo II electronic pipette and mixing by swirling the pipette tips. This resulted in testing the Zenobia compounds at 5 mM and the Life Chemicals compounds at 1.25 mM, and all assay wells contained 2.5% DMSO. On all plates, at least 8 wells tested buffer alone and at least 24 wells tested 2.5% DMSO, with the latter used for the baseline measurement. Once a plate was set, it was sealed with foil and incubated with shaking for 1 hour at 25° C. Sypro Orange dye was added at 2×strength (Thermo S6650) using the ViaFlo pipette. The plate was sealed with clear adhesive, briefly shaken to disperse the dye, and measured using the BioRad C1000 thermal cycler with a CFX384 Touch RealTime detector. The measurement cycle was performed from 25° C. to 95° C. with a measurement interval of 0.5° C. One plate of fragments was tested on a given day. A commercially available library of over 1,000 compounds was tested on four plates.

Surface Plasmon Resonance (SPR)

Surface plasmon resonance (SPR) measures change in refractive index on a biosensor surface, as analytic mass accumulates via binding to an attached ligand. This allows derivation of affinity (K_{D}) from measured kinetic rates of association (kₐ) and dissociation (k_{d}).

All assays were run at 25° C. on a BiOptix 404pi. An NTA biosensor chip was used in this study to pre-concentrate 3 μM MBP-Sb9 via an N-terminal His-tag. MBP-Sb9 was covalently immobilized with EDC/NHS in 20 mM HEPES pH 8.0, 150 mM NaCl, 0.05% Tween-20, 5 mM β-ME to 10,000 to 15,000 RUs. MBP was similarly immobilized on a separate channel as a negative control.

Compounds were injected as 3-fold serial dilutions in duplicate starting at 200 μM. Injections were done at 50 μL/min for 60 seconds, followed by a 120 second dissociation in 20 mM HEPES, pH 8.0, 150 mM NaCl, 0.05% Tween-20, 5 mM j-ME, 1% DMSO. A series of five DMSO standards between 0.5% and 1.5% were included with each run to correct for bulk refractive index. Sensorgrams were analyzed using Scrubber 2 software with a double reference and fit to a 1:1 Langmuir model to determine the interaction parameters K_{D}, kₐ, and k_{d}.

Caspase-3 Activity Assay

The EnzChek® Caspase-3 Assay Kit was used to detect apoptosis by assaying for increases in caspase-3 activities. The basis for the assay was rhodamine 110 bis-(N-CBZ-L-aspartyl-L-glutamyl-L-valyl-L-aspartic acid amide) (Z-DEVD-R110). This substrate is a bisamide derivative of rhodamine 110 (R110) containing DEVD peptides covalently linked to each of R110's amino groups, thereby suppressing the dye's visible absorption and its fluorescence. Upon enzymatic cleavage, the nonfluorescent bisamide substrate is converted in a two-step process first to a fluorescent monoamide and then to an even more fluorescent R110.

At least 1×10⁶ cells of the lysate were used for each reaction. B16 cells were treated with compounds to induce apoptosis. A negative control was prepared by incubating cells in the absence of compounds. The cells were incubated with compounds for 24 hrs and washed in PBS. Each cell sample or control was resuspended in 50 μL of the 1×cell lysis buffer. Cells were lysed by incubating on ice for 30 minutes, and the lysed cells were centrifuged to pellet the cellular debris. 50 μL of the supernatant was transferred from each sample to individual microplate wells. 50 μL of the 1× cell lysis buffer was used as a no-enzyme control to determine the background fluorescence of the substrate. 50 μL of the 2×substrate working solution was added to each sample and control. The microplate was covered, and the samples were incubated at room temperature for approximately 30 minutes. The fluorescence was measured by a fluorescence microplate reader (excitation/emission ˜496/520 nm) using excitation and emission filters.

Granzyme B Activity Assay

The goal of these experiments was to find a compound that deters the ability of Sb9 to inhibit granzyme B proteolysis. To that end, a granzyme B activity assay was developed to test a number of compounds from the fragment screen hits. The assay was a commercially available kit, the Granzyme B Colorimetric Drug Discovery Kit by Enzo Life Sciences (BML-AK711). In this assay, granzyme B cleaves the peptide substrate Ac-IEPD-pNA to produce the chromophore p-nitroaniline, which is measured by absorbance at 405 nm. The measurements were performed with a LabSystems Multiskan RC plate reader.

The appropriate volume of assay buffer was added to the wells of the microplate. 10 μL of tested compound was added to the designated wells. Then, 10 μL of Sb9 protein was added into the designated wells and pre-incubated with the compounds for 1 or 2 hrs. 15 μL of GrB was added to the designated wells. Then this mixture was incubated with GrB for 30 min. The reaction was started by adding 50 μL of the 2×Ac-IEPD-pNA substrate solution. 100 μL of pNA at 50 μM was added as a calibration standard, which produced an A405 of ˜0.3. The microplate was read continuously at A405 in a microplate-reading spectrophotometer. The reactions were run for 2 hrs, and the reading interval was 10 min. Lastly, the raw data was obtained and data analysis was performed.

Saturation-Transfer Difference (STD) NMR Assay

The Saturation-Transfer Difference (STD) NMR assay was used to evaluate the binding of the compound of Formula (III) to Sb9. The STD NMR experiment was performed using 420 μM of the compound of Formula (III) and 45 μM Sb9 protein in PBS buffer, pH 7.0 with 10% D₂O at 25° C., on a 600 MHz Bruker Avance II spectrometer equipped with a Prodigy Cryoprobe. The saturation period was 3.0 seconds with irradiation at 0 ppm (on-resonance excitation) and −20 ppm (off-resonance excitation). The number of scans was 160 for the on and off-resonance spectra, respectively. Spectra were processed using Topspin software and apodized with exponential multiplication with 1.0 Hz line-broadening.

Statistical Analysis

All experiments were repeated at least three times, each done in triplicate. The statistical significance between two groups was determined by Student's t-test, whereas the comparisons of multiple groups were carried out by one-way ANOVA, followed by Bonferroni's post-test using GraphPad Prism 7 software (GraphPad Software, Inc., CA). A probability value of *P<0.05 was considered to be significant.

Chemical Synthesis

The compound of Formula (I) (methyl 5-amino-2-((tert-butoxycarbonyl)amino)-4-hydroxybenzoate) was prepared as follows.

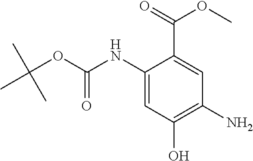 [SMILES: COC(=O)c1cc(N)c(O)cc1NC(=O)OC(C)(C)C]

Step A: Methyl 2-amino-4-hydroxybenzoate (50 g, 300 mmol, 1 eqv.) was dissolved in concentrated sulfuric acid (500 mL) while keeping the temperature below 0-5° C. Potassium nitrate (40 g, 387 mmol, 1.2 eqv.) was added slowly at a temperature maintained at 0-5° C. Then the reaction mixture stirred overnight at room temperature and was quenched after with ice/water. The liquid phase was separated, washed with water and dried over anhydrous sodium sulfate. Then the Na₂SO₄ was filtered and the organic solution concentrated under reduced pressure to afford methyl 2-amino-4-hydroxy-5-nitrobenzoate (55 g, 259 mmol, 86.4% yield).

Step B: To a suspension of methyl 2-amino-4-hydroxy-5-nitrobenzoate (55 g, 259 mmol, 1 eqv.) in dichloromethane (400 mL) was added triethylamine (31.4 g, 311 mmol, 1.2 eqv.), and the mixture was cooled to 0° C. upon stirring. Then di-tert-butyl dicarbonate (62.16 g, 285 mmol, 1.1 eqv.) was added portion-wise. The cooling bath was removed and the solution was stirred overnight at room temperature. The reaction was then concentrated under reduced pressure and water (100 mL) was added followed by extraction of the mixture with ethyl acetate (3×100 mL). The combined organic phases were washed with brine (2×100 mL), dried over sodium sulfate, filtered and concentrated. The residue was purified by flash chromatography (water-acetonitrile) to give methyl 2-[(tert-butoxy)carbonyl]amino-4-hydroxy-5-nitrobenzoate (20 g, 64 mmol, 25% yield).

Step C: A mixture of methyl 2-[(tert-butoxy)carbonyl]amino-4-hydroxy-5-nitrobenzoate (20 g, 64 mmol, 1 eqv.) and palladium on activated carbon Pd/C (10%, 2.0 g, 18.79 mmol) in methanol (250 mL) was stirred overnight at room temperature under a hydrogen atmosphere (10 atm). Then the mixture was filtered and the organic solution concentrated under reduced pressure. The residue was purified by flash chromatography (water-acetonitrile) to afford the title compound methyl 5-amino-2-[(tert-butoxy)carbonyl]amino-4-hydroxybenzoate (5.2 g, 95% purity, 18.44 mmol, 28.8% yield).

LCMS (ESI): m/z [M+H] calcd for C₁₃H₁₈N₂O₅: 282.29; found: 282.13.

¹H NMR (400 MHz, DMSO-d₆) δ=10.2 (s, 1H), 7.75 (s, 1H), 7.6 (s, 1H), 6.5 (s, 2H), 6.15 (s, 1H), 3.7 (s, 3H), 1.5 (s, 9H).

The compound of Formula (II) (methyl 5-amino-2-benzamido-4-hydroxybenzoate) was prepared as follows.

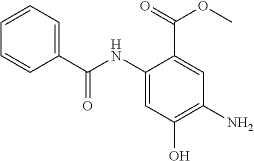 [SMILES: COC(=O)c1cc(N)c(O)cc1NC(=O)c1ccccc1]

Step A: 2-Amino-4-hydroxy-5-nitrobenzoic acid (15 g, 75.7 mmol, 1 eqv.) was dissolved in methanol (250 mL). The reaction mixture was cooled to 0° C. and thionyl chloride (22.5 g, 189 mmol, 2.5 eqv.) was added dropwise and then the reaction mixture was refluxed for 72 hours, then cooled and evaporated. Methyl 2-amino-4-hydroxy-5-nitrobenzoate (17.4 g, 70.4 μmol, 93% yield) was obtained as a hydrochloride salt, which was used in the next step without further purification.

Step B: To a suspension of methyl 2-amino-4-hydroxy-5-nitrobenzoate hydrochloride (17.4 g, 70 mmol, 1 eqv.) in dichloromethane (200 mL) was added triethylamine (17.6 g, 175 mmol, 2.5 eqv.), and the mixture was cooled to 0° C. with stirring. Then benzoyl chloride (10.78 g, 77 mmol, 1.1 eqv.) was added portion-wise. The cooling bath was removed and the solution was stirred overnight at room temperature. The reaction was then concentrated under reduced pressure and water (100 mL) was added followed by extraction of the mixture with ethyl acetate (3×100 mL). The combined organic phases were washed with brine (2×100 mL), dried over sodium sulfate, filtered and concentrated. The residue was purified by flash chromatography (water-acetonitrile) to give methyl 2-benzamido-4-hydroxy-5-nitrobenzoate (13 g, 41.1 mmol, 58.77% yield).

Step C: A mixture of methyl 2-benzamido-4-hydroxy-5-nitrobenzoate (13 g, 41.1 mmol, 1 eqv.) and palladium on activated carbon Pd/C (10%, 2.0 g, 18.79 mmol) in methanol (250 mL) was stirred overnight at room temperature under hydrogen atmosphere (10 atm). Then the mixture was filtered and the filtrate was evaporated under reduced pressure to give crude methyl 5-amino-2-benzamido-4-hydroxybenzoate (9.6 g, 95% purity, 33.56 mmol, 81.6% yield).

LCMS (ESI): m/z [M+H] calcd for C₁₅H₁₄N₂O₄: 286.28; found: 286.1.

¹H NMR (400 MHz, DMSO-d₆) δ=10.45 (br s, 1H), 9.35 (s, 1H), 7.95 (d, 3H), 7.5 (tt, 3H), 6.55 (s, 2H), 6.25 (s, 1H), 3.7 (s, 3H).

Exemplary synthetic routes for preparing Compound 27 and Compound 227 are provided below.

N-ethyl-N-methyl-2-phenyl-1,3-benzoxazole-6-carboxamide (Compound 27): N-ethyl-N-methyl-2-phenyl-1,3-benzoxazole-6-carboxamide (Compound 27, as shown in Table 1) was prepared according to Scheme 3.

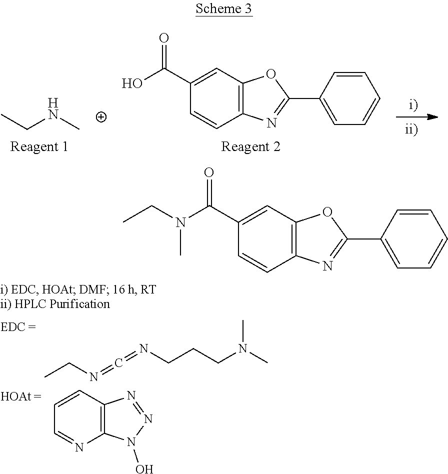

Ethyl(methyl)amine (Reagent 1, 18 mg, 0.305 mmol), 2-phenyl-1,3-benzoxazole-6-carboxylic acid (Reagent 2, 90 mg, 0.376 mmol), 1-ethyl-3-(3-dimethylaminopropyl)carbodiimide (EDC) (58 mg, 0.374 mmol), and 1-hydroxy-7-azabenzotriazole (HOAt) (43.5 mg, 0.32 mmol) were mixed in dry DMF* (appr. 0.5 mL per 100 mg of product). The reaction mixture was sealed and left at ambient temperature for 18 hours. Then the solvent was evaporated under reduced pressure and the residue was dissolved in the DMSO. The DMSO solution was filtered, analyzed by LCMS, and transferred for HPLC purification (Instrument specifications: Agilent 1260 Infinity systems equipped with DAD and mass-detector; Temperature: 25° C.; Column: Waters SunFire C18 OBD Prep Column, 100 A, 5 μm, 19 mm×100 mm with SunFire C18 Prep Guard Cartridge, 100 A, 10 μm, 19 mm×10 mm; Detection: DAD—DAD1A 215 nm, DAD1B 254 nm. MSD—single quadrupole, AP-ESI; Mobile phases: A—Deionized water (100%). B—HPLC-grade MeOH (100%); Gradient: from A-50%: B-50% to A-0%: B-100%). Yield: 53.2 mg (55.5%). Beige powder. LCMS purity: 100% (Instrument specifications: Agilent 1100 Series LC/MSD system with DAD\ELSD Alltech 2000ES and Agilent LC\MSD VL (G1956B), SL (G1956B) mass-spectrometer, Agilent 1200 Series LC/MSD system with DAD\ELSD Alltech 3300 and Agilent LC\MSD G6130A, G6120B mass-spectrometer, Agilent Technologies 1260 Infinity LC/MSD system with DAD\ELSD Alltech 3300 and Agilent LC\MSD G6120B mass-spectrometer, Agilent Technologies 1260 Infinity II LC/MSD system with DAD\ELSD G7102A 1290 Infinity II and Agilent LC\MSD G6120B mass-spectrometer; Detection: DAD—DAD1A 215 nm, DAD1B 254 nm MSD—single quadrupole, AP-ESI (positive/negative mode switching); Temperature: 25° C.; Column: InfinityLab Poroshell 120 SB-C18 4.6×30 mm 2.7 Micron with Guard: UHPLC Guard 3PK InfinityLab Poroshell 120 SB-C18 4.6×5 mm 2.7 Micron; Mobile phases: A—Deionized water: Formic acid (99.9:0.1%). B—HPLC-grade MeCN: (Deionized water: Formic acid (99.9:0.1%)) (95:5%); Gradient: from A—99%, B—1% to A—1%, B—99%). EI MS m/z: pos. 281.1 (MH⁺).

### N-benzyl-4-ethoxy-3-hydroxybenzamide (Compound 227)

N-benzyl-4-ethoxy-3-hydroxybenzamide (Compound 227, as shown in Table 2) was prepared according to Scheme 4.

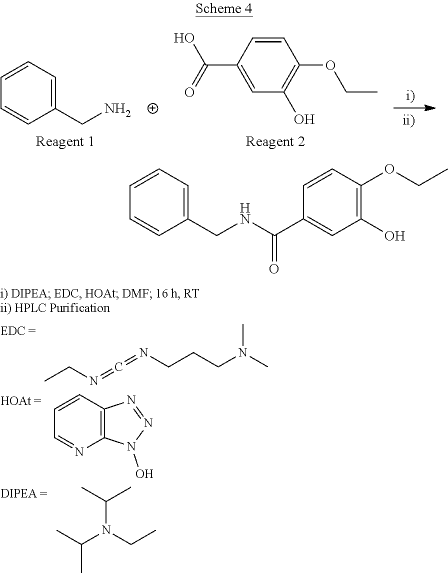

1-phenylmethanamine (Reagent 1, 39 mg, 0.364 mmol), 4-ethoxy-3-hydroxybenzoic acid (Reagent 2, 68 mg, 0.373 mmol), N,N-diisopropylethylamine (DIPEA) (114 mg, 0.882 mmol), 1-ethyl-3-(3-dimethylaminopropyl)carbodiimide (EDC) (59 mg, 0.38 mmol), and 1-hydroxy-7-azabenzotriazole (HOAt) (53.3 mg, 0.392 mmol) were mixed in dry DMF* (appr. 0.5 mL per 100 mg of product). The reaction mixture was sealed and left at ambient temperature for 18 hours. Then the solvent was evaporated under reduced pressure and the residue was dissolved in the DMSO (appr. 1 mL up to 300 mg of product). The DMSO solution was filtered, analyzed by LCMS, and transferred for HPLC purification (Instrument specifications: Agilent 1260 Infinity systems equipped with DAD and mass-detector; Temperature: 25° C.; Column: Waters Sunfire C18 OBD Prep Column, 100 A, 5 μm, 19 mm×100 mm with SunFire C18 Prep Guard Cartridge, 100 A, 10 μm, 19 mm×10 mm; Detection: DAD—DAD1A 215 nm, DAD1B 254 nm. MSD —single quadrupole, AP-ESI; Mobile phases: A—Deionized water (100%). B—IPLC-grade MeOH (100%); Gradient: from A-50%: B-50% to A-0%: B-100%). Yield: 19 mg (18.8%). Yellow powder. LCMS purity: 100% (Instrument specifications: Agilent 1100 Series LC/MSD system with DAD\ELSD Alltech 2000ES and Agilent LC\MSD VL (G1956B), SL (G1956B) mass-spectrometer, Agilent 1200 Series LC/MSD system with DAD\ELSD Alltech 3300 and Agilent LC\MSD G6130A, G6120B mass-spectrometer, Agilent Technologies 1260 Infinity LC/MSD system with DAD\ELSD Alltech 3300 and Agilent LC\MSD G6120B mass-spectrometer, Agilent Technologies 1260 Infinity II LC/MSD system with DAD\ELSD G7102A 1290 Infinity II and Agilent LC\MSD G6120B mass-spectrometer; Detection: DAD—DAD1A 215 nm, DAD1B 254 nm MSD—single quadrupole, AP-ESI (positive/negative mode switching); Temperature: 25° C.; Column: InfinityLab Poroshell 120 SB-C18 4.6×30 mm 2.7 Micron with Guard: UHPLC Guard 3PK InfinityLab Poroshell 120 SB-C18 4.6×5 mm 2.7 Micron; Mobile phases: A—Deionized water:Formic acid (99.9:0.1%). B—HPLC-grade MeCN:(Deionized water:Formic acid (99.9:0.1%)) (95:5%); Gradient: from A—99%, B—1% to A—1%, B—99%). EI MS m/z: pos. 272.1 (MH⁺).

### NMR:

Ligand-focused NMR. To test the binding of Compound 27 and Compound 225 to SERPINB9, we used three ligand-detected nuclear magnetic resonance (NMR) methods: differential line broadening (DLB), saturation transfer difference (STD), and Carr-Purcell-Meiboom-Gill-transverse relaxation rate (CPMG-R2). First, significant ligand line broadening and shifting was observed when SERPINB9 was present, which suggested a bound state for these compounds. Second, on-resonance saturation of SERPINB9 decreased the intensity of compound peaks in the STD experiment, indicating transmission of magnetization energy from SERPINB9 to proximal ligands. Finally, as ligands engaging the target protein relax faster (compared to free small molecules that have slow transverse relaxation rates in CPMG-R2 experiments), we saw a decreased relaxation time in the presence of a protein directly indicating binding of the ligand to that protein. Together, these results establish Compound 27 and Compound 227 as SERPINB9 binders.

### ITC:

A concentration of 20 μM SERPINB9 in a buffer containing 20 mM HEPES, pH 7.5, 150 mM NaCl, and 0.5 mM TCEP, with 2% DMSO was titrated with 200 μM Compound 27 and Compound 227 in the same buffer. Resulting isotherms were fitted with a single site model to yield thermodynamic parameters of ΔH, ΔS, stoichiometry, and K_{d} of 21.5 μM (Compound 27) and 28.8 μM (Compound 227).

### Results

### Example 1. Sb9 is Required to Protect Melanoma Tumors from GrB-Induced Apoptosis

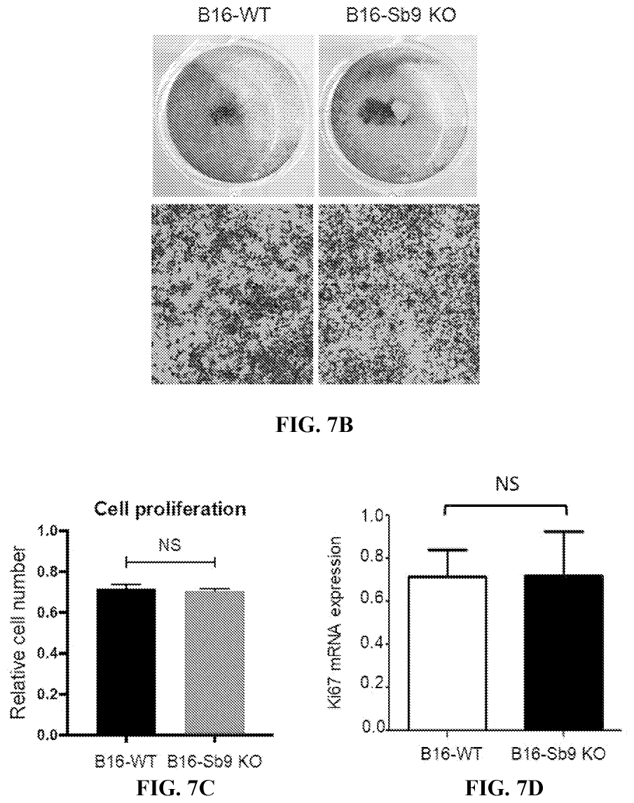

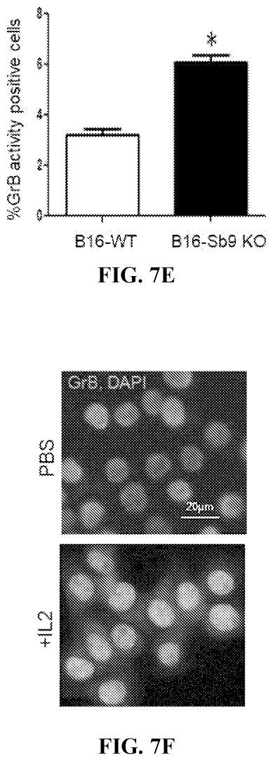

The expression of Sb9 and GrB in a number of primary human tumors and cancer cell lines was examined. Both Sb9 and GrB were expressed ubiquitously in primary human malignant melanoma, breast adenocarcinoma, lung adenocarcinoma, and colorectal carcinoma (FIG. 1A). Immunohistochemical analysis demonstrated the expression of Sb9 in primary human pancreatic adenocarcinoma, Hodgkin lymphoma, prostate adenocarcinoma, and ovarian carcinoma (FIG. 7A). Inhibition of GrB by Sb9 via a classic serpin mechanism resulted in SDS-resistant 62 kD Sb9-GrB complex in B16-F10 mouse melanoma cells (FIGS. 1B-1C) (Mangan et al., 2016; Zhang et al., 2006). Western blot demonstrated that Sb9 protein was expressed mainly as a 47 kD unbound monomer, while the remaining part of Sb9 protein formed a 62 kD Sb9-GrB complex in B16 cells (FIG. 1B) (Mangan et al., 2016). Interestingly, almost all of the GrB protein was observed to exist as a 62 KD complex with Sb9, in contrast to the 26 kD monomeric GrB protein found in B16 cells (FIG. 1C). The GrB RNAscope results demonstrated the gene expression of GrB in both B16 and 4T1 mouse breast tumor cells (FIG. 1D). Sb9 was co-expressed with the stem cell markers CD271 and Nestin, so indicating that Sb9 might identify a population of cancer stem cells within the B16 melanoma population (FIG. 1E) (Gonzalez-Garza et al., 2018; Neradil and Veselska, 2015).

To address the protection of Sb9 from GrB in melanoma cells, the serpinb9 gene was disrupted using the CRISPR/Cas9 gene editing system. The 20-nucleotide sequence upstream of the protospacer-adjacent motif (PAM) sequence in exon 6 of the serpinb9 gene was targeted (FIG. 1F). Deletion of Sb9 expression in the B16 melanoma cells was confirmed by Western Blot (FIG. 1G). The complex formation was hampered and suggested that all GrB in the cell was no longer captured by Sb9. Residual Sb9 protein expression by the B16-Sb9 KO cells is not an infrequent observation (FIG. 1G), due to common tetraploidy in these cells (Kendal et al., 1987; Smits et al., 2019).

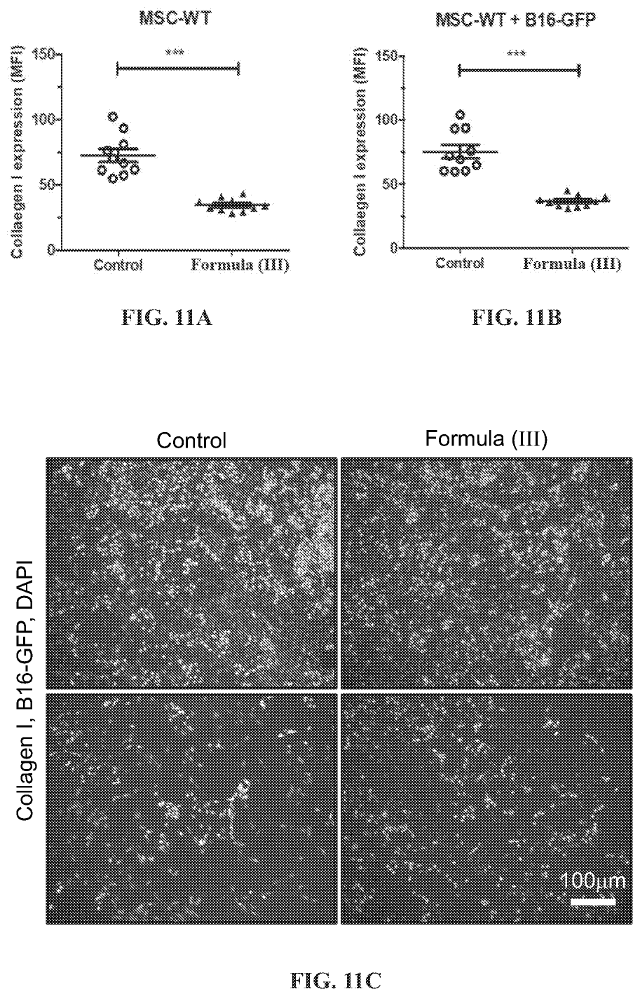

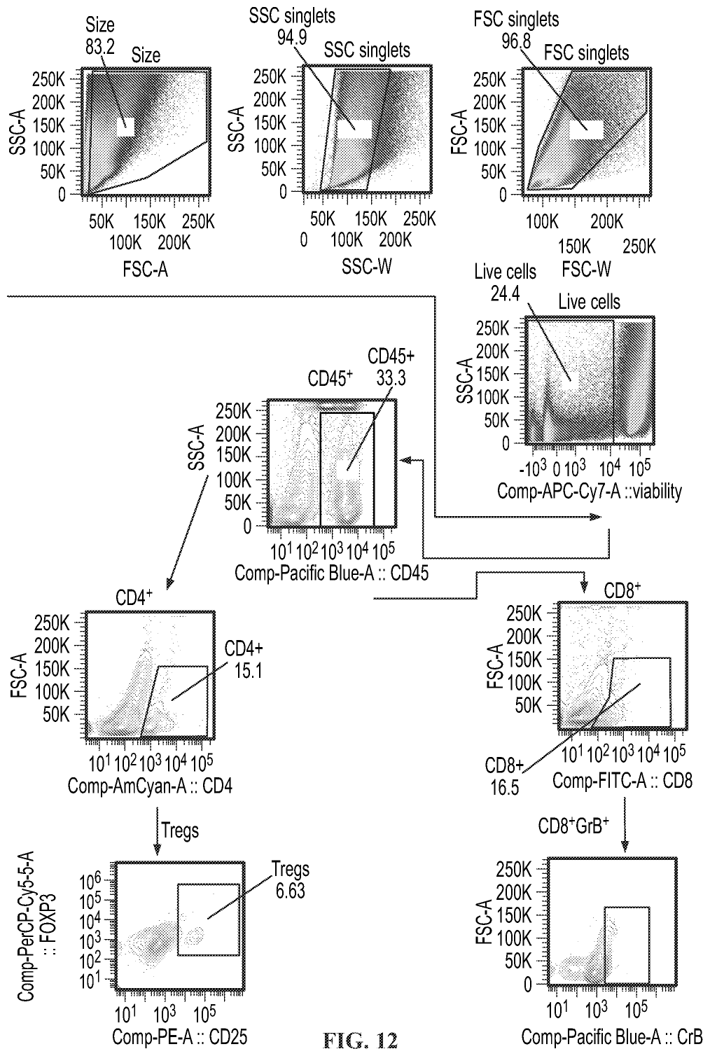

However, Sb9 knockout did not affect the proliferation of B16 cells, as evidenced by colony formation assay and the expression of the Ki67 gene (FIGS. 7B-7D) (Li et al., 2015). Sb9 knockout resulted in 2-fold increase (p=0.016) in GrB activity (FIG. 7E) and a corresponding 1.8-fold (p=0.017) increase in GrB-specific apoptosis in B16-Sb9 KO cells compared to B16-WT cells (FIGS. 1J-1K). Previous studies have reported that IL2 induces GrB expression in cytotoxic T cells (CTL) and Natural Killer (NK) CLs (Tamang et al., 2006). Here it was found that the expression of GrB was also elevated significantly (p=0.0002) in IL2-treated B16-WT and B16-Sb9 KO cells (FIGS. 1H and 7F). Sb9 knockout resulted in GrB-mediated apoptosis (FIGS. 11-1K). Collectively, these results demonstrated that Sb9 is required to protect tumors from GrB-induced apoptosis.

### Example 2. Sb9 Acts Cell Intrinsically to Control Tumor Growth In Vivo

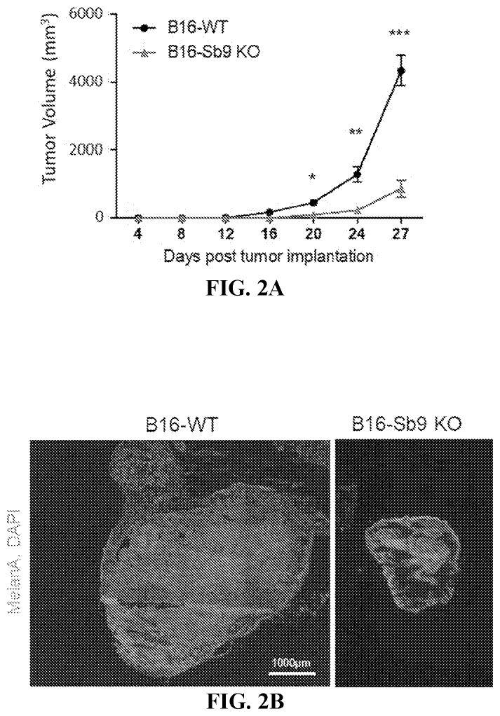

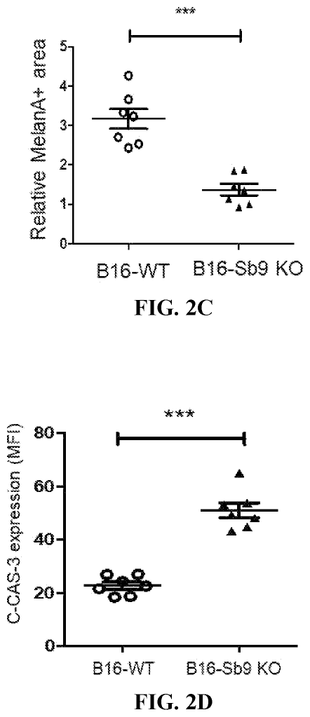

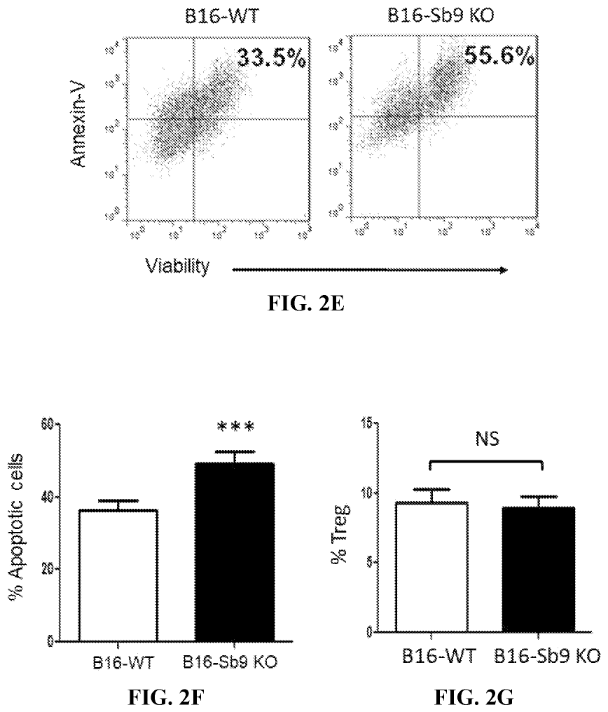

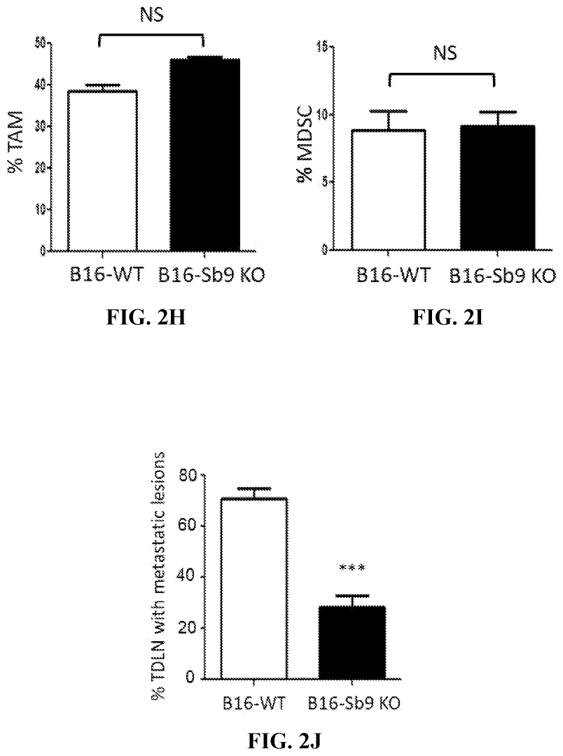

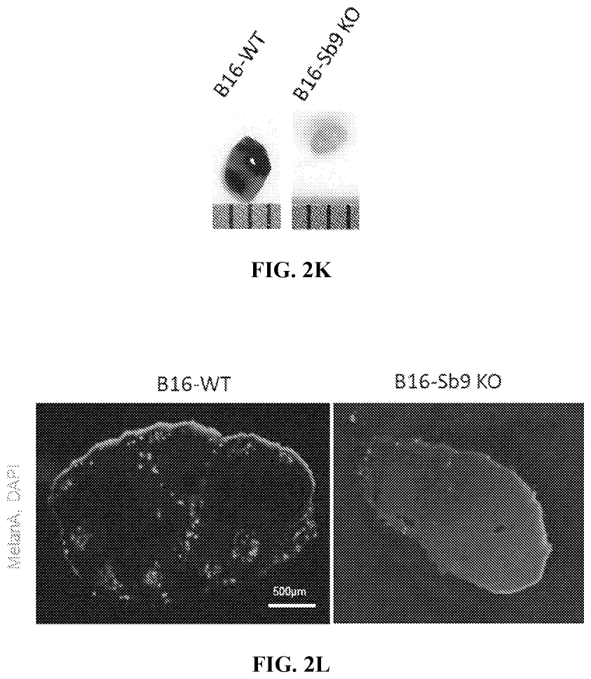

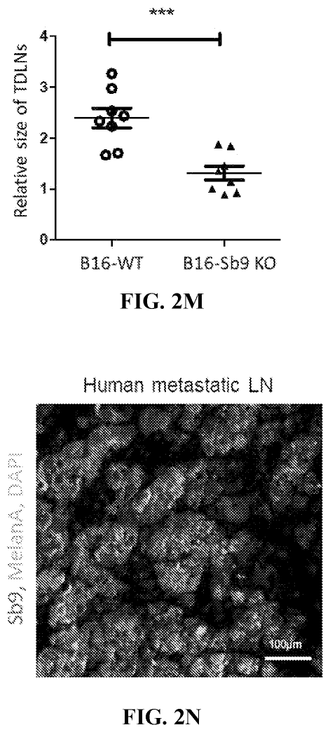

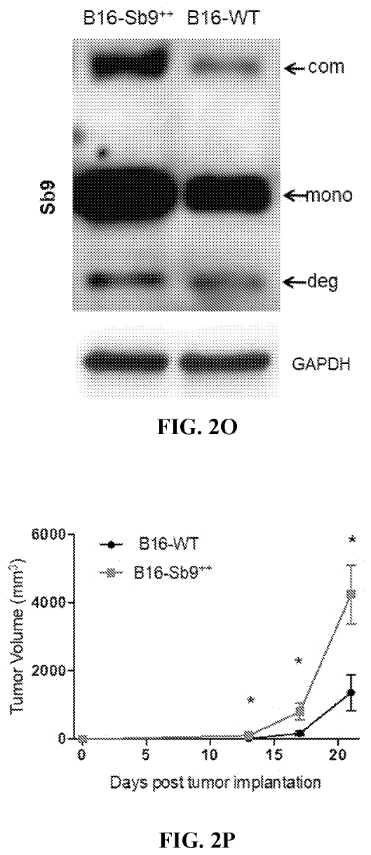

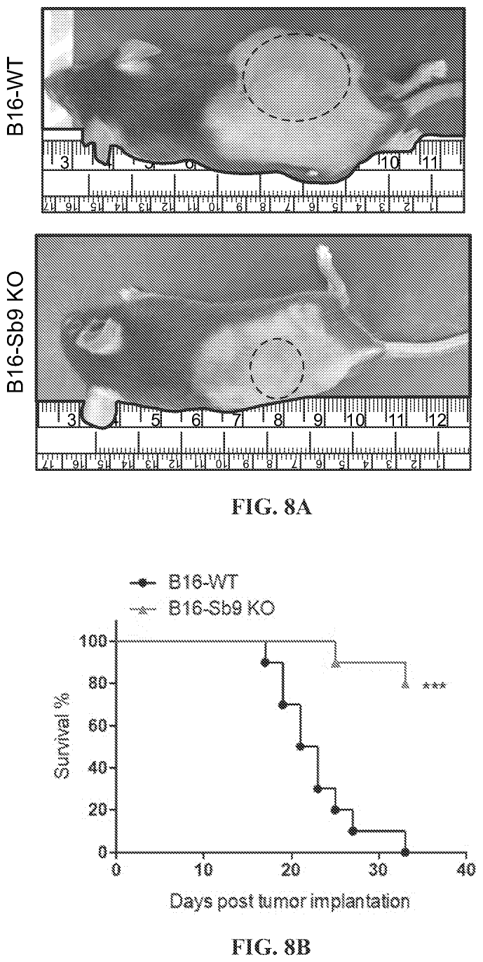

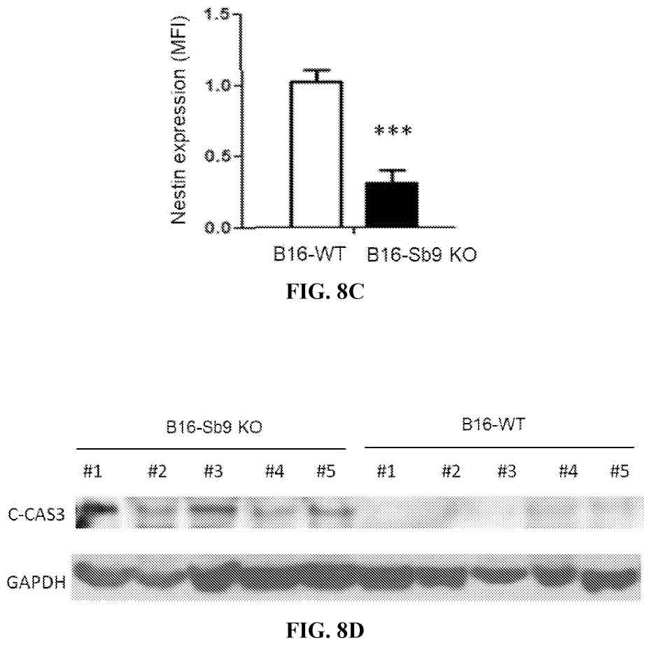

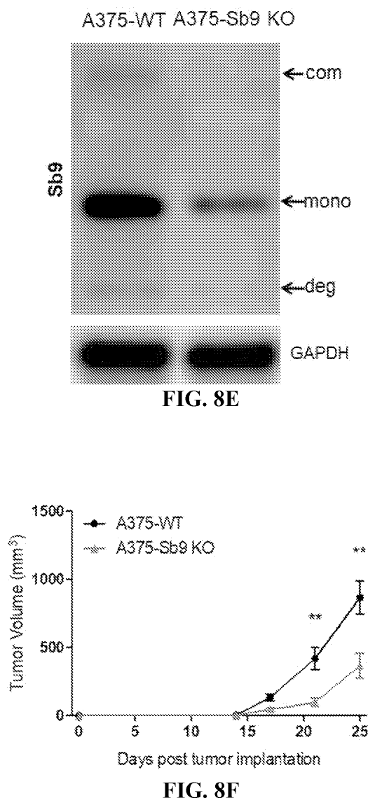

To investigate the role of Sb9 in tumor progression and metastasis in vivo, B16-WT or B16-Sb9 KO cells were injected subcutaneously into C57BL/6 mice, then tumor growth was monitored. The Sb9-disrupted melanoma tumors were ˜4-fold smaller (p=0.0001) than the B16-WT group at 4 weeks post-implantation (FIGS. 2A and 8A). Staining of the melanoma sections with the melanoma marker MelanA at 17 days following implantation revealed a much smaller MelanA⁺ area in the B16-Sb9 KO group than the B16-WT group (FIGS. 2B-2C). The median survival time (MST) of mice bearing B16-Sb9 KO tumors was significantly longer (p<0.0001) than that of mice bearing B16-WT tumors (FIG. 8B). Interestingly, the population of Nestin⁺ cells was also lower in B16-Sb9 KO melanoma sections (FIG. 8C). B16-Sb9 KO tumors exhibited more apoptosis and higher expression of cleaved caspase-3 than those from the B16-WT group (FIGS. 2D-2F and 8D).

However, there were no differences in the levels of Tregs, TAMs, and MDSCs between the B16-WT and B16-Sb9 KO groups (FIGS. 2G-2I), indicating that Sb9 tumor expression had minimal impact on the proportion of immunosuppressive cell populations in the TME. Metastases of B16-Sb9 KO tumors was also decreased, as evidenced by the decreased levels of melanoma cells in tumor-draining lymph nodes (TDLNs) (FIGS. 2J-2L). The size of TDLNs in the B16-Sb9 KO group was significantly (p=0.0006) smaller than that in the B16-WT group (FIGS. 2K-2M). Additionally, co-expression of Sb9 and MelanA in metastatic lesions of human melanoma in the LN was observed (FIG. 2N).

B16 cells expressing transgenic Sb9 (B16-Sb9⁺⁺ cells) were generated and Sb9 over-expression was confirmed by Western blotting (FIG. 2O). Following subcutaneous injection into C57BL/6 mice, the B16-Sb9⁺⁺ tumors grew significantly more rapidly (3.2-fold, p=0.017) at 21 days post-implantation in comparison to the B16-WT group (FIG. 2P). CRISPR/Cas9 was also utilized to ablate Sb9 in the A375 human melanoma cell line (FIG. 8E) and significant inhibition (4.3-fold, p=0.0035, Day-21; 2.4-fold, p=0.0069, Day-25) on the growth of Sb9-disrupted human melanoma tumors after transplantation was observed (FIG. 8F). Taken together, these findings strongly suggest that expression of Sb9 promotes tumor growth and metastases in vivo in a cell-intrinsic manner.

### Example 3. Sb9 Deletions Restore Host Immunity to Tumors and Disrupt Stroma in the TME

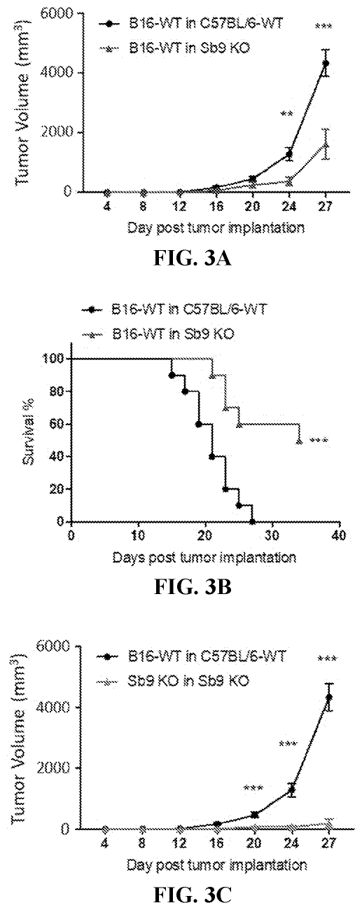

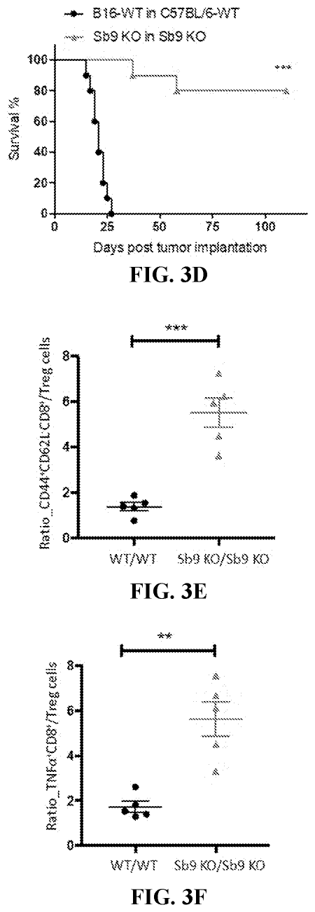

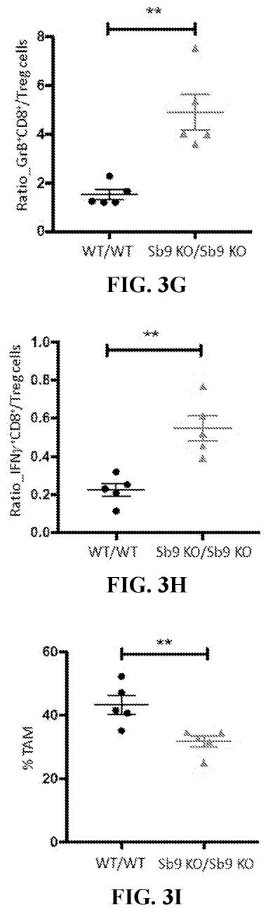

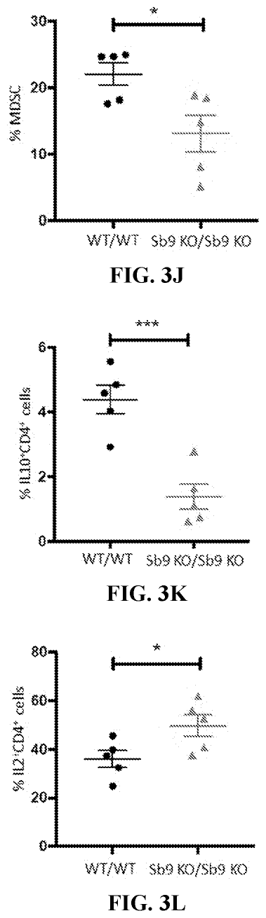

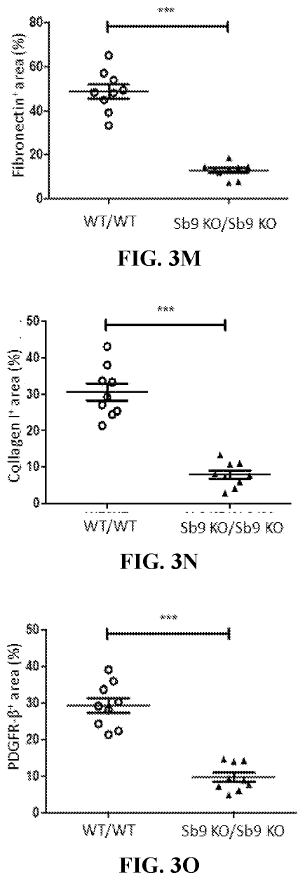

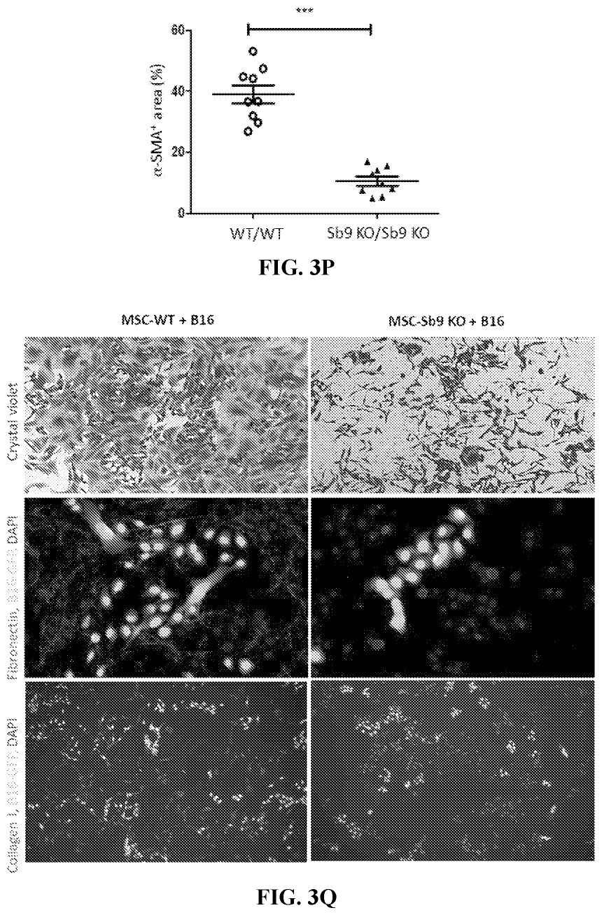

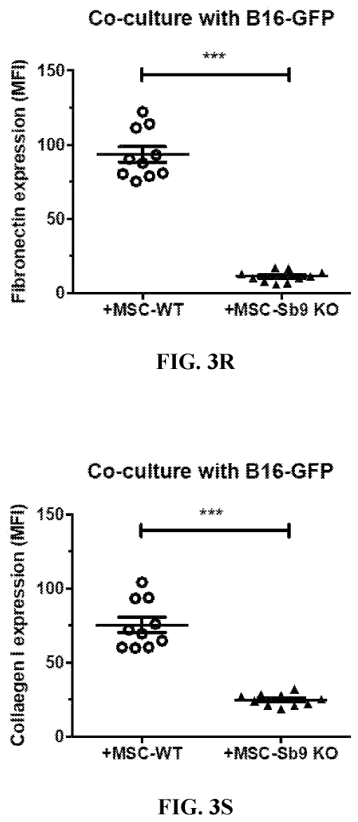

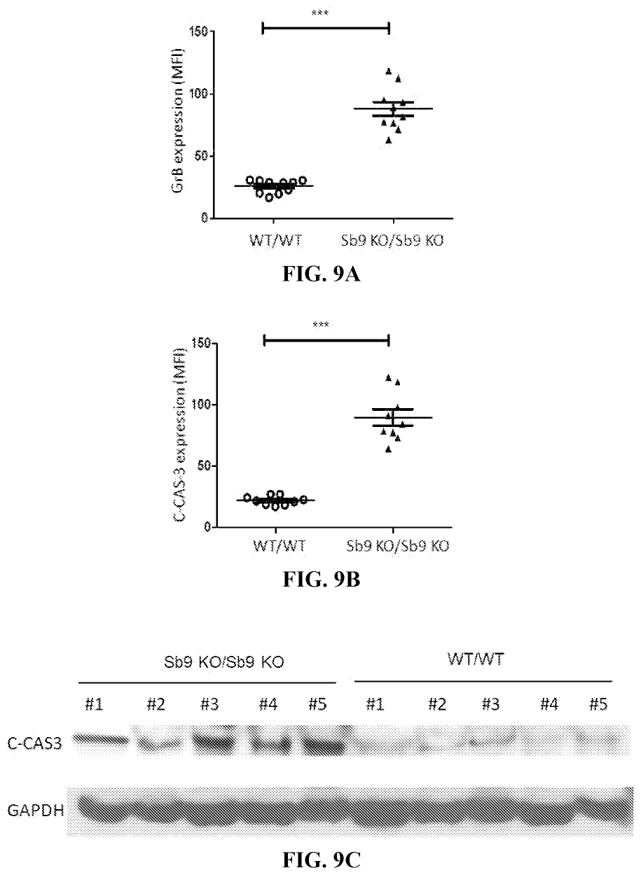

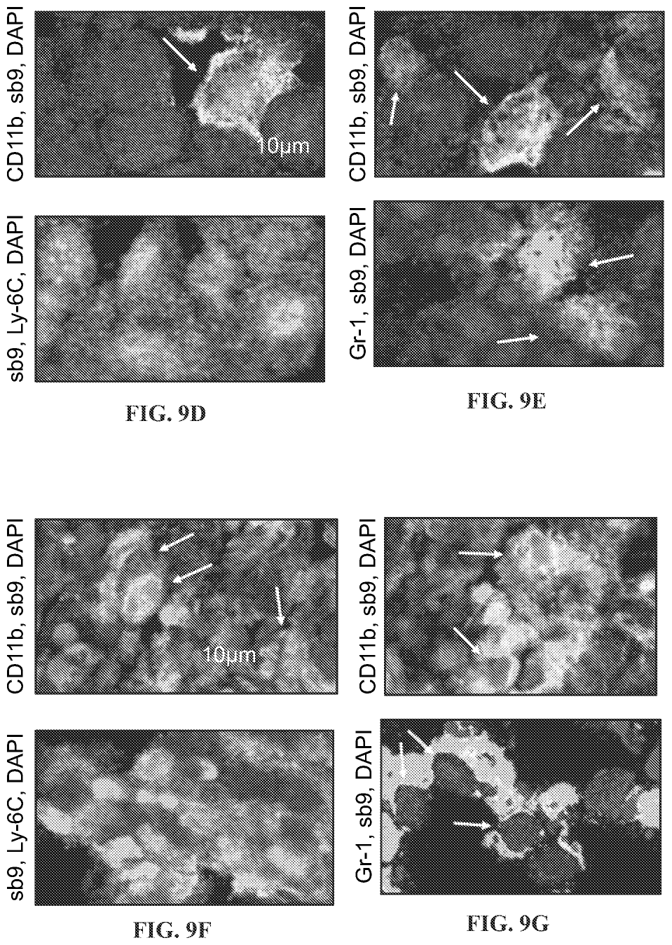

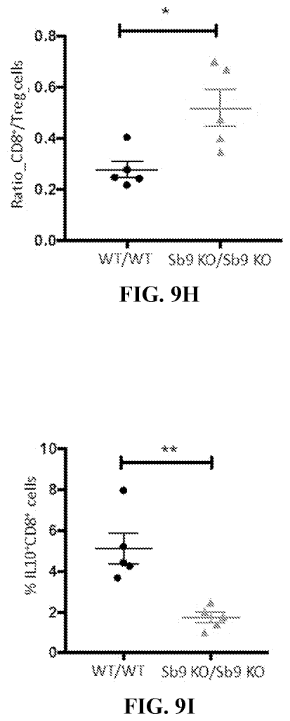

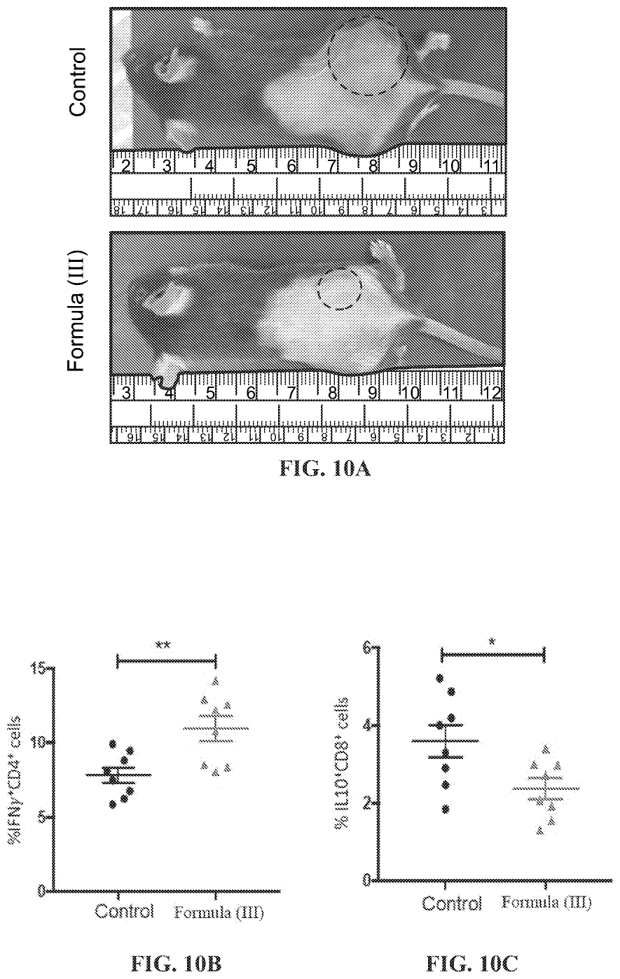

The role of Sb9 in the host immune response to tumors in Sb9 KO mice was investigated (Zhang et al., 2006). Melanoma tumors grew more slowly (2.7-fold, p=0.0009, Day-27) in Sb9 KO mice, as compared with C57BL/6-WT mice (FIG. 3A), resulting in a significantly longer (p=0.0008) MST in Sb9 KO mice bearing melanoma than WT mice (FIG. 3B). However, maximal protection against melanoma development was observed when both tumor and host were deficient in Sb9 (FIG. 3C). The MST of Sb9 KO mice implanted with Sb9 KO tumors (Sb9 KO/Sb9 KO group) was much longer (p<0.0001) than that of WT mice implanted with WT tumors (WT/WT group) (MST>110 days vs. MST=21 days) (FIG. 3D). Sb9 deficiency resulted in increased GrB and caspase-3 expression by melanoma tumors from the Sb9 KO/Sb9 KO group, as compared with those from the WT/WT group (FIGS. 9A-9C).

Next, the effect of Sb9 host deficiency on the TME of implanted melanomas was examined. Tregs are potent suppressors of anti-tumor effector T cells and are protected from cell-intrinsic GrB by endogenous Sb9 (Azzi et al., 2013). The ratios of effector CD8⁺ to Treg cells (p=0.0156), CD44⁺ CD62L⁻ (effector memory) CD8⁺ to Treg cells (p=0.0003), effector TNF-α+CD8⁺ to Treg cells (p=0.0012), effector GrB⁺ CD8⁺ to Treg cells (p=0.002), and effector IFNγ⁺ CD8⁺ to Treg cells (p=0.0023) were all significantly higher in the melanoma sections from the Sb9 KO/Sb9 KO group, as compared with those from the WT/WT group (FIGS. 3E-3H and 9H).

In addition to Tregs, the recruitment of immunosuppressive TAMs and MDSCs also played a critical role in tumor progression (Kitamura et al., 2015; Ugel et al., 2015). Fluorescence micrographs demonstrated the expression of Sb9 in the TAMs (CD11b⁺, Ly-6C⁻) and MDSCs (CD11b⁺, Gr-1⁺) within melanoma and breast tumors (FIGS. 9D-9G). The levels of TAMs (p=0.0093) and MDSCs (p=0.025) were also significantly lower in melanoma sections from the Sb9 KO/Sb9 KO group, as compared to the WT/WT group (FIGS. 3I-3J). It is believed that loss of Sb9 in Tregs, TAMs and MDSCs exposed them to GrB-mediated killing within the TME, ultimately leading to restoration of the antitumor immune response and inhibition of tumor progression. This effect on immunosuppressive cells overall is apparently more dominant for anti-tumor immunity than any decrease in the survival of CLs. Furthermore, an increased level of effector IL2⁺ CD4⁺ cells and a decreased level of immunosuppressive IL10⁺CD4⁺ cells and IL10⁺CD8⁺ cells were found in the TME sections from the Sb9 KO/Sb9 KO group (FIGS. 3K-3L and 9I).

CAFs inhibit the host resolution of solid cancers by preventing access of effector T cells to tumor cells (Kalluri, 2016). The number of CAFs in melanomas from the Sb9 KO/Sb9 KO group, as shown by the expression of fibronectin (4.1-fold, p<0.0001), collagen-I (3.7-fold, p=0.0002), PDGFR-β (3.3-fold, p=0.0002), and α-SMA (3.9-fold, p<0.0001), were all dramatically reduced compared to the WT/WT group (FIGS. 3M-3P). To further assess the effect of Sb9 expression in the tumor stroma on the growth of melanoma tumors, B16-GFP cells were co-cultured with the mesenchymal stem cells (MSCs) isolated from the bone marrow of either C57BL/6-WT mice or Sb9 KO mice (Zhu et al., 2010). Whether MSC-WT or MSC-Sb9 KO cells differ in their capacity to support B16 melanoma cells in vitro was investigated by assessing their growth together in culture plates. Micrographs of the co-cultured MSC-WT and B16-GFP cells revealed multiple dense clusters comprised of both MSC-WT and B16 tumor cells, while the MSC-Sb9 KO and B16 cells were scattered and not situated in proximity to each other (FIG. 3Q, top). The accumulation of fibronectin and collagen I as surrogate markers of a fibrotic tumor microenvironment, which is a major barrier against the penetration of chemotherapeutic agents and constitutes a significant obstacle to their efficacy, was assessed. It was observed that MSC-WT cells produced fibronectin and collagen I more readily in co-culture with B16-GFP melanoma cells than the MSC-Sb9 KO cells group (FIG. 3Q, middle and bottom; FIGS. 3R-3S). These data indicate that the presence of Sb9 in stromal cells promoted their growth, synthesis of ECM fibers, and proliferative effect on B16 melanoma cells.

### Example 4. Small Molecules Evoke Protective Immunity to Tumors

Two small molecules (3,4-dihydroxybenzoic acid and 3-oxo-1-indancarboxylic acid) were identified as having a dose-dependent Tₘ1 shift (FIG. 4A). A small molecule inhibitor of Sb9, the compound of Formula (III) (1,3-benzoxazole-6-carboxylic acid; Enamine, Kyiv, Ukraine), exhibited specific binding to Sb9 with a K_{D} of 273 μM (FIG. 4B). The ability of this small molecule inhibitor to inhibit intracellular Sb9 was then evaluated. As a marker for cell penetration, the induction of caspase-3 activity in B16 melanoma cells as a surrogate marker for increased GrB activity was measured. Treatment with the compound of Formula (III) exhibited a˜4-fold increase in caspase-3 activity (p=0.0003) compared to 3,4-dihydroxybenzoic acid, a compound having a MBP-Sb9 binding affinity of K_{D} of 88 μM (FIGS. 4B and 4C).

Saturation-Transfer Difference (STD) NMR assays demonstrating the binding affinity of the compound of Formula (III) to MBP-Sb9 protein revealed that this interaction was enhanced significantly at lower pH in a pH-dependent manner (FIGS. 4D-4E). Lysosomes have a pH of 4 and are the most acidic compartment within cells. Therefore, it was hypothesized that lysosomes contain an environment that is conducive for the binding between the compound of Formula (III) and Sb9. Both Sb9 and GrB were co-localized in the lysosomes of B16 melanoma cells (FIG. 4F). Furthermore, the compound of Formula (III) reduced the expression of the Sb9-GrB complex by B16 cells in a concentration-dependent manner (FIG. 4G). Interestingly, the compound of Formula (III) significantly increased the LAMP1 expressions and expanded the lysosome areas, indicating that the compound of Formula (III) might bind to more Sb9 proteins within larger lysosome areas (FIG. 4H).

The induction of caspase-3 activity in B16 melanoma cells as a surrogate marker for increased GrB activity was also measured for other small molecules (compounds of Formulas (V) and (VI) as described herein). After treatment, many of the compounds exhibited similar or increased caspase-3 induction activity compared to the compound of Formula (III) and/or the control (3,4-dihydroxybenzoic acid), as shown in Table 3. Table 4 shows the data as a ratio of caspase-3 activity of the small molecule compounds to the caspase-3 activity of Formula (III).

TABLE 3
Caspase-3 induction in tumor cells treated with the small molecules

| | Normalized | Normalized |
|---|---|---|
| | to Control | to Formula |
| Compound | (%) | (III) (%) |
| N-benzyl-2-(dimethylamino)-1,3- | 377.5569 | 61.2163 |
| benzoxazole-4-carboxamide | | |
| 2-cyclopropyl-N-ethyl-N-methyl-1,3- | 296.8445 | 33.9690 |
| benzoxazole-6-carboxamide | | |
| 2-cyclohexyl-N-cyclopentyl-1,3- | 273.9815 | 26.2507 |
| benzoxazole-6-carboxamide | | |
| N-benzyl-4-ethoxy-3-hydroxybenzamide | 252.7062 | 19.0685 |
| 1-(2-bromo-4,5-dimethoxybenzoyl)pyrrolidine | 250.9611 | 18.4794 |
| N-(3-methoxypropyl)-2-sulfanyl-1,3- | 241.3829 | 15.2459 |
| benzoxazole-6-carboxamide | | |
| N-ethyl-N-methyl-2-(oxolan-2-yl)-1,3- | 220.9670 | 8.3538 |
| benzoxazole-6-carboxamide | | |
| N-[2-(dimethylamino)ethyl]-3-methoxy- | 216.4600 | 6.8323 |
| 4-(3-methylbutoxy)benzamide | | |
| N-cyclopentyl-4-ethoxy-3-hydroxybenzamide | 208.4760 | 4.1370 |
| 2-(1,3-benzoxazol-5-yl)-1-(pyrrolidin- | 205.2680 | 3.0541 |
| 1-yl)ethan-1-one | | |
| N-[2-(dimethylamino)ethyl]-6-hydroxy- | 215.2015 | 2.3274 |
| 2H-1,3-benzodioxole-5-carboxamide | | |
| 2-cyclohexyl-N-ethyl-N-methyl-1,3- | 199.7507 | 1.1915 |
| benzoxazole-6-carboxamide | | |
| 4-hydroxy-3,5-dimethoxy-N-[(1- | 194.4499 | −0.59797 |
| methylcyclopropyl)methyl]benzamide | | |
| 2-bromo-4,5-dimethoxy-N-[(oxolan-3- | 189.8773 | −2.14161 |
| yl)methyl]benzamide | | |
| 3-methoxy-N-[(oxolan-3-yl)methyl]- | 188.3028 | −2.67313 |
| 4-(propan-2-yloxy)benzamide | | |
| N-ethyl-6-hydroxy-N-methyl-2H-1,3- | 186.0789 | −3.42391 |
| benzodioxole-5-carboxamide | | |
| 4-ethoxy-3-hydroxy-N-(2-methylpropyl)benzamide | 197.0696 | −3.55899 |
| 2-(1,3-benzoxazol-5-yl)-N-cyclopentylacetamide | 184.3141 | −4.01967 |
| 2-cyclopropyl-N-(3-methoxypropyl)-1,3- | 182.1229 | −4.75937 |
| benzoxazole-6-carboxamide | | |
| N-[2-(dimethylamino)ethyl]-4-hydroxy- | 179.138 | −5.76706 |
| 3,5-dimethoxybenzamide | | |
| N-ethyl-N-methyl-2-sulfanyl-1,3- | 177.8521 | −6.20114 |
| benzoxazole-6-carboxamide | | |
| 2-(3,5-dichlorophenyl)-6-(pyrrolidine-1- | 177.7668 | −6.22993 |
| carbonyl)-1,3-benzoxazole | | |
| N-benzyl-6-hydroxy-2H-1,3-benzodioxole-5- | 168.4773 | −9.36593 |
| carboxamide | | |
| N-benzyl-4-hydroxy-3,5-dimethoxybenzamide | 143.7709 | −17.7065 |
| N-benzyl-2-sulfanyl-1,3-benzoxazole-6- | 143.7053 | −17.7286 |
| carboxamide | | |
| N-cyclopentyl-2-(dimethylamino)-1,3- | 143.3673 | −20.9929 |
| benzoxazole-4-carboxamide | | |
| N,N-dimethyl-7-(pyrrolidine-1-carbonyl)- | 143.2496 | −21.0312 |
| 1,3-benzoxazol-2-amine | | |
| 2-ethoxy-5-(pyrrolidine-1-carbonyl)phenol | 142.0304 | −21.427 |
| N-cyclopentyl-2-cyclopropyl-1,3- | 141.7844 | −21.5068 |
| benzoxazole-6-carboxamide | | |
| N-ethyl-N-methyl-2-oxo-2,3-dihydro-1,3- | 141.1722 | −21.7056 |
| benzoxazole-5-carboxamide | | |
| N,N-dimethyl-4-(pyrrolidine-1-carbonyl)- | 137.2292 | −22.9856 |
| 1,3-benzoxazol-2-amine | | |
| N-(3-methoxypropyl)-2-(1,1,2,2,2- | 136.5306 | −23.2124 |
| pentafluoroethyl)-1,3-benzoxazole-6- | | |
| carboxamide | | |
| 4-hydroxy-3,5-dimethoxy-N-(3- | 135.3558 | −23.5938 |
| methoxypropyl)benzamide | | |
| N-cyclopentyl-4-hydroxy-3,5- | 132.3234 | −24.5783 |
| dimethoxybenzamide | | |
| 4-ethoxy-3-hydroxy-N-[(oxolan-3- | 131.3161 | −24.9053 |
| yl)methyl]benzamide | | |
| 4-hydroxy-3,5-dimethoxy-N-(2- | 121.3081 | −25.2896 |
| methylpropyl)benzamide | | |
| 2-(dimethylamino)-N-ethyl-N-methyl-1,3- | 128.9351 | −25.6782 |
| benzoxazole-4-carboxamide | | |
| N-benzyl-2-(dimethylamino)-1,3- | 116.5453 | −26.8974 |
| benzoxazole-7-carboxamide | | |
| 2-cyclopropyl-6-(pyrrolidine-1-carbonyl)- | 123.5871 | −27.4144 |
| 1,3-benzoxazole | | |
| 4,5-dihydroxy-2-methyl-N-(2- | 122.0617 | −27.9096 |
| methylpropyl)benzamide | | |
| N-ethyl-N-methyl-2-(1,1,2,2,2- | 120.4003 | −28.449 |
| pentafluoroethyl)-1,3-benzoxazole-6- | | |
| carboxamide | | |
| 2-cyclohexyl-6-(pyrrolidine-1-carbonyl)- | 116.5411 | −29.7019 |
| 1,3-benzoxazole | | |
| N-cyclopentyl-2-methyl-1,3-benzoxazole-4- | 114.7357 | −30.2879 |
| carboxamide | | |
| 6-(pyrrolidine-1-carbonyl)-1,3-benzoxazole- | 113.3883 | −30.7254 |
| 2-thiol | | |
| 4-ethoxy-3-hydroxy-N-[(oxan-4- | 113.0141 | −30.8469 |
| yl)methyl]benzamide | | |
| 2-(dimethylamino)-N-ethyl-N-methyl-1,3- | 112.5589 | −30.9946 |
| benzoxazole-7-carboxamide | | |
| 2-(dimethylamino)-N-(3-methoxypropyl)-1,3- | 112.3914 | −31.049 |
| benzoxazole-4-carboxamide | | |
| 2-(l,3-benzoxazol-5-yl)-N-(3- | 111.9231 | −31.2011 |
| methoxypropyl)acetamide | | |
| 6-hydroxy-N-(2-methylpropyl)-2H-1,3- | 103.7132 | −31.2294 |
| benzodioxole-5-carboxamide | | |
| 2-(3,5-dichlorophenyl)-N-(3-methoxypropyl)- | 110.7248 | −31.5901 |
| 1,3-benzoxazole-6-carboxamide | | |
| 3-methoxy-4-(3-methylbutoxy)-N-[(oxan-4- | 101.9288 | −31.8318 |
| yl)methyl]benzamide | | |
| N-benzyl-2-cyclopropyl-1,3-benzoxazole-6- | 106.7452 | −32.882 |
| carboxamide | | |
| N-ethyl-4,5-dihydroxy-N,2-dimethylbenzamide | 105.7300 | −33.2116 |
| N-ethyl-4-hydroxy-3-methoxy-N-methylbenzamide | 105.1753 | −33.3917 |
| 2-(1,3-benzoxazol-5-yl)-N-benzylacetamide | 101.7792 | −34.4942 |
| N-benzyl-2-(oxolan-2-yl)-1,3-benzoxazole- | 101.3501 | −34.6335 |
| 6-carboxamide | | |
| N-ethyl-4-hydroxy-3,5-dimethoxy-N- | 93.6233 | −34.6356 |
| methylbenzamide | | |
| 4-ethoxy-3-hydroxy-N-(3- | 98.1214 | −35.6816 |
| methoxypropyl)benzamide | | |
| 6-(pyrrolidine-1-carbonyl)-2H-1,3- | 97.82313 | −35.7785 |
| benzodioxol-5-ol | | |
| N-benzyl-2-(3,5-dichlorophenyl)-1,3- | 96.97802 | −36.0528 |
| benzoxazole-6-carboxamide | | |
| 2-(1,1,2,2,2-pentafluoroethyl)-6- | 82.81834 | −38.2832 |
| (pyrrolidine-1-carbonyl)-1,3-benzoxazole | | |
| N-[2-(dimethylamino)ethyl]-4-ethoxy- | 89.36682 | −38.5237 |
| 3-hydroxybenzamide | | |
| 2-(dimethylamino)-N-(3-methoxypropyl)-1,3- | 78.97396 | −39.581 |
| benzoxazole-7-carboxamide | | |
| 4-methyl-5-(pyrrolidine-1-carbonyl)benzene- | 83.32548 | −40.485 |
| 1,2-diol | | |
| 6-hydroxy-N-(3-methoxypropyl)-2H-1,3- | 78.98482 | −41.8942 |
| benzodioxole-5-carboxamide | | |
| 2-cyclohexyl-N-(3-methoxypropyl)-1,3- | 78.10047 | −42.1813 |
| benzoxazole-6-carboxamide | | |
| N-benzyl-4,5-dihydroxy-2-methylbenzamide | 73.67347 | −43.6184 |
| 2-bromo-4,5-dimethoxy-N-[(oxan-4- | 73.6002 | −43.6422 |
| yl)methyl]benzamide | | |
| N-benzyl-2-methyl-1,3-benzoxazole-4- | 65.70382 | −46.2057 |
| carboxamide | | |
| N-cyclopentyl-2-(dimethylamino)-1,3- | 61.10413 | −47.699 |
| benzoxazole-7-carboxamide | | |
| N-benzyl-2-(1,1,2,2,2-pentafluoroethyl)- | 58.97436 | −48.3904 |
| 1,3-benzoxazole-6-carboxamide | | |
| 2-bromo-N-cyclopentyl-4,5- | 54.35113 | −49.8913 |
| dimethoxybenzamide | | |
| N-(cyclopentylmethyl)-3-methoxy-4-(3- | 53.69963 | −50.1028 |
| methylbutoxy)benzamide | | |
| 4,5-dihydroxy-N-(3-methoxypropyl)-2- | 36.3423 | −55.7377 |
| methylbenzamide | | |
| 2-bromo-4,5-dimethoxy-N-(2- | 26.40241 | −58.9646 |
| methylpropyl)benzamide | | |
| N-benzyl-2-cyclohexyl-1,3-benzoxazole-6- | 19.3878 | −61.2418 |
| carboxamide | | |
| N-cyclopentyl-6-hydroxy-2H-1,3- | 18.14757 | −61.6444 |
| benzodioxole-5-carboxamide | | |
| N-ethyl-N,2-dimethyl-1,3-benzoxazole-5- | 1.699141 | −65.6678 |
| carboxamide | | |
| 3-methoxy-N-[(oxan-4-yl)methyl]-4- | 3.872318 | −66.2788 |
| (propan-2-yloxy)benzamide | | |
| N-cyclopentyl-4,5-dihydroxy-2- | −0.82005 | −66.5183 |
| methylbenzamide | | |
| N-(3-methoxypropyl)-2-(oxolan-2-yl)-1,3- | −10.7722 | −69.878 |
| benzoxazole-6-carboxamide | | |
| 4-ethoxy-N-ethyl-3-hydroxy-N- | −10.805 | −69.889 |
| methylbenzamide | | |
| 2-bromo-N-[2-(dimethylamino)ethyl]- | −11.5266 | −70.1327 |
| 4,5-dimethoxybenzamide | | |
| N-(3-methoxypropyl)-2-methyl-1,3- | −8.84877 | −70.4086 |
| benzoxazole-4-carboxamide | | |
| 6-(pyrrolidine-1-carbonyl)-2- | −11.9048 | −71.4007 |
| (trifluoromethyl)-1,3-benzoxazole | | |
| 2-bromo-N-ethyl-4,5-dimethoxy-N- | −23.0138 | −74.0106 |
| methylbenzamide | | |
| N-ethyl-N-methyl-1,3-benzoxazole-5- | −25.8939 | −74.9828 |
| carboxamide | | |
| 4-hydroxy-3-methoxy-N-[(oxan-4- | −27.0199 | −76.3077 |
| yl)methyl]benzamide | | |
| N-ethyl-N-methyl-2-phenyl-1,3-benzoxazole- | −31.5227 | −76.883 |
| 6-carboxamide | | |
| 4-hydroxy-3,5-dimethoxy-N-[(oxan-4- | −37.0852 | −78.7609 |
| yl)methyl]benzamide | | |
| N-cyclopentyl-2-sulfanyl-1,3-benzoxazole- | −44.6848 | −81.3264 |
| 6-carboxamide | | |
| 2-(3,5-dichlorophenyl)-N-ethyl-N-methyl- | −55.8082 | −85.6535 |
| l,3-benzoxazole-6-carboxamide | | |
| 2-bromo-4,5-dimethoxy-N-(3- | −56.759 | −85.9622 |
| methoxypropyl)benzamide | | |

TABLE 4
Caspase-3 induction in tumor cells treated with the small molecules

| | Ratio of caspase-3 |
|---|---|
| | activity to Formula |
| Compound | (III) |
| 1-(2-phenyl-1,3-benzoxazole-6- | 1.326852 |
| carbonyl)piperidin-4-ol | |
| 3-[(2-phenyl-1,3-benzoxazol-6- | 1.370985 |
| yl)formamido]-N-(propan-2- | |
| yl)propanamide | |
| N-(5,5-dimethyloxolan-3-yl)-2-phenyl-1,3- | 0.093074 |
| benzoxazole-6-carboxamide | |
| [(2S,4S)-4-fluoro-1-(2-phenyl-1,3- | 0.097225 |
| benzoxazole-6-carbonyl)pyrrolidin-2- | |
| yl]methanol | |
| 6-{6-oxa-1-azaspiro[3.4]octane-1- | 1.434127 |
| carbonyl}-2-phenyl-1,3-benzoxazole | |
| N-(2,6-dioxopiperidin-3-yl)-2-phenyl-1,3- | 0.698929 |
| benzoxazole-6-carboxamide | |
| 2-(3-fluorophenyl)-N-[3-(1H-pyrazol-1- | 0.911296 |
| yl)propyl]-1,3-benzoxazole-5-carboxamide | |
| 2-(1-{[2-(3-fluorophenyl)-1,3-benzoxazol- | 1.153813 |
| 5-yl]carbonyl}-2-piperidinyl)ethanol | |
| methyl 4-({[2-(3-methoxyphenyl)-1,3- | 0.385842 |
| benzoxazol-6-yl]carbonyl}amino)butanoate | |
| 2-(3-fluorophenyl)-N-(3-pyridinylmethyl)-1,3- | 0.107712 |
| benzoxazole-5-carboxamide | |
| 2-(3-fluorophenyl)-N-[2-(1H-pyrazol-1- | 0.469303 |
| yl)ethyl]-1,3-benzoxazole-5-carboxamide | |
| 1-{[2-(3-methoxyphenyl)-1,3-benzoxazol-6- | 0.820406 |
| yl]carbonyl}-3-piperidinol | |
| 2-(3-fluorophenyl)-N-[(1-methyl-1H-pyrazol-4- | 0.100503 |
| yl)methyl]-1,3-benzoxazole-5-carboxamide | |
| 2-(3-methoxyphenyl)-N-(tetrahydro-2H-pyran-2- | 1.424951 |
| ylmethyl)-1,3-benzoxazole-6-carboxamide | |
| N-[(1,5-dimethyl-1H-pyrazol-3-yl)methyl]- | 0.123006 |
| 2-(3-fluorophenyl)-1,3-benzoxazole-5-carboxamide | |
| 2-(3-methoxyphenyl)-N-(tetrahydro-2H-pyran-4- | 0.092856 |
| ylmethyl)-1,3-benzoxazole-6-carboxamide | |
| ethyl N-{[2-(3-fluorophenyl)-1,3-benzoxazol- | 0.093293 |
| 5-yl]carbonyl}-beta-alaninate | |
| 2-(3-fluorophenyl)-N-[(2-methyl-1,3-thiazol-4- | 0.097881 |
| yl)methyl]-1,3-benzoxazole-5-carboxamide | |
| 2-(3-fluorophenyl)-N-[2-(2-furyl)ethyl]- | 0.094385 |
| 1,3-benzoxazole-5-carboxamide | |
| 2-(3-methoxyphenyl)-N-(tetrahydro-2- | 0.430413 |
| furanylmethyl)-1,3-benzoxazole-6-carboxamide | |
| 2-(3-fluorophenyl)-N-methyl-N-(1H-pyrazol-5- | 0.293861 |
| ylmethyl)-1,3-benzoxazole-5-carboxamide | |
| N-(2-hydroxyethyl)-2-[4-(trifluoromethyl)phenyl]- | 0.384313 |
| 1,3-benzoxazole-5-carboxamide | |
| 1-{[2-(3-fluorophenyl)-1,3-benzoxazol-5- | 0.788071 |
| yl]carbonyl}-N-methylpyrrolidine-3-carboxamide | |
| N-(1,1-dioxido-2,3-dihydro-3-thienyl)-2-(3- | 0.097007 |
| fluorophenyl)-1,3-benzoxazole-5-carboxamide | |
| 1-({2-[4-(trifluoromethyl)phenyl]-1,3- | 0.131964 |
| benzoxazol-5-yl}carbonyl)azetidin-3-ol | |
| N-(3-hydroxypropyl)-2-[4- | 1.003059 |
| (trifluoromethyl)phenyl]-1,3-benzoxazole-5- | |
| carboxamide | |
| N-(2-hydroxyethyl)-N-methyl-2-[4- | 1 |
| (trifluoromethyl)phenyl]-1,3-benzoxazole- | |
| 5-carboxamide | |
| 2-(3-fluorophenyl)-N-[2-(4-methyl-4H-1,2,4- | 0.713349 |
| triazol-3-yl)ethyl]-1,3-benzoxazole-5-carboxamide | |
| 2-(3-methoxyphenyl)-N-(1H-tetrazol-5-ylmethyl)- | 0.391086 |
| 1,3-benzoxazole-6-carboxamide | |
| 1-{[2-(3-fluorophenyl)-1,3-benzoxazol-5- | 0.085864 |
| yl]carbonyl}-L-prolinamide | |
| 2-(3-fluorophenyl)-5-(thiomorpholin-4-ylcarbonyl)- | 1.016386 |
| 1,3-benzoxazole | |
| 2-(3-methoxyphenyl)-N-[(5-methylpyrazin-2- | 0.138737 |
| yl)methyl]-1,3-benzoxazole-6-carboxamide | |
| 2-(3-fluorophenyl)-N-methyl-N-[(5- | 0.10662 |
| methylisoxazol-3-yl)methyl]-1,3-benzoxazole- | |
| 5-carboxamide | |
| 2-(3-fluorophenyl)-N-(isoxazol-5-ylmethyl)-1,3- | 0.095259 |
| benzoxazole-5-carboxamide | |
| 1-{[2-(3-fluorophenyl)-1,3-benzoxazol-5- | 1.0721 |
| yl]carbonyl}-2-methyl-1,4-diazepan-5-one | |
| N-(2-hydroxypropyl)-2-[4- | 0.0922 |
| (trifluoromethyl)phenyl]-1,3-benzoxazole-5- | |
| carboxamide | |
| 2-(3-fluorophenyl)-N-[1-methyl-2-(1H-1,2,4- | 0.101376 |
| triazol-1-yl)ethyl]-1,3-benzoxazole-5-carboxamide | |
| 5-[(1R*,5S*)-6-azabicyclo[3.2.1]oct-6-ylcarbonyl]- | 0.210618 |
| 2-(3-fluorophenyl)-1,3-benzoxazole | |
| 5-[(4-ethyl-1-piperazinyl)carbonyl]-2-(3- | 0.65414 |
| fluorophenyl)-1,3-benzoxazole | |
| 2-(3-fluorophenyl)-N-methyl-N-(tetrahydro-2H- | 0.828053 |
| pyran-2-ylmethyl)-1,3-benzoxazole-5-carboxamide | |
| N-isopropyl-2-(3-methoxyphenyl)-N-methyl-1,3- | 1.45816 |
| benzoxazole-6-carboxamide | |
| ethyl N-{[2-(3-methoxyphenyl)-1,3-benzoxazol-6- | 0.104872 |
| yl]carbonyl}-beta-alaninate | |
| 2-(3-fluorophenyl)-N-[2-(1-methyl-1H-pyrazol-4- | 0.447455 |
| yl)ethyl]-1,3-benzoxazole-5-carboxamide | |
| (1-{[2-(3-fluorophenyl)-1,3-benzoxazol-5- | 1.133057 |
| yl]carbonyl}-2-piperidinyl)methanol | |
| 2-(3-fluorophenyl)-N-[1-(1H-1,2,4-triazol-5- | 0.115796 |
| yl)ethyl]-1,3-benzoxazole-5-carboxamide | |
| 2-(3-fluorophenyl)-N-[2-(1H-pyrazol-4-yl)ethyl]- | 0.492462 |
| 1,3-benzoxazole-5-carboxamide | |
| N-(3-hydroxy-2,2-dimethylpropyl)-2-(3- | 0.671619 |
| methoxyphenyl)-1,3-benzoxazole-6-carboxamide | |
| N-isopropyl-2-[4-(trifluoromethyl)phenyl]-1,3- | 0.284684 |
| benzoxazole-5-carboxamide | |
| 2-(3-methoxyphenyl)-N-(2,2,2-trifluoroethyl)-1,3- | 0.095259 |
| benzoxazole-6-carboxamide | |
| N-[(1-ethyl-1H-pyrazol-4-yl)methyl]-2-(3- | 1.162989 |
| fluorophenyl)-1,3-benzoxazole-5-carboxamide | |
| 2-(3-fluorophenyl)-N-[(3S)-2-oxo-3-azepanyl]-1,3- | 0.771029 |
| benzoxazole-5-carboxamide | |
| 2-(3-fluorophenyl)-N-[(3S*,4S*)-4-methoxy-1- | 0.08936 |
| methylpyrrolidin-3-yl]-1,3-benzoxazole-5- | |
| carboxamide | |
| 1-{[2-(3-methoxyphenyl)-1,3-benzoxazol-6- | 0.990824 |
| yl]carbonyl}-N,N-dimethyl-3-pyrrolidinamine | |
| N-cyclobutyl-2-[4-(trifluoromethyl)phenyl]-1,3- | 1.279878 |
| benzoxazole-5-carboxamide | |
| N-(2,3-dihydroxypropyl)-N-methyl-2-[4- | 0.134969 |
| (trifluoromethyl)phenyl]-1,3-benzoxazole-5- | |
| carboxamide | |
| 2-(3-fluorophenyl)-N-methyl-N-[(5- | 0.169519 |
| methylisoxazol-3-yl)methyl]-1,3-benzoxazole-5- | |
| carboxamide | |
| 1-{[2-(3-fluorophenyl)-1,3-benzoxazol-5- | 0.274459 |
| yl]carbonyl}-L-prolinamide | |
| 2-(3-fluorophenyl)-N-(3-hydroxy-3-phenylpropyl)- | 0.128834 |
| N-methyl-1,3-benzoxazole-5-carboxamide | |
| N-[(5-ethyl-2-pyridinyl)methyl]-2-(3- | 0.132386 |
| fluorophenyl)-N-methyl-1,3-benzoxazole-5- | |
| carboxamide | |
| ethyl 1-{[2-(3-methoxyphenyl)-1,3-benzoxazol-6- | 0.136261 |
| yl]carbonyl}-3-piperidinecarboxylate | |
| N-(2-hydroxyethyl)-N-methyl-2-[4- | 0.125283 |
| (trifluoromethyl)phenyl]-1,3-benzoxazole-5- | |
| carboxamide | |
| 1-{[2-(3-fluorophenyl)-1,3-benzoxazol-5- | 0.145625 |
| yl]carbonyl}-2-methyl-1,4-diazepan-5-one | |
| 2-(3-fluorophenyl)-N-isopropyl-N-[(1-methyl-1H- | 1.634485 |
| imidazol-2-yl)methyl]-1,3-benzoxazole-5- | |
| carboxamide | |
| N-[(1-ethyl-1H-imidazol-2-yl)methyl]-N-methyl-2- | 0.156603 |
| [4-(trifluoromethyl)phenyl]-1,3-benzoxazole-5- | |
| carboxamide | |
| 6-[(4-cyclopentyl-1-piperazinyl)carbonyl]-2-(3- | 0.158218 |
| methoxyphenyl)-1,3-benzoxazole | |
| 5-(4-morpholinylcarbonyl)-2-[4- | 0.13174 |
| (trifluoromethyl)phenyl]-1,3-benzoxazole | |
| 4-{[2-(3-methoxyphenyl)-1,3-benzoxazol-6- | 0.161124 |
| yl]carbonyl}morpholine-2-carboxamide | |
| 2-(3-fluorophenyl)-5-(thiomorpholin-4-ylcarbonyl)- | 0.161447 |
| 1,3-benzoxazole | |
| (3S)-1-({2-[4-(trifluoromethyl)phenyl]-1,3- | 0.138198 |
| benzoxazol-5-yl}carbonyl)-3-pyrrolidinol | |
| N,N-diethyl-1-{[2-(3-methoxyphenyl)-1,3- | 0.146271 |
| benzoxazol-6-yl]carbonyl}-3- | |
| piperidinecarboxamide | |
| 2-(3-fluorophenyl)-5-{[4-(2-methoxyethoxy)-1- | 0.131418 |
| piperidinyl]carbonyl}-1,3-benzoxazole | |
| 2-(1-{[2-(3-fluorophenyl)-1,3-benzoxazol-5- | 0.126251 |
| yl]carbonyl}-2-piperidinyl)ethanol | |
| N-isopropyl-2-(3-methoxyphenyl)-N-methyl-1,3- | 0.143042 |
| benzoxazole-6-carboxamide | |
| 2-(3-methoxyphenyl)-N-methyl-N-[(5- | 0.240232 |
| methylisoxazol-3-yl)methyl]-1,3-benzoxazole-6- | |
| carboxamide | |
| 6-[(1,1-dioxidothiomorpholin-4-yl)carbonyl]-2-(3- | 0.708105 |
| methoxyphenyl)-1,3-benzoxazole | |
| 1-{[2-(3-fluorophenyl)-1,3-benzoxazol-5- | 0.138844 |
| yl]carbonyl}-N-methylpyrrolidine-3-carboxamide | |
| 1-({2-[4-(trifluoromethyl)phenyl]-1,3-benzoxazol- | 0.153697 |
| 5-yl}carbonyl)azetidin-3-ol | |
| N-methyl-N-(3-pyridinylmethyl)-2-[4- | 0.166936 |
| (trifluoromethyl)phenyl]-1,3-benzoxazole-5- | |
| carboxamide | |
| 4-{[2-(3-fluorophenyl)-1,3-benzoxazol-5- | 0.162738 |
| yl]carbonyl}-2,3,4,5-tetrahydro-1,4-benzoxazepine | |
| N-ethyl-N-(2-hydroxyethyl)-2-[4- | 0.143687 |
| (trifluoromethyl)phenyl]-1,3-benzoxazole-5- | |
| carboxamide | |
| N-methyl-N-[2-(4-morpholinyl)ethyl]-2-[4- | 0.132709 |
| (trifluoromethyl)phenyl]-1,3-benzoxazole-5- | |
| carboxamide | |
| N-(cyclopropylmethyl)-2-(3-methoxyphenyl)-N- | 0.136907 |
| (tetrahydro-2-furanylmethyl)-1,3-benzoxazole-6- | |
| carboxamide | |
| N-methyl-N-[(4-methyl-1H-imidazol-2-yl)methyl]- | 0.132709 |
| 2-[4-(trifluoromethyl)phenyl]-1,3-benzoxazole-5- | |
| carboxamide | |
| 1-(1-{[2-(3-methoxyphenyl)-1,3-benzoxazol-6- | 0.134646 |
| yl]carbonyl}-3-piperidinyl)-1-propanone | |
| 5-{[3-(methoxymethyl)-1-pyrrolidinyl]carbonyl}- | 0.142396 |
| 2-[4-(trifluoromethyl)phenyl]-1,3-benzoxazole | |
| 2-(3-fluorophenyl)-N-methyl-N-[2-(1- | 0.140459 |
| piperidinyl)ethyl]-1,3-benzoxazole-5-carboxamide | |
| N-methyl-N-(4-pyrimidinylmethyl)-2-[4- | 0.144333 |
| (trifluoromethyl)phenyl]-1,3-benzoxazole-5- | |
| carboxamide | |
| 1-{[2-(3-fluorophenyl)-1,3-benzoxazol-5- | 0.177268 |
| yl]carbonyl}-4-phenyl-4-piperidinol | |
| 2-(2-chlorophenyl)-N-[2-(dimethylamino)ethyl]-N- | 1.105586 |
| ethyl-3-methyl-3H-imidazo[4,5-b]pyridine-6- | |
| carboxamide | |
| 2-(3-methoxyphenyl)-N-methyl-N-[2-(tetrahydro- | 0.140459 |
| 2H-pyran-2-yl)ethyl]-1,3-benzoxazole-6- | |
| carboxamide | |
| N-(2-hydroxy-2-phenylethyl)-2-(3- | 0.158863 |
| methoxyphenyl)-N-methyl-1,3-benzoxazole-6- | |
| carboxamide | |
| ethyl (1-{[2-(3-methoxyphenyl)-1,3-benzoxazol-6- | 0.147885 |
| yl]carbonyl}-2-piperidinyl)acetate | |
| 2-(3-methoxyphenyl)-N-methyl-N-[(5-propyl-1H- | 0.153051 |
| pyrazol-3-yl)methyl]-1,3-benzoxazole-6- | |
| carboxamide | |
| 2-(3-methoxyphenyl)-6-[(3-propoxy-1- | 0.1495 |
| piperidinyl)carbonyl]-1,3-benzoxazole | |
| 2-(3-fluorophenyl)-N-methyl-N-(1H-pyrazol-5- | 0.143042 |
| ylmethyl)-1,3-benzoxazole-5-carboxamide | |
| 5-[(1R*,5S*)-6-azabicyclo[3.2.1]oct-6-ylcarbonyl]- | 0.143042 |
| 2-(3-fluorophenyl)-1,3-benzoxazole | |
| 1-{[2-(3-methoxyphenyl)-1,3-benzoxazol-6- | 0.141104 |
| yl]carbonyl}-3-piperidinol | |
| 2-(3-fluorophenyl)-N-methyl-N-(tetrahydro-2H- | 0.138521 |
| pyran-2-ylmethyl)-1,3-benzoxazole-5-carboxamide | |
| 2-(3-methoxyphenyl)-N-methyl-N-[(5-methyl- | 0.709073 |
| 1,3,4-oxadiazol-2-yl)methyl]-1,3-benzoxazole-6- | |
| carboxamide | |
| 4-({2-[4-(trifluoromethyl)phenyl]-1,3-benzoxazol- | 1.698095 |
| 5-yl}carbonyl)-2-piperazinone | |
| [(2S)-1-({2-[4-(trifluoromethyl)phenyl]-1,3- | 0.135292 |
| benzoxazol-5-yl}carbonyl)-2-pyrrolidinyl]methanol | |
| N-[(1-ethyl-1H-imidazol-2-yl)methyl]-2-(3- | 1.071359 |
| methoxyphenyl)-N-methyl-1,3-benzoxazole-6- | |
| carboxamide | |
| N-[(3,5-dimethyl-1H-pyrazol-4-yl)methyl]-2-(3- | 0.132386 |
| methoxyphenyl)-N-methyl-1,3-benzoxazole-6- | |
| carboxamide | |
| N-ethyl-2-(3-methoxyphenyl)-N-[2-(1H-pyrazol-1- | 0.136261 |
| yl)ethyl]-1,3-benzoxazole-6-carboxamide | |
| N-(2-hydroxyethyl)-N-isopropyl-2-[4- | 0.132386 |
| (trifluoromethyl)phenyl]-1,3-benzoxazole-5- | |
| carboxamide | |
| (3S)-1-ethyl-4-{[2-(3-methoxyphenyl)-1,3- | 0.143365 |
| benzoxazol-6-yl]carbonyl}-3-methyl-2- | |
| piperazinone | |
| methyl 1-{[2-(3-methoxyphenyl)-1,3-benzoxazol- | 0.137552 |
| 6-yl]carbonyl}-4-piperidinecarboxylate | |
| N-methyl-N-(1H-pyrazol-5-ylmethyl)-2-[4- | 0.141427 |
| (trifluoromethyl)phenyl]-1,3-benzoxazole-5- | |
| carboxamide | |
| 3-(4-{[2-(3-methoxyphenyl)-1,3-benzoxazol-6- | 1.077494 |
| yl]carbonyl}-1-piperazinyl)propanamide | |
| 2-(3-methoxyphenyl)-6-{[4-(3-pyridinylmethyl)-1- | 0.141104 |
| piperazinyl]carbonyl}-1,3-benzoxazole | |

### Example 5. Treatment with a Small Molecule Suppresses Melanoma Progression In Vivo

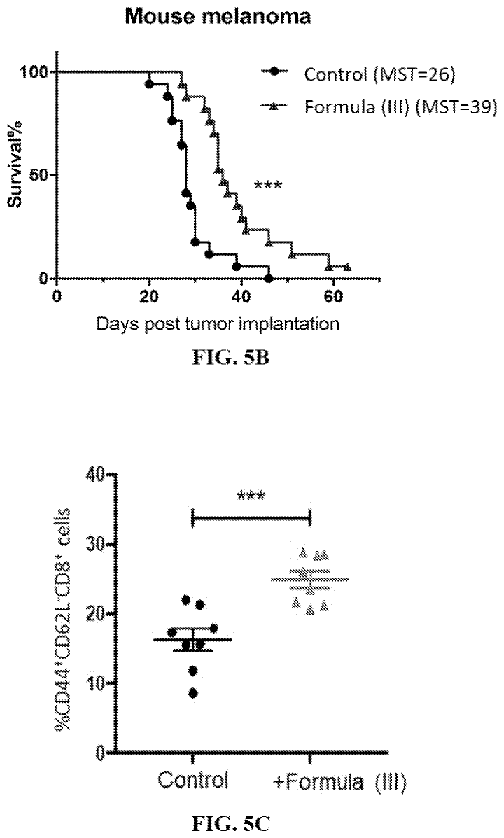

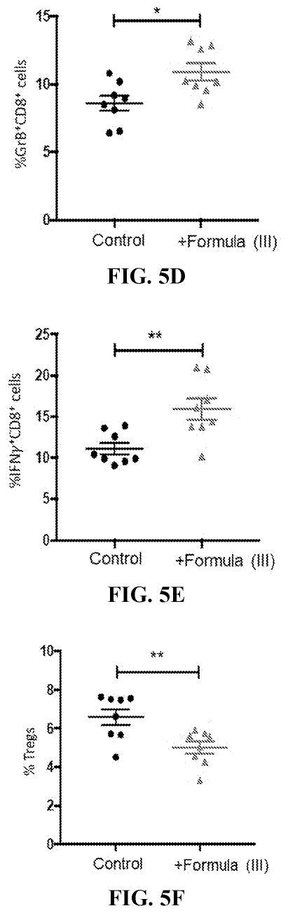

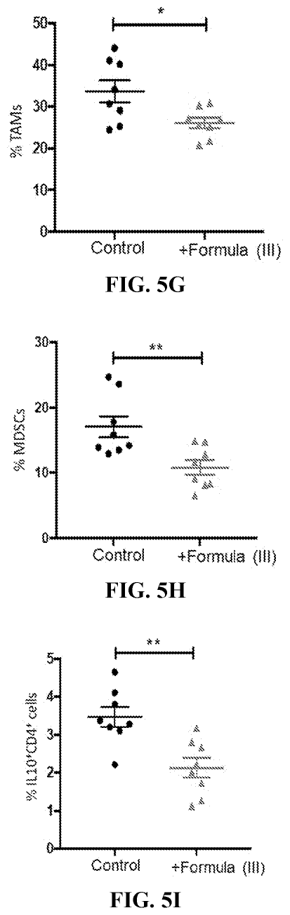

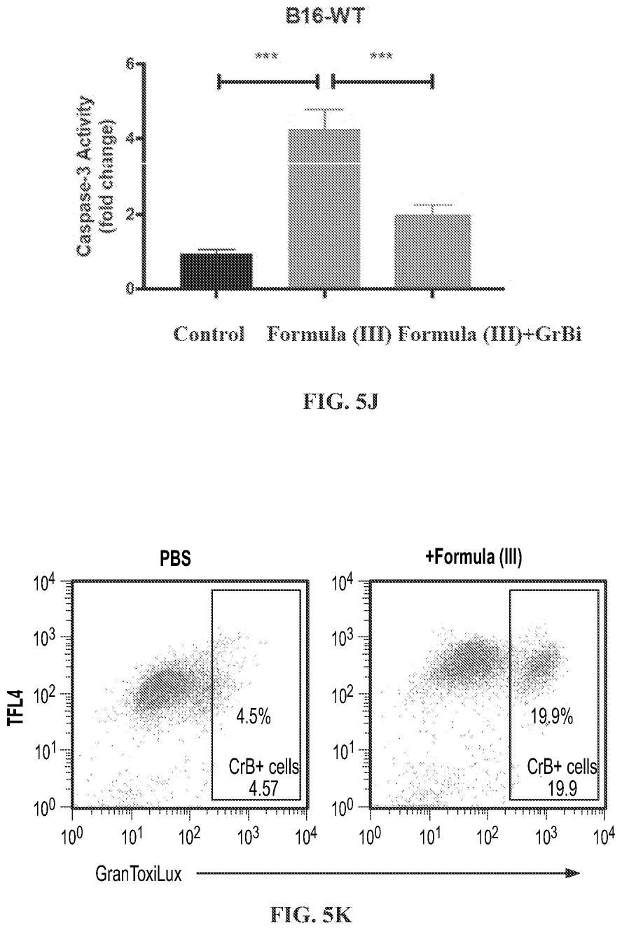

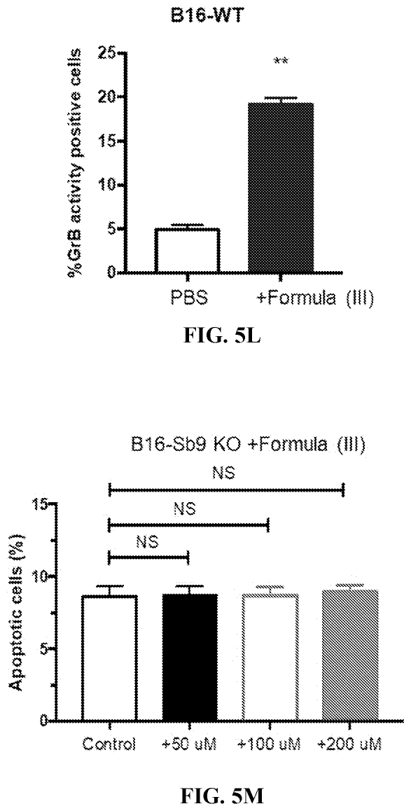

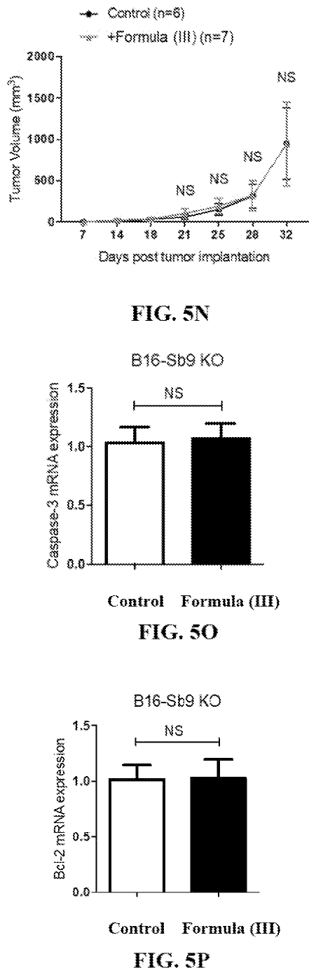

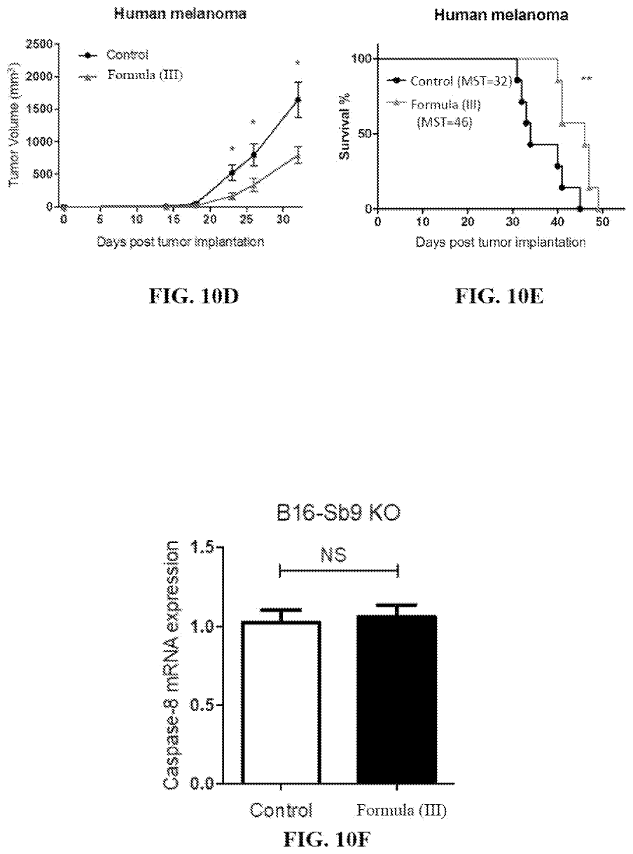

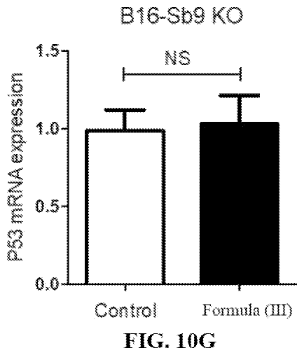

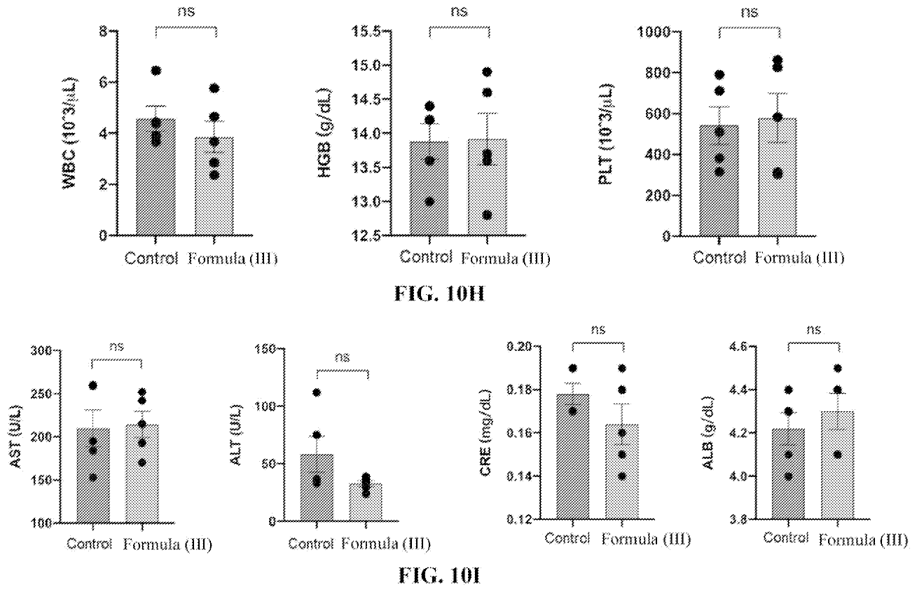

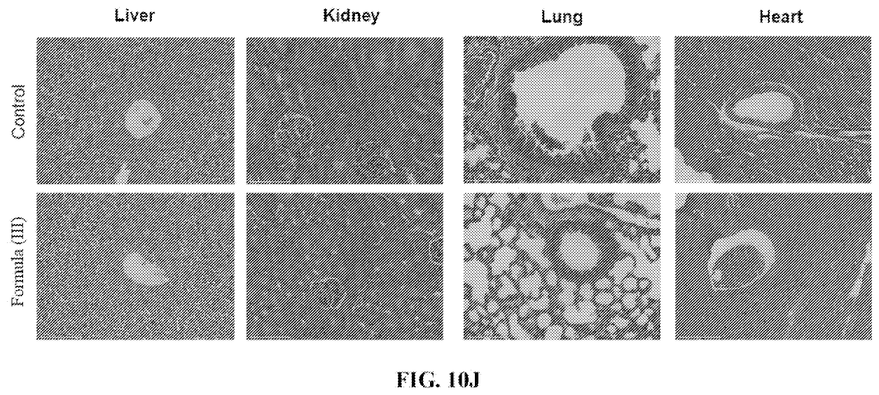

The anti-tumor activity of the compound of Formula (III) in vivo was assessed. The injections of the compound of Formula (III) significantly reduced (3.7-fold, p=0.0014) the size of B16 tumors implanted in the flanks of mice, compared to the control injected group (FIGS. 5A and 10A). Treatment with the compound of Formula (III) increased the median survival time (MST) of mice bearing B 16-WT mouse melanoma tumors and receiving treatment with the compound of Formula (III) as compared to mice bearing B16-WT melanoma tumors and not receiving treatment (MST=39 days vs MST=26 days, respectively) (FIG. 51B). Next, the effect of the compound of Formula (III) on tumor immunity was examined. The populations of CD44⁺ CD62L⁻ (effector memory) CD8⁺, effector GrB⁺ CD8⁺, and effector IFNγ⁺ CD8⁺ were all significantly higher in the melanoma sections from the compound of Formula (III)-treatment group, as compared with those from the control group (FIGS. 5C-5E). The levels of immunosuppressive Tregs, TAMs, and MDSCs (p=0.025) were significantly lower in melanoma sections from the compound of Formula (III)-treatment group, as compared to the control group (FIGS. 5F-5H). Furthermore, an increased level of effector IFNγ⁺ CD4⁺ cells and a decreased level of immunosuppressive IL10⁺CD4⁺ cells and IL10⁺CD8⁺ cells were found in the TME sections from the compound of Formula (III)-treatment group (FIGS. 5I and 10B-10C).

To assess if the anti-tumor effects of the compound of Formula (III) were generalizable to human diseases, the efficacy in an NSG humanized mouse model of A375 human melanoma was also tested. The compound of Formula (III) reduced the size of human melanoma tumors significantly (2.1-fold, p=0.0158) compared to controls (FIG. 10D) and increased MST (FIG. 10E). The compound of Formula (III) induced GrB-mediated apoptosis in B16 melanoma cells (FIG. 5J). Grantoxilux assay demonstrated the increased activity of GrB in B16 cells treated with the compound of Formula (III) (FIGS. 5K-5L). Thus, the compound of Formula (III) can inhibit Sb9 in melanoma tumor cells and render them susceptible to GrB-mediated apoptosis.

To evaluate the off-target effects of the compound of Formula (III) to Sb9 proteins, the B16-Sb9 KO melanoma cells were treated with various concentrations of the compound of Formula (III). No additional apoptotic effect was observed (FIG. 5M). Furthermore, the antitumor effects of the compound of Formula (III) on the Sb9 KO mice bearing B16-Sb9 KO tumors, as compared to control group were not observed (FIG. 5N). RT-PCR results showed that the compound of Formula (III) has no significant effect on the gene expression of caspase-3/8, P53, and Bcl-2 in the B16-Sb9 KO cells (FIGS. 5O-5P and 10F-10G).

To further assess the in vivo toxicity of the compound of Formula (III), 300 μg of the compound of Formula (III) was administered twice a day intraperitoneally for 14 days. The CBC results showed no significant difference in WBC, HGB, and PLT between control and treatment groups (FIG. 10H). The serum cells suggested that AST, ALT, CRE, and ALB levels were similar between the two groups (FIG. 10I). H&E staining demonstrated that the compound of Formula (III) showed no toxicity to liver, kidney, lung, and heart tissues in mice (FIG. 10J).

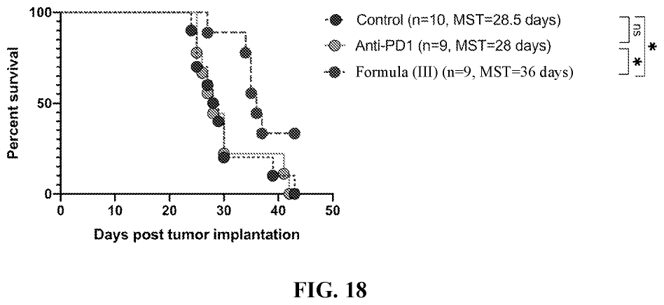

The efficacy of the compound of Formula (III) was assessed in C57BL/6J mice (FIG. 18). B16 mouse melanoma cells were implanted subcutaneously into the right flanks of C57BL/6J mice, and the mice were randomized into three groups: (1) no treatment (control), (2) treatment with anti-PD1, and (3) treatment with Formula (III). The mice in the anti-PD1 group received 200 μg of anti-PD1 iv every 3 days, and those in the Formula (III) group received 300 μg of Formula (III) twice a day consecutively for 14 days. As shown in FIG. 18, median survival time (MST) for the mice that received Formula (III) (36 days) was significantly longer than those that received either no treatment (28.5 days) or anti-PD1 (28 days) (*p<0.05).

### Example 6. Treatment with a Small Molecule Restrains Breast Tumor Growth In Vivo

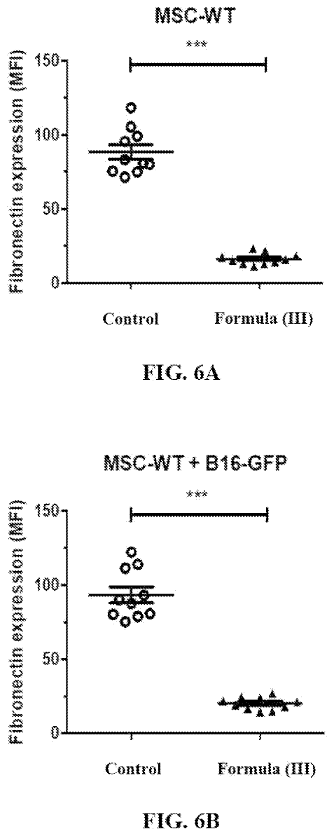

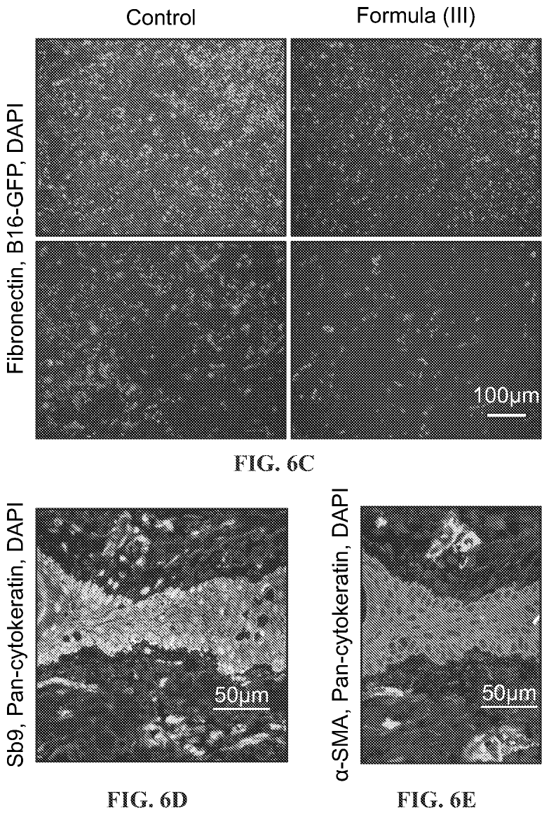

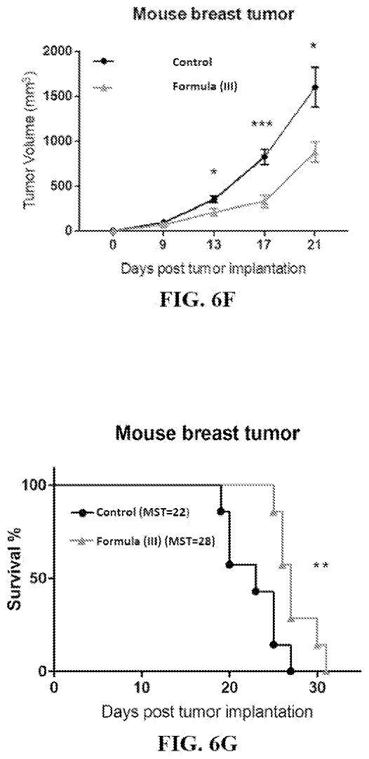

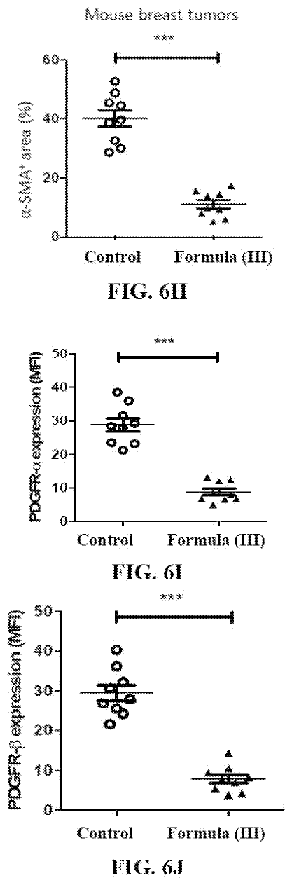

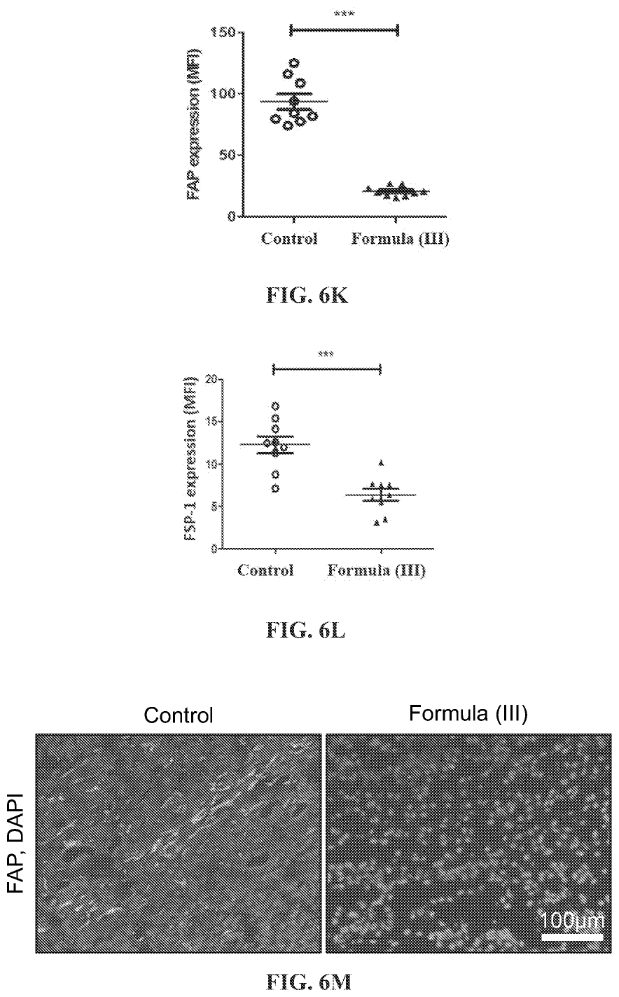

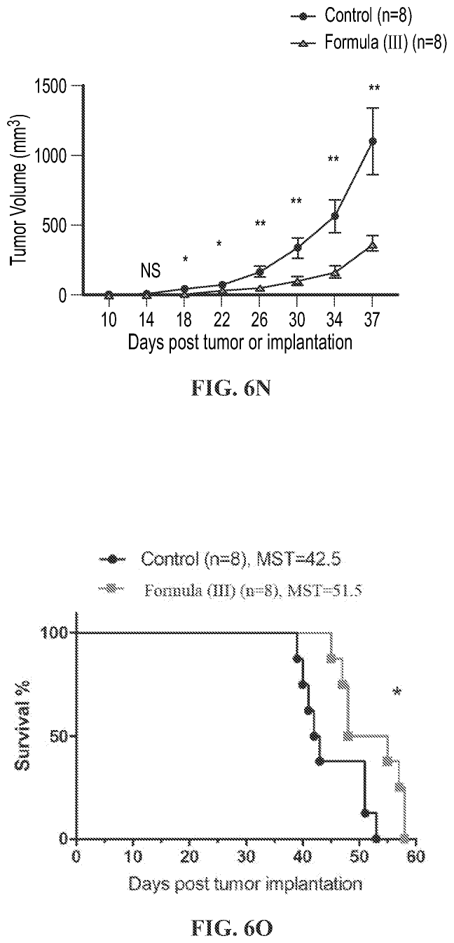

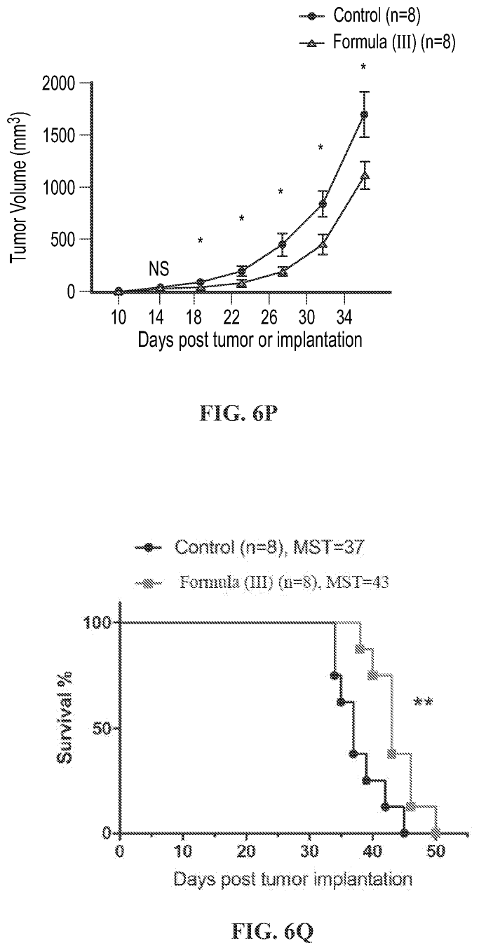

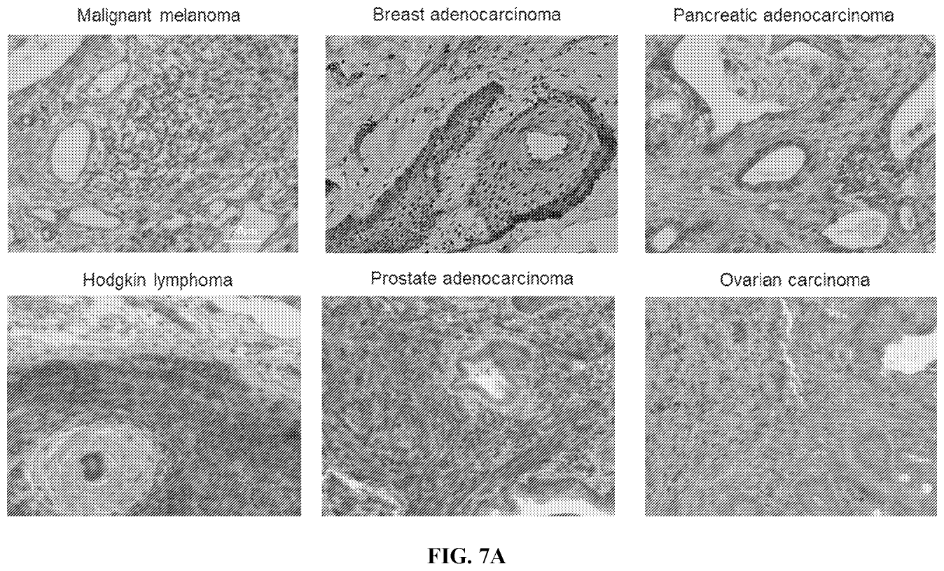

To investigate the effect of the compound of Formula (III) on the tumor stroma, MSC-WT cells were treated with the compound of Formula (III) for 48 hrs. Interestingly, treatment with the compound of Formula (III) reduced the production of fibronectin and collagen I by MSC-WT cells significantly (FIGS. 6A and 11A). As expected, the compound of Formula (III) decreased the expression of fibronectin and collagen I by MSC-WT cells that were co-cultured with B16 cells (FIGS. 6B-6C and 11B-11C).

Breast cancers exhibit a high degree of stromal immune-suppression cancer (Kalluri, 2016; Lauricella et al., 2016). Sb9 is highly expressed in human primary breast tumor cells (FIG. 6D) and in the stromal cells within the TME (FIGS. 6D-6E). Whether the compound of Formula (III) could be used to treat mice implanted with 4T1 breast tumor cells was therefore determined. Treatment with the compound of Formula (III) significantly reduced (2.5-fold, p=0.0006) the size of 4T1-derived tumors (FIG. 6F) in comparison to the control group. Treatment with the compound of Formula (III) increased the median survival time (MST) of mice bearing 4T1 breast tumors and receiving treatment with the compound of Formula (III) as compared to mice bearing 4T1 breast tumors and not receiving treatment (MST=28 days vs MST=22 days, respectively) (FIG. 6G). Treatment with the compound of Formula (III) also resulted in reduced levels of stromal cells and CAFs (FIGS. 6H-6M).

Furthermore, similar antitumor effects and increased MST using the compound of Formula (III) in two other in vivo tumor models were observed: Renca renal cell carcinoma and LLC1 lung cancer (FIGS. 6M-6Q). Thus, inhibition of Sb9 by the compound of Formula (III) has potent anti-tumor effects in a broad spectrum of tumors, including melanomas as well as breast, lung, and kidney tumors.

### Example 7. The Compounds of Formulas (I) and (II) Bind Sb9, Increase Expression of Caspase-3, and Significantly Decrease Tumor Size In Vivo

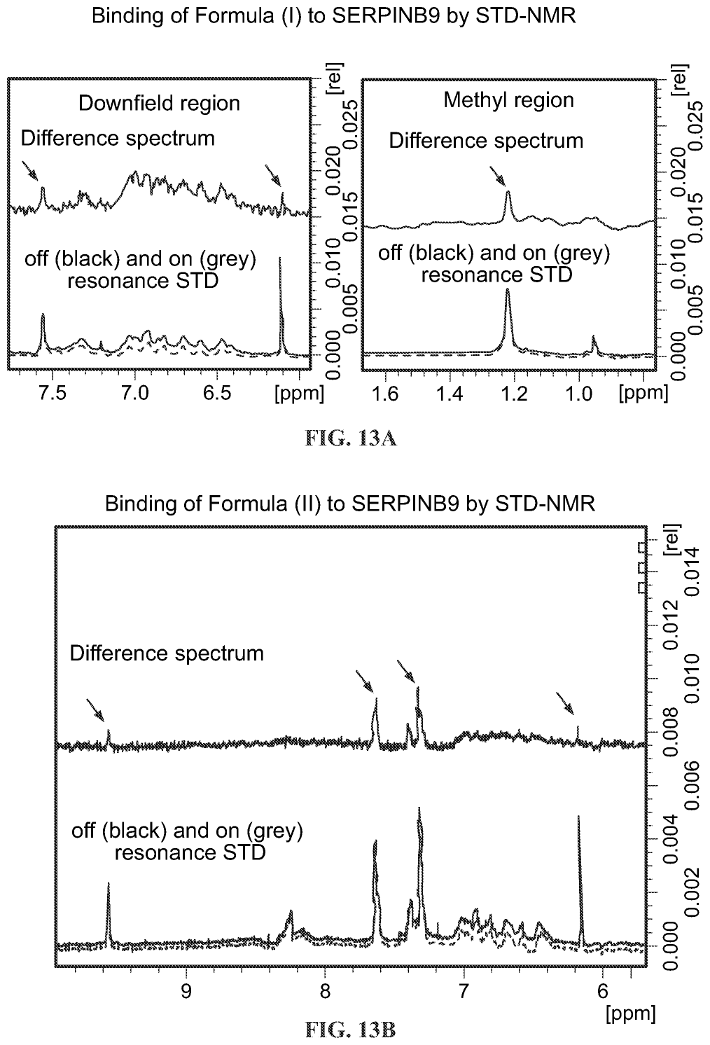

The Saturation-Transfer Difference (STD) NMR assay was used to evaluate the binding of the compounds of Formulas (I) and (II) to Sb9. The STD NMR experiment was performed using 420 μM of the compounds of Formulas (I) and (II) with 45 μM Sb9 protein in PBS buffer, pH 7.0 with 10% D₂O at 25° C., on a 600 MHz Bruker Avance II spectrometer with a Prodigy Cryoprobe. The saturation period was 3.0 seconds with irradiation at 0.7 ppm (on-resonance excitation) and 20 ppm (off-resonance excitation). The number of scans was 800. The spectra were processed using Topspin software with 1.0 Hz line-broadening. Saturation-Transfer Difference (STD) NMR assays in FIGS. 13A and 13B confirmed the binding activities of the compounds of Formulas (I) and (II) to MBP-Sb9.

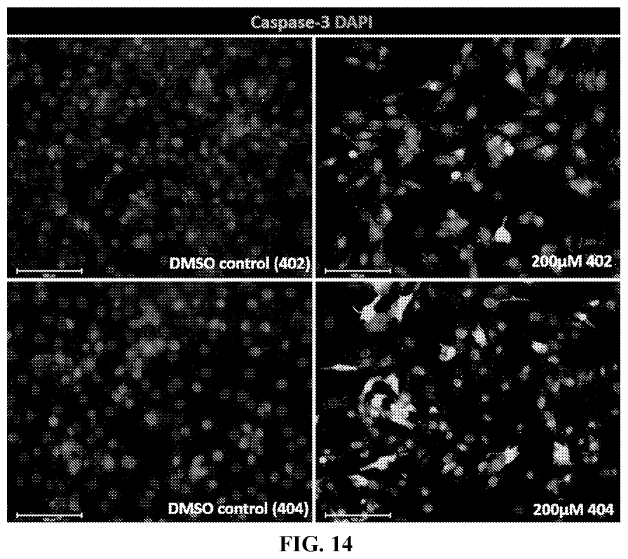

1×10⁴ cancer cells were seeded in an 8-well chamber and treated with 200 μM of the compounds of Formulas (I) and (II) for 48 hrs. Controls were the equal amount of DMSO. Cells were stained with anti-caspase3. DAPI was used to counterstain the cell nuclei. The stained tissue sections were visualized using an EVOS™ FL Auto 2 Imaging System. The fluorescence images in FIG. 14 showed that the compounds of Formulas (I) and (II) at 200 μM significantly increased caspase-3 expression, compared to the equal amount of DMSO. The increase of caspase-3 expression in the group treated with the compound of Formula (II) was higher than the group treated with the compound of Formula (I).

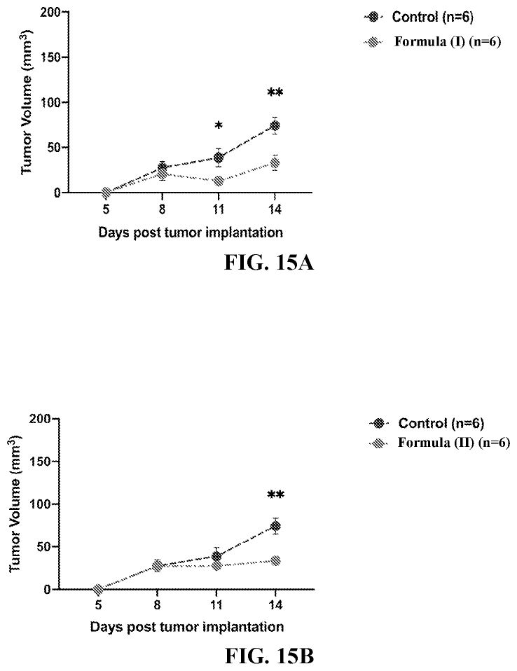

For mouse Breast Cancer model, 1×10⁵ 4T1 cells were subcutaneously implanted into the fourth mammary pad of BABL/c mice, and mice were separated randomly into three groups: Control, compound of Formula (I), and compound of Formula (II). In the compound of Formula (I) group, mice were treated with the compound of Formula (I) for 14 days consecutively (300 g/mouse, i.p, twice a day). In the compound of Formula (II) group, mice were administrated the compound of Formula (II) 14 days consecutively (300 μg/mouse, i.p, twice a day). Tumor sizes were measured every 3 days from day 5 post-tumor implantation and calculated with formula length×width²/2. As shown in FIG. 15A, tumor size was significantly decreased in the compound of Formula (I) group compared to Control group at Day 11, Day 14 post-tumor implantation. As shown in FIG. 15B, tumor size was significantly decreased in the compound of Formula (II) group compared to Control group at Day 14 post-tumor implantation.

### Example 8. The Compound of Formula (IV) Binds Sb9

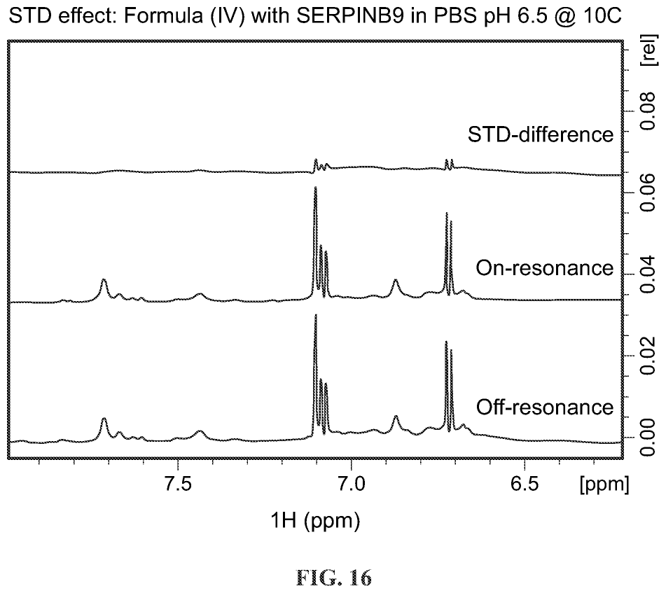

The Saturation-Transfer Difference (STD) NMR assay as described herein was used to evaluate the binding of the compound of Formula (III) to Sb9. The STD NMR assay confirmed the binding activity of the compound of Formula (III) to MBP-Sb9 (FIG. 16). The STD difference spectrum (top) obtained by subtracting on-resonance saturation spectrum (middle) from off-resonance reference spectrum (bottom) clearly show inter-molecular interaction of all aromatic peaks from the compound of Formula (IV).

### Example 9. Treatment of Mice Having B16 Melanoma with the Compound of Formula (IV) Increases MST and Suppresses Melanoma Progression In Vivo

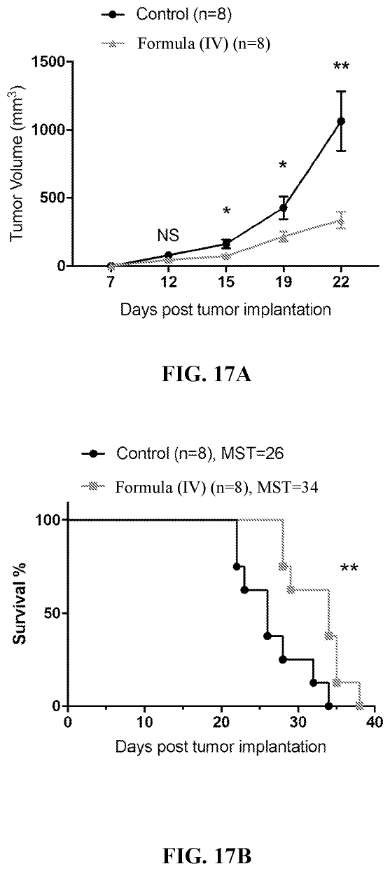

A survival study using the compound of Formula (IV) in mice harboring B16 melanoma was performed. Mice implanted with B16 tumors were treated with the compound of Formula (IV) (n=8) and compared to a control group having B16 tumors and not receiving treatment with the compound of Formula (IV) (n=8). Treatment with the compound of Formula (IV) suppressed melanoma progression in vivo (FIG. 17A) and increased the median survival time (MST) of mice bearing B16 tumors and receiving treatment with the compound of Formula (IV) as compared to mice bearing B16 tumors and not receiving treatment (MST=34 days vs MST=26 days, respectively) (FIG. 17B).

### REFERENCES

Annand, R. R., Dahlen, J. R., Sprecher, C. A., De Dreu, P., Foster, D. C., Mankovich, J. A., Talanian, R. V., Kisiel, W., and Giegel, D. A. (1999). Caspase-1 (interleukin-1beta-converting enzyme) is inhibited by the human serpin analogue proteinase inhibitor 9. Biochem J 342 Pt 3, 655-665.
Azzi, J., Skartsis, N., Mounayar, M., Magee, C. N., Batal, I., Ting, C., Moore, R., Riella, L. V., Ohori, S., Abdoli, R., et al. (2013). Serine protease inhibitor 6 plays a critical role in protecting murine granzyme B-producing regulatory T cells. J Immunol 191, 2319-2327.
Bird, C. H., Sutton, V. R., Sun, J., Hirst, C. E., Novak, A., Kumar, S., Trapani, J. A., and Bird, P. I. (1998). Selective regulation of apoptosis: the cytotoxic lymphocyte serpin proteinase inhibitor 9 protects against granzyme B-mediated apoptosis without perturbing the Fas cell death pathway. Mol Cell Biol 18, 6387-6398.
Bots, M., and Medema, J. P. (2008). Serpins in T cell immunity. J Leukoc Biol 84, 1238-1247.
Bots, M., Offringa, R., and Medema, J. P. (2006). Does the serpin PI-9 protect tumor cells? Blood 107, 4974-4975; author reply 4975.
Csermely, P., Agoston, V., and Pongor, S. (2005). The efficiency of multi-target drugs: the network approach might help drug design. Trends Pharmacol Sci 26, 178-182.
El Haddad, N., Heathcote, D., Moore, R., Yang, S., Azzi, J., Mfarrej, B., Atkinson, M., Sayegh, M. H., Lee, J. S., Ashton-Rickardt, P. G., et al. (2011a). Mesenchymal stem cells express serine protease inhibitor to evade the host immune response. Blood 117, 1176-1183.
El Haddad, N., Moore, R., Heathcote, D., Mounayar, M., Azzi, J., Mfarrej, B., Batal, I., Ting, C., Atkinson, M., Sayegh, M. H., et al. (2011b). The novel role of SERPINB9 in cytotoxic protection of human mesenchymal stem cells. J Immunol 187, 2252-2260.
Gonzalez-Garza, M. T., Cruz-Vega, D. E., Cardenas-Lopez, A., de la Rosa, R. M., and Moreno-Cuevas, J. E. (2018). Comparing stemness gene expression between stem cell subpopulations from peripheral blood and adipose tissue. Am J Stem Cells 7, 38-47.
Hirst, C. E., Buzza, M. S., Bird, C. H., Warren, H. S., Cameron, P. U., Zhang, M., Ashton-Rickardt, P. G., and Bird, P. I. (2003). The intracellular granzyme B inhibitor, proteinase inhibitor 9, is up-regulated during accessory cell maturation and effector cell degranulation, and its overexpression enhances CTL potency. J Immunol 170, 805-815.
Huntington, J. A., Read, R. J., and Carrell, R. W. (2000). Structure of a serpin-protease complex shows inhibition by deformation. Nature 407, 923-926.
Kalluri, R. (2016). The biology and function of fibroblasts in cancer. Nat Rev Cancer 16, 582-598.
Kather, J. N., Suarez-Carmona, M., Charoentong, P., Weis, C. A., Hirsch, D., Bankhead, P., Homing, M., Ferber, D., Kel, I., Herpel, E., et al. (2018). Topography of cancer-associated immune cells in human solid tumors. Elife 7.
Kendal, W. S., Wang, R. Y., Hsu, T. C., and Frost, P. (1987). Rate of generation of major karyotypic abnormalities in relationship to the metastatic potential of B16 murine melanoma. Cancer Res 47, 3835-3841.
Kim, M. T., and Harty, J. T. (2014). Impact of Inflammatory Cytokines on Effector and Memory CD8+ T Cells. Front Immunol 5, 295.
Kitamura, T., Qian, B. Z., and Pollard, J. W. (2015). Immune cell promotion of metastasis. Nat Rev Immunol 15, 73-86.
Kumar, V., Patel, S., Tcyganov, E., and Gabrilovich, D. I. (2016). The Nature of Myeloid-Derived Suppressor Cells in the Tumor Microenvironment. Trends Immunol 37, 208-220.
Lauricella, M., Carlisi, D., Giuliano, M., Calvaruso, G., Cernigliaro, C., Vento, R., and D'Anneo, A. (2016). The analysis of estrogen receptor-alpha positive breast cancer stem-like cells unveils a high expression of the serpin proteinase inhibitor PI-9: Possible regulatory mechanisms. Int J Oncol 49, 352-360.
Li, L. T., Jiang, G., Chen, Q., and Zheng, J. N. (2015). Ki67 is a promising molecular target in the diagnosis of cancer (review). Mol Med Rep 11, 1566-1572.
Lindau, D., Gielen, P., Kroesen, M., Wesseling, P., and Adema, G. J. (2013). The immunosuppressive tumour network: myeloid-derived suppressor cells, regulatory T cells and natural killer T cells. Immunology 138, 105-115.
Lipton, S. A. (2006). Paradigm shift in neuroprotection by NMDA receptor blockade: memantine and beyond. Nat Rev Drug Discov 5, 160-170.
Mangan, M. S., Bird, C. H., Kaiserman, D., Matthews, A. Y., Hitchen, C., Steer, D. L., Thompson, P. E., and Bird, P. I. (2016). A Novel Serpin Regulatory Mechanism: SerpinB9 IS REVERSIBLY INHIBITED BY VICINAL DISULFIDE BOND FORMATION IN THE REACTIVE CENTER LOOP. J Biol Chem 291, 3626-3638.
Mangan, M. S., Kaiserman, D., and Bird, P. I. (2008). The role of serpins in vertebrate immunity. Tissue Antigens 72, 1-10.
Medema, J. P., de Jong, J., Peltenburg, L. T., Verdegaal, E. M., Gorter, A., Bres, S. A., Franken, K. L., Hahne, M., Albar, J. P., Melief, C. J., et al. (2001a). Blockade of the granzyme B/perforin pathway through overexpression of the serine protease inhibitor PI-9/SPI-6 constitutes a mechanism for immune escape by tumors. Proc Natl Acad Sci USA 98, 11515-11520.
Medema, J. P., Schuurhuis, D. H., Rea, D., van Tongeren, J., de Jong, J., Bres, S. A., Laban, S., Toes, R. E., Toebes, M., Schumacher, T. N., et al. (2001b). Expression of the serpin serine protease inhibitor 6 protects dendritic cells from cytotoxic T lymphocyte-induced apoptosis: differential modulation by T helper type 1 and type 2 cells. J Exp Med 194, 657-667.
Mestres, J., and Gregori-Puigjane, E. (2009). Conciliating binding efficiency and polypharmacology. Trends Pharmacol Sci 30, 470-474.
Neradil, J., and Veselska, R. (2015). Nestin as a marker of cancer stem cells. Cancer Sci 106, 803-811.
Nilendu, P., Sarode, S. C., Jahagirdar, D., Tandon, I., Patil, S., Sarode, G. S., Pal, J. K., and Sharma, N. K. (2018). Mutual concessions and compromises between stromal cells and cancer cells: driving tumor development and drug resistance. Cell Oncol (Dordr) 41, 353-367.
Peoples, G. E. (2019). Improving the outcomes of checkpoint inhibitors in breast cancer. Lancet Oncol 20, 316-318.
Phillips, T., Opferman, J. T., Shah, R., Liu, N., Froelich, C. J., and Ashton-Rickardt, P. G. (2004). A role for the granzyme B inhibitor serine protease inhibitor 6 in CD8+ memory cell homeostasis. J Immunol 173, 3801-3809.
Pinkoski, M. J., Waterhouse, N.J., Heibein, J. A., Wolf, B. B., Kuwana, T., Goldstein, J. C., Newmeyer, D. D., Bleackley, R. C., and Green, D. R. (2001). Granzyme B-mediated apoptosis proceeds predominantly through a Bcl-2-inhibitable mitochondrial pathway. J Biol Chem 276, 12060-12067.
Postow, M. A., Sidlow, R., and Hellmann, M. D. (2018). Immune-Related Adverse Events Associated with Immune Checkpoint Blockade. N Engl J Med 378, 158-168.
Quail, D. F., and Joyce, J. A. (2013). Microenvironmental regulation of tumor progression and metastasis. Nat Med 19, 1423-1437.
Rizzitelli, A., Meuter, S., Vega Ramos, J., Bird, C. H., Mintern, J. D., Mangan, M. S., Villadangos, J., and Bird, P. I. (2012). Serpinb9 (Spi6)-deficient mice are impaired in dendritic cell-mediated antigen cross-presentation. Immunol Cell Biol 90, 841-851.
Silverman, G. A., Bird, P. I., Carrell, R. W., Church, F. C., Coughlin, P. B., Gettins, P. G., Irving, J. A., Lomas, D. A., Luke, C. J., Moyer, R. W., et al. (2001). The serpins are an expanding superfamily of structurally similar but functionally diverse proteins. Evolution, mechanism of inhibition, novel functions, and a revised nomenclature. J Biol Chem 276, 33293-33296.
Smits, A. H., Ziebell, F., Joberty, G., Zinn, N., Mueller, W. F., Clauder-Munster, S., Eberhard, D., Falth Savitski, M., Grandi, P., Jakob, P., et al. (2019). Biological plasticity rescues target activity in CRISPR knock outs. Nat Methods 16, 1087-1093.
Sun, J., Bird, C. H., Sutton, V., McDonald, L., Coughlin, P. B., De Jong, T. A., Trapani, J. A., and Bird, P. I. (1996). A cytosolic granzyme B inhibitor related to the viral apoptotic regulator cytokine response modifier A is present in cytotoxic lymphocytes. J Biol Chem 271, 27802-27809.
Sun, J., Ooms, L., Bird, C. H., Sutton, V. R., Trapani, J. A., and Bird, P. I. (1997). A new family of 10 murine ovalbumin serpins includes two homologs of proteinase inhibitor 8 and two homologs of the granzyme B inhibitor (proteinase inhibitor 9). J Biol Chem 272, 15434-15441.
Tamang, D. L., Redelman, D., Alves, B. N., Vollger, L., Bethley, C., and Hudig, D. (2006). Induction of granzyme B and T cell cytotoxic capacity by IL-2 or IL-15 without antigens: multiclonal responses that are extremely lytic if triggered and short-lived after cytokine withdrawal. Cytokine 36, 148-159.
Trujillo, J. A., Sweis, R. F., Bao, R., and Luke, J. J. (2018). T Cell-Inflamed versus Non-T Cell-Inflamed Tumors: A Conceptual Framework for Cancer Immunotherapy Drug Development and Combination Therapy Selection. Cancer Immunol Res 6, 990-1000.
Ugel, S., De Sanctis, F., Mandruzzato, S., and Bronte, V. (2015). Tumor-induced myeloid deviation: when myeloid-derived suppressor cells meet tumor-associated macrophages. J Clin Invest 125, 3365-3376.
Wei, L., Ye, H., Li, G., Lu, Y., Zhou, Q., Zheng, S., Lin, Q., Liu, Y., Li, Z., and Chen, R. (2018). Cancer-associated fibroblasts promote progression and gemcitabine resistance via the SDF-1/SATB-1 pathway in pancreatic cancer. Cell Death Dis 9, 1065.
Young, J. L., Sukhova, G. K., Foster, D., Kisiel, W., Libby, P., and Schonbeck, U. (2000). The serpin proteinase inhibitor 9 is an endogenous inhibitor of interleukin 1beta-converting enzyme (caspase-1) activity in human vascular smooth muscle cells. J Exp Med 191, 1535-1544.
Zhang, M., Park, S. M., Wang, Y., Shah, R., Liu, N., Murmann, A. E., Wang, C. R., Peter, M. E., and Ashton-Rickardt, P. G. (2006). Serine protease inhibitor 6 protects cytotoxic T cells from self-inflicted injury by ensuring the integrity of cytotoxic granules. Immunity 24, 451-461.
Zhu, H., Guo, Z. K., Jiang, X. X., Li, H., Wang, X. Y., Yao, H. Y., Zhang, Y., and Mao, N. (2010). A protocol for isolation and culture of mesenchymal stem cells from mouse compact bone. Nat Protoc 5, 550-560.

### OTHER EMBODIMENTS

It is to be understood that while the compounds and methods have been described in conjunction with the detailed description thereof, the foregoing description is intended to illustrate and not limit the scope, which is defined by the scope of the appended claims. Other aspects, advantages, and modifications are within the scope of the following claims.

What is claimed is:

1. A compound of Formula (I) 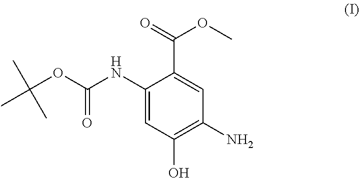 [SMILES: COC(=O)c1cc(N)c(O)cc1NC(=O)OC(C)(C)C]
or a pharmaceutically acceptable salt thereof.

2. The compound of claim 1 that is methyl 5-amino-2-((tert-butoxycarbonyl)amino)-4-hydroxybenzoate.

3. The compound of claim 1 that is a pharmaceutically acceptable salt of methyl 5-amino-2-((tert-butoxycarbonyl)amino)-4-hydroxybenzoate.

4. A compound of Formula (II) 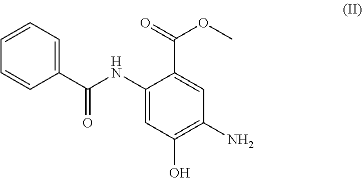 [SMILES: COC(=O)c1cc(N)c(O)cc1NC(=O)c1ccccc1]
or a pharmaceutically acceptable salt thereof.

5. The compound of claim 4 that is methyl 5-amino-2-benzamido-4-hydroxybenzoate.

6. The compound of claim 4 that is a pharmaceutically acceptable salt of methyl 5-amino-2-benzamido-4-hydroxybenzoate.

7. A pharmaceutical composition comprising a compound according to any one of claims 1 to 6, and a pharmaceutically acceptable carrier.

8. A method of treating a cancer in an individual in need thereof, comprising administering a therapeutically effective amount of a compound according to any one of claims 1 to 6 or a pharmaceutical composition according to claim 7.

9. The method according to any one of claim 8, wherein the cancer is a solid tumor.

10. The method according to claim 8, wherein the cancer is selected from melanoma, colorectal, pancreatic, lung, non-small cell lung cancer, breast, kidney, thyroid, lymphoid, gastrointestinal, genitourinary tract cancer, Hodgkin lymphoma, colon, renal cell carcinoma, ovarian, prostate cancer and/or testicular tumors, small intestine, and esophagus cancer.

11. A method of treating a cancer in a subject in need thereof, comprising administering a therapeutically effective amount of a compound of Formula (III) 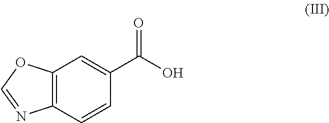 [SMILES: O=C(O)c1ccc2ncoc2c1]
or a pharmaceutically acceptable salt thereof.

12. A method of treating a cancer in a subject in need thereof, comprising administering a therapeutically effective amount of a compound of Formula (IV) 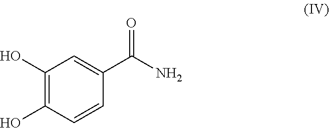 [SMILES: NC(=O)c1ccc(O)c(O)c1]
or a pharmaceutically acceptable salt thereof.

13. A method of treating a cancer in a subject in need thereof, comprising administering a therapeutically effective amount of a compound of Formula (V) 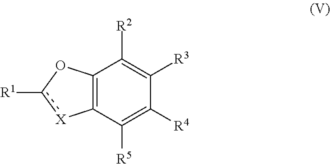
or a pharmaceutically acceptable salt thereof, wherein:
X is N, NH, or O; wherein when X is N, [symbol] is a double bond and when X is NH or O, [symbol] is a single bond;
R¹ is H, —C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂,
wherein the phenyl is optionally substituted with 1-5 substituents independently selected from halogen, C₁-C₃ alkyl, —O—(C₁-C₃ alkyl), and —C₁-C₃ haloalkyl;
one of R², R³, R⁴, and R⁵ is —(CH₂)ₙC(═O)NR⁶R⁷ and the others are each independently H or OH;
R⁶ is H, —C₁-C₃ alkyl, or —(C₁-C₃ alkylene)-(C₃-C₆ cycloalkyl);
R⁷ is —C₁-C₆ alkyl, —C₁-C₃ haloalkyl, —(C₁-C₃ alkylene)-O—(C₁-C₃ alkyl), —(C₁-C₃ alkylene)-N(C₁-C₃ alkyl)₂, —(C₁-C₃ alkylene)C(═O)NR⁸R⁹, —(C₁-C₃ alkylene)-phenyl, —C₃-C₆ cycloalkyl, 4-7 membered heterocyclic ring, —(C₁-C₃ alkylene)-(4-7 membered heterocyclic ring), —(C₁-C₃ alkylene)-NR⁸R⁹, —(C₁-C₃ alkylene)-C(═O)—O—(C₁-C₃ alkyl), or —OH,
wherein the —C₁-C₆ alkyl, —C₁-C₃ alkylene, and 4-7 membered heterocyclic ring are each optionally substituted with 1-2 substituents independently selected from —C₁-C₃ alkyl, OH, oxo (═O), and —O—(C₁-C₃ alkyl);
or R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, C₃-C₆ cycloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹;
R⁸ and R⁹ are each independently H or —C₁-C₃ alkyl; and
n is 0 or 1.

14. The method of claim 13, wherein R¹ is H, —C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂, wherein the phenyl is optionally substituted with 1-2 substituents independently selected from halogen, methyl, methoxy, and trifluoromethyl.

15. The method of claim 13 or 14, wherein R¹ is phenyl optionally substituted with 1-2 substituents independently selected from halogen, methyl, methoxy, and trifluoromethyl.

16. The method of any one of claims 13-15, wherein R³ is —C(═O)NR⁶R⁷ and R², R⁴, and R⁵ are each independently H.

17. The method of any one of claims 13-16, wherein R⁶ is H, methyl, ethyl, propyl, or —(CH₂)-(cyclopropyl).

18. The method of any one of claims 13-17, wherein R⁷ is —(C₁-C₃ alkylene)-(4-7 membered heterocyclic ring), wherein the 4-7 membered heterocyclic ring is optionally substituted with 1-2 —C₁-C₃ alkyl.

19. The method of claim 18, wherein the 4-7 membered heterocyclic ring is pyrazolyl, wherein the pyrazolyl is optionally substituted with 1-2 substituents independently selected from methyl and ethyl.

20. The method of any one of claims 13-17, wherein R⁷ is —C₁-C₆ alkyl optionally substituted with 1-2 OH substituents.

21. The method of any one of claims 13-17, wherein R⁷ is —C₁-C₃ alkylene-phenyl, wherein the —C₁-C₃ alkylene is optionally substituted with 1-2 OH substituents.

22. The method of any one of claims 13-17, wherein R⁷ is a 4-7 membered heterocyclic ring optionally substituted with 1-2 substituents independently selected from —C₁-C₃ alkyl, OH, oxo (═O), and —O—(C₁-C₃ alkyl).

23. The method of any one of claims 13-16, wherein R⁶ is C₁-C₃ alkyl and R⁷ is C₁-C₆ alkyl.

24. The method of any one of claims 13-16, wherein R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, C₃-C₆ cycloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹.

25. The method of any one of claims 13-16, wherein R⁶ and R⁷, together with the nitrogen to which they are attached, form a 4-6 membered heterocyclic ring optionally substituted with 1-3 substituents independently selected from —C₁-C₃ alkyl, —OH, —C₁-C₃ haloalkyl, C₃-C₆ cycloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹.

26. The method of claim 25, wherein R⁶ and R⁷, together with the nitrogen to which they are attached, form a heterocyclic ring selected from pyrrolidinyl, piperidinyl, and piperazinyl;
wherein the pyrrolidinyl is optionally substituted with 1-2 substituents independently selected from OH, —(C₁-C₃ alkylene)-OH, halogen, —C(═O)NR⁸R⁹, —NR⁸R⁹, and —(C₁-C₃ alkylene)-O—(C₁-C₃ alkyl);
wherein the piperidinyl is optionally substituted with 1-2 substituents independently selected from OH, phenyl, —(C₁-C₃ alkylene)-OH, —C(═O)NR⁸R⁹, —C(═O)—O—(C₁-C₃ alkyl), —O—(C₁-C₃ alkylene)-O—(C₁-C₃ alkyl), —C(═O)—(C₁-C₃ alkyl), —(C₁-C₃ alkylene)-C(═O)—O—(C₁-C₃ alkyl), and —O—(C₁-C₃ alkyl); and
wherein the piperazinyl ring is optionally substituted with 1-3 substituents independently selected from —C₁-C₃ alkyl, —C₁-C₃ haloalkyl, C₃-C₆ cycloalkyl, oxo (C═O), —(C₁-C₃ alkylene)-(4-6 membered heterocyclic ring) and —(C₁-C₃ alkylene)-C(═O)NR⁸R⁹.

27. The method of claim 13, wherein:
X is N and [symbol] is a double bond;
R¹ is phenyl optionally substituted with 1-5 halogen;
one of R², R³, R⁴, and R⁵ is —(CH₂)ₙC(═O)NR⁶R⁷ and the others are each H;
R⁶ is C₁-C₃ alkyl;
R⁷ is —C₂-C₄ alkyl; and
n is 0 or 1.

28. The method of claim 13, wherein X is N; R¹ is phenyl; R², R⁴, and R⁵ are each H; R³ is —C(═O)NR⁶R⁷; and R⁶ and R⁷ are each —C₁-C₃ alkyl.

29. The method of claim 13, wherein the compound is selected from: 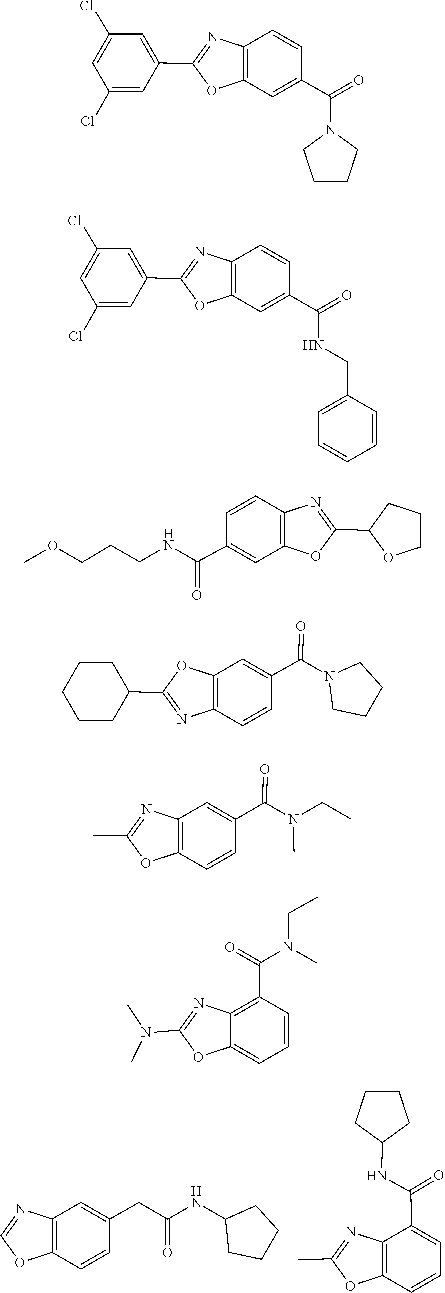 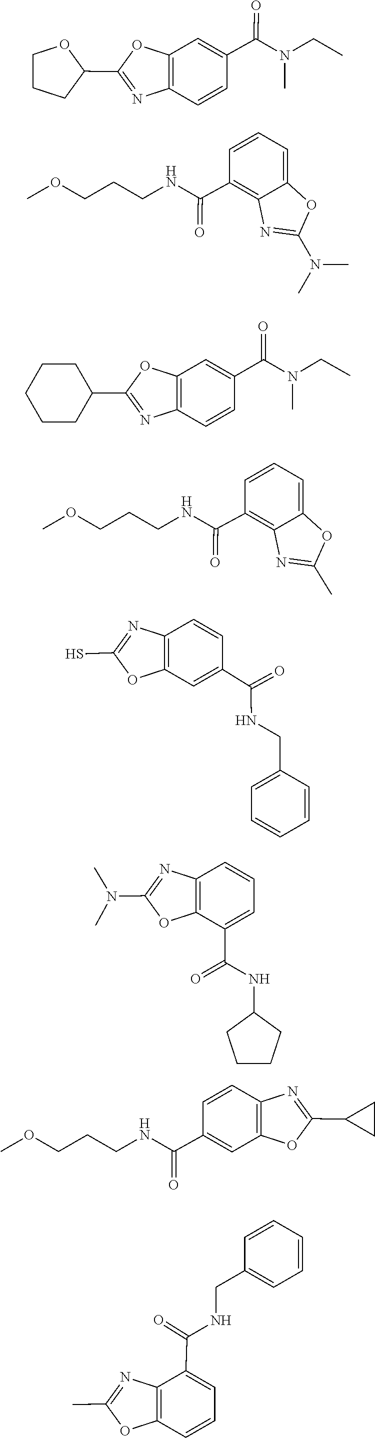 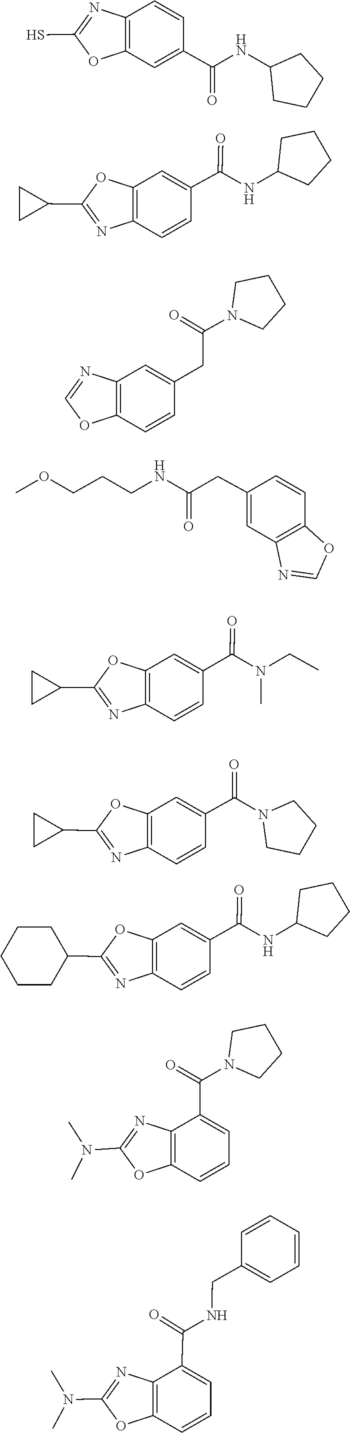 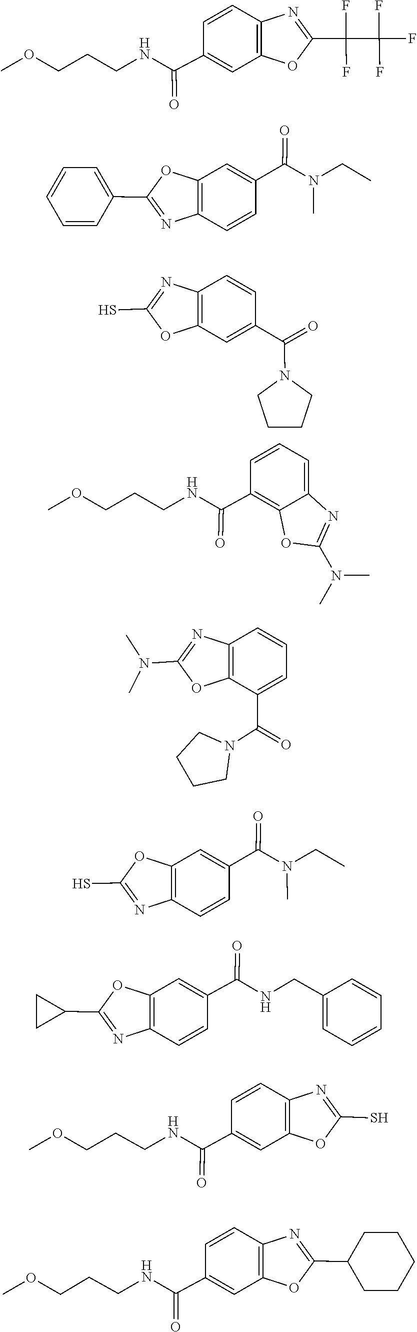 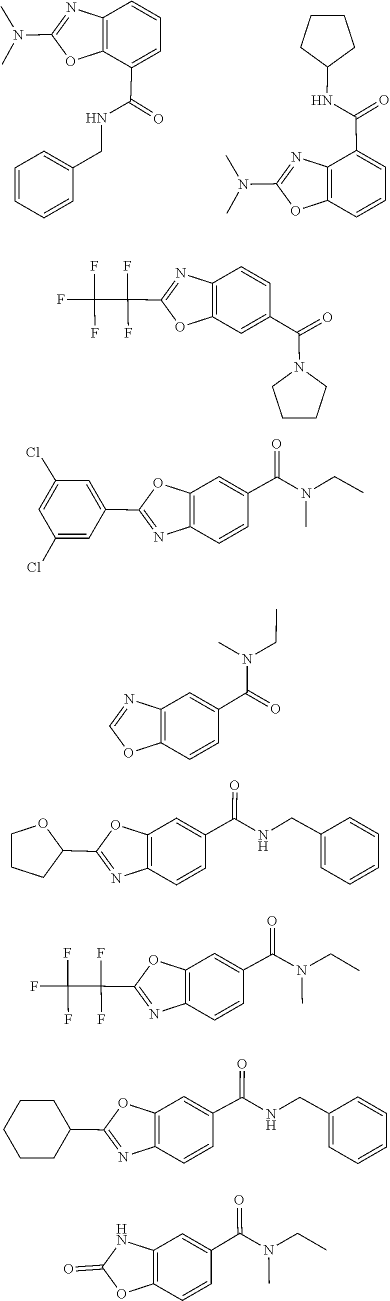 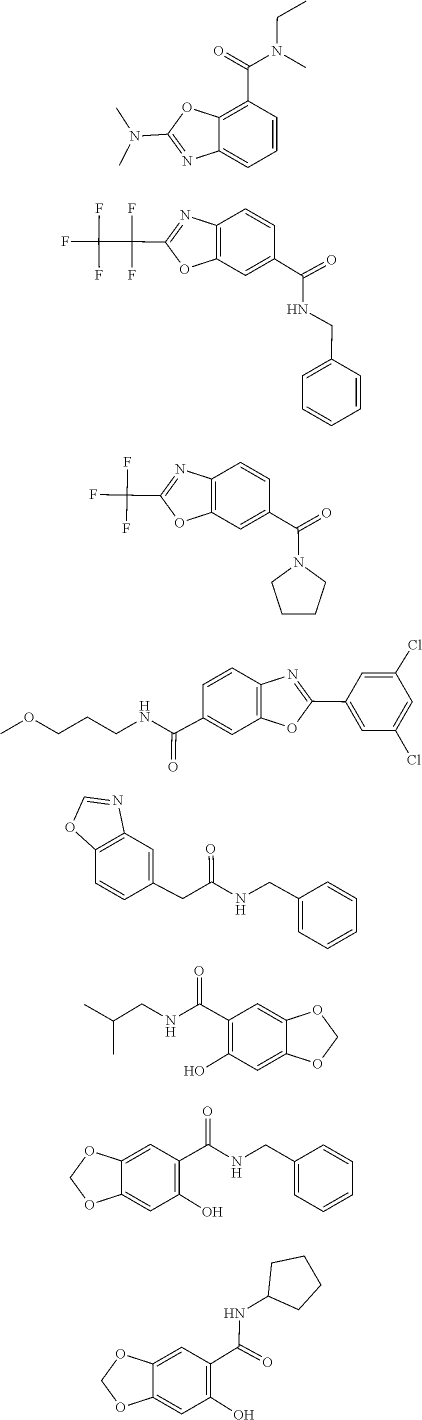 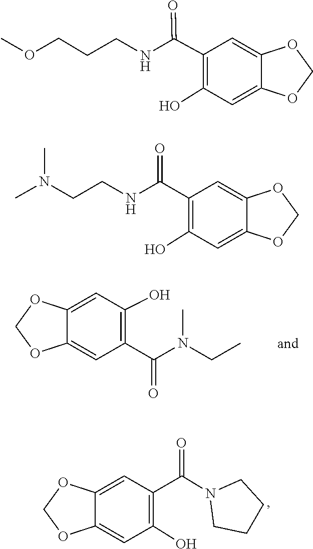
or a pharmaceutically acceptable salt thereof.

30. A method of treating a cancer in a subject in need thereof, comprising administering a therapeutically effective amount of a compound of Formula (Va) 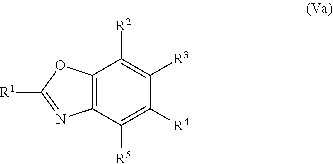
or a pharmaceutically acceptable salt thereof, wherein:
R¹ is H, —C₁-C₃ alkyl, —C₁-C₃ haloalkyl, —C₃-C₆ cycloalkyl, 4-6 membered heterocyclic ring, phenyl, —SH, oxo (═O), or —N(C₁-C₃ alkyl)₂,
wherein the phenyl is optionally substituted with 1-5 substituents independently selected from halogen, C₁-C₃ alkyl, —O—(C₁-C₃ alkyl), and —C₁-C₃ haloalkyl;
one of R², R³, R⁴, and R⁵ is —(CH₂)ₙC(═O)NR⁶R⁷ and the others are each independently H or —OH;
R⁶ is H, —C₁-C₃ alkyl, or —(C₁-C₃ alkylene)-(C₃-C₆ cycloalkyl);
R⁷ is —C₁-C₆ alkyl, —C₁-C₃ haloalkyl, —(C₁-C₃ alkylene)-O—(C₁-C₃ alkyl), —(C₁-C₃ alkylene)-N(C₁-C₃ alkyl)₂, —(C₁-C₃ alkylene)C(═O)NR⁸R⁹, —(C₁-C₃ alkylene)-phenyl, —C₃-C₆ cycloalkyl, 4-7 membered heterocyclic ring, —(C₁-C₃ alkylene)-(4-7 membered heterocyclic ring), —(C₁-C₃ alkylene)-NR⁸R⁹, —(C₁-C₃ alkylene)-C(═O)—O—(C₁-C₃ alkyl), or —OH,
wherein the —C₁-C₆ alkyl, —C₁-C₃ alkylene, and 4-7 membered heterocyclic ring are each optionally substituted with 1-2 substituents independently selected from —C₁-C₃ alkyl, OH, oxo (═O), and —O—(C₁-C₃ alkyl);
or R⁶ and R⁷, together with the nitrogen to which they are attached, form a 4-11 membered heterocyclic ring optionally substituted with 1-3 substituents independently selected from —C₁-C₃ alkyl, —OH, —C₁-C₃ haloalkyl, phenyl, —(C₁-C₃ alkylene)-OH, halogen, oxo (C═O), —C(═O)NR⁸R⁹, —NR⁸R⁹, —C(═O)—O—(C₁-C₃ alkyl), 4-6 membered heterocyclic ring, —(C₁-C₃ alkylene)-(4-6 membered heterocyclic ring), —O—(C₁-C₃ alkylene)-O—(C₁-C₃ alkyl), —C(═O)—(C₁-C₃ alkyl), —(C₁-C₃ alkylene)-O—(C₁-C₃ alkyl), —(C₁-C₃ alkylene)-C(═O)—O—(C₁-C₃ alkyl), —O—(C₁-C₃ alkyl), and —(C₁-C₃ alkylene)-C(═O)NR⁸R⁹;
R⁸ and R⁹ are each independently H or —C₁-C₃ alkyl; and
n is 0 or 1.

31. A method of treating a cancer in a subject in need thereof, comprising administering a therapeutically effective amount of a compound of Formula (Vb) 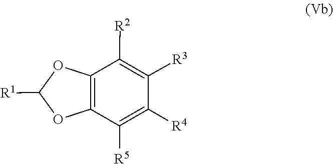
or a pharmaceutically acceptable salt thereof, wherein:
R¹ is H, —C₁-C₃ alkyl, —C₁-C₃ haloalkyl, —C₃-C₆ cycloalkyl, 4-6 membered heterocyclic ring, phenyl optionally substituted with 1-5 halogen, —SH, oxo (═O), or —N(C₁-C₃ alkyl)₂;
one of R², R³, R⁴, and R⁵ is —(CH₂)ₙC(═O)NR⁶R⁷ and the others are each independently H or —OH;
R⁶ is H or —C₁-C₃ alkyl;
R⁷ is —C₂-C₄ alkyl, —(C₁-C₃ alkylene)-O—(C₁-C₃ alkyl), —(C₁-C₃ alkylene)-N(C₁-C₃ alkyl)₂, —C₁-C₃ alkylene-phenyl, —C₃-C₆ cycloalkyl, or —OH;
or R⁶ and R⁷, together with the nitrogen to which they are attached, form a 4-6 membered heterocyclic ring; and
n is 0 or 1.

32. A method of treating a cancer in a subject in need thereof, comprising administering a therapeutically effective amount of a compound of Formula (VI) 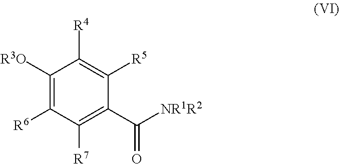
or a pharmaceutically acceptable salt thereof, wherein:
R¹ is H or —C₁-C₃ alkyl;
R² is —C₂-C₄ alkyl, —(C₁-C₃ alkylene)-O—(C₁-C₃ alkyl), —(C₁-C₃ alkylene)-N(C₁-C₃ alkyl)₂, —(C₁-C₃ alkylene)-(C₃-C₆ cycloalkyl), —(C₁-C₃ alkylene)-(4-6 membered heterocyclic ring), —C₁-C₃ alkylene-phenyl, or —C₃-C₆ cycloalkyl; or
or R¹ and R², taken together with the nitrogen to which they are attached, form a 4-6 membered heterocyclic ring;
R³ is H or —C₁-C₆ alkyl;
R⁴ is H, —OH, or —O—C₁-C₃ alkyl;
R⁵ is H, C₁-C₃ alkyl, or halogen;
R⁶ is H, —OH, or —O—C₁-C₃ alkyl; and
R⁷ is H, C₁-C₃ alkyl, or halogen.

33. The method of claim 32, wherein:
R¹ is H;
R² is —C₁-C₃ alkylene-phenyl;
R³ is —C₁-C₆ alkyl;
R⁴ is H;
R⁵ is H;
R⁶ is —OH; and
R⁷ is H.

34. The method of claim 32, wherein R¹ is H; R² is —(CH₂)-phenyl; R³ is C₁-C₃ alkyl; R⁴, R⁵, and R⁷ are each H; and R⁶ is —OH.

35. The method of claim 32, wherein the compound is selected from: 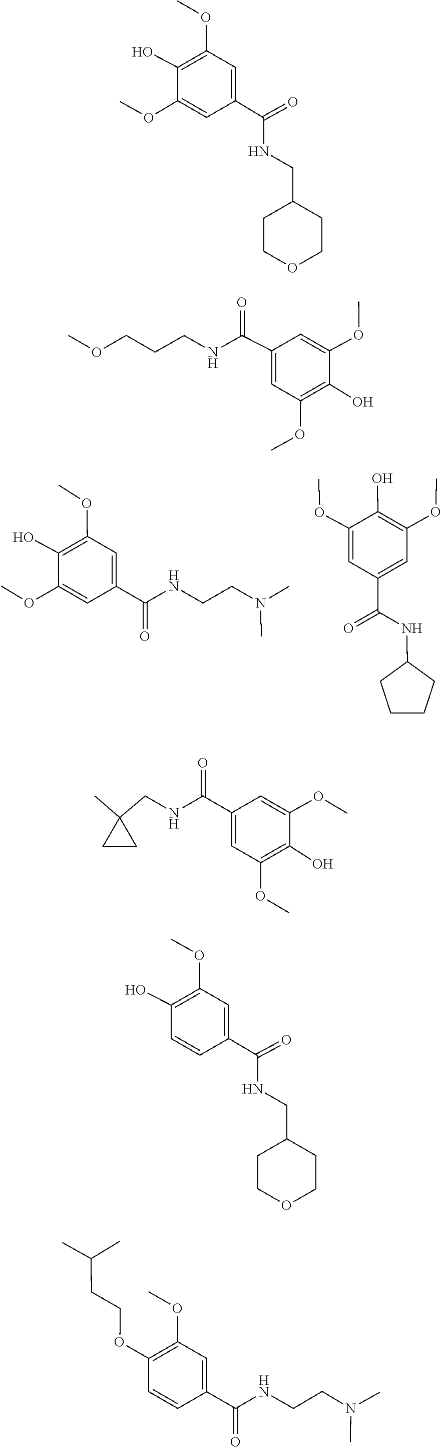 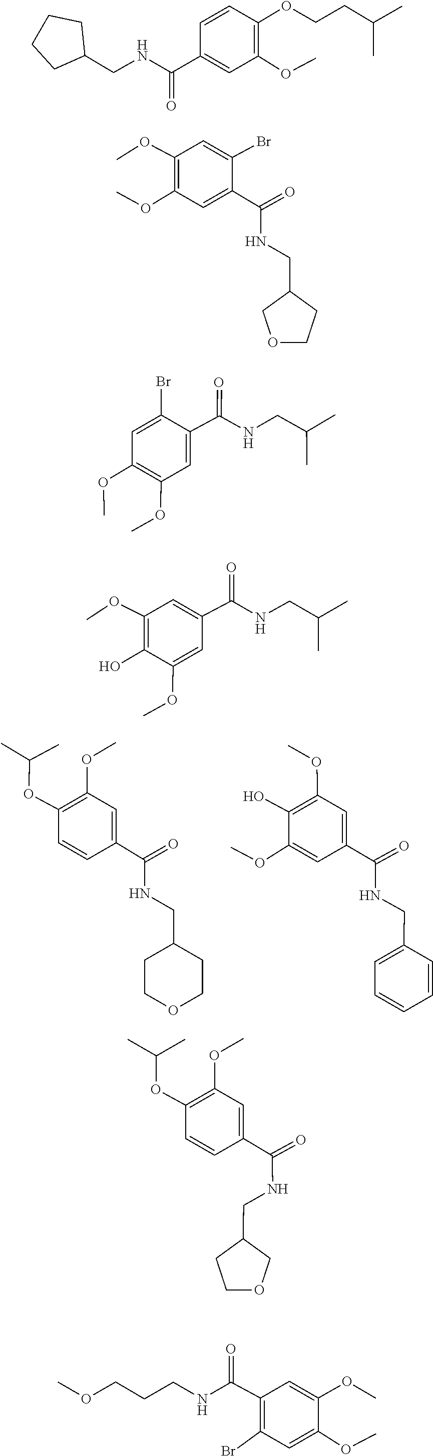 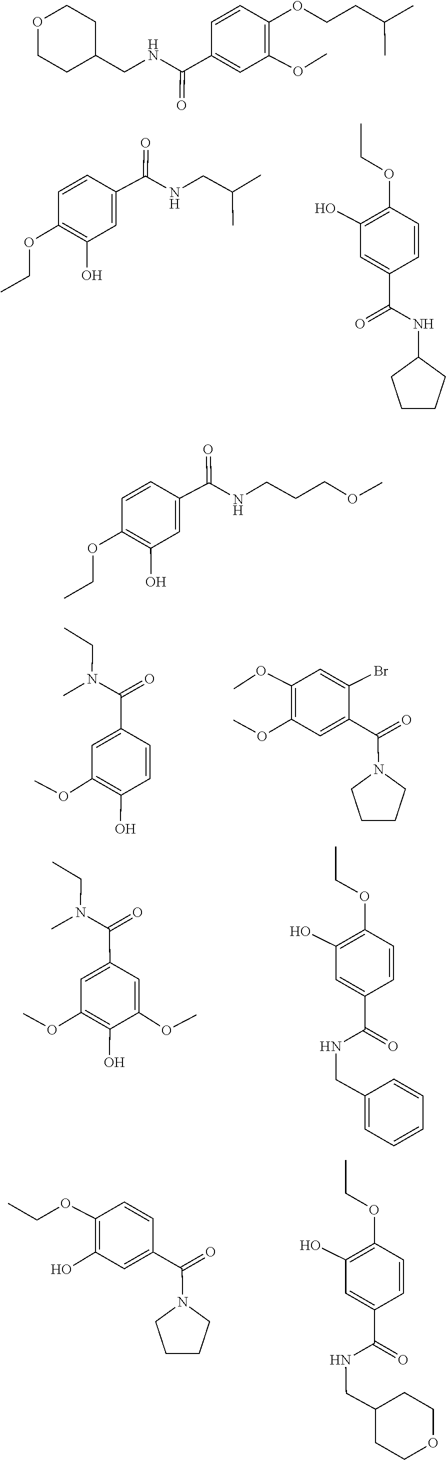 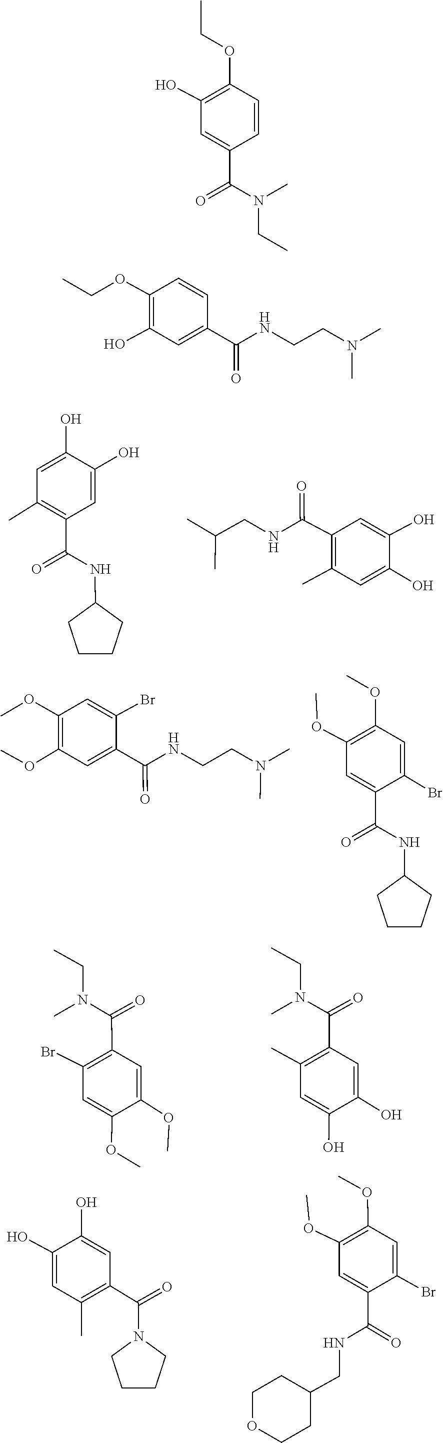 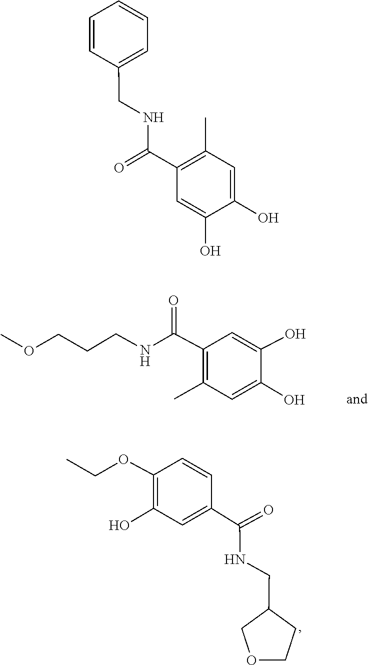 [SMILES: Cc1cc(O)c(O)cc1C(=O)NCc1ccccc1 ; COCCCNC(=O)c1cc(O)c(O)cc1C ; CCOc1ccc(C(=O)NCC2CCOC2)cc1O]
or a pharmaceutically acceptable salt thereof.

36. A method of treating a cancer in a subject in need thereof, comprising administering a therapeutically effective amount of a compound selected from:
methyl 5-amino-2-((tert-butoxycarbonyl)amino)-4-hydroxybenzoate;
methyl 5-amino-2-benzamido-4-hydroxybenzoate;
1,3-benzoxazole-6-carboxylic acid;
3,4-dihydroxybenzamide;
N-benzyl-2-(dimethylamino)-1,3-benzoxazole-4-carboxamide;
2-cyclopropyl-N-ethyl-N-methyl-1,3-benzoxazole-6-carboxamide;
2-cyclohexyl-N-cyclopentyl-1,3-benzoxazole-6-carboxamide;
N-benzyl-4-ethoxy-3-hydroxybenzamide;
1-(2-bromo-4,5-dimethoxybenzoyl)pyrrolidine;
N-(3-methoxypropyl)-2-sulfanyl-1,3-benzoxazole-6-carboxamide;
N-ethyl-N-methyl-2-(oxolan-2-yl)-1,3-benzoxazole-6-carboxamide;
N-[2-(dimethylamino)ethyl]-3-methoxy-4-(3-methylbutoxy)benzamide;
N-cyclopentyl-4-ethoxy-3-hydroxybenzamide;
2-(1,3-benzoxazol-5-yl)-1-(pyrrolidin-1-yl)ethan-1-one;
N-[2-(dimethylamino)ethyl]-6-hydroxy-2H-1,3-benzodioxole-5-carboxamide;
2-cyclohexyl-N-ethyl-N-methyl-1,3-benzoxazole-6-carboxamide;
4-hydroxy-3,5-dimethoxy-N-[(1-methylcyclopropyl)methyl]benzamide;
2-bromo-4,5-dimethoxy-N-[(oxolan-3-yl)methyl]benzamide;
3-methoxy-N-[(oxolan-3-yl)methyl]-4-(propan-2-yloxy)benzamide;
N-ethyl-6-hydroxy-N-methyl-2H-1,3-benzodioxole-5-carboxamide;
4-ethoxy-3-hydroxy-N-(2-methylpropyl)benzamide;
2-(1,3-benzoxazol-5-yl)-N-cyclopentylacetamide;
2-cyclopropyl-N-(3-methoxypropyl)-1,3-benzoxazole-6-carboxamide;
N-[2-(dimethylamino)ethyl]-4-hydroxy-3,5-dimethoxybenzamide;
N-ethyl-N-methyl-2-sulfanyl-1,3-benzoxazole-6-carboxamide;
2-(3,5-dichlorophenyl)-6-(pyrrolidine-1-carbonyl)-1,3-benzoxazole;
N-benzyl-6-hydroxy-2H-1,3-benzodioxole-5-carboxamide;
N-benzyl-4-hydroxy-3,5-dimethoxybenzamide;
N-benzyl-2-sulfanyl-1,3-benzoxazole-6-carboxamide;
N-cyclopentyl-2-(dimethylamino)-1,3-benzoxazole-4-carboxamide;
N,N-dimethyl-7-(pyrrolidine-1-carbonyl)-1,3-benzoxazol-2-amine;
2-ethoxy-5-(pyrrolidine-1-carbonyl)phenol;
N-cyclopentyl-2-cyclopropyl-1,3-benzoxazole-6-carboxamide;
N-ethyl-N-methyl-2-oxo-2,3-dihydro-1,3-benzoxazole-5-carboxamide;
N,N-dimethyl-4-(pyrrolidine-1-carbonyl)-1,3-benzoxazol-2-amine;
N-(3-methoxypropyl)-2-(1,1,2,2,2-pentafluoroethyl)-1,3-benzoxazole-6-carboxamide;
4-hydroxy-3,5-dimethoxy-N-(3-methoxypropyl)benzamide;
N-cyclopentyl-4-hydroxy-3,5-dimethoxybenzamide;
4-ethoxy-3-hydroxy-N-[(oxolan-3-yl)methyl]benzamide;
4-hydroxy-3,5-dimethoxy-N-(2-methylpropyl)benzamide;
2-(dimethylamino)-N-ethyl-N-methyl-1,3-benzoxazole-4-carboxamide;
N-benzyl-2-(dimethylamino)-1,3-benzoxazole-7-carboxamide;
2-cyclopropyl-6-(pyrrolidine-1-carbonyl)-1,3-benzoxazole;
4,5-dihydroxy-2-methyl-N-(2-methylpropyl)benzamide;
N-ethyl-N-methyl-2-(1,1,2,2,2-pentafluoroethyl)-1,3-benzoxazole-6-carboxamide;
2-cyclohexyl-6-(pyrrolidine-1-carbonyl)-1,3-benzoxazole;
N-cyclopentyl-2-methyl-1,3-benzoxazole-4-carboxamide;
6-(pyrrolidine-1-carbonyl)-1,3-benzoxazole-2-thiol;
4-ethoxy-3-hydroxy-N-[(oxan-4-yl)methyl]benzamide;
2-(dimethylamino)-N-ethyl-N-methyl-1,3-benzoxazole-7-carboxamide;
2-(dimethylamino)-N-(3-methoxypropyl)-1,3-benzoxazole-4-carboxamide;
2-(1,3-benzoxazol-5-yl)-N-(3-methoxypropyl)acetamide;
6-hydroxy-N-(2-methylpropyl)-2H-1,3-benzodioxole-5-carboxamide;
2-(3,5-dichlorophenyl)-N-(3-methoxypropyl)-1,3-benzoxazole-6-carboxamide;
3-methoxy-4-(3-methylbutoxy)-N-[(oxan-4-yl)methyl]benzamide;
N-benzyl-2-cyclopropyl-1,3-benzoxazole-6-carboxamide;
N-ethyl-4,5-dihydroxy-N,2-dimethylbenzamide;
N-ethyl-4-hydroxy-3-methoxy-N-methylbenzamide;
2-(1,3-benzoxazol-5-yl)-N-benzylacetamide;
N-benzyl-2-(oxolan-2-yl)-1,3-benzoxazole-6-carboxamide;
N-ethyl-4-hydroxy-3,5-dimethoxy-N-methylbenzamide;
4-ethoxy-3-hydroxy-N-(3-methoxypropyl)benzamide;
6-(pyrrolidine-1-carbonyl)-2H-1,3-benzodioxol-5-ol;
N-benzyl-2-(3,5-dichlorophenyl)-1,3-benzoxazole-6-carboxamide;
2-(1,1,2,2,2-pentafluoroethyl)-6-(pyrrolidine-1-carbonyl)-1,3-benzoxazole;
N-[2-(dimethylamino)ethyl]-4-ethoxy-3-hydroxybenzamide;
2-(dimethylamino)-N-(3-methoxypropyl)-1,3-benzoxazole-7-carboxamide;
4-methyl-5-(pyrrolidine-1-carbonyl)benzene-1,2-diol;
6-hydroxy-N-(3-methoxypropyl)-2H-1,3-benzodioxole-5-carboxamide;
2-cyclohexyl-N-(3-methoxypropyl)-1,3-benzoxazole-6-carboxamide;
N-benzyl-4,5-dihydroxy-2-methylbenzamide;
2-bromo-4,5-dimethoxy-N-[(oxan-4-yl)methyl]benzamide;
N-benzyl-2-methyl-1,3-benzoxazole-4-carboxamide;
N-cyclopentyl-2-(dimethylamino)-1,3-benzoxazole-7-carboxamide;
N-benzyl-2-(1,1,2,2,2-pentafluoroethyl)-1,3-benzoxazole-6-carboxamide;
2-bromo-N-cyclopentyl-4,5-dimethoxybenzamide;
N-(cyclopentylmethyl)-3-methoxy-4-(3-methylbutoxy)benzamide;
4,5-dihydroxy-N-(3-methoxypropyl)-2-methylbenzamide;
2-bromo-4,5-dimethoxy-N-(2-methylpropyl)benzamide;
N-benzyl-2-cyclohexyl-1,3-benzoxazole-6-carboxamide;
N-cyclopentyl-6-hydroxy-2H-1,3-benzodioxole-5-carboxamide;
N-ethyl-N,2-dimethyl-1,3-benzoxazole-5-carboxamide;
3-methoxy-N-[(oxan-4-yl)methyl]-4-(propan-2-yloxy)benzamide;
N-cyclopentyl-4,5-dihydroxy-2-methylbenzamide;
N-(3-methoxypropyl)-2-(oxolan-2-yl)-1,3-benzoxazole-6-carboxamide;
4-ethoxy-N-ethyl-3-hydroxy-N-methylbenzamide;
2-bromo-N-[2-(dimethylamino)ethyl]-4,5-dimethoxybenzamide;
N-(3-methoxypropyl)-2-methyl-1,3-benzoxazole-4-carboxamide;
6-(pyrrolidine-1-carbonyl)-2-(trifluoromethyl)-1,3-benzoxazole;
2-bromo-N-ethyl-4,5-dimethoxy-N-methylbenzamide;
N-ethyl-N-methyl-1,3-benzoxazole-5-carboxamide;
4-hydroxy-3-methoxy-N-[(oxan-4-yl)methyl]benzamide;
N-ethyl-N-methyl-2-phenyl-1,3-benzoxazole-6-carboxamide;
4-hydroxy-3,5-dimethoxy-N-[(oxan-4-yl)methyl]benzamide;
N-cyclopentyl-2-sulfanyl-1,3-benzoxazole-6-carboxamide;
2-(3,5-dichlorophenyl)-N-ethyl-N-methyl-1,3-benzoxazole-6-carboxamide; and
2-bromo-4,5-dimethoxy-N-(3-methoxypropyl)benzamide;
or a pharmaceutically acceptable salt thereof.

37. A method of treating a cancer in a subject in need thereof, comprising administering a therapeutically effective amount of a compound selected from:
1-(2-phenyl-1,3-benzoxazole-6-carbonyl)piperidin-4-ol;
3-[(2-phenyl-1,3-benzoxazol-6-yl)formamido]-N-(propan-2-yl)propanamide;
N-(5,5-dimethyloxolan-3-yl)-2-phenyl-1,3-benzoxazole-6-carboxamide;
[(2S,4S)-4-fluoro-1-(2-phenyl-1,3-benzoxazole-6-carbonyl)pyrrolidin-2-yl]methanol;
6-{6-oxa-1-azaspiro[3.4]octane-1-carbonyl}-2-phenyl-1,3-benzoxazole;
N-(2,6-dioxopiperidin-3-yl)-2-phenyl-1,3-benzoxazole-6-carboxamide;
2-(3-fluorophenyl)-N-[3-(1H-pyrazol-1-yl)propyl]-1,3-benzoxazole-5-carboxamide;
2-(1-{[2-(3-fluorophenyl)-1,3-benzoxazol-5-yl]carbonyl}-2-piperidinyl)ethanol;
methyl 4-({[2-(3-methoxyphenyl)-1,3-benzoxazol-6-yl]carbonyl}amino)butanoate;
2-(3-fluorophenyl)-N-(3-pyridinylmethyl)-1,3-benzoxazole-5-carboxamide;
2-(3-fluorophenyl)-N-[2-(1H-pyrazol-1-yl)ethyl]-1,3-benzoxazole-5-carboxamide;
1-{[2-(3-methoxyphenyl)-1,3-benzoxazol-6-yl]carbonyl}-3-piperidinol;
2-(3-fluorophenyl)-N-[(1-methyl-1H-pyrazol-4-yl)methyl]-1,3-benzoxazole-5-carboxamide;
2-(3-methoxyphenyl)-N-(tetrahydro-2H-pyran-2-ylmethyl)-1,3-benzoxazole-6-carboxamide;
N-[(1,5-dimethyl-1H-pyrazol-3-yl)methyl]-2-(3-fluorophenyl)-1,3-benzoxazole-5-carboxamide;
2-(3-methoxyphenyl)-N-(tetrahydro-2H-pyran-4-ylmethyl)-1,3-benzoxazole-6-carboxamide;
ethyl N-{[2-(3-fluorophenyl)-1,3-benzoxazol-5-yl]carbonyl}-beta-alaninate;
2-(3-fluorophenyl)-N-[(2-methyl-1,3-thiazol-4-yl)methyl]-1,3-benzoxazole-5-carboxamide;
2-(3-fluorophenyl)-N-[2-(2-furyl)ethyl]-1,3-benzoxazole-5-carboxamide;
2-(3-methoxyphenyl)-N-(tetrahydro-2-furanylmethyl)-1,3-benzoxazole-6-carboxamide;
2-(3-fluorophenyl)-N-methyl-N-(1H-pyrazol-5-ylmethyl)-1,3-benzoxazole-5-carboxamide;
N-(2-hydroxyethyl)-2-[4-(trifluoromethyl)phenyl]-1,3-benzoxazole-5-carboxamide;
1-{[2-(3-fluorophenyl)-1,3-benzoxazol-5-yl]carbonyl}-N-methylpyrrolidine-3-carboxamide;
N-(1,1-dioxido-2,3-dihydro-3-thienyl)-2-(3-fluorophenyl)-1,3-benzoxazole-5-carboxamide;
1-({2-[4-(trifluoromethyl)phenyl]-1,3-benzoxazol-5-yl}carbonyl)azetidin-3-ol;
N-(3-hydroxypropyl)-2-[4-(trifluoromethyl)phenyl]-1,3-benzoxazole-5-carboxamide;
N-(2-hydroxyethyl)-N-methyl-2-[4-(trifluoromethyl)phenyl]-1,3-benzoxazole-5-carboxamide;
2-(3-fluorophenyl)-N-[2-(4-methyl-4H-1,2,4-triazol-3-yl)ethyl]-1,3-benzoxazole-5-carboxamide;
2-(3-methoxyphenyl)-N-(1H-tetrazol-5-ylmethyl)-1,3-benzoxazole-6-carboxamide;
1-{[2-(3-fluorophenyl)-1,3-benzoxazol-5-yl]carbonyl}-L-prolinamide;
2-(3-fluorophenyl)-5-(thiomorpholin-4-ylcarbonyl)-1,3-benzoxazole;
2-(3-methoxyphenyl)-N-[(5-methylpyrazin-2-yl)methyl]-1,3-benzoxazole-6-carboxamide;
2-(3-fluorophenyl)-N-methyl-N-[(5-methylisoxazol-3-yl)methyl]-1,3-benzoxazole-5-carboxamide;
2-(3-fluorophenyl)-N-(isoxazol-5-ylmethyl)-1,3-benzoxazole-5-carboxamide;
1-{[2-(3-fluorophenyl)-1,3-benzoxazol-5-yl]carbonyl}-2-methyl-1,4-diazepan-5-one;
N-(2-hydroxypropyl)-2-[4-(trifluoromethyl)phenyl]-1,3-benzoxazole-5-carboxamide;
2-(3-fluorophenyl)-N-[1-methyl-2-(1H-1,2,4-triazol-1-yl)ethyl]-1,3-benzoxazole-5-carboxamide;
5-[(1R*,5S*)-6-azabicyclo[3.2.1]oct-6-ylcarbonyl]-2-(3-fluorophenyl)-1,3-benzoxazole;
5-[(4-ethyl-1-piperazinyl)carbonyl]-2-(3-fluorophenyl)-1,3-benzoxazole;
2-(3-fluorophenyl)-N-methyl-N-(tetrahydro-2H-pyran-2-ylmethyl)-1,3-benzoxazole-5-carboxamide;
N-isopropyl-2-(3-methoxyphenyl)-N-methyl-1,3-benzoxazole-6-carboxamide;
ethyl N-{[2-(3-methoxyphenyl)-1,3-benzoxazol-6-yl]carbonyl}-beta-alaninate;
2-(3-fluorophenyl)-N-[2-(1-methyl-1H-pyrazol-4-yl)ethyl]-1,3-benzoxazole-5-carboxamide;
(1-{[2-(3-fluorophenyl)-1,3-benzoxazol-5-yl]carbonyl}-2-piperidinyl)methanol;
2-(3-fluorophenyl)-N-[1-(1H-1,2,4-triazol-5-yl)ethyl]-1,3-benzoxazole-5-carboxamide;
2-(3-fluorophenyl)-N-[2-(1H-pyrazol-4-yl)ethyl]-1,3-benzoxazole-5-carboxamide;
N-(3-hydroxy-2,2-dimethylpropyl)-2-(3-methoxyphenyl)-1,3-benzoxazole-6-carboxamide;
N-isopropyl-2-[4-(trifluoromethyl)phenyl]-1,3-benzoxazole-5-carboxamide;
2-(3-methoxyphenyl)-N-(2,2,2-trifluoroethyl)-1,3-benzoxazole-6-carboxamide;
N-[(1-ethyl-1H-pyrazol-4-yl)methyl]-2-(3-fluorophenyl)-1,3-benzoxazole-5-carboxamide;
2-(3-fluorophenyl)-N-[(3S)-2-oxo-3-azepanyl]-1,3-benzoxazole-5-carboxamide;
2-(3-fluorophenyl)-N-[(3S*,4S*)-4-methoxy-1-methylpyrrolidin-3-yl]-1,3-benzoxazole-5-carboxamide;
1-{[2-(3-methoxyphenyl)-1,3-benzoxazol-6-yl]carbonyl}-N,N-dimethyl-3-pyrrolidinamine;
N-cyclobutyl-2-[4-(trifluoromethyl)phenyl]-1,3-benzoxazole-5-carboxamide;
N-(2,3-dihydroxypropyl)-N-methyl-2-[4-(trifluoromethyl)phenyl]-1,3-benzoxazole-5-carboxamide;
2-(3-fluorophenyl)-N-methyl-N-[(5-methylisoxazol-3-yl)methyl]-1,3-benzoxazole-5-carboxamide;
1-{[2-(3-fluorophenyl)-1,3-benzoxazol-5-yl]carbonyl}-L-prolinamide;
2-(3-fluorophenyl)-N-(3-hydroxy-3-phenylpropyl)-N-methyl-1,3-benzoxazole-5-carboxamide;
N-[(5-ethyl-2-pyridinyl)methyl]-2-(3-fluorophenyl)-N-methyl-1,3-benzoxazole-5-carboxamide;
ethyl 1-{[2-(3-methoxyphenyl)-1,3-benzoxazol-6-yl]carbonyl}-3-piperidinecarboxylate;
N-(2-hydroxyethyl)-N-methyl-2-[4-(trifluoromethyl)phenyl]-1,3-benzoxazole-5-carboxamide;
1-{[2-(3-fluorophenyl)-1,3-benzoxazol-5-yl]carbonyl}-2-methyl-1,4-diazepan-5-one;
2-(3-fluorophenyl)-N-isopropyl-N-[(1-methyl-1H-imidazol-2-yl)methyl]-1,3-benzoxazole-5-carboxamide;
N-[(1-ethyl-1H-imidazol-2-yl)methyl]-N-methyl-2-[4-(trifluoromethyl)phenyl]-1,3-benzoxazole-5-carboxamide;
6-[(4-cyclopentyl-1-piperazinyl)carbonyl]-2-(3-methoxyphenyl)-1,3-benzoxazole;
5-(4-morpholinylcarbonyl)-2-[4-(trifluoromethyl)phenyl]-1,3-benzoxazole;
4-{[2-(3-methoxyphenyl)-1,3-benzoxazol-6-yl]carbonyl}morpholine-2-carboxamide;
2-(3-fluorophenyl)-5-(thiomorpholin-4-ylcarbonyl)-1,3-benzoxazole;
(3S)-1-({2-[4-(trifluoromethyl)phenyl]-1,3-benzoxazol-5-yl}carbonyl)-3-pyrrolidinol;
N,N-diethyl-1-{[2-(3-methoxyphenyl)-1,3-benzoxazol-6-yl]carbonyl}-3-piperidinecarboxamide;
2-(3-fluorophenyl)-5-{[4-(2-methoxyethoxy)-1-piperidinyl]carbonyl}-1,3-benzoxazole;
2-(1-{[2-(3-fluorophenyl)-1,3-benzoxazol-5-yl]carbonyl}-2-piperidinyl)ethanol;
N-isopropyl-2-(3-methoxyphenyl)-N-methyl-1,3-benzoxazole-6-carboxamide;
2-(3-methoxyphenyl)-N-methyl-N-[(5-methylisoxazol-3-yl)methyl]-1,3-benzoxazole-6-carboxamide;
6-[(1,1-dioxidothiomorpholin-4-yl)carbonyl]-2-(3-methoxyphenyl)-1,3-benzoxazole;
1-{[2-(3-fluorophenyl)-1,3-benzoxazol-5-yl]carbonyl}-N-methylpyrrolidine-3-carboxamide;
1-({2-[4-(trifluoromethyl)phenyl]-1,3-benzoxazol-5-yl}carbonyl)azetidin-3-ol;
N-methyl-N-(3-pyridinylmethyl)-2-[4-(trifluoromethyl)phenyl]-1,3-benzoxazole-5-carboxamide;
4-{[2-(3-fluorophenyl)-1,3-benzoxazol-5-yl]carbonyl}-2,3,4,5-tetrahydro-1,4-benzoxazepine;
N-ethyl-N-(2-hydroxyethyl)-2-[4-(trifluoromethyl)phenyl]-1,3-benzoxazole-5-carboxamide;
N-methyl-N-[2-(4-morpholinyl)ethyl]-2-[4-(trifluoromethyl)phenyl]-1,3-benzoxazole-5-carboxamide;
N-(cyclopropylmethyl)-2-(3-methoxyphenyl)-N-(tetrahydro-2-furanylmethyl)-1,3-benzoxazole-6-carboxamide;
N-methyl-N-[(4-methyl-1H-imidazol-2-yl)methyl]-2-[4-(trifluoromethyl)phenyl]-1,3-benzoxazole-5-carboxamide;
1-(1-{[2-(3-methoxyphenyl)-1,3-benzoxazol-6-yl]carbonyl}-3-piperidinyl)-1-propanone;
5-{[3-(methoxymethyl)-1-pyrrolidinyl]carbonyl}-2-[4-(trifluoromethyl)phenyl]-1,3-benzoxazole;
2-(3-fluorophenyl)-N-methyl-N-[2-(1-piperidinyl)ethyl]-1,3-benzoxazole-5-carboxamide;
N-methyl-N-(4-pyrimidinylmethyl)-2-[4-(trifluoromethyl)phenyl]-1,3-benzoxazole-5-carboxamide;
1-{[2-(3-fluorophenyl)-1,3-benzoxazol-5-yl]carbonyl}-4-phenyl-4-piperidinol;
2-(3-methoxyphenyl)-N-methyl-N-[2-(tetrahydro-2H-pyran-2-yl)ethyl]-1,3-benzoxazole-6-carboxamide;
N-(2-hydroxy-2-phenylethyl)-2-(3-methoxyphenyl)-N-methyl-1,3-benzoxazole-6-carboxamide;
ethyl (1-{[2-(3-methoxyphenyl)-1,3-benzoxazol-6-yl]carbonyl}-2-piperidinyl)acetate;
2-(3-methoxyphenyl)-N-methyl-N-[(5-propyl-1H-pyrazol-3-yl)methyl]-1,3-benzoxazole-6-carboxamide;
2-(3-methoxyphenyl)-6-[(3-propoxy-1-piperidinyl)carbonyl]-1,3-benzoxazole;
2-(3-fluorophenyl)-N-methyl-N-(1H-pyrazol-5-ylmethyl)-1,3-benzoxazole-5-carboxamide;
5-[(1R*,5S*)-6-azabicyclo[3.2.1]oct-6-ylcarbonyl]-2-(3-fluorophenyl)-1,3-benzoxazole;
1-{[2-(3-methoxyphenyl)-1,3-benzoxazol-6-yl]carbonyl}-3-piperidinol;
2-(3-fluorophenyl)-N-methyl-N-(tetrahydro-2H-pyran-2-ylmethyl)-1,3-benzoxazole-5-carboxamide;
2-(3-methoxyphenyl)-N-methyl-N-[(5-methyl-1,3,4-oxadiazol-2-yl)methyl]-1,3-benzoxazole-6-carboxamide;
4-({2-[4-(trifluoromethyl)phenyl]-1,3-benzoxazol-5-yl}carbonyl)-2-piperazinone;
[(2S)-1-({2-[4-(trifluoromethyl)phenyl]-1,3-benzoxazol-5-yl}carbonyl)-2-pyrrolidinyl]methanol;
N-[(1-ethyl-1H-imidazol-2-yl)methyl]-2-(3-methoxyphenyl)-N-methyl-1,3-benzoxazole-6-carboxamide;
N-[(3,5-dimethyl-1H-pyrazol-4-yl)methyl]-2-(3-methoxyphenyl)-N-methyl-1,3-benzoxazole-6-carboxamide;
N-ethyl-2-(3-methoxyphenyl)-N-[2-(1H-pyrazol-1-yl)ethyl]-1,3-benzoxazole-6-carboxamide;
N-(2-hydroxyethyl)-N-isopropyl-2-[4-(trifluoromethyl)phenyl]-1,3-benzoxazole-5-carboxamide;
(3S)-1-ethyl-4-{[2-(3-methoxyphenyl)-1,3-benzoxazol-6-yl]carbonyl}-3-methyl-2-piperazinone;
methyl 1-{[2-(3-methoxyphenyl)-1,3-benzoxazol-6-yl]carbonyl}-4-piperidinecarboxylate;
N-methyl-N-(1H-pyrazol-5-ylmethyl)-2-[4-(trifluoromethyl)phenyl]-1,3-benzoxazole-5-carboxamide;
3-(4-{[2-(3-methoxyphenyl)-1,3-benzoxazol-6-yl]carbonyl}-1-piperazinyl)propanamide;
2-(3-methoxyphenyl)-6-{[4-(3-pyridinylmethyl)-1-piperazinyl]carbonyl}-1,3-benzoxazole;
(3-isopropyl-4-methyl-piperazin-1-yl)-(8-phenyl-7-oxa-9-aza-bicyclo[4.3.0]nona-1(6),2,4,8-tetraen-3-yl)-methanone;
(4-methyl-piperazin-1-yl)-(8-phenyl-7-oxa-9-aza-bicyclo[4.3.0]nona-1(6),2,4,8-tetraen-3-yl)-methanone;
(4-methyl-3-(trifluoro-methyl)-piperazin-1-yl)-(8-phenyl-7-oxa-9-aza-bicyclo[4.3.0]nona-1(6),2,4,8-tetraen-3-yl)-methanone;
(8-phenyl-7-oxa-9-aza-bicyclo[4.3.0]nona-1(6),2,4,8-tetraen-3-yl)-(3,3,4-trimethyl-piperazin-1-yl)-methanone;
(4-methyl-4,7-diaza-spiro[2.5]octan-7-yl)-(8-phenyl-7-oxa-9-aza-bicyclo[4.3.0]nona-1(6),2,4,8-tetraen-3-yl)-methanone;
(8-(3-fluoro-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-fluoro-phenyl)-7-oxa-9-aza-bicyclo[4.3.0]nona-1(6),2,4,8-tetraen-3-yl)-(3,3,4-trimethyl-piperazin-1-yl)-methanone;
(8-(3-fluoro-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(4-bromo-phenyl)-7-oxa-9-aza-bicyclo[4.3.0]nona-1(6),2,4,8-tetraen-3-yl)-(3-isopropyl-4-methyl-piperazin-1-yl)-methanone;
(8-(4-bromo-phenyl)-7-oxa-9-aza-bicyclo[4.3.0]nona-1(6),2,4,8-tetraen-3-yl)-(4-methyl-piperazin-1-yl)-methanone;
(8-(4-bromo-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(4-bromo-phenyl)-7-oxa-9-aza-bicyclo[4.3.0]nona-1(6),2,4,8-tetraen-3-yl)-(3,3,4-trimethyl-piperazin-1-yl)-methanone;
(8-(4-bromo-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3-chloro-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3-chloro-phenyl)-7-oxa-9-aza-bicyclo[4.3.0]nona-1(6),2,4,8-tetraen-3-yl)-(4-methyl-piperazin-1-yl)-methanone;
(8-(3-chloro-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-chloro-phenyl)-7-oxa-9-aza-bicyclo[4.3.0]nona-1(6),2,4,8-tetraen-3-yl)-(3,3,4-trimethyl-piperazin-1-yl)-methanone;
(8-(3-chloro-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(3-isopropyl-4-methyl-piperazin-1-yl)-(8-m-tolyl-7-oxa-9-aza-bicyclo[4.3.0]nona-1(6),2,4,8-tetraen-3-yl)-methanone;
(4-methyl-piperazin-1-yl)-(8-m-tolyl-7-oxa-9-aza-bicyclo[4.3.0]nona-1(6),2,4,8-tetraen-3-yl)-methanone;
(4-methyl-3-(trifluoro-methyl)-piperazin-1-yl)-(8-m-tolyl-7-oxa-9-aza-bicyclo[4.3.0]nona-1(6),2,4,8-tetraen-3-yl)-methanone;
(8-m-tolyl-7-oxa-9-aza-bicyclo[4.3.0]nona-1(6),2,4,8-tetraen-3-yl)-(3,3,4-trimethyl-piperazin-1-yl)-methanone;
(4-methyl-4,7-diaza-spiro[2.5]octan-7-yl)-(8-m-tolyl-7-oxa-9-aza-bicyclo[4.3.0]nona-1(6),2,4,8-tetraen-3-yl)-methanone;
(3-isopropyl-4-methyl-piperazin-1-yl)-(8-(3-methoxy-phenyl)-7-oxa-9-aza-bicyclo[4.3.0]nona-1(6),2,4,8-tetraen-3-yl)-methanone;
(8-(3-methoxy-phenyl)-7-oxa-9-aza-bicyclo[4.3.0]nona-1(6),2,4,8-tetraen-3-yl)-(4-methyl-piperazin-1-yl)-methanone;
(8-(3-methoxy-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3-methoxy-phenyl)-7-oxa-9-aza-bicyclo[4.3.0]nona-1(6),2,4,8-tetraen-3-yl)-(3,3,4-trimethyl-piperazin-1-yl)-methanone;
(8-(3-methoxy-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3,5-dimethoxy-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3,5-dimethoxy-phenyl)-7-oxa-9-aza-bicyclo[4.3.0]nona-1(6),2,4,8-tetraen-3-yl)-(3,3,4-trimethyl-piperazin-1-yl)-methanone;
(8-(3,5-dimethoxy-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
(8-(3,5-dimethyl-phenyl)-7-oxa-9-aza-bicyclo[4.3.0]nona-1(6),2,4,8-tetraen-3-yl)-(3-isopropyl-4-methyl-piperazin-1-yl)-methanone;
(8-(3,5-dimethyl-phenyl)-7-oxa-9-aza-bicyclo[4.3.0]nona-1(6),2,4,8-tetraen-3-yl)-(4-methyl-piperazin-1-yl)-methanone;
(8-(3,5-dimethyl-phenyl)-7-oxa-9-aza-bicyclo[4.3.0]nona-1(6),2,4,8-tetraen-3-yl)-(4-methyl-3-(trifluoro-methyl)-piperazin-1-yl)-methanone;
(8-(3,5-dimethyl-phenyl)-7-oxa-9-aza-bicyclo[4.3.0]nona-1(6),2,4,8-tetraen-3-yl)-(3,3,4-trimethyl-piperazin-1-yl)-methanone; and
(8-(3,5-dimethyl-phenyl)-7-oxa-9-aza-bicyclo[4.3.0]nona-1(6),2,4,8-tetraen-3-yl)-(4-methyl-4,7-diaza-spiro[2.5]octan-7-yl)-methanone;
or a pharmaceutically acceptable salt thereof.

38. The method according to any one of claims 11-37, wherein the cancer is selected from melanoma, colorectal, pancreatic, lung, non-small cell lung cancer, breast, kidney, thyroid, lymphoid, gastrointestinal, genitourinary tract cancer, Hodgkin lymphoma, colon, renal cell carcinoma, ovarian, prostate cancer and/or testicular tumors, small intestine, and esophagus cancer.

39. The method according to any one of claims 8 to 38, further comprising administering one or more additional treatment modalities.

40. The method according to claim 39, wherein the additional treatment modality is selected from chemotherapy and immunotherapy.

41. The method according to claim 40, comprising administering a checkpoint inhibitor.

42. The method according to claim 41, wherein the checkpoint inhibitor is selected from an anti-PD-1 antibody, an anti-CD40 antibody, a CTLA-4 antibody, an anti-Tim3 antibody, and an anti-Lag3 antibody.

43. The method according to any one of claims 39-42, wherein the additional treatment modality is administered prior to, after, or concurrently with administration of the compound.
